(19) United States
(12) Patent Application Publication
(10) Pub. No.: US 2026/0308616 A1
(43) Pub. Date: Oct. 8, 2026

(54) PURIFICATION OF VIRUSES AND VIRAL LIKE PARTICLES

(71) Applicant: Interius Biotherapeutics, Inc., Philadelphia, PA (US)

(72) Inventors: PHILIP R. JOHNSON, Philadelphia, PA (US); Bruce C. Schnepp, Philadelphia, PA (US); Ronnie M. Russell, Philadelphia, PA (US)

(21) Appl. No.: 19/476,495

(22) PCT Filed: Apr. 18, 2024
(86) PCT No.: PCT/US2024/025166

§ 371 Date: Oct. 17, 2025

Related U.S. Application Data
(60) Provisional application No. 63/497,100, filed on Apr. 19, 2023.

(51) Int. Cl. C12N 15/86; C07K 14/005; C07K 16/28

(52) U.S. Cl. CPC C12N 15/86; C07K 14/005; C07K 16/2803; C07K 2317/53; C07K 2317/622; C07K 2317/92; C12N 2740/15043; C12N 2740/15052; C12N 2760/14122; C12N 2760/20222

(57) ABSTRACT

Provided for herein are pseudotyped viral constructs comprising affinity binding polypeptides fused with glycoproteins, targeting moieties, or a combination thereof. Also provided for herein are methods of purifying and using the viral constructs provided for herein.

### RELATED APPLICATIONS

This application claims the benefit of U.S. Provisional Application Ser. No. 63/497,100 filed Apr. 19, 2023, which is hereby incorporated by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. Said XML copy, created Apr. 16, 2024, is named “INH-015WO_SL.xml” and is 243,632 bytes in size.

### FIELD

Embodiments provided herein relate to the purification of viral vectors.

### BACKGROUND

Lentiviruses are common vectors used in gene therapy because they can transduce non-dividing cells and offer stable integration into a target cell's genome. The host range of lentivirus vectors can be altered by pseudotyping with glycoproteins derived from other viruses. Such pseudotyped lentiviral vectors then exhibit a receptor phenotype similar to the virus from which the envelope protein was derived. Depending on the host range of said virus, the pseudotyped retroviral vectors will then have a broadened or narrowed host range as compared to vector particles having the incorporated homologous retroviral envelope proteins. Current viral purification schemes can include high speed centrifugation and density gradient purification schemes, such as a sucrose gradient, that may not be easily scalable or easily adapted for Good Manufacturing Practice (GMP) protocols. Therefore, there is still a need to overcome the shortcomings of current viral purification schemes. The embodiments provided herein fulfill these needs as well as others.

### BRIEF SUMMARY

In some embodiments, a viral-like particle or viral vector is provided. In some embodiments, the viral-like particle or viral vector comprises a recombinant virus glycoprotein, a targeting moiety for binding to a target cell, and at least a first affinity binding polypeptide, wherein the affinity binding polypeptide is fused to the glycoprotein, the targeting moiety, or any combination thereof; and a nucleic acid molecule encoding a heterologous molecule of interest. In some embodiments, the affinity binding polypeptide is fused to the glycoprotein.

In some embodiments, the affinity binding polypeptide is fused to the targeting moiety. In some embodiments, the at least a first affinity binding polypeptide is an affinity tag selected from the group consisting of a polyhistidine tag, a polyarginine tag, a FLAG tag, a streptavidin tag, a calmodulin binding peptide, or a variant or combination thereof.

In some embodiments, the streptavidin tag is selected from the group consisting of a streptavidin-binding peptide, a streptavidin binding tag, a strep-tag II, a twin-strep tag, or a variant or combination there.

In some embodiments, the at least a first affinity binding polypeptide is fused to the glycoprotein. In some embodiments, the at least a first affinity binding polypeptide is located at the N-terminus, at the C-terminus, or within the glycoprotein. In some embodiments, the glycoprotein is selected from group consisting of an Ebola Virus glycoprotein, a NiV-G protein, a NiV-F protein, a MeV-H protein, a MeV-F protein, a VSV-G protein, a SVCV-G protein, or any variant thereof.

In some embodiments, the targeting moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanfitin, or any combination thereof. In some embodiments, the targeting moiety is fused to the viral glycoprotein. In some embodiments, the targeting moiety is not fused to the viral glycoprotein.

In some embodiments, the targeting moiety binds CD7. In some embodiments, the targeting moiety comprises an amino acid sequence having at least 85% similarity to SEQ ID NO: 101. In some embodiments, the targeting moiety comprises an amino acid sequence having at least 85% similarity to SEQ ID NO: 102.

In some embodiments, the targeting moiety binds CD8. In some embodiments, the targeting moiety comprises an amino acid sequence having at least 85% similarity to SEQ ID NO: 119. In some embodiments, the targeting moiety comprises an amino acid sequence having at least 85% similarity to SEQ ID NO: 120.

In some embodiments, the at least a first affinity binding polypeptide is fused to the targeting moiety. In some embodiments, the targeting moiety is as provided for herein. In some embodiments, the targeting moiety binds to CD7 as provided for herein. In some embodiments, the targeting moiety binds to CD8 as provided for herein.

In some embodiments, a pseudotyped viral-like particle or viral vector is provided. In some embodiments, the pseudotyped viral-like particle or viral vector comprises a recombinant viral glycoprotein, an engineered targeting moiety for binding to a target cell, and at least a first affinity binding polypeptide, wherein the at least a first affinity binding polypeptide is fused to the glycoprotein, and the glycoprotein comprises an amino acid sequence as set forth in SEQ ID NO: 25, SEQ ID NO: 29, SEQ ID NO: 30, SEQ ID NO: 33, SEQ ID NO: 34, SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 66, SEQ ID NO: 67, SEQ ID NO: 74, SEQ ID NO: 77, SEQ ID NO: 79, or SEQ ID NO: 80; and a nucleic acid encoding a heterologous molecule of interest.

In some embodiments, a pseudotyped viral-like particle or viral vector is provided. In some embodiments, the pseudotyped viral-like particle or viral vector comprises a recombinant viral glycoprotein, an engineered targeting moiety for binding to a target cell, and at least a first affinity binding polypeptide, wherein the at least a first affinity binding polypeptide is fused to the glycoprotein, and the at least a first affinity binding polypeptide fused to the glycoprotein comprises an amino acid sequence as set forth in SEQ ID NO: 26, SEQ ID NO: 27, SEQ ID NO: 28, SEQ ID NO: 31, SEQ ID NO: 32, SEQ ID NO: 35, SEQ ID NO: 68, SEQ ID NO: 69, SEQ ID NO: 70, SEQ ID NO: 71, SEQ ID NO: 72, SEQ ID NO: 73, SEQ ID NO: 75, SEQ ID NO: 76, SEQ ID NO: 78, SEQ ID NO: 81, SEQ ID NO: 132, SEQ ID NO: 133, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 166, SEQ ID NO: 167, SEQ ID NO: 168, SEQ ID NO: 170, SEQ ID NO: 172, SEQ ID NO: 188, SEQ ID NO: 189, SEQ ID NO: 190, or SEQ ID NO: 191; and a nucleic acid molecule encoding a heterologous molecule of interest.

In some embodiments, a pseudotyped viral-like particle or viral vector is provided comprising: an envelope comprising a recombinant virus glycoprotein, a targeting moiety for binding to a target cell, and at least a first affinity binding polypeptide, wherein the affinity binding polypeptide is fused to the glycoprotein, the targeting moiety, or any combination thereof; and a nucleic acid molecule encoding a heterologous molecule of interest, wherein the at least a first affinity binding polypeptide is fused to the targeting moiety. In some embodiments, the virus glycoprotein is derived from a virus from the group consisting of Human Immunodeficiency Virus (HIV), Simian Immunodeficiency Virus (SIV), Ebola virus (EbV), Nipah Virus (NiV), Measles virus (MeV), Vesicular stomatitis virus (VSV), Spring viremia of carp virus (SVCV) or a combination thereof. In some embodiments, the virus glycoprotein is HIV glycoprotein gp120, SIV glycoprotein gp120, EbV glycoprotein, NiV-G, NiV-F, MeV-H, MeV-F, VSV-G, SVCV-G, any variant thereof, or any combination thereof. In some embodiments, the viral-like particle is a retroviral-like particle or retroviral vector. In some embodiments, the retroviral-like particle is a lentivirus based viral particle or viral vector. In some embodiments, the at least a first affinity binding polypeptide is an affinity tag selected from the group consisting of a polyhistidine tag, a polyarginine tag, a FLAG tag, a streptavidin tag, a calmodulin binding peptide, or a variant or combination thereof. In some embodiments, the streptavidin tag is selected from the group consisting of a streptavidin-binding peptide, a streptavidin binding tag, a strep-tag II, a twin-strep tag, or a variant or combination thereof. In some embodiments, the at least a first affinity binding polypeptide is a strep-tag II polypeptide sequence.

In some embodiments, a pseudotyped viral-like particle or viral vector is provided. In some embodiments, the pseudotyped viral-like particle or viral vector comprises a recombinant viral glycoprotein, an engineered targeting moiety for binding to a target cell, and at least a first affinity binding polypeptide, wherein the targeting moiety is an scFv comprising an amino acid sequence of SEQ ID NO: 101, SEQ ID NO: 102, SEQ ID NO: 119, or SEQ ID NO: 120, and the at least a first affinity binding polypeptide is fused to the targeting moiety; and a nucleic acid molecule encoding a heterologous molecule of interest.

In some embodiments, a pseudotyped viral-like particle or viral vector is provided. In some embodiments, the pseudotyped viral-like particle or viral vector comprises a recombinant viral glycoprotein, an engineered targeting moiety for binding to a target cell, and at least a first affinity binding polypeptide, wherein the targeting moiety is an scFv, and wherein the at least a first affinity binding polypeptide is fused to the targeting moiety and comprises an amino acid sequence of SEQ ID NO: 121, SEQ ID NO: 126, SEQ ID NO: 129, SEQ ID NO: 130, SEQ ID NO: 131, SEQ ID NO: 174, SEQ ID NO: 175, SEQ ID NO: 176, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, or SEQ ID NO: 187; and a nucleic acid molecule encoding a heterologous molecule of interest.

In some embodiments, a pseudotyped viral-like particle or viral vector is provided. In some embodiments, the pseudotyped viral-like particle or viral vector comprises a recombinant virus glycoprotein, an engineered targeting moiety for binding to a target cell, and at least two affinity binding polypeptides, wherein the recombinant virus glycoprotein is fused to a first affinity binding polypeptide and comprises an amino acid sequence of SEQ ID NO: 26, SEQ ID NO: 27, SEQ ID NO: 28, SEQ ID NO: 31, SEQ ID NO: 32, SEQ ID NO: 35; SEQ ID NO: 68, SEQ ID NO: 69, SEQ ID NO: 70, SEQ ID NO: 71, SEQ ID NO: 72, SEQ ID NO: 73, SEQ ID NO: 75, SEQ ID NO: 76, SEQ ID NO: 78, SEQ ID NO: 81, SEQ ID NO: 132, SEQ ID NO: 133, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 166, SEQ ID NO: 167, SEQ ID NO: 168, SEQ ID NO: 170, SEQ ID NO: 172, SEQ ID NO: 188, SEQ ID NO: 189, SEQ ID NO: 190, or SEQ ID NO: 191; wherein the targeting moiety is an scFv; wherein the targeting moiety is fused to a second affinity binding polypeptide and comprises an amino acid sequence of SEQ ID NO: 121, SEQ ID NO: 126, SEQ ID NO: 129, SEQ ID NO: 130, SEQ ID NO: 131, SEQ ID NO: 174, SEQ ID NO: 175, SEQ ID NO: 176, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, or SEQ ID NO: 187; and a nucleic acid encoding a heterologous molecule of interest.

In some embodiments, a polypeptide molecule is provided, the molecule comprising a virus glycoprotein, wherein the glycoprotein further comprises at least a first affinity binding polypeptide sequence.

In some embodiments, a polypeptide molecule is provided, the molecule comprising a targeting moiety, wherein the targeting moiety further comprises at least a first affinity binding polypeptide sequence.

In some embodiments, a nucleic acid molecule is provided. In some embodiments, the nucleic acid molecule encodes for a polypeptide as provided for herein.

In some embodiments, a method of making a pseudotyped viral-like particle or viral vector as provided for herein is provided. In some embodiments, the method comprises transfecting or transducing a packaging cell line with a nucleic acid molecule as provided for herein under conditions sufficient to produce the viral like particle or viral vector.

In some embodiments, a method of purifying a pseudotyped viral-like particle or viral vector is provided. In some embodiments, the method comprises transfecting or transducing a packaging cell line with a nucleic acid molecule encoding for a recombinant virus glycoprotein, an engineered targeting moiety for binding to a target cell, and at least a first affinity binding polypeptide, wherein the affinity binding polypeptide is fused to the glycoprotein, the targeting moiety, or any combination thereof; culturing the packaging cell line under conditions sufficient to produce the psedotyped viral-like particle or viral vector; isolating the pseudotyped viral like-particle or viral vector; and purifying the pseudotyped viral-like particle or viral vector via use of the affinity binding polypeptide included in the glycoprotein, the targeting moiety, or both. In some embodiments, the step of purifying the pseudotyped viral-like particle or viral vector comprises incubating the isolated pseudotyped viral-like particle or viral vector with a stationary phase comprising an interacting molecule which interacts with the affinity binding polypeptide or the glycoprotein, the targeting moiety, or both; washing the stationary phase with an appropriate wash buffer; and eluting the pseudotyped viral-like particle or viral vector, thereby purifying the psuedotyped viral-like particle or viral vector. In some embodiments, the pseudotyped viral-like particle or viral vector is as provided for herein.

### BRIEF DESCRIPTION OF THE FIGURES

FIG. 1 depicts a schematic of exemplary targeting moiety polypeptides comprising an affinity binding polypeptide at the N terminus (N term tag), within the linker connecting the VH and VL domains (scFv linker tag), or at the IgG hinge region (Hinge tag).

FIG. 2A and FIG. 2B illustrate the effect an affinity binding polypeptide on the infectivity of various lentiviral constructs. Affinity binding polypeptides were located within the glycoprotein or targeting moiety with or without a short linker sequence. FIG. 2A illustrates the transduction of a CAR20 transgene with the various constructs. FIG. 2B illustrates the particle:infectivity ratio (particle:TU) as compared to historical data of viral constructs lacking an affinity binding polypeptide.

FIG. 3 illustrates that biotin in the buffer does not affect viral titer.

FIG. 4A and FIG. 4B illustrate the result of purifying viral constructs harboring no affinity binding tag or an affinity binding polypeptide fused to a viral glycoprotein via a Strep-Tactin column. FIG. 4A illustrates the results as a function of titer/mL. FIG. 4B illustrates the eluate data as a function of % recovery as compared to input.

FIG. 5 illustrates the result of purifying viral constructs harboring no affinity binding tag, an affinity binding polypeptide fused to a viral glycoprotein, or an affinity binding polypeptide fused to a targeting moiety via a Strep-Tactin column.

FIG. 6 illustrates the result of purifying viral constructs harboring no affinity binding tag, an affinity binding polypeptide fused to a viral glycoprotein, or an affinity binding polypeptide fused to a targeting moiety via a streptavidin coated magnetic beads.

FIG. 7 illustrates the binding kinetics of several viral constructs as provided for herein. Binding kinetics determined via a label free device.

### DETAILED DESCRIPTION

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, ±5%, ±1%, or ±0.1% from the specified value, as such variations are appropriate to perform the disclosed methods.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. The term “antigen” can also refer to a molecule that an antibody or antibody-like molecule can bind to or is recognized by the antibody or antibody-like molecule.

The term “antibody molecule,” “antibody” or antigen binding domain, as that term is used herein, refers to a polypeptide, e.g., an immunoglobulin chain or fragment thereof, comprising at least one functional immunoglobulin variable domain sequence. An antibody molecule encompasses antibodies (e.g., full-length antibodies) and antibody fragments. In some embodiments, an antibody molecule comprises an antigen binding or functional fragment of a full-length antibody, or a full-length immunoglobulin chain. For example, a full-length antibody is an immunoglobulin (Ig) molecule (e.g., an IgG antibody) that is naturally occurring or formed by normal immunoglobulin gene fragment recombinatorial processes. In embodiments, an antibody molecule refers to an immunologically active, antigen binding portion of an immunoglobulin molecule, such as an antibody fragment. An antibody fragment, e.g., functional fragment, comprises a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues.

Exemplary antibody molecules include full-length antibodies and antibody fragments, e.g., dAb (domain antibody), single chain, Fab, Fab′, and F(ab′)2 fragments, and single chain variable fragments (scFvs).

The term “antibody molecule” also encompasses whole or antigen binding fragments of domain, or single domain, antibodies, which can also be referred to as “sdAb” or “VHH.” Domain antibodies comprise either VH or VL that can act as stand-alone, antibody fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. (“Sequence of proteins of immunological interest,” US Public Health Services, NIH Bethesda, MD, Publication No. 91, which is hereby incorporated by reference). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113. Domain antibodies are also described in WO2004041862 and WO2016065323, each of which is hereby incorporated by reference. The domain antibodies can be a targeting moiety as described herein.

Antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, or hexavalent), or with higher orders of specificity (e.g, tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material, such as a cell, derived from a subject to which it is later to be re-introduced into the same subject.

As used herein, the term “allogeneic” is meant to refer to material, such as a cell, derived from one subject that is later introduced into a different subject.

As used herein, the term “cargo” is meant to refer to any product that may be encoded by a nucleic acid molecule. As non-limiting examples, “cargo” may refer to an siRNA, an shRNA, a peptide, a polypeptide, a protein, a viral payload, a viral genome, or a combination thereof. In some embodiments, the polypeptide is a chimeric antigen receptor (“CAR”).

A “chimeric antigen receptor” or “CAR” as used herein refers to an antigen-binding domain that is fused to an intracellular signaling domain capable of activating or stimulating an immune cell. Most commonly, the CAR's extracellular binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). In various embodiments, this scFv is fused to a transmembrane domain and then to an intracellular signaling domain. However, the antigen binding domain can be any molecule that can bind to the to target on the cell. For example, the antigen binding domain of a CAR can be an antibody, a scFv antibody, an antigen binding domain, an ankyrin repeat (e.g. DARPIN), a VHH domain antibody, a nanobody, single domain antibody, a FN3 domain, or any combination thereof. In some embodiments, a CAR includes those that solely provide CD3(signals upon antigen binding. In some embodiments, the CAR includes those that provide both costimulation (e.g. CD28 or CD137) and activation (CD3ζ). In some embodiments, the CARs include those that provide multiple costimulation (e.g. CD28 and CD137) and activation (CD3.zeta.). In various embodiments, the CAR is selected to have high affinity or avidity for the antigen. In some embodiments, the CAR comprises the 4-1BB domain as well. These are merely illustrative in nature and are not limiting to the present embodiments and any chimeric antigen receptor can be delivered in conjunction with the viral particles and vectors provided for herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of” or “consists.”

As used herein, the term “contacting” means bringing together of two elements in an in vitro system or an in vivo system. For example, “contacting” a vector with a cell or with an individual or patient or cell includes the administration of the vector to an individual or patient, such as a human, as well as, for example, introducing a compound into a sample containing a cellular or purified preparation containing the cell.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. In some embodiments, the epitope is about 4-18 amino acids, about 5-16 amino acids, about 6-14 amino acids, about 7-12, or about 8-10 amino acids. One skilled in the art understands that generally the overall three-dimensional structure, rather than the specific linear sequence of the molecule, is the main criterion of antigenic specificity and, therefore, distinguishes one epitope from another.

Based on the present disclosure, a peptide can be an epitope. “Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide. As used herein, the phrase “ex vivo” in reference to a cell being transduced, transfected or transformed ex vivo, refers to a cell being transduced, transfected or transformed outside of the subject, that is with the cells being removed from the subject before such cells are transduced, transfected or transformed.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another. In some embodiments, the various domains or proteins provided for herein are linked or fused directly to one another or a linker sequences, such as a glycine/serine sequence link the two domains together.

“Identity” as used herein refers to the subunit sequence identity between two polymeric molecules such as between two nucleic acid or amino acid molecules, such as, between two polynucleotide or polypeptide molecules. When two amino acid sequences have the same residues at the same positions; e.g., if a position in each of two polypeptide molecules is occupied by an Arginine, then they are identical at that position. The identity or extent to which two amino acid or two nucleic acid sequences have the same residues at the same positions in an alignment is often expressed as a percentage. The identity between two amino acid or two nucleic acid sequences is a direct function of the number of matching or identical positions; e.g., if half of the positions in two sequences are identical, the two sequences are 50% identical; if 90% of the positions (e.g., 9 of 10), are matched or identical, the two amino acids sequences are 90% identical.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity can be measured/determined using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e³ and e¹⁰⁰ indicating a closely related sequence. In some embodiments, sequence identity is determined by using BLAST with the default settings.

To the extent embodiments provided for herein, includes composition comprising various proteins, these proteins may, in some instances, comprise amino acid sequences that have sequence identity to the amino acid sequences disclosed herein. Therefore, in certain embodiments, depending on the particular sequence, the degree of sequence identity is preferably greater than 50% (e.g. 60%, 70%, 75%, 80%, 85%, 90%, 91%, 92%, 93%, 94%, 95%, 96%, 97%, 98%, 99% or more) to the SEQ ID NOs disclosed herein. These proteins may include homologs, orthologues, allelic variants and functional mutants. Typically, 50% identity or more between two polypeptide sequences is considered to be an indication of functional equivalence. Identity between polypeptides is preferably determined by the Smith-Waterman homology search algorithm as implemented in the MPSRCH program (Oxford Molecular), using an affine gap search with parameters gap open penalty—12 and gap extension penalty=1.

These proteins may, compared to the disclosed proteins, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, Substitution of single amino acids within these families does not have a major effect on the biological activity. The proteins may have one or more (e.g. 1, 2, 3, 4, 5, 6, 7, 8, 9, 10, etc.) single amino acid deletions relative to the disclosed protein sequences. The proteins may also include one or more (e.g. 1, 2, 3, 4, 5, 6, 7, 8, 9, 10, etc.) insertions (e.g. each of 1, 2, 3, 4 or 5 amino acids) relative to the disclosed protein sequences.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family that is able to infect non-dividing cells. Non-limiting examples of lentiviruses are HIV, SIV, and FIV. Vectors or viral-like particles derived from lentiviruses can be used to transduce cells and deliver genes or other molecules and have them expressed in a cell either in vitro (ex-vivo) or in vivo.

By the term “modified” as used herein, is meant a changed state or structure of a molecule or cell as provided herein. Molecules may be modified in many ways, including chemically, structurally, and functionally, such as mutations, substitutions, insertions, or deletions (e.g. internal deletions truncations). Cells may be modified through the introduction of nucleic acids or the expression of heterologous proteins.

By the term “modulating,” as used herein, is meant mediating an increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, such as, a human.

As used herein, the following abbreviations for the commonly occurring nucleic acid bases are used: “A” refers to adenosine, “C” refers to cytosine, “G” refers to guanosine, “T” refers to thymidine, and “U” refers to uridine.

The “Nipah virus” (NiV) is member of the family Paramyxoviridae, genus Henipavirus. Nipah virus is an enveloped virus with negative-stranded polarity and a non-segmented RNA genome consisting of helical nucleocapsids. Two strains of Nipah virus include, but are not limited to, the Malaysian (MY) and the Bangladesh (BD) strains.

The “Measles virus” (MeV) is a member of the family Paramyxoviridae, genus Morbillivirus. Measles virus is a single-stranded, negative-sense, enveloped, non-segmented RNA virus. The two envelope glycoproteins of the viral surface are the humagglutinin (H) and membrane fusion (F) proteins. The H protein mediates receptor attachment and the F protein causes fusion of the viral envelope and cellular membrane.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “oligonucleotide” typically refers to short polynucleotides. It will be understood that when a nucleotide sequence is represented by a DNA sequence (i.e., A, T, C, G), this also provides the corresponding RNA sequence (i.e., A, U, C, G) in which “U” replaces “T.”

“Parenteral” administration of a composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, the terms “nucleic acids” and “polynucleotides” as used herein are interchangeable. As used herein polynucleotides include, but are not limited to, all nucleic acid sequences which are obtained by any methods available in the art, including, without limitation, recombinant methods, i.e., the cloning of nucleic acid sequences from a recombinant library or a cell genome, using cloning technology and PCR, and the like, and by synthetic means.

As used herein, the terms “peptide,” “polypeptide,” and “protein” are used interchangeably, and refer to a compound comprised of a plurality of amino acid residues covalently linked by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “pseudotyped” or “pseudotyped viral particle”, as used herein, refers to a viral particle bearing glycoproteins derived from other viruses having envelopes or a viral vector encoding envelope glycoproteins from a virus that is different from the parental virus. The host range of the vector particles can thus be expanded or altered depending on the type of cell surface receptor used by the glycoprotein. For example, a HIV lentiviral vector can have the HIV envelope glycoprotein be replaced with another viruses glycoprotein. For example, the envelope glycoprotein of the Nipah virus can be used. Therefore, in some embodiments, the viral particle is encoded by a lentivirus that encodes the Nipah viral envelope glycoprotein. In some embodiments, the Nipah viral envelope glycoprotein is glycoprotein F or as otherwise provided for herein. In some embodiments, the Nipah viral envelope glycoprotein is glycoprotein G. In some embodiments, the pseudotyped viral vector encodes both the Nipah viral glycoprotein F and glycoprotein G. In some embodiments, the pseudotyped viral particle expresses one or both of the Nipah viral glycoprotein F and glycoprotein G. Other embodiments of pseudotyping are also provided for herein and can be used.

In certain embodiments herein, pseudotyped viral constructs are provided wherein an affinity binding polypeptide as provided for herein is fused or linked to a viral glycoprotein, a targeting moiety, or a combination thereof, and the affinity binding polypeptide is located at the N-terminus, the C-terminus, or within the viral glycoprotein or targeting moiety. In the context of the present application, N-terminus is understood to mean the first amino acid residue of the glycoprotein or targeting moiety. Accordingly, in embodiments wherein the affinity binding polypeptide is located at the N-terminus, it is to be understood that the affinity binding polypeptide is fused or linked to the glycoprotein or targeting moiety such that the affinity binding polypeptide immediately precedes the first amino acid residue of the glycoprotein or targeting moiety. An affinity binding polypeptide that is located near the N-terminus such that it is between the 1^{st} and 2^{nd} amino acids, between the 2^{nd} and 3^{rd} amino acids, between the 3^{rd} and 4^{th} amino acids, etc., would not be considered to be located at the N-terminus, but rather would be considered to be within the glycoprotein or targeting moiety.

Similarly, in the context of the present application, C-terminus is understood to mean the last amino acid residue of the glycoprotein or targeting moiety. Accordingly, in embodiments wherein the affinity binding polypeptide is located at the C-terminus, it is to be understood that the affinity binding polypeptide is fused or linked to the glycoprotein or targeting moiety such that the affinity binding polypeptide is located after the last amino acid residue of the glycoprotein or targeting moiety. An affinity binding polypeptide located near the C-terminus such that it is between the last and next to last residue, the next to last and second to last residue, the second to last and third to last residue, etc., would not be considered to be located at the C-terminus, but rather would be considered to be within the glycoprotein or targeting moiety. Accordingly, and affinity binding polypeptide that is flanked its N and C termni by at least one amino acid residue of the glycoprotein or targeting moiety is considered to be within the glycoprotein or targeting moiety.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “transfected” or “transformed” or “transduced” as used herein refers to a process by which exogenous nucleic acid is transferred or introduced into a cell. A “transfected” or “transformed” or “transduced” cell is one which has been transfected, transformed or transduced with exogenous nucleic acid. The cell includes the primary subject cell and its progeny. In some embodiments, the transfection, transformation, or transduction is performed or occurs in vivo.

As used herein, the term “variant” when used in conjunction to an amino acid sequence refers to a sequence that is at least, or about, 85%, 90%, 91%, 92%, 93%, 94%, 95%, 96%, 97%, 98%, or 99% identical to the reference sequence. In some embodiments, the variant comprises 1, 2, 3, 4, 5, 6, 7, 8, 9, or 10 substitutions. In some embodiments, the substitution is a conservative substitution.

A “vector” is a composition of matter which comprises an isolated nucleic acid encoding a protein or a peptide. Numerous vectors are known in the art including, but not limited to, linear polynucleotides, plasmids, DNA, and RNA. Examples of viral vectors include, but are not limited to, Sendai viral vectors, adenoviral vectors, adeno-associated virus vectors, retroviral vectors, lentiviral vectors, and the like.

A “carrier” or “delivery vehicle” includes viral particles, viruses, polylysine compounds, and liposomes, which facilitate transfer of nucleic acid into cells. A carrier or delivery vehicle can also be used to deliver a protein or peptide to a cell.

Ranges: throughout this disclosure, various aspects of the embodiments can be presented in a range format. It should be understood that the description in range format is merely for convenience and brevity and should not be construed as an inflexible limitation.

Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range. Unless otherwise explicitly stated to the contrary, a range that is disclosed also includes the endpoints of the range.

Without being bound to any particular theory, the embodiments provided for herein have been found to simplify the purification of viral particles which allows for better scalability and increased production. Previous viral purification techniques utilized schemes that can include high speed centrifugation and density gradient purification, such as a sucrose gradient, that may not be easily scalable or easily adapted for Good Manufacturing Practice (GMP) protocols. By incorporating affinity purification tags into the glycoproteins, the targeting moieties, or a combination thereof as presented on the lentiviral envelope, virus can be purified via affinity stationary phases in a much less harsh and scalable manner. Further, the incorporation of affinity binding polypeptides into a universal targeting moiety stalk allows for flexibility in scFv selection, but also reproducibility and consistency in viral purifications.

### Viruses

In some embodiments, the virus particle is a lentiviral particle. Lentiviral particles are derived from lentiviruses, which are retroviruses that, in addition to the common retroviral genes gag, pol, and env, contain other genes with regulatory or structural function (see, e.g., U.S. Pat. Nos. 6,013,516 and 5,994,136). Some examples of lentiviruses include the Human Immunodeficiency Viruses (HIV-1, HIV-2) and the Simian Immunodeficiency Virus (SIV). Lentiviral particles have been generated by multiply attenuating the HIV virulence genes, for example, the genes env, vif vpr, vpu and nef are deleted making the vector biologically safe. Lentiviral particles are capable of infecting non-dividing cells and can be used for both in vivo and ex vivo gene transfer and expression, e.g., of a nucleic acid encoding a CAR (see, e.g., U.S. Pat. No. 5,994,136).

Retrovirus expression vectors are capable of integrating into the host genome, delivering a large amount of foreign genetic material, infecting a broad spectrum of species and cell types and being packaged in special cell lines. The retroviral particle is constructed by inserting a nucleic acid (e.g., a nucleic acid encoding a CAR) into the viral genome at certain locations to produce a virus that is replication defective. Though the retroviral particles are able to infect a broad variety of cell types, integration and stable expression of the nucleic acid cargo (e.g. CAR) requires the division of host cells.

### Engineered Viral Particles

In some embodiments, the virus, viral particles, viral like particles, or viral vectors have been engineered to expand or alter the host range of the viral particles. This is accomplished by pseudotyping the viral particles by adding an envelope glycoprotein from another virus or substituting the envelope glycoprotein of the present virus with another virus's glycoprotein. Accordingly, in some embodiments, a psuedotyped viral-like particle or viral vector is provided. In some embodiments, the recombinant viral-like particle or viral vector comprises an envelope comprising a recombinant virus glycoprotein, a targeting moiety for binding to a target cell, at least a first binding polypeptide, and a nucleic acid molecule encoding a polypeptide of interest. In some embodiments, the at least a first affinity binding polypeptide is fused to the glycoprotein, the targeting moiety, or any combination thereof. In some embodiments, the at least a first affinity binding polypeptide is fused to the glycoprotein. In some embodiments, the at least a first affinity binding polypeptide is fused to the targeting moiety. In some embodiments, the at least a first affinity binding polypeptide is fused to the glycoprotein and the targeting moiety.

The virus glycoprotein can be derived from any appropriate virus glycoprotein to achieve the desired host range of the viral particles. In some embodiments, the virus glycoprotein is derived from a virus from the group including, but not limited to, Human Immunodeficiency Virus (HIV), Simian Immunodeficiency Virus (SIV), Ebola virus (EbV), Nipah Virus (NiV), Measles Virus (MeV), Vesicular stomatitis virus (VSV), Spring viremia of carp virus (SVCV), or combination thereof. In some embodiments, the virus glycoprotein is derived from HIV. In some embodiments, the virus glycoprotein is derived from SIV. In some embodiments, the virus glycoprotein is derived from EbV. In some embodiments, the virus glycoprotein is derived from NiV. In some embodiments, the virus glycoprotein is derived from MeV. In some embodiments, the virus glycoprotein is derived from VSV. In some embodiments, the virus glycoprotein is derived from SVCV. In some embodiments, the virus glycoprotein is derived from any combination of HIV, SIV, EbV, NiV, MeV, VSV, and SVCV.

In some embodiments, the virus glycoprotein is selected from the group including, but not limited to, HIV glycoprotein gp120, SIV glycoprotein gp120, EbV glycoprotein, NiV attachment protein (NiV-G), NiV fusion protein (NiV-F), MeV attachment protein (MeV-H), MeV fusion protein (MeV-F), VSV glycoprotein (VSV-G), SVCV G protein (SVCV-G), or any variant thereof, or any combination thereof. In some embodiments, the virus glycoprotein is HIV glycoprotein gp120, or any variant thereof. In some embodiments, the virus glycoprotein is SIV glycoprotein gp120, or any variant thereof. In some embodiments, the virus glycoprotein is EbV glycoprotein or any variant thereof. In some embodiments, the virus glycoprotein is NiV-G, or any variant thereof. In some embodiments, the virus glycoprotein is NiV-F, or any variant thereof. In some embodiments, the virus glycoprotein is MeV-H, or any variant thereof. In some embodiments, the virus glycoprotein is MeV-F, or any variant thereof. In some embodiments, the virus glycoprotein is VSV-G, or any variant thereof. In some embodiments, the virus glycoprotein is SVCV-G, or any variant thereof. One of skill in the art will recognize that any of the above recited glycoproteins may contain conservative amino acid substitutions that do not drastically change the function or the properties of the glycoproteins. Such conservative amino acid substitutions are as provided for herein. Further, one of skill in the art will recognize that certain amino acid substitutions, repetitions, or deletions may enhance the function or the properties of the glycoproteins. Accordingly, in some embodiments, the virus glycoprotein is substantially similar to HIV glycoprotein gp120. In some embodiments, the virus glycoprotein is substantially similar to SIV glycoprotein gp120. In some embodiments, the virus glycoprotein is substantially similar to EbV glycoprotein. In some embodiments, the virus glycoprotein is substantially similar to NiV-G. In some embodiments, the virus glycoprotein is substantially similar to NiV-F. In some embodiments, the virus glycoprotein is substantially similar to MeV-H. In some embodiments, the virus glycoprotein is substantially similar to MeV-F. In some embodiments, the virus glycoprotein is substantially similar to VSV-G. In some embodiments, the virus glycoprotein is substantially similar to SVCV-G. In any embodiment, a substantially similar sequence is as provided for herein.

In some embodiments, the viral-like particle or viral vector is a retro-viral like particle or a retroviral vector. In some embodiments, the retro-viral like particle or retroviral vector is any retro-viral like particle or retroviral vector. In some embodiments, the retro-viral like particle or retroviral vector is selected from the group including, but not limited to, alpharetrovirus, betaretrovirus, deltaretrovirus, epsilonretrovirus, gammaretrovirus, and lentivirus. In some embodiment, the retroviral-like particle is a lentivirus based viral particle or viral vector.

In some embodiments, the at least a first affinity binding polypeptide is any polypeptide sequence that will bind to a binder. For example, the affinity binding polypeptide may be any peptide or protein and the binder may be an antibody that specifically binds to the binding polypeptide. For example, the affinity binding polypeptide may comprise of GFP and the binder may comprise of an anti-GFP antibody. In some embodiments, the affinity binding polypeptide may bind to a non-proteinaceous binder. For example, the affinity binding polypeptide may be a polyhistidine sequence and the binder may be a nickel resin. Accordingly, in some embodiments, the at least a first affinity binding polypeptide is an affinity tag selected from the group including, but not limited to a polyhistidine tag, a polyarginine tag, a FLAG tag, a streptavidin tag, a calmodulin binding peptide, or any variant thereof, or any combination thereof. In some embodiments, the at least a first affinity binding polypeptide is a polyhistidine tag. In some embodiments, the at least a first affinity binding polypeptide is a polyarginine tag. In some embodiments, the at least a first affinity binding polypeptide is a FLAG tag. In some embodiments, the at least a first affinity binding polypeptide is a streptavidin tag. In some embodiments, the at least a first affinity binding polypeptide is a calmodulin binding peptide. In some embodiments, the at least a first affinity binding polypeptide is any variation of a recited affinity binding polypeptide. Such variations may include one or more amino acid substitutions, deletions, or additions in order to alter the binding affinity of the at least a first affinity binding polypeptide for the corresponding binder. Any such known variation is within the scope of the present application.

In some embodiments, the at least a first affinity binding polypeptide is a streptavidin tag. In some embodiments, the streptavidin tag is selected from the group consisting of a streptavidin-binding peptide, a streptavidin binding tag, a strep-tag II, a twin-strep tag, or any variation thereof, or any combination thereof. In some embodiments, the streptavidin tag consists of a streptavidin-binding peptide. In some embodiments, the streptavidin tag consists of a streptavidin binding tag. In some embodiments, the streptavidin tag consists of a strep-tag II. In some embodiments, the streptavidin tag consists of a twin-strep tag. In some embodiments, the streptavidin tag comprises the formula of Formula I:

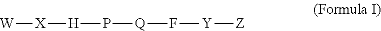

wherein X can be any amino acid and Y and Z can either both be G, or Y is E and Z is R or K. In some embodiments, X is A. In some embodiments, X is R. In some embodiments, X is N. In some embodiments, X is D. In some embodiments, X is C. In some embodiments, X is Q. In some embodiments, X is E. In some embodiments, X is G. In some embodiments, X is H. In some embodiments, X is I. In some embodiments, X is L. In some embodiments, X is K. In some embodiments, X is M. In some embodiments, X is F. In some embodiments, X is P. In some embodiments, X is S. In some embodiments, X is T. In some embodiments, X is W. In some embodiments, X is Y. In some embodiments, X is V. In some embodiments, Y is G and Z is G. In some embodiments, Y is E and Z is R. In some embodiments, Y is E and Z is K. In some embodiments, the streptavidin tag of Formula I is given by the sequence WSHPQFEK (SEQ ID NO: 1). In some embodiments, the sequence of strep-tag II is the sequence of SEQ ID NO: 1.

In some embodiments, the streptavidin tag comprises the formula of Formula II:

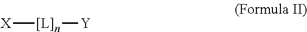

wherein X is a sequence of Formula I, Y is a sequence of Formula I, L is a linker peptide sequence, and n is an integer from 1 to 10. In some embodiments, X and Y comprise the same sequence. In some embodiments, X and Y comprises unique sequences of the formula of Formula I. In some embodiment, n is 1. In some embodiment, n is 2. In some embodiment, n is 3. In some embodiment, n is 4. In some embodiment, n is 5. In some embodiment, n is 6. In some embodiment, n is 7. In some embodiment, n is 8. In some embodiment, n is 9. In some embodiment, n is 10. It is to be understood that the portion of Formula II represented by [L]n is not meant to indicate that the linker L is to be repeated n times, but rather, when n is greater than 1, each instance of L may be the same or a unique peptide linker sequence. Thus, for example, in embodiments where n is 3, one could envisage the formula of Formula II to be written thusly:

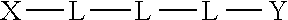

wherein each L, may comprise the same or a unique peptide linker sequence. For clarity, one could also envisage the formula of Formula II to be written thusly when n is 3:

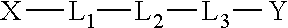

wherein each L₁, L₂, and L₃ may comprise the same or a unique peptide linker sequence. This clarifying representation of the formula of Formula II holds true for each value of n. In some embodiments, each L is the same peptide linker sequence. In some embodiments, each L is, individually, a unique linker peptide sequence. In some embodiments, the linker is a flexible peptide linker. In some embodiments, the linker is a non-cleavable peptide linker. In some embodiments the linker is a cleavable peptide linker. Non-limiting examples of linkers are provided in the following table:

TABLE 1

| Type | Sequence |
|---|---|
| Flexible | GGS |
| Flexible | GGGGS (SEQ ID NO: 2) |
| Flexible | (GGGGS)3 (SEQ ID NO: 3) |
| Flexible | (GGGGS)n (n=1, 2, 3, 4) (SEQ ID NO: 4) |
| Flexible | (Gly): (SEQ ID NO: 5) |
| Flexible | (Gly)6 (SEQ ID NO: 6) |
| Rigid | (EAAAK)3 (SEQ ID NO: 7) |
| Rigid | (EAAK)n (n=1-3) (SEQ ID NO: 8) |
| Rigid | A(EAAAK)4ALEA(EAAAK)4A (SEQ ID NO: 9) |
| Rigid | AEAAAKEAAAKA (SEQ ID NO: 10) |
| Rigid | PAPAP (SEQ ID NO: 11) |
| Rigid | (Ala-Pro), (10-34 aa) (SEQ ID NO: 12) |
| Cleavable | Disulfide |
| Cleavable | VSQTSKLTRAETVFPDV (SEQ ID NO: 13) |
| Cleavable | PLGLWA (SEQ ID NO: 14) |
| Cleavable | RVLAEA (SEQ ID NO: 15) |
| Cleavable | EDVVCCSMSY (SEQ ID NO: 16) |
| Cleavable | GGIEGRGS (SEQ ID NO: 17) |
| Cleavable | TRHRQPRGWE (SEQ ID NO: 18) |
| Cleavable | AGNRVRRSVG (SEQ ID NO: 19) |
| Cleavable | RRRRRRRRR (SEQ ID NO: 20) |
| Cleavable | GFLG (SEQ ID NO: 21) |
| Dipeptide | LE |
| Dipeptide | SA |

In some embodiments, the linker is given by the sequence GGGS (SEQ ID NO: 22). In some embodiments, the linker is given by the sequence GGSA (SEQ ID NO: 23). It is to be understood, for example, that when n is 3 or greater, the identity of the linker can be identical or unique between any two or more linkers. Thus, for example, when n is 3, L₁, L₂, and L₃ can all be the same; L₁, L₂, and L₃ can all be different; L₁ and L₂ can be the same while L₃ is different; L₁ and L₃ can be the same while L₂ is different; or L₂ and L₃ can be the same while L₁ is different. Such optionality likewise holds true for embodiments when n is 4 or greater. In some embodiments, X is given by SEQ ID NO 1, Y is given by Formula I, n is an integer between 1 and 10 and each L is, independently, a linker as provided for herein. In some embodiments, X is given by Formula I, Y is given by SEQ ID NO: 1, n is an integer between 1 and 10, and each L is, independently, a linker as provided for herein. In some embodiments, X is given by SEQ ID NO: 1, Y is given by SEQ ID NO: 1, n is 3, L₁ is given by SEQ ID NO: 22, L₂ is given by SEQ ID NO: 22, and L₃ is given by SEQ ID NO: 23. In some embodiments, the streptavidin tag of Formula II comprises the sequence WSHPQFEKGGGSGGGSGGSAWSHPQFEK (SEQ ID NO: 24). In some embodiments, the sequence of the twin-strep tag is the sequence of SEQ ID NO: 24.

In some embodiments, the at least a first affinity binding polypeptide is fused to the glycoprotein. The at least a first affinity binding polypeptide can be inserted at any position in the glycoprotein that does not substantially negatively affect the function or purpose of the glycoprotein. Accordingly, in some embodiments, the at least a first affinity binding polypeptide is located at the N-terminus, at the C-terminus, or within the glycoprotein. In some embodiments, the at least a first affinity binding polypeptide is located at the N-terminus of the glycoprotein. In some embodiments, the at least a first affinity binding polypeptide is located at the C-terminus of the glycoprotein. In some embodiments, the at least a first affinity binding polypeptide is located within the glycoprotein. In some embodiments, the at least a first affinity binding polypeptide is fused directly to the glycoprotein. In some embodiments, the at least a first affinity binding polypeptide is fused indirectly to the glycoprotein via, for example, a peptide linker as provided for herein. In embodiments where the at least a first affinity binding polypeptide is located within the glycoprotein, the affinity binding polypeptide may be fused to the glycoprotein directly, fused directly at the N-terminus of the affinity binding polypeptide and fused indirectly at the C-terminus of the affinity binding polypeptide via, for example, a peptide linker as provided for herein, fused indirectly at the N-terminus of the affinity binding polypeptide via, for example, a peptide linker as provided for herein and fused directly at the C-terminus of the affinity binding polypeptide, or fused indirectly at both the N and C terminus of the affinity binding polypeptide via, for example, a polypeptide linker as provided for herein.

In some embodiments, the pseudotyped viral-like particle or viral vector comprises at least a second affinity binding polypeptide fused to the glycoprotein. The at least a second affinity binding polypeptide can be inserted at any position in the glycoprotein that does not affect the function or purpose of the glycoprotein. Further, the position of the at least a second affinity binding polypeptide is independent of the position of the at least a first affinity binding polypeptide, so long as the combination of first and second affinity binding polypeptide does not negatively affect the function or purpose of the glycoprotein. Accordingly, in some embodiments, the at least a second affinity binding polypeptide is located at the N-terminus, at the C-terminus, or within the glycoprotein. In some embodiments, the at least a second affinity binding polypeptide is located at the N-terminus of the glycoprotein. In some embodiments, the at least a second affinity binding polypeptide is located at the C-terminus of the glycoprotein. In some embodiments, the at least a second affinity binding polypeptide is located within the glycoprotein. In some embodiments, the at least a second affinity binding polypeptide is fused directly to the glycoprotein. In some embodiments, the at least a second affinity binding polypeptide is fused indirectly to the glycoprotein via, for example, a peptide linker as provided for herein. In embodiments where the at least a second affinity binding polypeptide is located within the glycoprotein, the affinity binding polypeptide may be fused to the glycoprotein directly, fused directly at the N-terminus of the affinity binding polypeptide and fused indirectly at the C-terminus of the affinity binding polypeptide via, for example, a peptide linker as provided for herein, fused indirectly at the N-terminus of the affinity binding polypeptide via, for example, a peptide linker as provided for herein and fused directly at the C-terminus of the affinity binding polypeptide, or fused indirectly at both the N and C terminus of the affinity binding polypeptide via, for example, a polypeptide linker as provided for herein.

In some embodiments, the pseudotyped viral-like particle or viral vector comprises at least a third, at least a fourth, or at least a fifth affinity binding polypeptide fused to the glycoprotein. In some embodiments, the pseudotyped viral-like particle or viral vector comprises at least a third affinity binding polypeptide fused to the glycoprotein. In some embodiments, the position of the at least a third affinity binding polypeptide is as provided for herein for the at least a first and the at least a second affinity binding polypeptide. In some embodiments, the pseudotyped viral-like particle or viral vector comprises at least a fourth affinity binding polypeptide fused to the glycoprotein. In some embodiments, the position of the at least a fourth affinity binding polypeptide is as provided for herein for the at least a first and the at least a second affinity binding polypeptide. In some embodiments, the pseudotyped viral-like particle or viral vector comprises at least a fifth affinity binding polypeptide fused to the glycoprotein. In some embodiments, the position of the at least a fifth affinity binding polypeptide is as provided for herein for the at least a first and the at least a second affinity binding polypeptide. The number of affinity binding polypeptides fused to the glycoprotein is only limited in that the fusion constructs should not negatively affect the function or purpose of the glycoprotein. Thus, in some embodiments, the glycoprotein further comprises at least one affinity binding polypeptide. In some embodiments, the glycoprotein further comprises at least two affinity binding polypeptides. In some embodiments, the glycoprotein further comprises at least three affinity binding polypeptides. In some embodiments, the glycoprotein further comprises at least four affinity binding polypeptides. In some embodiments, the glycoprotein further comprises at least five affinity binding polypeptides. In some embodiments, the glycoprotein further comprises up to five affinity binding polypeptides. In some embodiments, the glycoprotein further comprises up to ten affinity binding polypeptides. In some embodiments, the glycoprotein further comprises up to twenty affinity binding polypeptides.

In some embodiments, affinity binding peptides as provided for herein are fused to Ebola glycoprotein. In some embodiments, the unmodified Ebola glycoprotein comprises a signal peptide, a receptor binding domain, a cathepsin cleavage loop, a glycan cap, a mucin-like domain (MLD), a furin cleavage site, a fusion loop, a heptad repeat 1 (HR1), a heptad repeat 2 (HR2), a transmembrane domain, and a cytoplasmic tail. (Lee J. E., et al., Structure of the Ebola virus glycoprotein bound to an antibody from a human survivor. Nature Vol 454, 177-183 (2008)) In some embodiments, the Ebola virus glycoprotein comprises an amino acid sequence having at least 70% identity to SEQ ID NO: 25:

(SEQ ID NO: 25)
MGVTGILQLPRDRFKRTSFFLWVIILFORTFSIPLGVIHNSTLQVSDVDK
LVCRDKLSSTNQLRSVGLNLEGNGVATDVPSATKRWGFRSGVPPKVVNYE
AGEWAENCYNLEIKKPDGSECLPAAPDGIRGEPRCRYVHKVSGTGPCAGD
FAFHKEGAFFLYDRLASTVIYRGTTFAEGVVAFLILPQAKKDFFSSHPLR
EPVNATEDPSSGYYSTTIRYQATGFGTNETEYLFEVDNLTYVQLESRFTP
QFLLQLNETIYISGKRSNTTGKLIWKVNPEIDTTIGEWAFWETKKNLTRK
IRSEELSFTVVSNGAKNISGQSPARTSSDPGINTTTEDHKIMASENSSAM
VQVHSQGREAAVSHLTTLATISTSPQSLTTKPGPDNSTHNTPVYKLDISE
ATQVEQHHRRTDNDSTASDTPSATTAAGPPKAENTNTSKSTDELDPATTT
SPQNHSETAGNNNTHHQDTGEESASSGKLGLITNTIAGVAGLITGGRRTR
REAIVNAQPKCNPNLHYWTTQDEGAAIGLAWIPYFGPAAEGIYIEGLMHN
QDGLICGLROLANETTQALQLFLRATTELRTFSILNRKAIDFLLORWGGT
CHILGPDCCIEPHDWTKNITDKIDQIIHDFVDKILPDQGDNDNWWTGWRQ
WIPAGIGVIGVIIAVIALFCICKFVF

or is substantially similar to SEQ ID NO: 25, or is an active fragment of SEQ ID NO: 25. In some embodiments, the Ebola virus glycoprotein comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 25. In some embodiments, the Ebola virus glycoprotein comprises an amino acid sequence of SEQ ID NO: 25.

In some embodiments, the Ebola virus glycoprotein comprises a sequence comprising a deletion, an insertion, a mutation, or any combination thereof as compared to SEQ ID NO: 25. In some embodiments, the deletion comprises an amino acid deletion of a glycan cap sequence, a mucin-like domain (MLD) sequence, or any combination thereof. In some embodiments, the deletion comprises an amino acid deletion from or between positions 213-306, 305-484, 213-484, 213-497, and 232-497, as compared to SEQ ID NO: 25. In some embodiments, the deletion comprises a deletion of the glycan cap amino acid sequence. In some embodiments, the deletion of the glycan cap sequence comprises a deletion of the amino acid residues from or between positions 213 and 306 as compared to SEQ ID NO: 25. In some embodiments, the deletion comprises a deletion of a portion of the glycan cap amino acid sequence. In some embodiments, the deletion of a portion of the glycan cap amino acid sequence comprises deletion of amino acid residues from or between positions 232 and 306 as compared to SEQ ID NO: 25. In some embodiments, the deletion comprises a deletion of the MLD amino acid sequence. In some embodiments, the deletion of the MLD comprises a deletion of amino acid residues from or between positions 305 and 497 as compared to SEQ ID NO: 25. In some embodiments, the deletion comprises a deletion of a portion of the MLD amino acid sequence. In some embodiments, the deletion of a portion of the MLD amino acid sequence comprises a deletion of amino acid residues from or between positions 305 and 484 as compared to SEQ ID NO: 25. In some embodiments, the deletion comprises a deletion of the glycan cap amino acid sequence and the MLD amino acid sequence. In some embodiments, the deletion of the glycan cap and the MLD comprises a deletion of amino acid residues from or between positions 213 and 484 as compared to SEQ ID NO: 25. In some embodiments, the deletion of the glycan cap and the MLD comprises a deletion of amino acid residues from or between positions 232 and 497 as compared to SEQ ID NO: 25. In some embodiments, the deletion comprises an amino acid deletion from or between positions 213-306, 305-484, 213-484, 213-497, and 232-497, as compared to SEQ ID NO: 25.

In some embodiments, the insertion comprises an insertion in place of the glycan cap amino acid sequence, a mucin-like domain (MLD) amino acid sequence, or any combination thereof. In some embodiments, the insertion comprises an insertion in place of the glycan cap amino acid sequence. In some embodiments, the insertion in place of the glycan cap amino acid sequence comprises an insertion from or between amino acid residues at positions 213 and 306 as compared to SEQ ID NO: 25. In some embodiments, the insertion in place of the glycan cap amino acid sequence comprises an insertion from or between amino acid residues at positions 232 and 306 as compared to SEQ ID NO: 25. In some embodiments, the insertion comprises an insertion in place of the MLD amino acid sequence. In some embodiments, the insertion in place of the MLD amino acid sequence comprises an insertion from or between amino acid residues at positions 305 and 497 as compared to SEQ ID NO: 25. In some embodiments, the insertion in place of the MLD amino acid sequence comprises an insertion from or between amino acid residues at positions 305 and 484 as compared to SEQ ID NO: 25. In some embodiments, the insertion comprises an insertion in place of the glycan cap amino acid sequence and the MLD amino acid sequence. In some embodiments, the insertion in place of the glycan cap amino acid sequence and the MLD amino acid sequence comprises an insertion from or between amino acid residues at positions 213 and 484 as compared to SEQ ID NO: 25. In some embodiments, the insertion in place of the glycan cap amino acid sequence and the MLD amino acid sequence comprises an insertion from or between amino acid residues at positions 213 and 497 as compared to SEQ ID NO: 25. In some embodiments, the insertion in place of the glycan cap amino acid sequence and the MLD amino acid sequence comprises an insertion from or between amino acid residues at positions 232 and 497 as compared to SEQ ID NO: 25.

In some embodiments, the mutation comprises an insertion of the targeting moiety amino acid sequence in place of the glycan cap amino acid sequence, a mucin-like domain (MLD) amino acid sequence, or any combination thereof. In some embodiments, the mutation comprises an insertion of the targeting moiety amino acid sequence in place of the glycan cap amino acid sequence. In some embodiments, the mutation comprises an insertion of a targeting moiety amino acid sequence in place of the amino acid sequence from or between positions 213-306 as compared to SEQ ID NO: 25; an insertion of a targeting moiety amino acid sequence in place of the amino acid sequence from or between positions 305-484 as compared to SEQ ID NO: 25; an insertion of a targeting moiety amino acid sequence in place of the amino acid sequence from or between positions 213-484 as compared to SEQ ID NO: 25; an insertion of a targeting moiety amino acid sequence in place of the amino acid sequence from or between positions 213-497 as compared to SEQ ID NO: 25; or an insertion of a targeting moiety amino acid sequence in place of the amino acid sequence from or between positions 232-497 as compared to SEQ ID NO: 25. In some embodiments, the insertion of the targeting moiety amino acid sequence in place of the glycan cap amino acid sequence comprises an insertion from or between amino acid residues at positions 213 and 306 as compared to SEQ ID NO: 25. In some embodiments, the insertion of the targeting moiety amino acid sequence in place of the glycan cap amino acid sequence comprises an insertion from or between amino acid residues at positions 232 and 306 as compared to SEQ ID NO: 25. In some embodiments, the mutation comprises an insertion of the targeting moiety amino acid sequence in place of the MLD amino acid sequence. In some embodiments, the insertion of the targeting moiety amino acid sequence in place of the MLD amino acid sequence comprises an insertion from or between amino acid residues at positions 305 and 497 as compared to SEQ ID NO: 25. In some embodiments, the insertion of the targeting moiety amino acid sequence in place of the MLD amino acid sequence comprises an insertion from or between amino acid residues at positions 305 and 484 as compared to SEQ ID NO: 25. In some embodiments, the mutation comprises an insertion of the targeting moiety amino acid sequence in place of the glycan cap amino acid sequence and the MLD amino acid sequence. In some embodiments, the insertion of the targeting moiety amino acid sequence in place of the glycan cap amino acid sequence and the MLD amino acid sequence comprises an insertion from or between amino acid residues at positions 213 and 484 as compared to SEQ ID NO: 25. In some embodiments, the insertion of the targeting moiety amino acid sequence in place of the glycan cap amino acid sequence and the MLD amino acid sequence comprises an insertion from or between amino acid residues at positions 213 and 497 as compared to SEQ ID NO: 25. In some embodiments, the insertion of the targeting moiety amino acid sequence in place of the glycan cap amino acid sequence and the MLD amino acid sequence comprises an insertion from or between amino acid residues at positions 232 and 497 as compared to SEQ ID NO: 25. In some embodiments, the mutation comprises an amino acid insertion at any position from or between positions 213-306, 305-484, 213-484, 213-497, or 232-497, as compared to SEQ ID NO: 25.

In some embodiments, the affinity binding polypeptide is fused at the N-terminus of SEQ ID NO: 25, or a variant thereof as provided for herein. In some embodiments, the affinity binding polypeptide is fused at the C-terminus of SEQ ID NO: 25, or a variant thereof as provided for herein. In some embodiments, the one or more affinity binding polypeptides are fused at any location within or at the termini's of SEQ ID NO: 25, or any variant thereof as provided for herein, provided that the inclusion does not substantially negatively affect the purpose of the glycoprotein of SEQ ID NO: 25. For example, the one or more affinity binding polypeptides may be fused within the signal peptide, within the receptor binding domain, within the cathepsin cleavage loop, within the glycan cap, within the mucin-like domain, within the furin cleavage site, within the fusion loop, within heptad repeat 1, within heptad repeat 2, within the transmembrane domain, within the cytoplasmic tail, between any two adjoining regions, or any combination thereof. In some embodiments, the Ebola glycoprotein fused to the affinity binding polypeptide comprises an amino acid sequence having at least 70% identity to SEQ ID NO: 26:

(SEQ ID NO: 26)
MGVTGILQLPRDRFWSHPQFEKGGSKRTSFFLWVIILFORTFSIPL
GVIHNSTLQVSDVDKLVCRDKLSSTNQLRSVGLNLEGNGVATDVPS
ATKRWGFRSGVPPKVVNYEAGEWAENCYNLEIKKPDGSECLPAAPD
GIRGFPRCRYVHKVSGTGPCAGDFAFHKEGAFFLYDRLASTVIYRG
TTFAEGVVAFLILPQAKKDFFSSHPLREPVNATEDPSSGYYSTTIR
YQATGFGTNETEYLFEVDNLTYVQLESRFTPQFLLQLNETIYTSGK
RSNTTGKLIWKVNPEIDTTIGEWAFWETKKNLTRKIRSEELSFTVV
SNGAKNISGQSPARTSSDPGTNTTTEDHKIMASENSSAMVOVHSQG
REAAVSHLITLATISTSPQSLTTKPGPDNSTHNTPVYKLDISEATQ
VEQHHRRTDNDSTASDTPSATTAAGPPKAENTNTSKSTDFLDPATT
TSPQNHSETAGNNNTHHQDTGEESASSGKLGLITNTIAGVAGLITG
GRRTRREAIVNAQPKCNPNLHYWTTQDEGAAIGLAWIPYFGPAAEG
IYIEGLMHNODGLICGLROLANETTQALQLFLRATTELRTFSILNR
KAIDFLLQRWGGTCHILGPDCCIEPHDWTKNITDKIDQIIHDFVDK
TLPDQGDNDNWWTGWRQWIPAGIGVIGVIIAVIALFCICKFVF

or is substantially similar to SEQ ID NO: 26. (Italicized amino acids represent the Ebola glycoprotein signal peptide, bold and underlined sequence represents the affinity binding polypeptide, bolded sequence no underline represents linker peptide). The sequence of SEQ ID NO: 26 contains the Ebola glycoprotein signal peptide MGVTGILQLPRDRF (SEQ ID NO: 146). Upon processing, the signal peptide may be cleaved leaving the sequence of SEQ ID NO: 147 (See Table 6). Thus, while embodiments may refer to further modifications of, mutations of, or percent identity to SEQ ID NO: 26, it is to be understood that the same modifications, mutations, and limitations to percent identity also apply to SEQ ID NO: 147. The sequence of SEQ ID NO: 26 may further be modified to remove the linker sequence (GGS), to move the linker sequence (GSS) to be on the N-terminus of the affinity binding polypeptide, or to include the linker sequence (GGS) at both the N-terminus and C-terminus of the affinity binding polypeptide. Further, the sequence of SEQ ID NO: 26 may comprise any of the mutations, deletions, substitutions or insertions described for the Ebola virus glycoprotein as provided for herein. Accordingly, in some embodiments, the Ebola virus glycoprotein fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 26. In some embodiments, the Ebola virus glycoprotein fused to the affinity binding polypeptide comprises an amino acid sequence of SEQ ID NO: 26.

In some embodiments, the Ebola glycoprotein fused to the affinity binding polypeptide comprises an amino acid sequence having at least 70% identity to SEQ ID NO: 27:

(SEQ ID NO: 27)
MGVTGILQLPRDREWSHPQFEKGGSKRISFFLWVIILFQRTFSIPL
GVIHNSTLQVSDVDKLVCRDKLSSTNOLRSVGLNLEGNGVATDVPS
ATKRWGFRSGVPPKVVNYEAGEWAENCYNLEIKKPDGSECLPAAPD
GIRGEPRCRYVHKVSGTGPCAGDFAFHKEGAFFLYDRLASTVIYRG
TTFAEGVVAFLILPQAKKDFFSSHPLREPVNATEDPSSGYYSTTIR
YQATGFGTNETEYLFEVDNLTYVQLESRFTPQFLLQLNETIYTSGK
RSNTTGKLIWKVNPEIDTTIGEWAFWETKKNLTRKIRSEESANTIA
GVAGLITGGRRIRREAIVNAQPKCNPNLHYWTTQDEGAAIGLAWIP
YFGPAAEGIYIEGLMHNQDGLICGLROLANETTQALQLFLRATTEL
RTFSILNRKAIDFLLQRWGGTCHILGPDCCIEPHDWTKNITDKIDQ
IIHDFVDKTLPDQGDNDNWWTGWRQWIPAGIGVTGVIIAVIALFCI
CKFVF

or is substantially similar to SEQ ID NO: 27. (Italicized amino acids represent the Ebola glycoprotein signal peptide, bold and underlined sequence represents the affinity binding polypeptide, bolded sequence no underline represents linker peptide). The sequence of SEQ ID NO: 27 contains the Ebola glycoprotein signal peptide MGVTGILQLPRDRF (SEQ ID NO: 146). Upon processing, the signal peptide may be cleaved leaving the sequence of SEQ ID NO: 148 (See Table 6). Thus, while embodiments may refer to further modifications of, mutations of, or percent identity to SEQ ID NO: 27, it is to be understood that the same modifications, mutations, and limitations to percent identity also apply to SEQ ID NO: 148. The sequence of SEQ ID NO: 27 may further be modified to remove the linker sequence (GGS), to move the linker sequence (GSS) to be on the N-terminus of the affinity binding polypeptide, or to include the linker sequence (GGS) at both the N-terminus and C-terminus of the affinity binding polypeptide. Further, the sequence of SEQ ID NO: 27 may comprise any of the mutations, deletions, substitutions or insertions described for the Ebola virus glycoprotein as provided for herein. Accordingly, in some embodiments, the Ebola virus glycoprotein fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 27. In some embodiments, the Ebola virus glycoprotein fused to the affinity binding polypeptide comprises an amino acid sequence of SEQ ID NO: 27.

In some embodiments, the affinity binding polypeptide is inserted into the Ebola virus glycoprotein in place of the glycan cap amino acid sequence, a mucin-like domain (MLD) amino acid sequence, or any combination thereof. In some embodiments, the affinity binding polypeptide replaces the amino acids between positions 213-306, 305-484, 213-484, 213-497, or 232-497, as compared to SEQ ID NO: 25. In some embodiments, at least one affinity binding polypeptide is inserted in place of the glycan cap amino acid sequence, a mucin-like domain (MLD) amino acid sequence, or any combination thereof. In some embodiments, the at least one affinity binding polypeptide replaces the amino acids between positions 213-306, 305-484, 213-484, 213-497, or 232-497, as compared to SEQ ID NO: 25. In some embodiments, at least one, at least two, at least three, at least four, at least five, or more than five affinity binding polypeptide is inserted in place of the glycan cap amino acid sequence, a mucin-like domain (MLD) amino acid sequence, or any combination thereof. In some embodiments, the at least one, at least two, at least three, at least four, at least five, or more than five affinity binding polypeptide replace the amino acids between positions 213-306, 305-484, 213-484, 213-497, or 232-497, as compared to SEQ ID NO: 25. In embodiments wherein more than one affinity binding polypeptide is inserted in place of the glycan cap amino acid sequence, a mucin-like domain (MLD) amino acid sequence, or any combination thereof, each affinity binding polypeptide may be, independently, the same or different than any previously inserted affinity binding polypeptide. In embodiments wherein more than one affinity binding polypeptide is inserted in place of the glycan cap amino acid sequence, a mucin-like domain (MLD) amino acid sequence, or any combination thereof, each affinity binding polypeptide may be fused directly to one other or indirectly fused to one other via, for example, a linker polypeptide as provided for herein.

In some embodiments, the affinity binding polypeptide is inserted into the Ebola virus glycoprotein in place of the MLD. In some embodiments, the MLD comprises amino acids 306-483 as compared to SEQ ID NO: 25. In some embodiments, the affinity binding polypeptide replaces the amino acids between positions 305-484 as compared to SEQ ID NO: 25. In some embodiments, the affinity binding polypeptide is inserted into the Ebola virus glycoprotein after residue E305 as compared to SEQ ID NO: 25. In some embodiments, the affinity binding polypeptide replaces the amino acids between residue E305 and N484 as compared to SEQ ID NO: 25. In some embodiments, the affinity binding polypeptide may further be flanked by one or more amino acids at the N-terminus, at the C-terminus, or at both the N-terminus and the C-terminus of the affinity binding polypeptide sequence inserted between residue E305 and N484 as compared to SEQ ID NO: 25. The one or more amino acids may be any amino acid. In some embodiments, the amino acid is S. In some embodiments, the amino acid is A. In some embodiments, the amino acid is T. In some embodiments, the amino acid is G. When more than one additional amino acid is added at the N-terminus, at the C-terminus, or at both the N-terminus and the C-terminus of the affinity binding polypeptide sequence inserted between residue E305 and N484 as compared to SEQ ID NO: 25, each amino acid may be, independently, any amino acid. When more than one additional amino acid is added at the N-terminus, at the C-terminus, or at both the N-terminus and the C-terminus of the affinity binding polypeptide sequence inserted between residue E305 and N484 as compared to SEQ ID NO: 25, each amino acid may be, independently, selected from the group comprising S, A, T, or G. In any of the embodiments provided herein, the one or more additional amino acids may be present or absent.

In some embodiments, the Ebola virus glycoprotein having the fused affinity binding polypeptide inserted in place of the MLD comprises an amino acid sequence having at least 70% identity to SEQ ID NO: 135:

(SQ ID NO: 135)
MGVTGILQLPRDRFKRTSFFLWVIILFQRTFSIPLGVIHNSTLQVS
DVDKLVCRDKLSSTNQLRSVGLNLEGNGVATDVPSATKRWGFRSGV
PPKVVNYEAGEWAENCYNLEIKKPDGSECLPAAPDGIRGFPRCRYV
HKVSGTGPCAGDFAFHKEGAFFLYDRLASTVIYRGITFAEGVVAFL
ILPQAKKDFFSSHPLREPVNATEDPSSGYYSTTIRYQATGFGTNET
EYLFEVDNLTYVQLESRFTPQFLLQLNETIYTSGKRSNTTGKLIWK
VNPEIDTTIGEWAFWETKKNLTRKIRSEESWSHPQFEKANTIAGVA
GLITGGRRTRREAIVNAQPKCNPNLHYWTTQDEGAAIGLAWIPYFG
PAAEGIYIEGLMHNQDGLICGLROLANETTQALQLFLRATTELRTF
SILNRKAIDELLORWGGTCHILGPDCCIEPHDWTKNITDKIDQIIH
DFVDKTLPDQGDNDNWWTGWRQWIPAGIGVTGVIIAVIALFCICKF
VF

or is substantially similar to SEQ ID NO: 135. (Italicized amino acids represent the Ebola glycoprotein signal peptide, bold and underlined sequence represents the affinity binding polypeptide). The sequence of SEQ ID NO: 135 contains the Ebola glycoprotein signal peptide MGVTGILQLPRDRF (SEQ ID NO: 146). Upon processing, the signal peptide may be cleaved leaving the sequence of SEQ ID NO: 149 (See Table 6). Thus, while embodiments may refer to further modifications of, mutations of, or percent identity to SEQ ID NO: 135, it is to be understood that the same modifications, mutations, and limitations to percent identity also apply to SEQ ID NO: 149. The sequence of SEQ ID NO: 135 may further be modified to insert a peptide linker sequence immediately upstream, immediately downstream, or both immediately upstream and immediately downstream of the affinity binding polypeptide sequence. The peptide linker may be any peptide linker as provided for herein. In some embodiments, the peptide linker is a flexible peptide linker as provided for herein. In some embodiments, the peptide linker is a glycine serine peptide linker as provided for herein. In some embodiments, the sequence of SEQ ID NO: 135 further comprises a deletion at position 306, 315, or a combination thereof as compared to SEQ ID NO: 135. In some embodiments, the deletion at position 306 comprises a S306 deletion. In some embodiments, the deletion at position 315 comprises a A315 deletion. In some embodiments, the Ebola virus glycoprotein having the fused affinity binding polypeptide inserted in place of the MLD further comprising a S306 and an A315 deletion comprises the amino acid sequence of SEQ ID NO: 152 (See Table 6). In some embodiments, the Ebola virus glycoprotein having the fused affinity binding polypeptide inserted in place of the MLD comprising a S306 and an A315 deletion as compared to SEQ ID NO: 135 and further comprising removal of the Ebola glycoprotein signal peptide comprises the amino acid sequence of SEQ ID NO: 153 (See Table 6). Further, the sequence of SEQ ID NO: 135 may comprise any of the mutations, deletions, substitutions or insertions described for the Ebola virus glycoprotein as provided for herein. Accordingly, in some embodiments, the Ebola virus glycoprotein having the fused affinity binding polypeptide inserted in place of the MLD comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 135. In some embodiments, the Ebola virus glycoprotein having the fused affinity binding polypeptide inserted in place of the MLD comprises an amino acid sequence of SEQ ID NO: 135.

In some embodiments, at least two affinity binding polypeptides are inserted into the Ebola virus glycoprotein in place of the MLD. In some embodiments, the MLD comprises amino acids 305-484 as compared to SEQ ID NO: 25. In some embodiments, the at least two affinity binding polypeptides replace the amino acids between positions 305-484 as compared to SEQ ID NO: 25. In some embodiments, the at least two affinity binding polypeptides are inserted into the Ebola virus glycoprotein after residue E305 as compared to SEQ ID NO: 25. In some embodiments, the at least two affinity binding polypeptides replace the amino acids between residue E305 and N484 as compared to SEQ ID NO: 25. The at least two affinity binding polypeptides may be the same or different. The at least to affinity binding polypeptides may be fused directly to one another or may be separated via a peptide linker as provided for herein. In some embodiments, the at least two affinity binding polypeptides may further be flanked by one or more amino acids at the N-terminus, at the C-terminus, or at both the N-terminus and the C-terminus of the at least two affinity binding polypeptide sequences inserted between residue E305 and N484 as compared to SEQ ID NO: 25. The one or more amino acids may be any amino acid. In some embodiments, the amino acid is S. In some embodiments, the amino acid is A. In some embodiments, the amino acid is T. In some embodiments, the amino acid is G. When more than one additional amino acid is added at the N-terminus, at the C-terminus, or at both the N-terminus and the C-terminus of the at least two affinity binding polypeptide sequences inserted between residue E305 and N484 as compared to SEQ ID NO: 25, each amino acid may be, independently, any amino acid. When more than one additional amino acid is added at the N-terminus, at the C-terminus, or at both the N-terminus and the C-terminus of the at least two affinity binding polypeptide sequences inserted between residue E305 and N484 as compared to SEQ ID NO: 25, each amino acid may be, independently, selected from the group comprising S, A, T, or G. In any of the embodiments provided herein, the one or more additional amino acids may be present or absent.

In some embodiments, the Ebola virus glycoprotein having the at least two fused affinity binding polypeptides inserted in place of the MLD comprises an amino acid sequence having at least 70% identity to SEQ ID NO: 136:

(SEQ ID NO: 136)
MGVTGILQLPRDRFKRTSFFLWVIILFQRTFSIPLGVIHNSTLQVSDVDK
LVCRDKLSSTNQLRSVGLNLEGNGVATDVPSATKRWGFRSGVPPKVVNYE
AGEWAENCYNLEIKKPDGSECLPAAPDGIRGFPRCRYVHKVSGTGPCAGD
FAFHKEGAFFLYDRLASTVIYRGTTFAEGVVAFLILPQAKKDFFSSHPLR
EPVNATEDPSSGYYSTTIRYQATGFGTNETEYLFEVDNLTYVQLESRFTP
QFLLQLNETIYTSGKRSNTTGKLIWKVNPEIDTTIGEWAFWETKKNLTRK
IRSEESWSHPQFEKKESGSVSSEQLAQFRSLDWSHPQFEKANTIAGVAGL
ITGGRRTRREAIVNAQPKCNPNLHYWITQDEGAAIGLAWIPYFGPAAEGI
YIEGLMHNQDGLICGLRQLANETTQALQLFLRATTELRIFSILNRKAIDF
LLQRWGGTCHILGPDCCIEPHDWTKNITDKIDQIIHDFVDKTLPDQGDND
NWWTGWRQWIPAGIGVTGVIIAVIALFCICKFVF

or is substantially similar to SEQ ID NO: 136. (Italicized amino acids represent the Ebola glycoprotein signal peptide, bold and underlined sequence represents the affinity binding polypeptide, bold no underline sequence represents the peptide linker). The sequence of SEQ ID NO: 136 contains the Ebola glycoprotein signal peptide MGVTGILQLPRDRF (SEQ ID NO: 146). Upon processing, the signal peptide may be cleaved leaving the sequence of SEQ ID NO: 150 (See Table 6). Thus, while embodiments may refer to further modifications of, mutations of, or percent identity to SEQ ID NO: 136, it is to be understood that the same modifications, mutations, and limitations to percent identity also apply to SEQ ID NO: 150. In some embodiments, the sequence of SEQ ID NO: 136 may further be modified to insert a peptide linker sequence immediately upstream of the first affinity binding polypeptide sequence. In some embodiments, the sequence of SEQ ID NO: 136 may further be modified to insert a peptide linker sequence immediately downstream of the second affinity binding polypeptide sequence. In some embodiments, the sequence of SEQ ID NO: 136 may further be modified to insert a peptide linker sequence immediately upstream of the first affinity binding polypeptide sequence and immediately downstream of the second affinity binding polypeptide sequence. In some embodiments, the peptide linker is a flexible peptide linker as provided for herein. In some embodiments, the peptide linker is a glycine serine peptide linker as provided for herein. In some embodiments, the sequence of SEQ ID NO: 136 further comprises a deletion at position 306, 341, or a combination thereof as compared to SEQ ID NO: 136. In some embodiments, the deletion at position 306 comprises a S306 deletion. In some embodiments, the deletion at position 341 comprises a A341 deletion. In some embodiments, the Ebola virus glycoprotein having the fused affinity binding polypeptide inserted in place of the MLD further comprising a S306 and an A341 deletion comprises the amino acid sequence of SEQ ID NO: 154 (See Table 6). In some embodiments, the Ebola virus glycoprotein having the fused affinity binding polypeptide inserted in place of the MLD comprising a S306 and an A341 deletion as compared to SEQ ID NO: 135 and further comprising removal of the Ebola glycoprotein signal peptide comprises the amino acid sequence of SEQ ID NO: 155 (See Table 6). Further, the sequence of SEQ ID NO: 135 may comprise any of the mutations, deletions, substitutions or insertions described for the Ebola virus glycoprotein as provided for herein. Accordingly, in some embodiments, the Ebola virus glycoprotein having the at least two fused affinity binding polypeptides inserted in place of the MLD comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 136. In some embodiments, the Ebola virus glycoprotein having the at least two fused affinity binding polypeptides inserted in place of the MLD comprises an amino acid sequence of SEQ ID NO: 136.

In some embodiments, the Ebola glycoprotein fused to the affinity binding polypeptide further comprises a mutation, wherein the mutation is an insertion of a targeting moiety as provided for herein. In some embodiments, the targeting moiety is as provided for herein. In some embodiments, the targeting moiety is inserted in place of the MLD as provided for herein. The targeting moiety may be flanked by one or more polypeptide linkers as provided for herein. In some embodiments, the polypeptide linkers are glycine serine polypeptide linkers as provided for herein. In some embodiments, the targeting moiety may further comprise additional dipeptides at the N-terminus, C-terminus, or both the N-terminus and C-terminus of the targeting moiety. In embodiments wherein polypeptide linkers are also present, additional dipeptides may also be present at the N-terminus, C-terminus, or both the N-terminus and C-terminus of the polypeptide linker. Examples of such dipeptides include, but are not limited to, SA, AS, and TG. In any of the embodiments provided herein, the dipeptides may be present or absent. In some embodiments, the affinity binding polypeptide is located at the N-terminus of the Ebola glycoprotein:targeting moiety fusion construct. In some embodiments, the affinity binding polypeptide is located at the C-terminus of the Ebola glycoprotein:targeting moiety fusion construct. In some embodiments, the affinity binding polypeptide is located between the Ebola glycoprotein and the targeting moiety. In some embodiments, the affinity binding polypeptide is located within the Ebola glycoprotein as provided for herein. In some embodiments, the affinity binding polypeptide is located within the targeting moiety as provided for herein.

In some embodiments, the Ebola glycoprotein fused to the affinity binding polypeptide further comprising a targeting moiety comprises an amino acid sequence having at least 70% identity to SEQ ID NO: 28:

(SEQ ID NO: 28)
MGVTGILQLPRDRFWSHPQFEKGGSKRTSFFLWVIILFQRTFSIPLGVIH
NSTLQVSDVDKLVCRDKLSSTNQLRSVGLNLEGNGVAIDVPSATKRWGER
SGVPPKVVNYEAGEWAENCYNLEIKKPDGSECLPAAPDGIRGFPRCRYVH
KVSGTGPCAGDFAFHKEGAFFLYDRLASTVIYRGTTFAEGVVAFLILPQA
KKDFFSSHPLREPVNATEDPSSGYYSTTIRYQATGFGTNETEYLFEVDNL
TYVQLESRFTPQFLLQLNETIYISGKRSNTTGKLIWKVNPEIDTTIGEWA
FWETKKNLTRKIRSEESAGGGGSGGGGSGGGGSASQVQLQQPGAELVKPG
ASVKLSCKASGYPFTSYWIHWVKQRPGRGLEWLGRIDPNSGDTKYNEKFK
NKATLTVDKSSTTAYMQLSSLTSEDSAVYYCARSPYYSNDNSMDYWGQGT
SVTVSSGGGGSGGGGSGGGGSGGGGSDILLTQSPAILSVSPGERVSFSCR
ASQSIGTSIHWYQQRINDSPRLLIKYASESISGIPSRFSGSGSGTDFTLS
INSVESEDIADYYCQQSNSWPTTFGGGTKLEIKRTGGGGGSGGGGSGGGG
SSANTIAGVAGLITGGRRTRREAIVNAQPKCNPNLHYWTTQDEGAAIGLA
WIPYFGPAAEGIYIEGLMHNQDGLICGLRQLANETTQALQLFLRATTELR
TFSILNRKAIDFLLQRWGGTCHILGPDCCIEPHDWTKNITDKIDQIIHDF
VDKTLPDQGDNDNWWTGWRQWIPAGIGVTGVIIAVIALFCICKFVF

or is substantially similar to SEQ ID NO: 28. (Italicized amino acids represent the Ebola glycoprotein signal peptide, bold and underlined sequence represents the affinity binding polypeptide, bolded sequence no underline represents linker peptide, underlined sequence represents targeting moiety). The sequence of SEQ ID NO: 28 contains the Ebola glycoprotein signal peptide MGVTGILQLPRDRF (SEQ ID NO: 146). Upon processing, the signal peptide may be cleaved leaving the sequence of SEQ ID NO: 151 (See Table 6). Thus, while embodiments may refer to further modifications of, mutations of, or percent identity to SEQ ID NO: 28, it is to be understood that the same modifications, mutations, and limitations to percent identity also apply to SEQ ID NO: 151. The sequence of SEQ ID NO: 28 may further be modified to remove the linker sequence (GGS), to move the linker sequence (GSS) to be on the N-terminus of the affinity binding polypeptide, or to include the linker sequence (GGS) at both the N-terminus and C-terminus of the affinity binding polypeptide. In some embodiments, the sequence of SEQ ID NO: 28 further comprises an amino acid deletion at position 317, 318, 334, 335, 535, 536, 552, 553, or a combination thereof as compared to SEQ ID NO: 28. In some embodiments, the deletion at position 317 comprises a S317 deletion. In some embodiments, the deletion at position 318 comprises a A318 deletion. In some embodiments, the deletion at position 334 comprises a A334 deletion. In some embodiments, the deletion at position 335 comprises a S335 deletion. In some embodiments, the deletion at position 535 comprises a T535 deletion. In some embodiments, the deletion at position 536 comprises a G536 deletion. In some embodiments, the deletion at position 552 comprises a S552 deletion. In some embodiments, the deletion at position 553 comprises a A553 deletion. In some embodiments, the Ebola glycoprotein fused to the affinity binding polypeptide further comprising a targeting moiety further comprising amino acid deletions at positions S317, A318, A334, S335, T535, G536, S552, and A553 as compared to SEQ ID NO: 28 comprises the amino acid sequence of SEQ ID NO: 137 (See Table 6). In some embodiments, the Ebola glycoprotein fused to the affinity binding polypeptide further comprising a targeting moiety comprising amino acid deletions at positions S317, A318, A334, S335, T535, G536, S552, and A553 as compared to SEQ ID NO: 28 and further comprising removal of the Ebola glycoprotein signal peptide comprises the amino acid sequence of SEQ ID NO: 156 (See Table 6). Further, the sequence of SEQ ID NO: 28 may comprise any of the mutations, deletions, substitutions or insertions described for the Ebola virus glycoprotein as provided for herein. Accordingly, in some embodiments, the Ebola virus glycoprotein fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 28. In some embodiments, the Ebola virus glycoprotein fused to the affinity binding polypeptide comprises an amino acid sequence of SEQ ID NO: 28.

In some embodiments, the Ebola glycoprotein fused to the affinity binding polypeptide further comprising a targeting moiety comprises an amino acid sequence having at least 70% identity to SEQ ID NO: 138:

(SEQ ID NO: 138)
MGVTGILQLPRDRFKRTSFFLWVIILFQRTFSIPLGVIHNSTLQVSDVDK
LVCRDKLSSTNQLRSVGLNLEGNGVATDVPSATKRWGFRSGVPPKVVNYE
AGEWAENCYNLEIKKPDGSECLPAAPDGIRGFPRCRYVHKVSGTGPCAGD
FAFHKEGAFFLYDRLASTVIYRGTTFAEGVVAFLILPQAKKDFFSSHPLR
EPVNATEDPSSGYYSTTIRYQATGFGTNETEYLFEVDNLTYVQLESRFTP
QFLLQLNETIYTSGKRSNTTGKLIWKVNPEIDTTIGEWAFWETKKNLTRK
IRSEESAGGGGSGGGGSGGGGSASQVQLQQPGAELVKPGASVKLSCKASG
YPFTSYWIHWVKQRPGRGLEWLGRIDPNSGDTKYNEKFKNKATLTVDKSS
TTAYMQLSSLTSEDSAVYYCARSPYYSNDNSMDYWGQGTSVTVSSGGGGS
GGGGSGGGGSGGGGSDILLTQSPAILSVSPGERVSFSCRASQSIGTSIHW
YQQRINDSPRLLIKYASESISGIPSRFSGSGSGTDFTLSINSVESEDIAD
YYCQQSNSWPTTFGGGTKLEIKRTGGGGGSGGGGSGGGGSWSHPQFEKKE
SGSVSSEQLAQFRSLDWSHPQFEKSANTIAGVAGLITGGRRTRREAIVNA
QPKCNPNLHYWTTQDEGAAIGLAWIPYFGPAAEGIYIEGLMHNQDGLICG
LRQLANETTQALQLFLRATTELRTFSILNRKAIDFLLQRWGGTCHILGPD
CCIEPHDWTKNITDKIDQIIHDFVDKTLPDQGDNDNWWTGWRQWIPAGIG
VTGVIIAVIALFCICKFVF

or is substantially similar to SEQ ID NO: 138. (Italicized amino acids represent the Ebola glycoprotein signal peptide, bold and underlined sequence represents the affinity binding polypeptide, bolded sequence no underline represents linker peptide, underlined sequence represents targeting moiety). The sequence of SEQ ID NO: 138 contains the Ebola glycoprotein signal peptide MGVTGILQLPRDRF (SEQ ID NO: 146). Upon processing, the signal peptide may be cleaved leaving the sequence of SEQ ID NO: 157 (See Table 6). Thus, while embodiments may refer to further modifications of, mutations of, or percent identity to SEQ ID NO: 138, it is to be understood that the same modifications, mutations, and limitations to percent identity also apply to SEQ ID NO: 157. The sequence of SEQ ID NO: 138 may further be modified to remove the polypeptide linker between the first and second affinity binding polypeptides, such that the affinity binding polypeptides are fused directly to one another. Alternatively, the sequence of SEQ ID NO: 138 may be modified such that the sequence within SEQ ID NO: 138 containing the first affinity binding polypeptide, the polypeptide linker, and the second affinity binding polypeptide is replaced with the sequence of SEQ ID NO: 24. The sequence of SEQ ID NO: 138 may also further be modified to insert a polypeptide linker sequence immediately downstream of the second affinity binding polypeptide sequence. In some embodiments, the peptide linker is a flexible peptide linker as provided for herein. In some embodiments, the peptide linker is a glycine serine peptide linker as provided for herein. In some embodiments, the sequence of SEQ ID NO: 138 further comprises an amino acid deletion at position 306, 307, 323, 324, 574, 575, 625, 626, or a combination thereof as compared to SEQ ID NO: 138. In some embodiments, the deletion at position 306 comprises a S306 deletion. In some embodiments, the deletion at position 307 comprises a A307 deletion. In some embodiments, the deletion at position 323 comprises a A323 deletion. In some embodiments, the deletion at position 324 comprises a S324 deletion. In some embodiments, the deletion at position 574 comprises a T574 deletion. In some embodiments, the deletion at position 575 comprises a G575 deletion. In some embodiments, the deletion at position 625 comprises a S625 deletion. In some embodiments, the deletion at position 626 comprises a A626 deletion. In some embodiments, the Ebola glycoprotein fused to the affinity binding polypeptide further comprising a targeting moiety further comprising amino acid deletions at positions S306, A307, A323, S324, T574, G575, S625, and A626 as compared to SEQ ID NO: 138 comprises the amino acid sequence of SEQ ID NO: 139 (See Table 6). In some embodiments, the Ebola glycoprotein fused to the affinity binding polypeptide further comprising a targeting moiety comprising amino acid deletions at positions S306, A307, A323, S324, T574, G575, S625, and A626 as compared to SEQ ID NO: 138 and further comprising removal of the Ebola glycoprotein signal peptide comprises the amino acid sequence of SEQ ID NO: 158 (See Table 6). Further, the sequence of SEQ ID NO: 138 may comprise any of the mutations, deletions, substitutions or insertions described for the Ebola virus glycoprotein as provided for herein. Accordingly, in some embodiments, the Ebola glycoprotein fused to the affinity binding polypeptide further comprising a targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 138. In some embodiments, the Ebola virus glycoprotein having the at least two fused affinity binding polypeptides inserted in place of the MLD comprises an amino acid sequence of SEQ ID NO: 138.

In some embodiments, the Ebola glycoprotein fused to the affinity binding polypeptide further comprising a targeting moiety comprises an amino acid sequence having at least 70% identity to SEQ ID NO: 140:

(SEQ ID NO: 140)
MGVTGILQLPRDRFKRTSFFLWVIILFQRTESIPLGVIHNSTLQVSDVDK
LVCRDKLSSTNQLRSVGLNLEGNGVATDVPSATKRWGFRSGVPPKVVNYE
AGEWAENCYNLEIKKPDGSECLPAAPDGIRGFPRCRYVHKVSGTGPCAGD
FAFHKEGAFFLYDRLASTVIYRGTTFAEGVVAFLILPQAKKDFFSSHPLR
EPVNATEDPSSGYYSTTIRYQATGFGTNETEYLFEVDNLTYVQLESRFTP
QFLLQLNETIYTSGKRSNTTGKLIWKVNPEIDTTIGEWAFWETKKNLTRK
IRSEESAGGGGSGGGGSGGGGSASQVQLQQPGAELVKPGASVKLSCKASG
YPFTSYWIHWVKQRPGRGLEWLGRIDPNSGDTKYNEKFKNKATLTVDKSS
TTAYMQLSSLTSEDSAVYYCARSPYYSNDNSMDYWGQGTSVTVSSGGGGS
GGGGSGGGGSGGGGSDILLTQSPAILSVSPGERVSFSCRASQSIGTSIHW
YQQRINDSPRLLIKYASESISGIPSRFSGSGSGTDFTLSINSVESEDIAD
YYCQQSNSWPTTFGGGTKLEIKRTGGGGGSGGGGSGGGGSWSHPQFEKSA
NTIAGVAGLITGGRRTRREAIVNAQPKCNPNLHYWTTQDEGAAIGLAWIP
YFGPAAEGIYIEGLMHNQDGLICGLRQLANETTQALQLFLRATTELRIFS
ILNRKAIDFLLQRWGGTCHILGPDCCIEPHDWTKNITDKIDQIIHDFVDK
ILPDQGDNDNWWIGWRQWIPAGIGVTGVIIAVIALFCICKFVF

or is substantially similar to SEQ ID NO: 140. (Italicized amino acids represent the Ebola glycoprotein signal peptide, bold and underlined sequence represents the affinity binding polypeptide, bolded sequence no underline represents linker peptide, underlined sequence represents targeting moiety). The sequence of SEQ ID NO: 140 contains the Ebola glycoprotein signal peptide MGVTGILQLPRDRF (SEQ ID NO: 146). Upon processing, the signal peptide may be cleaved leaving the sequence of SEQ ID NO: 159 (See Table 6). Thus, while embodiments may refer to further modifications of, mutations of, or percent identity to SEQ ID NO: 140, it is to be understood that the same modifications, mutations, and limitations to percent identity also apply to SEQ ID NO: 159. The sequence of SEQ ID NO: 140 may further be modified to insert a polypeptide linker sequence immediately upstream, immediately downstream, or both immediately upstream and immediately downstream of the affinity binding polypeptide sequence. In some embodiments, the peptide linker is a flexible peptide linker as provided for herein. In some embodiments, the peptide linker is a glycine serine peptide linker as provided for herein. In some embodiments, the sequence of SEQ ID NO: 140 further comprises an amino acid deletion at position 306, 307, 323, 324, 574, 575, 599, 600, or a combination thereof as compared to SEQ ID NO: 140. In some embodiments, the deletion at position 306 comprises a S306 deletion. In some embodiments, the deletion at position 307 comprises a A307 deletion. In some embodiments, the deletion at position 323 comprises a A323 deletion. In some embodiments, the deletion at position 324 comprises a S324 deletion. In some embodiments, the deletion at position 574 comprises a T574 deletion. In some embodiments, the deletion at position 575 comprises a G575 deletion. In some embodiments, the deletion at position 599 comprises a S599 deletion. In some embodiments, the deletion at position 600 comprises a A600 deletion. In some embodiments, the Ebola glycoprotein fused to the affinity binding polypeptide further comprising a targeting moiety further comprising amino acid deletions at positions S306, A307, A323, S324, T574, G575, S599, and A600 as compared to SEQ ID NO: 140 comprises the amino acid sequence of SEQ ID NO: 141 (See Table 6). In some embodiments, the Ebola glycoprotein fused to the affinity binding polypeptide further comprising a targeting moiety comprising amino acid deletions at positions S306, A307, A323, S324, T574, G575, S599, and A600 as compared to SEQ ID NO: 140 and further comprising removal of the Ebola glycoprotein signal peptide comprises the amino acid sequence of SEQ ID NO: 160 (See Table 6). Further, the sequence of SEQ ID NO: 140 may comprise any of the mutations, deletions, substitutions or insertions described for the Ebola virus glycoprotein as provided for herein. Accordingly, in some embodiments, the Ebola glycoprotein fused to the affinity binding polypeptide further comprising a targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 140. In some embodiments, the Ebola virus glycoprotein having the at least two fused affinity binding polypeptides inserted in place of the MLD comprises an amino acid sequence of SEQ ID NO: 140.

In some embodiments, the Ebola glycoprotein fused to the affinity binding polypeptide further comprising a targeting moiety comprises an amino acid sequence having at least 70% identity to SEQ ID NO: 142:

(SEQ ID NO: 142)
MGVIGILQLPRDRFKRTSFFLWVIILFQRTFSIPLGVIHNSTLQVSDVDK
LVCRDKLSSTNQLRSVGLNLEGNGVATDVPSATKRWGFRSGVPPKVVNYE
AGEWAENCYNLEIKKPDGSECLPAAPDGIRGFPRCRYVHKVSGTGPCAGD
FAFHKEGAFFLYDRLASTVIYRGTTFAEGVVAFLILPQAKKDFFSSHPLR
EPVNATEDPSSGYYSTTIRYQATGFGTNETEYLFEVDNLTYVQLESRFTP
QFLLQLNETIYTSGKRSNTTGKLIWKVNPEIDTTIGEWAFWETKKNLTRK
IRSEESAWSHPQFEKKESGSVSSEQLAQFRSLDWSHPQFEKGGGGSGGGG
SGGGGSASQVQLQQPGAELVKPGASVKLSCKASGYPFTSYWIHWVKQRPG
RGLEWLGRIDPNSGDTKYNEKFKNKATLTVDKSSTTAYMQLSSLTSEDSA
VYYCARSPYYSNDNSMDYWGQGTSVTVSSGGGGSGGGGSGGGGSGGGGSD
ILLTQSPAILSVSPGERVSFSCRASQSIGTSIHWYQQRINDSPRLLIKYA
SESISGIPSRFSGSGSGTDFTLSINSVESEDIADYYCQQSNSWPTTFGGG
TKLEIKRTGGGGGSGGGGSGGGGSSANTIAGVAGLITGGRRTRREAIVNA
QPKCNPNLHYWTTQDEGAAIGLAWIPYFGPAAEGIYIEGLMHNQDGLICG
LRQLANETTQALQLFLRATTELRTFSILNRKAIDFLLQRWGGTCHILGPD
CCIEPHDWTKNITDKIDQIIHDFVDKTLPDQGDNDNWWTGWRQWIPAGIG
VTGVIIAVIALFCICKEVE

or is substantially similar to SEQ ID NO: 142. (Italicized amino acids represent the Ebola glycoprotein signal peptide, bold and underlined sequence represents the affinity binding polypeptide, bold sequence no underline represents linker polypeptide, underlined sequence represents targeting moiety). The sequence of SEQ ID NO: 142 contains the Ebola glycoprotein signal peptide MGVTGILQLPRDRF (SEQ ID NO: 146). Upon processing, the signal peptide may be cleaved leaving the sequence of SEQ ID NO: 161 (See Table 6). Thus, while embodiments may refer to further modifications of, mutations of, or percent identity to SEQ ID NO: 142, it is to be understood that the same modifications, mutations, and limitations to percent identity also apply to SEQ ID NO: 161. The sequence of SEQ ID NO: 142 may further be modified to remove the polypeptide linker between the first and second affinity binding polypeptides, such that the affinity binding polypeptides are fused directly to one another. Alternatively, the sequence of SEQ ID NO: 142 may be modified such that the sequence within SEQ ID NO: 142 containing the first affinity binding polypeptide, the polypeptide linker, and the second affinity binding polypeptide is replaced with the sequence of SEQ ID NO: 24. The sequence of SEQ ID NO: 142 may also further be modified to insert a polypeptide linker sequence immediately upstream of the first affinity binding polypeptide, immediately downstream of the second affinity binding polypeptide, or both immediately upstream of the first affinity binding polypeptide and immediately downstream of the second affinity binding polypeptide. In some embodiments, the peptide linker is a flexible peptide linker as provided for herein. In some embodiments, the peptide linker is a glycine serine peptide linker as provided for herein. In some embodiments, the sequence of SEQ ID NO: 142 further comprises an amino acid deletion at position 306, 307, 357, 358, 608, 609, 625, 626, or a combination thereof as compared to SEQ ID NO: 142. In some embodiments, the deletion at position 306 comprises a S306 deletion. In some embodiments, the deletion at position 307 comprises a A307 deletion. In some embodiments, the deletion at position 357 comprises a A357 deletion. In some embodiments, the deletion at position 358 comprises a S358 deletion. In some embodiments, the deletion at position 608 comprises a T608 deletion. In some embodiments, the deletion at position 609 comprises a G609 deletion. In some embodiments, the deletion at position 625 comprises a S625 deletion. In some embodiments, the deletion at position 626 comprises a A626 deletion. In some embodiments, the Ebola glycoprotein fused to the affinity binding polypeptide further comprising a targeting moiety further comprising amino acid deletions at positions S306, A307, A357, S358, T608, G609, S625, and A626 as compared to SEQ ID NO: 142 comprises the amino acid sequence of SEQ ID NO: 143 (See Table 6). In some embodiments, the Ebola glycoprotein fused to the affinity binding polypeptide further comprising a targeting moiety comprising amino acid deletions at positions S306, A307, A357, S358, T608, G609, S625, and A626 as compared to SEQ ID NO: 142 and further comprising removal of the Ebola glycoprotein signal peptide comprises the amino acid sequence of SEQ ID NO: 162 (See Table 6). Further, the sequence of SEQ ID NO: 142 may comprise any of the mutations, deletions, substitutions or insertions described for the Ebola virus glycoprotein as provided for herein. Accordingly, in some embodiments, the Ebola glycoprotein fused to the affinity binding polypeptide further comprising a targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 142. In some embodiments, the Ebola virus glycoprotein having the at least two fused affinity binding polypeptides inserted in place of the MLD comprises an amino acid sequence of SEQ ID NO: 142.

In some embodiments, the Ebola glycoprotein fused to the affinity binding polypeptide further comprising a targeting moiety comprises an amino acid sequence having at least 70% identity to SEQ ID NO: 144:

(SEQ ID NO: 144)
MGVTGILQLPRDRFKRTSFFLWVIILFQRTFSIPLGVIHNSTLQVSDVDK
LVCRDKLSSTNQLRSVGLNLEGNGVATDVPSATKRWGFRSGVPPKVVNYE
AGEWAENCYNLEIKKPDGSECLPAAPDGIRGFPRCRYVHKVSGTGPCAGD
FAFHKEGAFFLYDRLASTVIYRGTTFAEGVVAFLILPQAKKDFFSSHPLR
EPVNATEDPSSGYYSTTIRYQATGFGTNETEYLFEVDNLTYVQLESRFTP
QFLLQLNETIYTSGKRSNTTGKLIWKVNPEIDTTIGEWAFWETKKNLTRK
IRSEESAWSHPQFEKGGGGSGGGGSGGGGSASQVQLQQPGAELVKPGASV
KLSCKASGYPFTSYWIHWVKQRPGRGLEWLGRIDPNSGDTKYNEKFKNKA
TLIVDKSSTTAYMQLSSLTSEDSAVYYCARSPYYSNDNSMDYWGQGTSVT
VSSGGGGSGGGGSGGGGSGGGGSDILLTQSPAILSVSPGERVSFSCRASQ
SIGTSIHWYQQRINDSPRLLIKYASESISGIPSRFSGSGSGTDFTLSINS
VESEDIADYYCQQSNSWPTTFGGGTKLEIKRTGGGGGSGGGGSGGGGSSA
NTIAGVAGLITGGRRTRREAIVNAQPKCNPNLHYWTTQDEGAAIGLAWIP
YFGPAAEGIYIEGLMHNQDGLICGLRQLANETTQALQLFLRATTELRIFS
ILNRKAIDFLLQRWGGTCHILGPDCCIEPHDWTKNITDKIDQIIHDFVDK
TLPDQGDNDNWWIGWRQWIPAGIGVTGVIIAVIALFCICKFVF

or is substantially similar to SEQ ID NO: 144. (Italicized amino acids represent the Ebola glycoprotein signal peptide, bold and underlined sequence represents the affinity binding polypeptide, bolded sequence no underline represents linker peptide, underlined sequence represents targeting moiety). The sequence of SEQ ID NO: 144 contains the Ebola glycoprotein signal peptide MGVTGILQLPRDRF (SEQ ID NO: 146). Upon processing, the signal peptide may be cleaved leaving the sequence of SEQ ID NO: 163 (See Table 6). Thus, while embodiments may refer to further modifications of, mutations of, or percent identity to SEQ ID NO: 144, it is to be understood that the same modifications, mutations, and limitations to percent identity also apply to SEQ ID NO: 163. The sequence of SEQ ID NO: 144 may further be modified to insert a polypeptide linker sequence immediately upstream, immediately downstream, or both immediately upstream and immediately downstream of the affinity binding polypeptide sequence. In some embodiments, the peptide linker is a flexible peptide linker as provided for herein. In some embodiments, the peptide linker is a glycine serine peptide linker as provided for herein. In some embodiments, the sequence of SEQ ID NO: 144 further comprises an amino acid deletion at position 306, 307, 331, 332, 582, 583, 599, 600, or a combination thereof as compared to SEQ ID NO: 144. In some embodiments, the deletion at position 306 comprises a S306 deletion. In some embodiments, the deletion at position 307 comprises a A307 deletion. In some embodiments, the deletion at position 331 comprises a A331 deletion. In some embodiments, the deletion at position 332 comprises a S332 deletion. In some embodiments, the deletion at position 582 comprises a T582 deletion. In some embodiments, the deletion at position 583 comprises a G583 deletion. In some embodiments, the deletion at position 599 comprises a S599 deletion. In some embodiments, the deletion at position 600 comprises a A600 deletion. In some embodiments, the Ebola glycoprotein fused to the affinity binding polypeptide further comprising a targeting moiety further comprising amino acid deletions at positions S306, A307, A331, S332, T582, G583, S599, and A600 as compared to SEQ ID NO: 144 comprises the amino acid sequence of SEQ ID NO: 145 (See Table 6). In some embodiments, the Ebola glycoprotein fused to the affinity binding polypeptide further comprising a targeting moiety comprising amino acid deletions at positions S306, A307, A331, S332, T582, G583, S599, and A600 as compared to SEQ ID NO: 144 and further comprising removal of the Ebola glycoprotein signal peptide comprises the amino acid sequence of SEQ ID NO: 164 (See Table 6). Further, the sequence of SEQ ID NO: 144 may comprise any of the mutations, deletions, substitutions or insertions described for the Ebola virus glycoprotein as provided for herein. Accordingly, in some embodiments, the Ebola glycoprotein fused to the affinity binding polypeptide further comprising a targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 144. In some embodiments, the Ebola virus glycoprotein having the at least two fused affinity binding polypeptides inserted in place of the MLD comprises an amino acid sequence of SEQ ID NO: 144.

In some embodiments, the Ebola glycoprotein fused to the affinity binding polypeptide further comprising a targeting moiety comprises an amino acid sequence having at least 70% identity to SEQ ID NO: 188:

(SEQ ID NO: 188)
MGVIGILQLPRDRFKRTSFFLWVIILFQRTFSIPLGVIHNSTLQVSDVDK
LVCRDKLSSTNQLRSVGLNLEGNGVATDVPSATKRWGFRSGVPPKVVNYE
AGEWAENCYNLEIKKPDGSECLPAAPDGIRGFPRCRYVHKVSGTGPCAGD
FAFHKEGAFFLYDRLASTVIYRGTTFAEGVVAFLILPQAKKDFFSSHPLR
EPVNATEDPSSGYYSTTIRYQATGFGTNETEYLFEVDNLTYVQLESRFTP
QFLLQLNETIYTSGKRSNTTGKLIWKVNPEIDTTIGEWAFWETKKNLTRK
IRSEESAWSHPQFEKGGGGSGGGGSGGGGSASQVQLQQPGAELVKPGASV
KLSCKASGYPFTSYWIHWVKQRPGRGLEWLGRIDPNSGDTKYNEKFKNKA
TLIVDKSSTTAYMQLSSLTSEDSAVYYCARSPYYSNDNSMDYWGQGTSVT
VSSGGGGSGGGGSGGGGSGGGGSDILLTQSPAILSVSPGERVSFSCRASQ
SIGTSIHWYQQRINDSPRLLIKYASESISGIPSRFSGSGSGTDFTLSINS
VESEDIADYYCQQSNSWPTTFGGGTKLEIKRTGGGGGSGGGGSGGGGSWS
HPQFEKSANTIAGVAGLITGGRRTRREAIVNAQPKCNPNLHYWTTQDEGA
AIGLAWIPYFGPAAEGIYIEGLMHNQDGLICGLRQLANETTQALQLFLRA
TTELRTFSILNRKAIDFLLQRWGGTCHILGPDCCIEPHDWTKNITDKIDQ
IIHDFVDKTLPDQGDNDNWWIGWRQWIPAGIGVTGVIIAVIALFCICKFVF

or is substantially similar to SEQ ID NO: 188. (Italicized amino acids represent the Ebola glycoprotein signal peptide, bold and underlined sequence represents the affinity binding polypeptide, bolded sequence no underline represents linker peptide, underlined sequence represents targeting moiety). sequence no underline represents linker peptide, underlined sequence represents targeting moiety). The sequence of SEQ ID NO: 188 contains the Ebola glycoprotein signal peptide MGVTGILQLPRDRF (SEQ ID NO: 146). Upon processing, the signal peptide may be cleaved leaving the sequence of SEQ ID NO: 189 (See Table 6). Thus, while embodiments may refer to further modifications of, mutations of, or percent identity to SEQ ID NO: 188, it is to be understood that the same modifications, mutations, and limitations to percent identity also apply to SEQ ID NO: 189. The sequence of SEQ ID NO: 188 may further be modified to insert a polypeptide linker sequence immediately upstream, immediately downstream, or both immediately upstream and immediately downstream of the affinity binding polypeptide sequence. In some embodiments, the peptide linker is a flexible peptide linker as provided for herein. In some embodiments, the peptide linker is a glycine serine peptide linker as provided for herein. In some embodiments, the sequence of SEQ ID NO: 188 further comprises an amino acid deletion at position 306, 307, 331, 332, 582, 583, 607, 608, or a combination thereof as compared to SEQ ID NO: 188. In some embodiments, the deletion at position 306 comprises a S306 deletion. In some embodiments, the deletion at position 307 comprises a A307 deletion. In some embodiments, the deletion at position 331 comprises a A331 deletion. In some embodiments, the deletion at position 332 comprises a S332 deletion. In some embodiments, the deletion at position 582 comprises a T582 deletion. In some embodiments, the deletion at position 583 comprises a G583 deletion. In some embodiments, the deletion at position 607 comprises a S607 deletion. In some embodiments, the deletion at position 608 comprises a A608 deletion. In some embodiments, the Ebola glycoprotein fused to the affinity binding polypeptide further comprising a targeting moiety further comprising amino acid deletions at positions S306, A307, A331, S332, T582, G583, S607, and A608 as compared to SEQ ID NO: 188 comprises the amino acid sequence of SEQ ID NO: 190 (See Table 6). In some embodiments, the Ebola glycoprotein fused to the affinity binding polypeptide further comprising a targeting moiety comprising amino acid deletions at positions S306, A307, A331, S332, T582, G583, S607, and A608 as compared to SEQ ID NO: 188 and further comprising removal of the Ebola glycoprotein signal peptide comprises the amino acid sequence of SEQ ID NO: 191 (See Table 6). Further, the sequence of SEQ ID NO: 188 may comprise any of the mutations, deletions, substitutions or insertions described for the Ebola virus glycoprotein as provided for herein. Accordingly, in some embodiments, the Ebola glycoprotein fused to the affinity binding polypeptide further comprising a targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 188. In some embodiments, the Ebola virus glycoprotein having the at least two fused affinity binding polypeptides inserted in place of the MLD comprises an amino acid sequence of SEQ ID NO: 188.

In some embodiments, affinity binding peptides as provided for herein are fused to NiV-G. In some embodiments, NiV-G comprises an amino acid sequence having at least 70% identity to SEQ ID NO 29:

(SEQ ID NO: 29)
MPTESKKVRFENTASDKGKNPSKVIKSYYGIMDIKKINEGLLDSKILSAF
NTVIALLGSIVIIVMNIMIIQNYTRSTDNQAMIKDALQSIQQQIKGLADK
IGTEIGPKVSLIDTSSTITIPANIGLLGSKISQSTASINENVNEKCKFTL
PPLKIHECNISCPNPLPFREYKPQTEGVSNLVGLPNNICLQKTSNQILKP
KLISYTLPVVGQSGTCITDPLLAMDEGYFAYSHLEKIGSCSRGVSKQRII
GVGEVLDRGDEVPSLFMTNVWTPSNPNTVYHCSAVYNNEFYYVLCAVSVV
GDPILNSTYWSGSLMMTRLAVKPKNNGESYNQHQFALRNIEKGKYDKVMP
YGPSGIKQGDTLYFPAVGFLVRIEFTYNDSNCPIAECQYSKPENCRLSMG
IRPNSHYILRSGLLKYNLSDEENSKIVFIEISDQRLSIGSPSKIYDSLGQ
PVFYQASFSWDIMIKFGDVQTVNPLVVNWRDNTVISRPGQSQCPRENKCP
EVCWEGVYNDAFLIDRINWISAGVELDSNQTAENPVFTVEKDNEVLYRAQ
LASEDTNAQKTIINCELLKNKIWCISLVEIYDTGDNVIRPKLFAVKIPEQ
CT

or is substantially similar to SEQ ID NO: 29, or is an active fragment of SEQ ID NO: 29. In some embodiments, the NiV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 29. In some embodiments, the NiV-G comprises an amino acid sequence of SEQ ID NO: 29. In some embodiments, the NiV-G protein comprises a deletion of at least 10 contiguous amino acid residues from the cytoplasmic tail. In some embodiments, the NiV-G protein comprises a deletion of at least 15 contiguous amino acid residues from the cytoplasmic tail. In some embodiments, the NiV-G protein comprises a deletion of at least 20 contiguous amino acid residues from the cytoplasmic tail. In some embodiments, the NiV-G protein comprises a deletion that is located within two amino acid residues of the N-terminus of the NiV-G protein. In some embodiments, the NiV-G protein comprises a deletion comprising amino acid residues 3-7, 3-12, 3-17, 3-22, or 3-27 of SEQ ID NO: 29.

In some embodiments, the NiV-G protein comprises a cytoplasmic tail truncation consisting or comprising of deletion of amino acid residues 2-34 of SEQ ID NO: 29, as shown in underline above. In some embodiments, NiV-G protein comprises an amino acid sequence having at least 70% identity to SEQ ID NO: 30:

(SEQ ID NO: 30)
MKKINEGLLDSKILSAFNTVIALLGSIVIIVMNIMIIQNYTRSTDNQAMI
KDALQSIQQQIKGLADKIGTEIGPKVSLIDTSSTITIPANIGLLGSKISQ
STASINENVNEKCKFTLPPLKIHECNISCPNPLPFREYKPQTEGVSNLVG
LPNNICLQKTSNQILKPKLISYTLPVVGQSGTCITDPLLAMDEGYFAYSH
LEKIGSCSRGVSKQRIIGVGEVLDRGDEVPSLFMTNVWTPSNPNTVYHCS
AVYNNEFYYVLCAVSVVGDPILNSTYWSGSLMMTRLAVKPKNNGESYNQH
QFALRNIEKGKYDKVMPYGPSGIKQGDTLYFPAVGFLVRTEFTYNDSNCP
IAECQYSKPENCRLSMGIRPNSHYILRSGLLKYNLSDEENSKIVFIEISD
QRLSIGSPSKIYDSLGQPVFYQASFSWDTMIKFGDVQTVNPLVVNWRDNT
VISRPGQSQCPRENKCPEVCWEGVYNDAFLIDRINWISAGVFLDSNQTAE
NPVFTVFKDNEVLYRAQLASEDTNAQKTITNCFLLKNKIWCISLVEIYDT
GDNVIRPKLFAVKIPEQCT

or is substantially similar to SEQ ID NO: 30, or is an active fragment of SEQ ID NO: 30. In some embodiments, the NiV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 30.

In some embodiments, the affinity binding polypeptide is fused at the N-terminus of SEQ ID NO: 29 or SEQ ID NO: 30, or a variant thereof as provided for herein. In some embodiments, the affinity binding polypeptide is fused at the C-terminus of SEQ ID NO: 29 or SEQ ID NO: 30, or a variant thereof as provided for herein. In some embodiments, the one or more affinity binding polypeptides are fused at any location within or at the termini's of SEQ ID NO: 29 or SEQ ID NO: 30, or a variant thereof as provided for herein, provided that the inclusion does not substantially negatively affect the purpose of the glycoprotein of SEQ ID NO: 29 or SEQ ID NO: 30.

In some embodiments, the NiV-G fused to the affinity binding polypeptide comprises an amino acid sequence having at least 70% identity to SEQ ID NO: 31:

(SEQ ID NO: 31)
MKKINEGLLDSKILSAFNTVIALLGSIVIIVMNIMIIQNYTRSTDNQAMI
KDALQSIQQQIKGLADKIGTEIGPKVSLIDTSSTITIPANIGLLGSKISQ
STASINENVNEKCKFTLPPLKIHECNISCPNPLPFREYKPQTEGVSNLVG
LPNNICLQKTSNQILKPKLISYTLPVVGQSGTCITDPLLAMDEGYFAYSH
LEKIGSCSRGVSKQRIIGVGEVLDRGDEVPSLFMTNVWTPSNPNTVYHCS
AVYNNEFYYVLCAVSVVGDPILNSTYWSGSLMMTRLAVKPKNNGESYNQH
QFALRNIEKGKYDKVMPYGPSGIKQGDTLYFPAVGFLVRTEFTYNDSNCP
IAECQYSKPENCRLSMGIRPNSHYILRSGLLKYNLSDEENSKIVFIEISD
QRLSIGSPSKIYDSLGQPVFYQASFSWDTMIKEGDVQTVNPLVVNWRDNT
VISRPGQSQCPRFNKCPVVCAEGVYNDAFLIDRINWISAGVELDSNATAA
NPVFTVEKDNEVLYRAQLASEDTNAQKTITNCFLLKNKIWCISLVEIYDT
GDNVIRPKLFAVKIPEQCTDIGGSWSHPQFEK

or is substantially similar to SEQ ID NO: 31. (Bold and underlined sequence represents the affinity binding polypeptide, bolded sequence no underline represents linker peptide). In some embodiments, the NiV-G fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 31. In some embodiments, the NiV-G fused to the affinity binding polypeptide comprises an amino acid sequence of SEQ ID NO: 31.

In some embodiments, a NiV-G fused to the affinity binding polypeptide further comprises a targeting moiety. In some embodiments, the NiV-G comprises an amino acid sequence as provided for herein. In some embodiments, the targeting moiety is fused directly to the NiV-G protein. In some embodiments, the targeting moiety is fused indirectly to the NiV-G protein via, for example, a peptide linker as provided for herein. In some embodiments, the affinity binding polypeptide is located on the N-terminus of the NiV-G:targeting moiety fusion construct. In some embodiments, the affinity binding polypeptide is located on the C-terminus of the NiV-G:targeting moiety fusion construct. In some embodiments, the affinity binding polypeptide is located between the NiV-G protein and the targeting moiety. In some embodiments, the affinity binding polypeptide is located within the NiV-G protein as provided for herein. In some embodiments, the affinity binding polypeptide is located within the targeting moiety as provided for herein. In some embodiments, the NiV-G fused to the affinity binding polypeptide further comprising a targeting moiety comprises an amino acid sequence having at least 70% identity to SEQ ID NO: 32:

(SEQ ID NO: 32)
MKKINEGLLDSKILSAFNTVIALLGSIVIIVMNIMIIQNYTRSTDNQAMI
KDALQSIQQQIKGLADKIGTEIGPKVSLIDTSSTITIPANIGLLGSKISQ
STASINENVNEKCKFTLPPLKIHECNISCPNPLPFREYKPQTEGVSNLVG
LPNNICLQKTSNQILKPKLISYTLPVVGQSGTCITDPLLAMDEGYFAYSH
LEKIGSCSRGVSKQRIIGVGEVLDRGDEVPSLFMTNVWTPSNPNTVYHCS
AVYNNEFYYVLCAVSVVGDPILNSTYWSGSLMMIRLAVKPKNNGESYNQH
QFALRNIEKGKYDKVMPYGPSGIKQGDTLYFPAVGFLVRTEFTYNDSNCP
IAECQYSKPENCRLSMGIRPNSHYILRSGLLKYNLSDEENSKIVFIEISD
QRLSIGSPSKIYDSLGQPVFYQASFSWDTMIKFGDVQTVNPLVVNWRDNT
VISRPGQSQCPRENKCPVVCAEGVYNDAFLIDRINWISAGVELDSNATAA
NPVFTVEKDNEVLYRAQLASEDTNAQKTITNCFLLKNKIWCISLVEIYDT
GDNVIRPKLFAVKIPEQCTDIGGGGSGGGGSGGGGSGGGGSQVQLQQPGA
ELVKPGASVKLSCKASGYPFTSYWIHWVKQRPGRGLEWLGRIDPNSGDTK
YNEKFKNKATLTVDKSSTTAYMQLSSLTSEDSAVYYCARSPYYSNDNSMD
YWGQGTSVTVSSGGGGSGGGGSGGGGSGGGGSDILLTQSPAILSVSPGER
VSFSCRASQSIGTSIHWYQQRINDSPRLLIKYASESISGIPSRFSGSGSG
TDFTLSINSVESEDIADYYCQQSNSWPITFGGGTKLEIKRGGSWSHPQFE
K

or is substantially similar to SEQ ID NO: 32. (Bold and underlined sequence represents the affinity binding polypeptide, bolded sequence no underline represents linker peptide, underlined sequence represents the targeting moiety). In some embodiments, the NiV-G fused to the affinity binding polypeptide further comprising a targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 32. In some embodiments, the NiV-G fused to the affinity binding polypeptide further comprising a targeting moiety comprises an amino acid sequence of SEQ ID NO: 32.

In some embodiments, affinity binding peptides as provided for herein are fused to NiV-F. In some embodiments, NiV-F comprises an amino acid sequence having at least 70% identity to SEQ ID NO 33:

(SEQ ID NO: 33)
MVVILDKRCYCNLLILILMISECSVGILHYEKLSKIGLVKGVTRKYKIKS
NPLTKDIVIKMIPNVSNMSQCTGSVMENYKTRLNGILTPIKGALEIYKNN
THDLVGDVRLAGVIMAGVAIGIATAAQITAGVALYEAMKNADNINKLKSS
IESTNEAVVKLQETAEKTVYVLTALQDYININLVPTIDKISCKQTELSLD
LALSKYLSDLLFVFGPNLQDPVSNSMTIQAISQAFGGNYETLLRTLGYAT
EDFDDLLESDSITGQIIYVDLSSYYIIVRVYFPILTEIQQAYIQELLPVS
FNNDNSEWISIVPNFILVRNTLISNIEIGFCLITKRSVICNQDYATPMIN
NMRECLTGSTEKCPRELVVSSHVPRFALSNGVLFANCISVTCQCQTTGRA
ISQSGEQTLLMIDNTTCPTAVLGNVIISLGKYLGSVNYNSEGIAIGPPVF
TDKVDISSQISSMNQSLQQSKDYIKEAQRLLDTVNPSLISMLSMIILYVL
SIASLCIGLITFISFIIVEKKRNTYSRLEDRRVRPTSSGDLYYIGT

or is substantially similar to SEQ ID NO: 33, or is an active fragment of SEQ ID NO: 33. In some embodiments, the NiV-F comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 33. In some embodiments, the NiV-F comprises an amino acid sequence of SEQ ID NO: 33.

In some embodiments, the NiV-F protein has a cytoplasmic tail truncation comprising the deletion of amino acid residues 526-546 of SEQ ID NO: 33. In some embodiments, the NiV-F protein comprises a cytoplasmic tail lacking amino residues 525-544 of SEQ ID NO: 33. In some embodiments, the NiV-F protein comprises amino acid residues 519-524 of SEQ ID NO: 33. In some embodiments, the NiV-F protein comprises amino acid residues 519-525 of SEQ ID NO: 33. In some embodiments, the NiV-F protein comprises amino acid residues 519-526 of SEQ ID NO: 33. In some embodiments, the NiV-F protein comprises a substitution of glutamine for asparagine at an amino acid position that corresponds to position 99 of SEQ ID NO: 33.

In some embodiments, NiV-F comprises an amino acid sequence having at least 70% identity to SEQ ID NO 34:

(SEQ ID NO: 34)
MVVILDKRCYCNLLILILMISECSVGILHYEKLSKIGLVKGVTRKYKIKS
NPLIKDIVIKMIPNVSNMSQCTGSVMENYKIRLNGILTPIKGALEIYKNN
THDLVGDVRLAGVIMAGVAIGIATAAQITAGVALYEAMKNADNINKLKSS
IESTNEAVVKLQETAEKTVYVLTALQDYININLVPTIDKISCKQTELSLD
LALSKYLSDLLFVFGPNLQDPVSNSMTIQAISQAFGGNYETLLRTLGYAT
EDFDDLLESDSITGQIIYVDLSSYYIIVRVYFPILTEIQQAYIQELLPVS
FNNDNSEWISIVPNFILVRNTLISNIEIGFCLITKRSVICNQDYATPMTN
NMRECLTGSTEKCPRELVVSSHVPRFALSNGVLFANCISVTCQCQTTGRA
ISQSGEQTLLMIDNTTCPTAVLGNVIISLGKYLGSVNYNSEGIAIGPPVE
TDKVDISSQISSMNQSLQQSKDYIKEAQRLLDTVNPSLISMLSMIILYVL
SIASLCIGLITFISFIIVEKKRNT

or is substantially similar to SEQ ID NO: 34, or is an active fragment of SEQ ID NO: 34. In some embodiments, the NiV-F comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 34. In some embodiments, the NiV-F comprises an amino acid sequence of SEQ ID NO: 34.

In some embodiments, the affinity binding polypeptide is fused at the N-terminus of SEQ ID NO: 33 or SEQ ID NO: 34, or a variant thereof as provided for herein. In some embodiments, the affinity binding polypeptide is fused at the C-terminus of SEQ ID NO: 33 or SEQ ID NO: 34, or a variant thereof as provided for herein. In some embodiments, the one or more affinity binding polypeptides are fused at any location within or at the termini's of SEQ ID NO: 33 or SEQ ID NO: 34, or a variant thereof as provided for herein, provided that the inclusion does not substantially negatively affect the purpose of the glycoprotein of SEQ ID NO: 33 or SEQ ID NO: 34.

In some embodiments, the NiV-F fused to the affinity binding polypeptide comprises an amino acid sequence having at least 70% identity to SEQ ID NO: 35:

(SEQ ID NO: 35)
MVVILDKRCYCNLLILILMISECSVGWSHPQFEKGGSILHYEKLSKIGLV
KGVTRKYKIKSNPLTKDIVIKMIPNVSNMSQCTGSVMENYKTRLNGILTP
IKGALEIYKNNTHDLVGDVRLAGVIMAGVAIGIATAAQITAGVALYEAMK
NADNINKLKSSIESTNEAVVKLQETAEKTVYVLTALQDYINTNLVPTIDK
ISCKQTELSLDLALSKYLSDLLFVFGPNLQDPVSNSMTIQAISQAFGGNY
ETLLRTLGYATEDFDDLLESDSITGQIIYVDLSSYYIIVRVYFPILTEIQ
QAYIQELLPVSFNNDNSEWISIVPNFILVRNTLISNIEIGFCLITKRSVI
CNQDYATPMINNMRECLIGSTEKCPRELVVSSHVPRFALSNGVLFANCIS
VTCQCQTTGRAISQSGEQTLLMIDNTTCPTAVLGNVIISLGKYLGSVNYN
SEGIAIGPPVFTDKVDISSQISSMNQSLQQSKDYIKEAQRLLDTVNPSLI
SMLSMIILYVLSIASLCIGLITFISFIIVEKKRNT

or is substantially similar to SEQ ID NO: 35. (Italicized amino acids represent the NiV-F signal peptide, bold and underlined sequence represents the affinity binding polypeptide, bolded sequence no underline represents linker peptide). The sequence of SEQ ID NO: 35 contains the NiV-F signal peptide MVVILDKRCYCNLLILILMISECSVG (SEQ ID NO: 165). Upon processing, the signal peptide may be cleaved leaving the sequence of SEQ ID NO: 166 (See Table 6). Thus, while embodiments may refer to further modifications of, mutations of, or percent identity to SEQ ID NO: 35, it is to be understood that the same modifications, mutations, and limitations to percent identity also apply to SEQ ID NO: 166. In some embodiments, the NiV-F fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 35. In some embodiments, the NiV-F fused to the affinity binding polypeptide comprises an amino acid sequence of SEQ ID NO: 35.

In some embodiments, affinity binding peptides as provided for herein are fused to VSV-G. Vesicular stomatitis virus (VSV) is an enveloped, negative-strand RNA virus that belongs to the Vesiculovirus genus of the Rhabdovirus family. It is an arbovirus which can infect insects, cattle, horses and pigs. VSV genome encodes five structural proteins among which include a single transmembrane glycoprotein (G). The glycoprotein is a classic type I membrane glycoprotein with an amino-terminal signal peptide, an ectodomain of about 450 amino acids, a single alpha helical transmembrane segment and a small intraviral carboxy-terminal domain. The signal peptide is cleaved in the lumen of the endoplasmic reticulum and the native glycoprotein consists in the ectodomain, the transmembrane domain and the intraviral domain. The following non-limiting embodiments will be recited without the inclusion of the amino-terminal signal peptide and thus for any reference to specific mutations the amino acid position will be the position excluding the amino-terminal signal peptide. However, it is to be understood that inclusion of the amino-terminal signal peptide is within the scope of the present application, and upon inclusion the amino acid position of particular mutations would be shifted accordingly. Further, there are several strains of VSV-G known, and all such strains are encompassed by the present application. Accordingly, in some embodiments, the VSV-G glycoprotein is of a vesicular stomatitis New Jersey virus strain, a vesicular stomatitis Indiana virus strain, a vesicular stomatitis Alagoas virus strain, a vesicular stromatitis Maraba virus strain, or a vesicular stomatitis Carajas virus strain, or any combination thereof.

In some embodiments, the VSV-G glycoprotein is of the Indiana strain and comprises an amino acid sequence having at least 70% identity to SEQ ID NO: 36:

(SEQ ID NO: 36)
MKCLLYLAFLFIGVNCKFTIVFPHNQKGNWKNVPSNYHYCPSSSDLNWHN
DLIGTALQVKMPKSHKAIQADGWMCHASKWVTTCDFRWYGPKYITHSIRS
FTPSVEQCKESIEQTKQGTWLNPGFPPQSCGYATVIDAEAVIVQVTPHHV
LVDEYTGEWVDSQFINGKCSNYICPTVHNSTTWHSDYKVKGLCDSNLISM
DITFFSEDGELSSLGKEGTGERSNYFAYETGGKACKMQYCKHWGVRLPSG
VWFEMADKDLFAAARFPECPEGSSISAPSQTSVDVSLIQDVERILDYSLC
QETWSKIRAGLPISPVDLSYLAPKNPGTGPAFTIINGTLKYFETRYIRVD
IAAPILSRMVGMISGITTERELWDDWAPYEDVEIGPNGVLRTSSGYKEPL
YMIGHGMLDSDLHLSSKAQVFEHPHIQDAASQLPDDESLFFGDTGLSKNP
IELVEGWFSSWKSSIASFFFIIGLIIGLFLVLRVGIHLCIKLKHTKKRQI
YTDIEMNRLGK

or is substantially similar to SEQ ID NO: 36, or is an active fragment of SEQ ID NO: 36. In some embodiments, the VSV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 36. In some embodiments, the VSV-G comprises an amino acid sequence of SEQ ID NO: 36.

In some embodiments, the VSV-G glycoprotein is of the Indiana strain and comprises an amino acid sequence having at least 70% identity to SEQ ID NO: 37:

(SEQ ID NO: 37)
KFTIVFPHNQKGNWKNVPSNYHYCPSSSDLNWHNDLIGTALQVKMPKSHK
AIQADGWMCHASKWVTTCDFRWYGPKYITHSIRSFTPSVEQCKESIEQTK
QGTWLNPGFPPQSCGYATVIDAEAVIVQVTPHHVLVDEYTGEWVDSQFIN
GKCSNYICPTVHNSTTWHSDYKVKGLCDSNLISMDITFFSEDGELSSLGK
EGTGFRSNYFAYETGGKACKMQYCKHWGVRLPSGVWFEMADKDLFAAARF
PECPEGSSISAPSQTSVDVSLIQDVERILDYSLCQETWSKIRAGLPISPV
DLSYLAPKNPGTGPAFTIINGTLKYFETRYIRVDIAAPILSRMVGMISGI
TTERELWDDWAPYEDVEIGPNGVLRTSSGYKFPLYMIGHGMLDSDLHLSS
KAQVFEHPHIQDAASQLPDDESLFFGDTGLSKNPIELVEGWESSWKSSIA
SFFFIIGLIIGLFLVLRVGIHLCIKLKHIKKRQIYTDIEMNRLGK

or is substantially similar to SEQ ID NO: 37, or is an active fragment of SEQ ID NO: 37. In some embodiments, the VSV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 37. In some embodiments, the VSV-G comprises an amino acid sequence of SEQ ID NO: 37.

In some embodiments, the VSV-G protein comprises a mutation at position 198 as compared to SEQ ID NO: 36 or at 182 compared to SEQ ID NO: 37. SEQ ID NO: 36 is the full length protein and SEQ ID NO: 37 is the ectodomain of the VSV-G protein. The 16-mer signal peptide of MKCLLYLAFLFIGVNC (SEQ ID NO: 38) as shown at the N-terminus of SEQ ID NO: 36 is cleaved leaving a protein of SEQ ID NO: 37. Thus, although a mutation may be referred to in the context of SEQ ID NO: 37, it should be understood to also be made in the context of SEQ ID NO: 36, which contains the leader sequence, and thus would be a position number that is 16 more than the position recited for SEQ ID NO: 37. In some embodiments, the mutation inhibits or decreases the binding of the VSV-G protein to the LDL receptor. In some embodiments the mutation is I182D as compared to SEQ ID NO: 37. In some embodiments, the sequence of the VSV-G protein comprising an I182D mutation as compared to SEQ ID NO: 37 is given by the sequence of SEQ ID NO: 40. In some embodiments, the mutation is 1182E as compared to SEQ ID NO: 37. In some embodiments, the sequence of the VSV-G protein comprising an I182E mutation as compared to SEQ ID NO: 37 is given by the sequence of SEQ ID NO: 41. In some embodiments, the mutation is I182A as compared to SEQ ID NO: 37. In some embodiments, the sequence of the VSV-G protein comprising an I182A mutation as compared to SEQ ID NO: 37 is given by the sequence of SEQ ID NO: 39. In some embodiments, the mutation at position 182 compared to SEQ ID NO: 37 is not alanine. In some embodiments, the mutation at position 182 as compared to SEQ ID NO: 37 is not valine. In some embodiments, the mutation at position 182 as compared to SEQ ID NO: 37 is 1182S, 1182H, 1182T, 1182Q, or 1182N.

A VSV-G protein comprising a mutation at position 182 as compared to SEQ ID NO: 37 can also comprise other mutations, such as those described in U.S. Patent Application Publication No. 20200216502, which is hereby incorporated by reference in its entirety. For example, the VSV-G protein can comprise a mutation at a position that corresponds to positions of 8, 47, 209 and/or 354 of SEQ ID NO: 37.

In some embodiments, the substitution at position 8 is by any amino acid different from the amino acid indicated at that position in the sequence SEQ ID NO: 37, except Y. In some embodiments, the substitution at position 209 is by any amino acid different from the amino acid indicated at that position in the sequence SEQ ID NO: 37, except H. In some embodiments, the substitution at position 47 is by any amino acid different from the amino acid indicated at that position in the sequence SEQ ID NO: 37, except K or R. In some embodiments, the substitution at position 354 is by any amino acid different from the amino acid indicated at that position in the sequence SEQ ID NO: 37, except K or R.

In some embodiments, the substitution is at position 47 or at position 354, or at both positions 47 and 354 are substituted by A, G, F or Q. In some embodiments, the substitution is A or Q.

In some embodiments, the substitution at position 8 is an alanine, i.e., H8A.

In some embodiments, the substitution at position 47 is Q or N, i.e., K47Q or K47N.

In some embodiments, the protein comprises a mutation (substitution) at position 10. In some embodiments, the substitution/mutation is Q10A, Q10R, or Q10K.

In some embodiments, the VSV-G protein comprising a mutation at a position corresponding to positions of 8, 47, 209 and/or 354 of SEQ ID NO: 37 are given by the following sequences:

### VSV-G Indiana Ectodomain H8A+K47Q:

(SEQ ID NO: 42)
KFTIVFPANQKGNWKNVPSNYHYCPSSSDLNWHNDLIGTALQVKMPQSHK
AIQADGWMCHASKWVTTCDFRWYGPKYITHSIRSFTPSVEQCKESIEQTK
QGTWLNPGFPPQSCGYATVIDAEAVIVQVTPHHVLVDEYTGEWVDSQFIN
GKCSNYICPTVHNSTTWHSDYKVKGLCDSNLISMDITFFSEDGELSSLGK
EGTGFRSNYFAYETGGKACKMQYCKHWGVRLPSGVWFEMADKDLFAAARF
PECPEGSSISAPSQTSVDVSLIQDVERILDYSLCQETWSKIRAGLPISPV
DLSYLAPKNPGTGPAFTIINGTLKYFETRYIRVDIAAPILSRMVGMISGT
TTERELWDDWAPYEDVEIGPNGVLRTSSGYKFPLYMIGHGMLDSDLHLSS
KAQVFEHPHIQDAASQLPDDESLFFGDTGLSKNPIELVEGWESSWKSSIA
SFFFIIGLIIGLFLVLRVGIHLCIKLKHTKKRQIYTDIEMNRLGK

VSV-G Indiana Ectodomain Q10A:
(SEQ ID NO: 43)
KFTIVFPHNAKGNWKNVPSNYHYCPSSSDLNWHNDLIGTALQVKMPKSHK
AIQADGWMCHASKWVTTCDFRWYGPKYITHSIRSFTPSVEQCKESIEQTK
QGTWLNPGFPPQSCGYATVIDAEAVIVQVTPHHVLVDEYTGEWVDSQFIN
GKCSNYICPTVHNSTTWHSDYKVKGLCDSNLISMDITFFSEDGELSSLGK
EGTGERSNYFAYETGGKACKMQYCKHWGVRLPSGVWFEMADKDLFAAARF
PECPEGSSISAPSQTSVDVSLIQDVERILDYSLCQETWSKIRAGLPISPV
DLSYLAPKNPGTGPAFTIINGILKYFETRYIRVDIAAPILSRMVGMISGT
TTERELWDDWAPYEDVEIGPNGVLRTSSGYKFPLYMIGHGMLDSDLHLSS
KAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK
VSV-G Indiana Ectodomain Q10R:
(SEQ ID NO: 44)
KFTIVFPHNRKGNWKNVPSNYHYCPSSSDLNWHNDLIGTALQVKMPKSHK
AIQADGWMCHASKWVTTCDFRWYGPKYITHSIRSFTPSVEQCKESIEQTK
QGTWLNPGFPPQSCGYATVIDAEAVIVQVTPHHVLVDEYTGEWVDSQFIN
GKCSNYICPTVHNSTTWHSDYKVKGLCDSNLISMDITFFSEDGELSSLGK
EGTGERSNYFAYETGGKACKMQYCKHWGVRLPSGVWFEMADKDLFAAARF
PECPEGSSISAPSQTSVDVSLIQDVERILDYSLCQETWSKIRAGLPISPV
DLSYLAPKNPGTGPAFTIINGTLKYFETRYIRVDIAAPILSRMVGMISGT
TTERELWDDWAPYEDVEIGPNGVLRISSGYKFPLYMIGHGMLDSDLHLSS
KAQVFEHPHIQDAASQLPDDESLFFGDTGLSKNPIELVEGWESSWKSSIA
SFFFIIGLIIGLFLVLRVGIHLCIKLKHTKKRQIYTDIEMNRLGK
VSV-G Indiana Ectodomain Q10K:
(SEQ ID NO: 45)
KFTIVFPHNKKGNWKNVPSNYHYCPSSSDLNWHNDLIGTALQVKMPKSHK
AIQADGWMCHASKWVTTCDFRWYGPKYITHSIRSFTPSVEQCKESIEQTK
QGTWLNPGFPPQSCGYATVIDAEAVIVQVTPHHVLVDEYTGEWVDSQFIN
GKCSNYICPTVHNSTTWHSDYKVKGLCDSNLISMDITFFSEDGELSSLGK
EGTGERSNYFAYETGGKACKMQYCKHWGVRLPSGVWFEMADKDLFAAARF
PECPEGSSISAPSQTSVDVSLIQDVERILDYSLCQETWSKIRAGLPISPV
DLSYLAPKNPGTGPAFTIINGTLKYFETRYIRVDIAAPILSRMVGMISGT
TTERELWDDWAPYEDVEIGPNGVLRTSSGYKFPLYMIGHGMLDSDLHLSS
KAQVFEHPHIQDAASQLPDDESLFFGDTGLSKNPIELVEGWESSWKSSIA
SFFFIIGLIIGLFLVLRVGIHLCIKLKHTKKRQIYTDIEMNRLGK

In some embodiments, a protein comprising a mutation at position 182 as compared to SEQ ID NO: 37 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 37. In some embodiments, the polypeptide comprises a I182D or 1182E mutation. In some embodiments, the VSV-G protein comprises a 1182S, 1182H, 1182T, 1182Q, or I182N mutation.

In some embodiments, the VSV-G glycoprotein is of the New Jersey strain and comprises an amino acid sequence having at least 70% identity to SEQ ID NO: 46:

(SEQ ID NO: 46)
MLSYLIFALVVSPILGKIEIVFPQHTTGDWKRVPHEYNYCPTSADKNSHG
TQTGIPVELTMPKGLTTHQVDGFMCHSALWMTTCDFRWYGPKYITHSIHN
EEPTDYQCLEAIKAYKDGVSFNPGFPPQSCGYGTVTDAEAHIVTVTPHSV
KVDEYTGEWIDPHFIGGRCKGQICETVHNSTKWFTSSDGESVCSQLFTLV
GGTFFSDSEEITSMGLPETGIRSNYFPYVSTEGICKMPFCRKPGYKLKND
LWFQITDPDLDKTVRDLPHIKDCDLSSSIVTPGEHATDISLISDVERILD
YALCQNTWSKIEAGEPITPVDLSYLGPKNPGAGPVFTIINGSLHYFMSKY
LRVELESPVIPRMEGKVAGTRIVRQLWDQWFPFGEVEIGPNGVLKTKQGY
KFPLHIIGTGEVDNDIKMERIVKHWEHPHIEAAQTFLKKDDTEEVLYYGD
TGVSKNPVELVEGWFSGWRSSIMGVLAVIIGFVILIFLIRLIGVLSSLER
QKRRPIYKSDVEMAHER

or is substantially similar to SEQ ID NO: 46, or is an active fragment of SEQ ID NO: 46. In some embodiments, the VSV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 46. In some embodiments, the VSV-G comprises an amino acid sequence of SEQ ID NO: 46.

In some embodiments, the VSV-G glycoprotein is of the New Jersey strain and comprises an amino acid sequence having at least 70% identity to SEQ ID NO: 47:

(SEQ ID NO: 47)
KIEIVFPQHTTGDWKRVPHEYNYCPTSADKNSHGTQTGIPVELTMPKGLT
THQVDGFMCHSALWMITCDFRWYGPKYITHSIHNEEPTDYQCLEAIKAYK
DGVSFNPGFPPQSCGYGTVTDAEAHIVTVTPHSVKVDEYTGEWIDPHFIG
GRCKGQICETVHNSTKWFISSDGESVCSQLFTLVGGIFFSDSEEITSMGL
PETGIRSNYFPYVSTEGICKMPFCRKPGYKLKNDLWFQITDPDLDKTVRD
LPHIKDCDLSSSIVTPGEHATDISLISDVERILDYALCQNTWSKIEAGEP
ITPVDLSYLGPKNPGAGPVFTIINGSLHYFMSKYLRVELESPVIPRMEGK
VAGTRIVRQLWDQWFPFGEVEIGPNGVLKTKQGYKFPLHIIGTGEVDNDI
KMERIVKHWEHPHIEAAQTFLKKDDTEEVLYYGDTGVSKNPVELVEGWFS
GWRSSIMGVLAVIIGFVILIFLIRLIGVLSSLFRQKRRPIYKSDVEMAHF
R

or is substantially similar to SEQ ID NO: 47, or is an active fragment of SEQ ID NO: 47. In some embodiments, the VSV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 47. In some embodiments, the VSV-G comprises an amino acid sequence of SEQ ID NO: 47.

In some embodiments, the VSV-G protein comprises a mutation at position 198 as compared to SEQ ID NO: 46 or at position 182 as compared to SEQ ID NO: 47. SEQ ID NO: 46 is the full length protein and SEQ ID NO: 47 is the ectodomain of the VSV-G protein. The 16-mer signal peptide of MLSYLIFALVVSPILG (SEQ ID NO: 48) as shown at the N-terminus of SEQ ID NO: 46 is cleaved leaving a protein of SEQ ID NO: 47. Thus, although a mutation may be referred to in the context of SEQ ID NO: 47, it should be understood to also be made in the context of SEQ ID NO: 46, which contains the leader sequence, and thus would be a position number that is 16 more than the position recited for SEQ ID NO: 47. In some embodiments, the mutation inhibits or decreases the binding of the VSV-G protein to the LDL receptor. In some embodiments the mutation is T182D as compared to SEQ ID NO: 47. In some embodiments, the mutation is Ti82E as compared to SEQ ID NO: 47.

In some embodiments, a protein comprising a mutation at position 182 as compared to SEQ ID NO: 47 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 47. In some embodiments, the polypeptide comprises a T182D or T182E mutation. In some embodiments, the VSV-G protein comprises a T182S, T182H, T182Q, or T182N mutation.

In some embodiments, the VSV-G glycoprotein is of the Marraba strain and comprises an amino acid sequence having at least 70% identity to SEQ ID NO: 49:

(SEQ ID NO: 49)
MLRLFLFCFLALGAHSKFTIVFPHHQKGNWKNVPSTYHYCPSSSDQNWHN
DLTGVSLHVKIPKSHKAIQADGWMCHAAKWVTTCDFRWYGPKYITHSIHS
MSPTLEQCKTSIEQTKQGVWINPGFPPQSCGYATVIDAEVVVVQATPHHV
LVDEYTGEWIDSQLVGGKCSKEVCQTVHNSTVWHADYKITGLCESNLASV
DITFFSEDGQKTSLGKPNTGERSNHFAYESGEKACRMQYCTQWGIRLPSG
VWFELVDKDLFQAAKLPECPRGSSISAPSQTSVDVSLIQDVERILDYSLC
QETWSKIRAKLPVSPVDLSYLAPKNPGSGPAFTIINGTLKYFETRYIRVD
ISNPIIPHMVGTMSGTTTERELWNDWYPYEDVEIGPNGVLKTPTGFKFPL
YMIGHGMLDSDLHKSSQAQVFEHPHAKDAASQLPDDETLFFGDTGLSKNP
VELVEGWFSSWKSTLASFFLIIGLGVALIFIIRIIVAIRYKYKGRKTQKI
YNDVEMSRLGNK

or is substantially similar to SEQ ID NO: 49, or is an active fragment of SEQ ID NO: 49. In some embodiments, the VSV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 49. In some embodiments, the VSV-G comprises an amino acid sequence of SEQ ID NO: 49.

In some embodiments, the VSV-G glycoprotein is of the Marraba strain and comprises an amino acid sequence having at least 70% identity to SEQ ID NO: 50:

(SEQ ID NO: 50)
KFTIVFPHHQKGNWKNVPSTYHYCPSSSDQNWHNDLTGVSLHVKIPKSHK
AIQADGWMCHAAKWVTTCDFRWYGPKYITHSIHSMSPTLEQCKTSIEQTK
QGVWINPGFPPQSCGYATVIDAEVVVVQATPHHVLVDEYTGEWIDSQLVG
GKCSKEVCQTVHNSTVWHADYKITGLCESNLASVDITFFSEDGQKTSLGK
PNTGERSNHFAYESGEKACRMQYCTQWGIRLPSGVWFELVDKDLFQAAKL
PECPRGSSISAPSQTSVDVSLIQDVERILDYSLCQETWSKIRAKLPVSPV
DLSYLAPKNPGSGPAFTIINGTLKYFETRYIRVDISNPIIPHMVGIMSGI
TTERELWNDWYPYEDVEIGPNGVLKTPTGFKFPLYMIGHGMLDSDLHKSS
QAQVFEHPHAKDAASQLPDDETLEFGDTGLSKNPVELVEGWFSSWKSTLA
SFFLIIGLGVALIFIIRIIVAIRYKYKGRKTQKIYNDVEMSRLGNK

or is substantially similar to SEQ ID NO: 50, or is an active fragment of SEQ ID NO: 50. In some embodiments, the VSV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 50. In some embodiments, the VSV-G comprises an amino acid sequence of SEQ ID NO: 50.

In some embodiments, the VSV-G protein comprises a mutation at position 198 as compared to SEQ ID NO: 49 or position 182 as compared to SEQ ID NO: 50. SEQ ID NO: 49 is the full length protein and SEQ ID NO: 50 is the ectodomain of the VSV-G protein. The 16-mer signal peptide of MLRLFLFCFLALGAHS (SEQ ID NO: 51) as shown at the N-terminus of SEQ ID NO: 49 is cleaved leaving a protein of SEQ ID NO: 50. Thus, although a mutation may be referred to in the context of SEQ ID NO: 50, it should be understood to also be made in the context of SEQ ID NO: 49, which contains the leader sequence, and thus would be a position number that is 16 more than the position recited for SEQ ID NO: 50. In some embodiments, the mutation inhibits or decreases the binding of the VSV-G protein to the LDL receptor. In some embodiments the mutation is A182D as compared to SEQ ID NO: 50. In some embodiments, the mutation is A182E as compared to SEQ ID NO: 50.

In some embodiments, a protein comprising a mutation at position 182 as compared to SEQ ID NO: 50 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 50. In some embodiments, the polypeptide comprises a A182D or A182E mutation. In some embodiments, the VSV-G protein comprises a A182S, A182H, A182T, A182Q, or A182N mutation.

In some embodiments, the VSV-G glycoprotein is of the Carajas strain and comprises an amino acid sequence having at least 70% identity to SEQ ID NO: 52:

(SEQ ID NO: 52)
MKMKMVIAGLILCIGILPAIGKITISFPQSLKGDWRPVPKGYNYCPTSAD
KNLHGDLIDIGLRLRAPKSFKGISADGWMCHAARWITTCDFRWYGPKYIT
HSIHSFRPSNDQCKEAIRLINEGNWINPGFPPQSCGYASVTDSESVVVTV
TKHQVLVDEYSGSWIDSQFPGGSCTSPICDTVHNSTLWHADHTLDSICDQ
EFVAMDAVLFTESGKFEEFGKPNSGIRSNYFPYESLKDVCQMDFCKRKGE
KLPSGVWFEIEDAEKSHKAQVELKIKRCPHGAVISAPNQNAADINLIMDV
ERILDYSLCQATWSKIQNKEALTPIDISYLGPKNPGPGPAFTIINGTLHY
FNTRYIRVDIAGPVTKEITGFVSGTSTSRVLWDQWFPYGENSIGPNGLLK
TASGYKYPLFMVGTGVLDADIHKLGEATVIEHPHAKEAQKVVDDSEVIFF
GDTGVSKNPVEVVEGWFSGWRSSLMSIFGIILLIVCLVLIVRILIALKYC
CVRHKKRTIYKEDLEMGRIPRRA

or is substantially similar to SEQ ID NO: 52, or is an active fragment of SEQ ID NO: 52. In some embodiments, the VSV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 52. In some embodiments, the VSV-G comprises an amino acid sequence of SEQ ID NO: 52.

In some embodiments, the VSV-G glycoprotein is of the Carajas strain and comprises an amino acid sequence having at least 70% identity to SEQ ID NO: 53:

(SEQ ID NO: 53)
KITISFPQSLKGDWRPVPKGYNYCPTSADKNLHGDLIDIGLRLRAPKSFK
GISADGWMCHAARWITTCDFRWYGPKYITHSIHSFRPSNDQCKEAIRLIN
EGNWINPGFPPQSCGYASVTDSESVVVTVTKHQVLVDEYSGSWIDSQFPG
GSCTSPICDTVHNSTLWHADHTLDSICDQEFVAMDAVLFTESGKFEEFGK
PNSGIRSNYFPYESLKDVCQMDFCKRKGFKLPSGVWFEIEDAEKSHKAQV
ELKIKRCPHGAVISAPNQNAADINLIMDVERILDYSLCQATWSKIQNKEA
LTPIDISYLGPKNPGPGPAFTIINGTLHYENTRYIRVDIAGPVTKEITGF
VSGTSTSRVLWDQWFPYGENSIGPNGLLKTASGYKYPLFMVGTGVLDADI
HKLGEATVIEHPHAKEAQKVVDDSEVIFFGDTGVSKNPVEVVEGWFSGWR
SSLMSIFGIILLIVCLVLIVRILIALKYCCVRHKKRTIYKEDLEMGRIPR
RA

or is substantially similar to SEQ ID NO: 53, or is an active fragment of SEQ ID NO: 53. In some embodiments, the VSV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 53. In some embodiments, the VSV-G comprises an amino acid sequence of SEQ ID NO: 53.

In some embodiments, the VSV-G protein comprises a mutation at position 203 as compared to SE ID NO: 52 or at position 182 as compared to SEQ ID NO: 53. SEQ ID NO: 52 is the full length protein and SEQ ID NO: 53 is the ectodomain of the VSV-G protein. The 21-mer signal peptide of MKMKMVIAGLILCIGILPAIG (SEQ ID NO: 54) as shown at the N-terminus of SEQ ID NO: 52 is cleaved leaving a protein of SEQ ID NO: 53. Thus, although a mutation may be referred to in the context of SEQ ID NO: 53, it should be understood to also be made in the context of SEQ ID NO: 52, which contains the leader sequence, and thus would be a position number that is 21 more than the position recited for SEQ ID NO: 53. In some embodiments, the mutation inhibits or decreases the binding of the VSV-G protein to the LDL receptor. In some embodiments the mutation is V182D as compared to SEQ ID NO: 53. In some embodiments, the mutation is V182E as compared to SEQ ID NO: 53.

In some embodiments, a protein comprising a mutation at position 182 as compared to SEQ ID NO: 53 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 53. In some embodiments, the polypeptide comprises a V182D or V182E mutation. In some embodiments, the VSV-G protein comprises a V182S, V182H, V182T, V182Q, or V182N mutation.

In some embodiments, the VSV-G glycoprotein is of the Alagoa strain and comprises an amino acid sequence having at least 70% identity to SEQ ID NO: 55:

(SEQ ID NO: 55)
MTPAFILCMLLAGSSWAKFTIVFPQSQKGDWKDVPPNYRYCPSSADQNWH
GDLLGVNIRAKMPKVHKAIKADGWMCHAAKWVTTCDYRWYGPQYITHSIH
SFIPTKAQCEESIKQTKEGVWINPGFPPKNCGYASVSDAESIIVQATAHS
VMIDEYSGDWLDSQFPTGRCTGSTCETIHNSTLWYADYQVTGLCDSALVS
TEVTFYSEDGLMISIGRQNTGYRSNYFPYEKGAAACRMKYCTHEGIRLPS
GVWFEMVDKELLESVQMPECPAGLTISAPTQTSVDVSLILDVERMLDYSL
CQETWSKVHSGLPISPVDLGYIAPKNPGAGPAFTIVNGTLKYEDTRYLRI
DIEGPVLKKMTGKVSGTPTKRELWTEWFPYDDVEIGPNGVLKTPEGYKFP
LYMIGHGLLDSDLQKTSQAEVFHHPQIAEAVQKLPDDETLFFGDTGISKN
PVEVIEGWFSNWRSSVMAIVFAILLLVITVLMVRLCVAFRHFCCQKRHKI
YNDLEMNQLRR

or is substantially similar to SEQ ID NO: 55, or is an active fragment of SEQ ID NO: 55. In some embodiments, the VSV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 55. In some embodiments, the VSV-G comprises an amino acid sequence of SEQ ID NO: 55.

In some embodiments, the VSV-G glycoprotein is of the Alagoa strain and comprises an amino acid sequence having at least 70% identity to SEQ ID NO: 56:

(SEQ ID NO: 56)
KFTIVFPQSQKGDWKDVPPNYRYCPSSADQNWHGDLLGVNIRAKMPKVHK
AIKADGWMCHAAKWVTTCDYRWYGPQYITHSIHSFIPTKAQCEESIKQTK
EGVWINPGFPPKNCGYASVSDAESIIVQATAHSVMIDEYSGDWLDSQFPT
GRCTGSTCETIHNSTLWYADYQVTGLCDSALVSTEVTFYSEDGLMTSIGR
QNTGYRSNYFPYEKGAAACRMKYCTHEGIRLPSGVWFEMVDKELLESVQM
PECPAGLTISAPTQTSVDVSLILDVERMLDYSLCQETWSKVHSGLPISPV
DLGYIAPKNPGAGPAFTIVNGTLKYFDTRYLRIDIEGPVLKKMTGKVSGT
PTKRELWTEWFPYDDVEIGPNGVLKTPEGYKFPLYMIGHGLLDSDLQKTS
QAEVFHHPQIAEAVQKLPDDETLFFGDIGISKNPVEVIEGWFSNWRSSVM
AIVFAILLLVITVLMVRLCVAFRHFCCQKRHKIYNDLEMNQLRR

or is substantially similar to SEQ ID NO: 56, or is an active fragment of SEQ ID NO: 56. In some embodiments, the VSV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 56. In some embodiments, the VSV-G comprises an amino acid sequence of SEQ ID NO: 56.

In some embodiments, the VSV-G protein comprises a mutation at position 199 as compared to SEQ ID NO: 55 or at position 182 as compared to SEQ ID NO: 56. SEQ ID NO: 55 is the full length protein and SEQ ID NO: 56 is the ectodomain of the VSV-G protein. The 17-mer signal peptide of MTPAFILCMLLAGSSWA (SEQ ID NO: 57) as shown at the N-terminus of SEQ ID NO: 55 is cleaved leaving a protein of SEQ ID NO: 56. Thus, although a mutation may be referred to in the context of SEQ ID NO: 56, it should be understood to also be made in the context of SEQ ID NO: 55, which contains the leader sequence, and thus would be a position number that is 17 more than the position recited for SEQ ID NO: 56. In some embodiments, the mutation inhibits or decreases the binding of the VSV-G protein to the LDL receptor. In some embodiments the mutation is V182D as compared to SEQ ID NO: 56. In some embodiments, the mutation is V182E as compared to SEQ ID NO: 56.

In some embodiments, a protein comprising a mutation at position 182 as compared to SEQ ID NO: 56 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 56. In some embodiments, the polypeptide comprises a V182D or V182E mutation. In some embodiments, the VSV-G protein comprises a V182S, V182H, V182T, V182Q, or V182N mutation.

In some embodiments, the VSV-G glycoprotein is of the Cocal strain and comprises an amino acid sequence having at least 70% identity to SEQ ID NO: 58:

(SEQ ID NO: 58)
MNFLLLTFIVLPLCSHAKESIVFPQSQKGNWKNVPSSYHYCPSSSDQNWH
NDLLGITMKVKMPKTHKAIQADGWMCHAAKWITTCDFRWYGPKYITHSIH
SIQPTSEQCKESIKQTKQGTWMSPGFPPQNCGYATVTDSVAVVVQATPHH
VLVDEYTGEWIDSQFPNGKCETEECETVHNSTVWYSDYKVTGLCDATLVD
TEITFFSEDGKKESIGKPNTGYRSNYFAYEKGDKVCKMNYCKHAGVRLPS
GVWFEFVDQDVYAAAKLPECPVGATISAPTQTSVDVSLILDVERILDYSL
CQETWSKIRSKQPVSPVDLSYLAPKNPGTGPAFTIINGTLKYFETRYIRI
DIDNPIISKMVGKISGSQTERELWTEWFPYEGVEIGPNGILKTPTGYKFP
LFMIGHGMLDSDLHKTSQAEVFEHPHLAEAPKQLPEEETLFFGDTGISKN
PVELIEGWFSSWKSTVVIFFFAIGVFILLYVVARIVIAVRYRYQGSNNKR
IYNDIEMSRFRK

or is substantially similar to SEQ ID NO: 58, or is an active fragment of SEQ ID NO: 58. In some embodiments, the VSV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 58. In some embodiments, the VSV-G comprises an amino acid sequence of SEQ ID NO: 58.

In some embodiments, the VSV-G glycoprotein is of the Cocal strain and comprises an amino acid sequence having at least 70% identity to SEQ ID NO: 59:

(SEQ ID NO: 59)
KFSIVFPQSQKGNWKNVPSSYHYCPSSSDQNWHNDLLGITMKVKMPKTHK
AIQADGWMCHAAKWITTCDFRWYGPKYITHSIHSIQPTSEQCKESIKQTK
QGTWMSPGFPPQNCGYATVTDSVAVVVQATPHHVLVDEYTGEWIDSQFPN
GKCETEECETVHNSTVWYSDYKVTGLCDATLVDTEITFFSEDGKKESIGK
PNTGYRSNYFAYEKGDKVCKMNYCKHAGVRLPSGVWFEFVDQDVYAAAKL
PECPVGATISAPTQTSVDVSLILDVERILDYSLCQETWSKIRSKQPVSPV
DLSYLAPKNPGTGPAFTIINGTLKYFETRYIRIDIDNPIISKMVGKISGS
QTERELWIEWFPYEGVEIGPNGILKTPTGYKFPLFMIGHGMLDSDLHKTS
QAEVFEHPHLAEAPKQLPEEETLFFGDIGISKNPVELIEGWFSSWKSTVV
TFFFAIGVFILLYVVARIVIAVRYRYQGSNNKRIYNDIEMSRFRK

or is substantially similar to SEQ ID NO: 59, or is an active fragment of SEQ ID NO: 59. In some embodiments, the VSV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 59. In some embodiments, the VSV-G comprises an amino acid sequence of SEQ ID NO: 59.

In some embodiments, the VSV-G protein comprises a mutation at position 199 as compared to SEQ ID NO: 58 or at position 182 as compared to SEQ ID NO: 59. SEQ ID NO: 58 is the full length protein and SEQ ID NO: 59 is the ectodomain of the VSV-G protein. The 17-mer signal peptide of MNFLLLTFIVLPLCSHA (SEQ ID NO: 60) as shown at the N-terminus of SEQ ID NO: 58 is cleaved leaving a protein of SEQ ID NO: 59. Thus, although a mutation may be referred to in the context of SEQ ID NO: 59, it should be understood to also be made in the context of SEQ ID NO: 58, which contains the leader sequence, and thus would be a position number that is 17 more than the position recited for SEQ ID NO: 59. In some embodiments, the mutation inhibits or decreases the binding of the VSV-G protein to the LDL receptor. In some embodiments the mutation is V182D as compared to SEQ ID NO: 59. In some embodiments, the mutation is V182E as compared to SEQ ID NO: 59.

In some embodiments, a protein comprising a mutation at position 182 as compared to SEQ ID NO: 59 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identical as compared to SEQ ID NO: 59. In some embodiments, the polypeptide comprises a V182D or V182E mutation. In some embodiments, the VSV-G protein comprises a V182S, V182H, V182T, V182Q, or V182N mutation.

In some embodiments, the VSV-G glycoprotein is of the Morreton strain and comprises an amino acid sequence having at least 70% identity to SEQ ID NO: 61:

(SEQ ID NO: 61)
MLVLYLLLSLLALGAQCKFTIVFPHNQKGNWKNVPANYQYCPSSSDLNWH
NGLIGTSLQVKMPKSHKAIQADGWMCHAAKWVTTCDFRWYGPKYVTHSIK
SMIPTVDQCKESIAQTKQGTWLNPGFPPQSCGYASVTDAEAVIVKATPHQ
VLVDEYTGEWVDSQFPTGKCNKDICPTVHNSTTWHSDYKVTGLCDANLIS
MDITFFSEDGKLISLGKEGTGFRSNYFAYENGDKACRMQYCKHWGVRLPS
GVWFEMADKDIYNDAKFPDCPEGSSIAAPSQTSVDVSLIQDVERILDYSL
CQETWSKIRAHLPISPVDLSYLSPKNPGTGPAFTIINGTILKYFETRYIR
VDIAGPIIPQMRGVISGTTTERELWTDWYPYEDVEIGPNGVLKTATGYKF
PLYMIGHGMLDSDLHISSKAQVFEHPHIQDAASQLPDDETLFFGDTGLSK
NPIELVEGWFSGWKSTIASFFFIIGLVIGLYLVLRIGIALCIKCRVQEKR
PKIYTDVEMNRLDR

or is substantially similar to SEQ ID NO: 61, or is an active fragment of SEQ ID NO: 61. In some embodiments, the VSV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 61. In some embodiments, the VSV-G comprises an amino acid sequence of SEQ ID NO: 61.

In some embodiments, the VSV-G glycoprotein is of the Morreton strain and comprises an amino acid sequence having at least 70% identity to SEQ ID NO: 62:

(SEQ ID NO: 62)
KFTIVFPHNQKGNWKNVPANYQYCPSSSDLNWHNGLIGTSLQVKMPKSHK
AIQADGWMCHAAKWVTTCDFRWYGPKYVTHSIKSMIPTVDQCKESIAQTK
QGTWLNPGFPPQSCGYASVIDAEAVIVKATPHQVLVDEYTGEWVDSQFPT
GKCNKDICPTVHNSTTWHSDYKVTGLCDANLISMDITFFSEDGKLTSLGK
EGTGFRSNYFAYENGDKACRMQYCKHWGVRLPSGVWFEMADKDIYNDAKF
PDCPEGSSIAAPSQTSVDVSLIQDVERILDYSLCQETWSKIRAHLPISPV
DLSYLSPKNPGTGPAFTIINGTLKYFETRYIRVDIAGPIIPQMRGVISGI
TTERELWIDWYPYEDVEIGPNGVLKTATGYKFPLYMIGHGMLDSDLHISS
KAQVFEHPHIQDAASQLPDDETLEFGDTGLSKNPIELVEGWFSGWKSTIA
SFFFIIGLVIGLYLVLRIGIALCIKCRVQEKRPKIYTDVEMNRLDR

or is substantially similar to SEQ ID NO: 62, or is an active fragment of SEQ ID NO: 62. In some embodiments, the VSV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 62. In some embodiments, the VSV-G comprises an amino acid sequence of SEQ ID NO: 62.

In some embodiments, the VSV-G protein comprises a mutation at position 199 as compared to SEQ ID NO: 61 or at position 182 as compared to SEQ ID NO: 61. SEQ ID NO: 61 is the full length protein and SEQ ID NO: 62 is the ectodomain of the VSV-G protein. The 17-mer signal peptide of MLVLYLLLSLLALGAQC (SEQ ID NO: 63) as shown at the N-terminus of SEQ ID NO: 61 is cleaved leaving a protein of SEQ ID NO: 62. Thus, although a mutation may be referred to in the context of SEQ ID NO: 62, it should be understood to also be made in the context of SEQ ID NO: 61, which contains the leader sequence, and thus would be a position number that is 17 more than the position recited for SEQ ID NO: 62. In some embodiments, the mutation inhibits or decreases the binding of the VSV-G protein to the LDL receptor. In some embodiments the mutation is I182D as compared to SEQ ID NO: 62. In some embodiments, the mutation is 1182E as compared to SEQ ID NO: 62.

In some embodiments, a protein comprising a mutation at position 182 as compared to SEQ ID NO: 62 comprises a mutation at position 182 and at least, or about, 70%, 71%, 72%, 73%, 74%, 75%, 76%, 77%, 78%, 79%, 80%, 81%, 82%, 83%, 84%, 85%, 86%, 87%, 88%, 89%,90%,91%,92%,93%,94%,95%,96%,97%,98%,99% or 100% identical as compared to SEQ ID NO: 62. In some embodiments, the polypeptide comprises a I182D or 1182E mutation. In some embodiments, the VSV-G protein comprises a I182S, 1182H, 1182T, 1182Q, or I182N mutation.

Although, the mutations may be described in reference to SEQ ID NO 37 which is the VSV-G protein from the Indiana strain, the mutation can also be used in other strains of the VSV-G protein. For example, the mutation can be made in the New Jersey Strain of VSV-G, the Marraba strain of VSV-G, the Carajas strain of VSV-G, the Alagoa strain of VSV-G, the Cocal strain of VSV-G, or the Morreton strain of VSV-G. In some embodiments, the sequences of each are as provided herein. Examples of these can be found, for example in U.S. Patent Application Publication No. 20200216502, which is hereby incorporated by reference. For example, the wild-type ectodomain of the New Jersey Strain of VSV-G is SEQ ID NO: 47, the wild-type ectodomain of Marraba strain of VSV-G is SEQ ID NO: 50, the wild-type ectodomain of Carajas strain of VSV-G is SEQ ID NO: 53, the wild-type ectodomain of Alagoa strain of VSV-G is SEQ ID NO: 56, the wild-type ectodomain of Cocal strain of VSV-G is SEQ ID NO: 59, or the wild-type ectodomain of Morreton strain of VSV-G is SEQ ID NO: 62.

In some embodiments, the VSV-G protein comprising a mutation at position 182 as compared to SEQ ID NO: 37 further comprises a mutation at position that corresponds to positions 214 and/or 352 of SEQ ID NO: 37. In some embodiments, the residue that corresponds to position 214 of SEQ ID NO: 37 is T214. In some embodiments, the residue that corresponds to position 352 of SEQ ID NO: 37 is T352. In some embodiments, the VSV-G protein comprises mutation that corresponds to T214N mutation as compared to SEQ ID NO: 37. In some embodiments, the VSV-G protein comprises mutation that corresponds to T352A mutation as compared to SEQ ID NO: 37. In some embodiments, the VSV-G protein comprises a T214N and T352A mutations as compared to SEQ ID NO: 37. These mutations can be combined with any other mutations as provided for herein. In some embodiments, the T214N and/or T352A mutations are combined with the 1182E or 1182D mutations. In some embodiments, a VSV-G protein comprises an amino acid sequence of SEQ ID NO: 64 or SEQ ID NO: 65, which combines the 1182D or 11 82E, respectively, with the T214N and T352A mutations. The sequences are also illustrated below with the leader sequences, which are removed during protein processing.

VSV-G Protein_I196D, T230N and T368A mutations
(with leader sequence and adjusted numbering)
(SEQ ID NO: 66)
MKCLLYLAFLFIGVNCKFTIVFPHNQKGNWKNVPSNYHYCPSSSDLNWHN
DLIGTALQVKMPKSHKAIQADGWMCHASKWVTTCDFRWYGPKYITHSIRS
FTPSVEQCKESIEQTKQGTWLNPGFPPQSCGYATVIDAEAVIVQVTPHHV
LVDEYTGEWVDSQFINGKCSNYICPTVHNSTTWHSDYKVKGLCDSNLDSM
DITFFSEDGELSSLGKEGTGFRSNYFAYENGGKACKMQYCKHWGVRLPSG
VWFEMADKDLFAAARFPECPEGSSISAPSQTSVDVSLIQDVERILDYSLC
QETWSKIRAGLPISPVDLSYLAPKNPGTGPAFTIINGTLKYFETRYIRVD
IAAPILSRMVGMISGTTAERELWDDWAPYEDVEIGPNGVLRTSSGYKFPL
YMIGHGMLDSDLHLSSKAQVFEHPHIQDAASQLPDDESLFFGDTGLSKNP
IELVEGWFSSWKSSIASFFFIIGLIIGLFLVLRVGIHLCIKLKHTKKRQI
YTDIEMNRLGK
VSV-G Protein_I182D, T214N and T352A mutations
(without leader sequence)
(SEQ ID NO: 64)
KFTIVFPHNQKGNWKNVPSNYHYCPSSSDLNWHNDLIGTALQVKMPKSHK
AIQADGWMCHASKWVTTCDFRWYGPKYITHSIRSFTPSVEQCKESIEQTK
QGTWLNPGFPPQSCGYATVTDAEAVIVQVTPHHVLVDEYTGEWVDSQFIN
GKCSNYICPTVHNSTTWHSDYKVKGLCDSNLDSMDITFFSEDGELSSLGK
EGTGFRSNYFAYENGGKACKMQYCKHWGVRLPSGVWFEMADKDLFAAARF
PECPEGSSISAPSQTSVDVSLIQDVERILDYSLCQETWSKIRAGLPISPV
DLSYLAPKNPGTGPAFTIINGTLKYFETRYIRVDIAAPILSRMVGMISGT
TAERELWDDWAPYEDVEIGPNGVLRTSSGYKFPLYMIGHGMLDSDLHLSS
KAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK
VSV-G Protein with I196E, T230N and T368A mutations
(with leader sequence and adjusted numbering)
(SEQ ID NO: 67)
MKCLLYLAFLFIGVNCKFTIVFPHNQKGNWKNVPSNYHYCPSSSDLNWHN
DLIGTALQVKMPKSHKAIQADGWMCHASKWVTTCDFRWYGPKYITHSIRS
FTPSVEQCKESIEQTKQGTWLNPGFPPQSCGYATVTDAEAVIVQVTPHHV
LVDEYTGEWVDSQFINGKCSNYICPTVHNSTTWHSDYKVKGLCDSNLESM
DITFFSEDGELSSLGKEGTGFRSNYFAYENGGKACKMQYCKHWGVRLPSG
VWFEMADKDLFAAARFPECPEGSSISAPSQTSVDVSLIQDVERILDYSLC
QETWSKIRAGLPISPVDLSYLAPKNPGTGPAFTIINGTLKYFETRYIRVD
IAAPILSRMVGMISGTTAERELWDDWAPYEDVEIGPNGVLRTSSGYKFPL
YMIGHGMLDSDLHLSSKAQVFEHPHIQDAASQLPDDESLFFGDTGLSKNP
IELVEGWFSSWKSSIASFFFIIGLIIGLFLVLRVGIHLCIKLKHTKKRQI
YTDIEMNRLGK
VSV-G Protein with I182E, T214N and T352A mutations
(without leader sequences)
(SEQ ID NO: 65)
KFTIVFPHNQKGNWKNVPSNYHYCPSSSDLNWHNDLIGTALQVKMPKSHK
AIQADGWMCHASKWVTTCDFRWYGPKYITHSIRSFTPSVEQCKESIEQTK
QGTWLNPGFPPQSCGYATVTDAEAVIVQVTPHHVLVDEYTGEWVDSQFIN
GKCSNYICPTVHNSTTWHSDYKVKGLCDSNLESMDITFFSEDGELSSLGK
EGTGFRSNYFAYENGGKACKMQYCKHWGVRLPSGVWFEMADKDLFAAARF
PECPEGSSISAPSQTSVDVSLIQDVERILDYSLCQETWSKIRAGLPISPV
DLSYLAPKNPGTGPAFTIINGTLKYFETRYIRVDIAAPILSRMVGMISGT
TAERELWDDWAPYEDVEIGPNGVLRTSSGYKFPLYMIGHGMLDSDLHLSS
KAQVFEHPHIQDAASQLPDDESLFFGDTGLSKNPIELVEGWFSSWKSSIA
SFFFIIGLIIGLFLVLRVGIHLCIKLKHTKKRQIYTDIEMNRLGK

In some embodiments, the VSV-G protein comprising a mutation at position 182 as compared to SEQ ID NO: 37 further comprises a mutation at position that corresponds to positions 38 and/or 320 or SEQ ID NO: 37. In some embodiments, the residue that corresponds to position 38 of SEQ ID NO: 37 is T38. In some embodiments, the residue that corresponds to position 320 of SEQ ID NO: 37 is T320. In some embodiments, the VSV-G protein comprises mutation that corresponds to T38A mutation as compared to SEQ ID NO: 37. In some embodiments, the VSV-G protein comprises mutation that corresponds to T320A mutation as compared to SEQ ID NO: 37. In some embodiments, the VSV-G protein comprises a T38A and T320A mutations as compared to SEQ ID NO: 37. These mutations can be combined with any other mutations as provided for herein.

In some embodiments, the other strains of the VSV-G protein as described herein can further comprise one or more mutations corresponding to any of the other mutations as compared to SEQ ID NO: 37 and as provided for herein. For example, the other strains of the VSV-G protein as described herein can also comprises the mutations that correspond to T38A, T214N, T320A, and/or T352A in SEQ ID NO: 37. In some embodiments, the other strains of the VSV-G protein as described herein can also comprises the mutations that correspond to T214N and/or T352A in SEQ ID NO: 37 and as illustrated in SEQ ID NO: 64 and SEQ ID NO: 65.

In some embodiments, the affinity binding polypeptide is fused at the N-terminus of any of SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 66, SEQ ID NO: 67, or any variant thereof as provided for herein. In some embodiments, the affinity binding polypeptide is fused at the C-terminus of any of SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 66, SEQ ID NO: 67, or any variant thereof as provided for herein. In some embodiments, the one or more affinity binding polypeptides are fused at any location within or at the termini's of any of SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 66, SEQ ID NO: 67 or any variant thereof as provided for herein, provided that the inclusion does not substantially negatively affect the purpose of the glycoprotein of any of SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 66, SEQ ID NO: 67.

In some embodiments, the VSV-G fused to the affinity binding polypeptide comprises an amino acid sequence having at least 70% identity to SEQ ID NO: 68:

(SEQ ID NO: 68)
MKCLLYLAFLFIGVNCWSHPQFEKGGSKFTIVFPHNQKGNWKNVPSNYHY
CPSSSDLNWHNDLIGTALQVKMPKSHKAIQADGWMCHASKWVTTCDFRWY
GPKYITHSIRSFTPSVEQCKESIEQTKQGTWLNPGFPPQSCGYATVTDAE
AVIVQVTPHHVLVDEYTGEWVDSQFINGKCSNYICPTVHNSTTWHSDYKV
KGLCDSNLESMDITFFSEDGELSSLGKEGTGFRSNYFAYENGGKACKMQY
CKHWGVRLPSGVWFEMADKDLFAAARFPECPEGSSISAPSQTSVDVSLIQ
DVERILDYSLCQETWSKIRAGLPISPVDLSYLAPKNPGTGPAFTIINGTL
KYFETRYIRVDIAAPILSRMVGMISGTTAERELWDDWAPYEDVEIGPNGV
LRTSSGYKFPLYMIGHGMLDSDLHLSSKAQVFEHPHIQDAASQLPDDESL
FFGDTGLSKNPIELVEGWFSSWKSSIASFFFIIGLIIGLFLVLRVGIHLC
IKLKHTKKRQIYTDIEMNRLGK

or is substantially similar to SEQ ID NO: 68. (Italicized amino acids represent the VSV-G signal peptide, bold and underlined sequence represents the affinity binding polypeptide, bolded sequence no underline represents linker peptide). In some embodiments, the VSV-G fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 68. In some embodiments, the VSV-G fused to the affinity binding polypeptide comprises an amino acid sequence of SEQ ID NO: 68.

In some embodiments, the VSV-G fused to the affinity binding polypeptide comprises an amino acid sequence having at least 70% identity to SEQ ID NO: 69:

(SEQ ID NO: 69)
WSHPQFEKGGSKFTIVFPHNQKGNWKNVPSNYHYCPSSSDLNWHNDLIGT
ALQVKMPKSHKAIQADGWMCHASKWVTTCDFRWYGPKYITHSIRSFTPSV
EQCKESIEQTKQGTWLNPGFPPQSCGYATVTDAEAVIVQVTPHHVLVDEY
TGEWVDSQFINGKCSNYICPTVHNSTTWHSDYKVKGLCDSNLESMDITFF
SEDGELSSLGKEGTGFRSNYFAYENGGKACKMQYCKHWGVRLPSGVWFEM
ADKDLFAAARFPECPEGSSISAPSQTSVDVSLIQDVERILDYSLCQETWS
KIRAGLPISPVDLSYLAPKNPGTGPAFTIINGTLKYFETRYIRVDIAAPI
LSRMVGMISGTTAERELWDDWAPYEDVEIGPNGVLRTSSGYKFPLYMIGH
GMLDSDLHLSSKAQVFEHPHIQDAASQLPDDESLFFGDTGLSKNPIELVE
GWFSSWKSSIASFFFIIGLIIGLFLVLRVGIHLCIKLKHTKKRQIYTDIE
MNRLGK

or is substantially similar to SEQ ID NO: 69. (Bold and underlined sequence represents the affinity binding polypeptide, bolded sequence no underline represents linker peptide). In some embodiments, the VSV-G fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 69. In some embodiments, the VSV-G fused to the affinity binding polypeptide comprises an amino acid sequence of SEQ ID NO: 69.

In some embodiments, the VSV-G fused to the affinity binding polypeptide comprises an amino acid sequence having at least 70% identity to SEQ ID NO: 70:

(SEQ ID NO: 70)
WSHPQFEKKFTIVFPHNQKGNWKNVPSNYHYCPSSSDLNWHNDLIGTALQ
VKMPKSHKAIQADGWMCHASKWVTTCDFRWYGPKYITHSIRSFTPSVEQC
KESIEQTKQGTWLNPGFPPQSCGYATVTDAEAVIVQVTPHHVLVDEYTGE
WVDSQFINGKCSNYICPTVHNSTTWHSDYKVKGLCDSNLESMDITFFSED
GELSSLGKEGTGFRSNYFAYENGGKACKMQYCKHWGVRLPSGVWFEMADK
DLFAAARFPECPEGSSISAPSQTSVDVSLIQDVERILDYSLCQETWSKIR
AGLPISPVDLSYLAPKNPGTGPAFTIINGILKYFETRYIRVDIAAPILSR
MVGMISGTTAERELWDDWAPYEDVEIGPNGVLRTSSGYKFPLYMIGHGML
DSDLHLSSKAQVFEHPHIQDAASQLPDDESLFFGDTGLSKNPIELVEGWF
SSWKSSIASFFFIIGLIIGLFLVLRVGIHLCIKLKHTKKRQIYTDIEMNR
LGK

or is substantially similar to SEQ ID NO: 70. (Bold and underlined sequence represents the affinity binding polypeptide). In some embodiments, the VSV-G fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 70. In some embodiments, the VSV-G fused to the affinity binding polypeptide comprises an amino acid sequence of SEQ ID NO: 70.

In some embodiments, the VSV-G fused to the affinity binding polypeptide comprises an amino acid sequence having at least 70% identity to SEQ ID NO: 71:

(SEQ ID NO: 71)
KWSHPQFEKFTIVFPHNQKGNWKNVPSNYHYCPSSSDLNWHNDLIGTALQ
VKMPKSHKAIQADGWMCHASKWVTTCDFRWYGPKYITHSIRSFTPSVEQC
KESIEQTKQGTWLNPGFPPQSCGYATVTDAEAVIVQVTPHHVLVDEYTGE
WVDSQFINGKCSNYICPTVHNSTTWHSDYKVKGLCDSNLESMDITFFSED
GELSSLGKEGTGFRSNYFAYENGGKACKMQYCKHWGVRLPSGVWFEMADK
DLFAAARFPECPEGSSISAPSQTSVDVSLIQDVERILDYSLCQETWSKIR
AGLPISPVDLSYLAPKNPGTGPAFTIINGTLKYFETRYIRVDIAAPILSR
MVGMISGTTAERELWDDWAPYEDVEIGPNGVLRTSSGYKFPLYMIGHGML
DSDLHLSSKAQVFEHPHIQDAASQLPDDESLFFGDTGLSKNPIELVEGWF
SSWKSSIASFFFIIGLIIGLFLVLRVGIHLCIKLKHTKKRQIYTDIEMNR
LGK

or is substantially similar to SEQ ID NO: 71. (Bold and underlined sequence represents the affinity binding polypeptide). It is to be understood that the sequence of SEQ ID NO: 71 may further comprise an appropriate signal peptide as provided for herein and the affinity binding polypeptide may be flanked on either or both sides by a linker peptide as provided for herein.

Accordingly, in some embodiments, the VSV-G fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 71. In some embodiments, the VSV-G fused to the affinity binding polypeptide comprises an amino acid sequence of SEQ ID NO: 71.

In some embodiments, the VSV-G fused to the affinity binding polypeptide comprises an amino acid sequence having at least 70% identity to SEQ ID NO: 72:

(SEQ ID NO: 72)
KFTIVFPWSHPQFEKHNQKGNWKNVPSNYHYCPSSSDLNWHNDLIGTALQ
VKMPKSHKAIQADGWMCHASKWVTTCDFRWYGPKYITHSIRSFTPSVEQC
KESIEQTKQGTWLNPGFPPQSCGYATVTDAEAVIVQVTPHHVLVDEYTGE
WVDSQFINGKCSNYICPTVHNSTTWHSDYKVKGLCDSNLESMDITFFSED
GELSSLGKEGTGFRSNYFAYENGGKACKMQYCKHWGVRLPSGVWFEMADK
DLFAAARFPECPEGSSISAPSQTSVDVSLIQDVERILDYSLCQETWSKIR
AGLPISPVDLSYLAPKNPGTGPAFTIINGTLKYFETRYIRVDIAAPILSR
MVGMISGTTAERELWDDWAPYEDVEIGPNGVLRTSSGYKFPLYMIGHGML
DSDLHLSSKAQVFEHPHIQDAASQLPDDESLFFGDTGLSKNPIELVEGWF
SSWKSSIASFFFIIGLIIGLFLVLRVGIHLCIKLKHTKKRQIYTDIEMNR
LGK

or is substantially similar to SEQ ID NO: 72. (Bold and underlined sequence represents the affinity binding polypeptide). It is to be understood that the sequence of SEQ ID NO: 72 may further comprise an appropriate signal peptide as provided for herein and the affinity binding polypeptide may be flanked on either or both sides by a linker peptide as provided for herein.

Accordingly, in some embodiments, the VSV-G fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 72. In some embodiments, the VSV-G fused to the affinity binding polypeptide comprises an amino acid sequence of SEQ ID NO: 72.

In some embodiments, the VSV-G fused to the affinity binding polypeptide comprises an amino acid sequence having at least 70% identity to SEQ ID NO: 73:

(SEQ ID NO: 73)
KFTIVFPHNQKGNWKNVPSNYHYCPSSSDLNWHNDLIGTALQVKMPKSHK
AIQADGWMCHASKWVTTCDFRWYGPKYITHSIRSFTPSVEQCKESIEQTK
QGTWLNPGFPPQSCGYATVIDAEAVIVQVTPHHVLVDEYTGEWVDSQFIN
GKCSNYICPTVHNSTTWHSDYKVKWSHPQFEKGLCDSNLESMDITFFSED
GELSSLGKEGTGFRSNYFAYENGGKACKMQYCKHWGVRLPSGVWFEMADK
DLFAAARFPECPEGSSISAPSQTSVDVSLIQDVERILDYSLCQETWSKIR
AGLPISPVDLSYLAPKNPGTGPAFTIINGTLKYFETRYIRVDIAAPILSR
MVGMISGTTAERELWDDWAPYEDVEIGPNGVLRTSSGYKFPLYMIGHGML
DSDLHLSSKAQVFEHPHIQDAASQLPDDESLFFGDTGLSKNPIELVEGWF
SSWKSSIASFFFIIGLIIGLFLVLRVGIHLCIKLKHTKKRQIYTDIEMNR
LGK

or is substantially similar to SEQ ID NO: 73. (Bold and underlined sequence represents the affinity binding polypeptide). It is to be understood that the sequence of SEQ ID NO: 73 may further comprise an appropriate signal peptide as provided for herein and the affinity binding polypeptide may be flanked on either or both sides by a linker peptide as provided for herein.

Accordingly, in some embodiments, the VSV-G fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 73. In some embodiments, the VSV-G fused to the affinity binding polypeptide comprises an amino acid sequence of SEQ ID NO: 73.

In some embodiments, the VSV-G fused to the affinity binding polypeptide comprises an amino acid sequence having at least 70% identity to SEQ ID NO: 132:

(SEQ ID NO: 132)
MKCLLYLAFLFIGVNCKFSGGWSHPQFEKGGSTIVFPHNQKGNWKNVPSN
YHYCPSSSDLNWHNDLIGTALQVKMPKSHKAIQADGWMCHASKWVTTCDF
RWYGPKYITHSIRSFTPSVEQCKESIEQTKQGTWLNPGFPPQSCGYATVI
DAEAVIVQVTPHHVLVDEYTGEWVDSQFINGKCSNYICPTVHNSTTWHSD
YKVKGLCDSNLESMDITFFSEDGELSSLGKEGTGFRSNYFAYENGGKACK
MQYCKHWGVRLPSGVWFEMADKDLFAAARFPECPEGSSISAPSQTSVDVS
LIQDVERILDYSLCQETWSKIRAGLPISPVDLSYLAPKNPGTGPAFTIIN
GTLKYFETRYIRVDIAAPILSRMVGMISGTTAERELWDDWAPYEDVEIGP
NGVLRTSSGYKFPLYMIGHGMLDSDLHLSSKAQVFEHPHIQDAASQLPDD
ESLFFGDIGLSKNPIELVEGWFSSWKSSIASFFFIIGLIIGLFLVLRVGI
HLCIKLKHTKKRQIYTDIEMNRLGK

or is substantially similar to SEQ ID NO: 132. (Italicized amino acids represent the VSV-G signal peptide, bold and underlined sequence represents the affinity binding polypeptide, bolded sequence no underline represents linker peptide). The sequence of SEQ ID NO: 132 contains the VSV-G signal peptide MKCLLYLAFLFIGVNC (SEQ ID NO: 38). Upon processing, the signal peptide may be cleaved leaving the sequence of SEQ ID NO: 167 (See Table 6). Thus, while embodiments may refer to further modifications of, mutations of, or percent identity to SEQ ID NO: 132, it is to be understood that the same modifications, mutations, and limitations to percent identity also apply to SEQ ID NO: 167. It is to be understood that the sequence of SEQ ID NO: 132 may further comprise an appropriate signal peptide as provided for herein and the affinity binding polypeptide may be flanked on either or both sides by a linker peptide as provided for herein. Accordingly, in some embodiments, the VSV-G fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 132. In some embodiments, the VSV-G fused to the affinity binding polypeptide comprises an amino acid sequence of SEQ ID NO: 132.

In some embodiments, the VSV-G fused to the affinity binding polypeptide comprises an amino acid sequence having at least 70% identity to SEQ ID NO: 133:

(SEQ ID NO: 133)
MKCLLYLAFLFIGVNCKFTIVFPSGGWSHPQFEKGGSHNQKGNWKNVPSN
YHYCPSSSDLNWHNDLIGTALQVKMPKSHKAIQADGWMCHASKWVTTCDF
RWYGPKYITHSIRSFTPSVEQCKESIEQTKQGTWLNPGFPPQSCGYATVI
DAEAVIVQVTPHHVLVDEYTGEWVDSQFINGKCSNYICPTVHNSTTWHSD
YKVKGLCDSNLESMDITFFSEDGELSSLGKEGTGFRSNYFAYENGGKACK
MQYCKHWGVRLPSGVWFEMADKDLFAAARFPECPEGSSISAPSQTSVDVS
LIQDVERILDYSLCQETWSKIRAGLPISPVDLSYLAPKNPGTGPAFTIIN
GTLKYFETRYIRVDIAAPILSRMVGMISGTTAERELWDDWAPYEDVEIGP
NGVLRTSSGYKFPLYMIGHGMLDSDLHLSSKAQVFEHPHIQDAASQLPDD
ESLFFGDTGLSKNPIELVEGWFSSWKSSIASFFFIIGLIIGLFLVLRVGI
HLCIKLKHTKKRQIYTDIEMNRLGK

or is substantially similar to SEQ ID NO: 133. (Italicized amino acids represent the VSV-G signal peptide, bold and underlined sequence represents the affinity binding polypeptide, bolded sequence no underline represents linker peptide). The sequence of SEQ ID NO: 133 contains the VSV-G signal peptide MKCLLYLAFLFIGVNC (SEQ ID NO: 38). Upon processing, the signal peptide may be cleaved leaving the sequence of SEQ ID NO: 168 (See Table 6). Thus, while embodiments may refer to further modifications of, mutations of, or percent identity to SEQ ID NO: 133, it is to be understood that the same modifications, mutations, and limitations to percent identity also apply to SEQ ID NO: 168. It is to be understood that the sequence of SEQ ID NO: 133 may further comprise an appropriate signal peptide as provided for herein and the affinity binding polypeptide may be flanked on either or both sides by a linker peptide as provided for herein. Accordingly, in some embodiments, the VSV-G fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 133. In some embodiments, the VSV-G fused to the affinity binding polypeptide comprises an amino acid sequence of SEQ ID NO: 133.

In some embodiments, affinity binding peptides as provided for herein are fused to MeV-H. In some embodiments, MeV-H comprises an amino acid sequence having at least 70% identity to SEQ ID NO 74:

(SEQ ID NO: 74)
MGSRIVINREHLMIDRPYVLLAVLFVMFLSLIGLLAIAGIRLHRAAIYTA
EIHKSLSTNLDVINSIEHQVKDVLTPLFKIIGDEVGLRTPQRFTDLVKFI
SDKIKFLNPDREYDFRDLTWCINPPERIKLDYDQYCADVAAEELMNALVN
STLLETRTTNQFLAVSKGNCSGPTTIRGQFSNMSLSLLDLYLGRGYNVSS
IVTMTSQGMYGGTYLVEKPNLSSKRSELSQLSMYRVFEVGVIRNPGLGAP
VFHMTNYLEQPVSNDLSNCMVALGELKLAALCHGEDSITIPYQGSGKGVS
FQLVKLGVWKSPTDMQSWVPLSTDDPVIDRLYLSSHRGVIADNQAKWAVP
TTRTDDKLRMETCFQQACKGKIQALCENPEWAPLKDNRIPSYGVLSVDLS
LTVELKIKIASGFGPLITHGSGMDLYKSNHNNVYWLTIPPMKNLALGVIN
TLEWIPRFKVSPALFNVPIKEAGEDCHAPTYLPAEVDGDVKLSSNLVILP
GQDLQYVLATYDTSAVEHAVVYYVYSPSRLSSYFYPFRLPIKGVPIELQV
ECFTWDQKLWCRHFCVLADSESGGHITHSGMVGMGVSCTVTREDGTNRRG
G

or is substantially similar to SEQ ID NO: 74, or is an active fragment of SEQ ID NO: 74. In some embodiments, the MeV-H comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 74. In some embodiments, the MeV-H comprises an amino acid sequence of SEQ ID NO: 74.

In some embodiments, the MeV-H protein of SEQ ID NO: 74 is truncated. In some embodiments, the truncated MeV-H protein comprises HcΔ14, HcΔ15, HcΔ16, HcΔ17, HcΔ18, HcΔ19, HcΔ20, HcΔ21+A and HcΔ24+4A as described in U.S. Pat. No. 10,415,057, which is hereby incorporated by reference.

In some embodiments, HcΔ14 refers to a truncated MeV-H protein wherein 14 residues of the cytoplasmic portion have been deleted. In some embodiments, HcΔ14 refers to a truncated MeV-H wherein amino acids 2-15 of MeV-H have been deleted. In some embodiments, HcΔ15 refers to a truncated MeV-H protein wherein 15 residues of the cytoplasmic portion have been deleted. In some embodiments, HcΔ15 refers to a truncated MeV-H wherein amino acids 2-16 of MeV-H have been deleted. In some embodiments, HcΔ16 refers to a truncated MeV-H protein wherein 16 residues of the cytoplasmic portion have been deleted. In some embodiments, HcΔ16 refers to a truncated MeV-H wherein amino acids 2-17 of MeV-H have been deleted. In some embodiments, HcΔ17 refers to a truncated MeV-H protein wherein 17 residues of the cytoplasmic portion have been deleted. In some embodiments, HcΔ17 refers to a truncated MeV-H wherein amino acids 2-18 of MeV-H have been deleted. In some embodiments, HcΔ18 refers to a truncated MeV-H protein wherein 18 residues of the cytoplasmic portion have been deleted. In some embodiments, HcΔ18 refers to a truncated MeV-H wherein amino acids 2-19 of MeV-H have been deleted. In some embodiments, HcΔ19 refers to a truncated MeV-H protein wherein 19 residues of the cytoplasmic portion have been deleted. In some embodiments, HcΔ19 refers to a truncated MeV-H wherein amino acids 2-20 of MeV-H have been deleted. In some embodiments, HcΔ20 refers to a truncated MeV-H protein wherein 20 residues of the cytoplasmic portion have been deleted. In some embodiments, HcΔ20 refers to a truncated MeV-H wherein amino acids 2-21 of MeV-H have been deleted.

In some embodiments, HcΔ21+A refers to a truncated MeV-H protein wherein 21 residues of the cytoplasmic portion have been deleted and an alanine has been inserted at the N-terminal portion of the remaining cytoplasmic portion. In some embodiments, HcΔ21+A refers to a truncated MeV-H protein wherein amine acids 2-22 of MeV-H have been deleted and an alanine has been inserted at the N-terminal portion of the remaining cytoplasmic portion. Accordingly, in some embodiments, HcΔ21+A may have the formula M-A-(MeV-H AA23), wherein M is methionine, A is the inserted alanine, and MeV-H AA23 is the 23^{rd} amino acid of the MeV-H full length protein. In some embodiments, HcΔ24+4A refers to a truncated MeV-H protein wherein 24 residues of the cytoplasmic portion have been deleted and four alanine residues have been inserted at the N-terminal portion of the remaining cytoplasmic portion. In some embodiments, HcΔ24+4A refers to a truncated MeV-H protein wherein amine acids 2-25 of MeV-H have been deleted and four alanine residues have been inserted at the N-terminal portion of the remaining cytoplasmic portion. Accordingly, in some embodiments, HcΔ24+A may have the formula M-AAAA-(MeV-H AA26), wherein M is methionine, AAAA (SEQ ID NO: 134) are the four inserted alanine residues, and MeV-H AA26 is the 26^{st} amino acid of the MeV-H full length protein.

In some embodiments, the truncated MeV-H protein comprises a sequence of SEQ ID NO: 74.

In some embodiments, the affinity binding polypeptide is fused at the N-terminus of SEQ ID NO: 74, or a variant thereof as provided for herein. In some embodiments, the affinity binding polypeptide is fused at the C-terminus of SEQ ID NO: 74, or a variant thereof as provided for herein. In some embodiments, the one or more affinity binding polypeptides are fused at any location within or at the termini's of SEQ ID NO: 74, or a variant thereof as provided for herein, provided that the inclusion does not substantially negatively affect the purpose of the glycoprotein of SEQ ID NO: 74.

In some embodiments, the MeV-H fused to the affinity binding polypeptide comprises an amino acid sequence having at least 70% identity to SEQ ID NO: 75:

(SEQ ID NO: 75)
MGSRIVINREHLMIDRPYVLLAVLFVMFLSLIGLLAIAGIRLHRAAIYTA
EIHKSLSTNLDVTNSIEHQVKDVLTPLFKIIGDEVGLRTPQRFTDLVKFI
SDKIKFLNPDREYDFRDLTWCINPPERIKLDYDQYCADVAAEELMNALVN
STLLETRTTNQFLAVSKGNCSGPTTIRGQFSNMSLSLLDLYLGRGYNVSS
IVTMTSQGMYGGTYLVEKPNLSSKRSELSQLSMYRVFEVGVIRNPGLGAP
VFHMTNYLEQPVSNDLSNCMVALGELKLAALCHGEDSITIPYQGSGKGVS
FQLVKLGVWKSPTDMQSWVPLSTDDPVIDRLYLSSHRGVIADNQAKWAVP
TTRIDDKLRMETCFQQACKGKIQALCENPEWAPLKDNRIPSYGVLSVDLS
LTVELKIKIASGFGPLITHGSGMDLYKSNHNNVYWLTIPPMKNLALGVIN
TLEWIPRFKVSPALFNVPIKEAGEDCHAPTYLPAEVDGDVKLSSNLVILP
GQDLQYVLATYDTSAVEHAVVYYVYSPSRLSSYFYPERLPIKGVPIELQV
ECFTWDQKLWCRHFCVLADSESGGHITHSGMVGMGVSCTVTREDGTNRRG
GDIGGSWSHPQFEK

or is substantially similar to SEQ ID NO: 75. (Bold and underlined sequence represents the affinity binding polypeptide, bolded sequence no underline represents linker peptide). In some embodiments, the MeV-H fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 75. In some embodiments, the MeV-H fused to the affinity binding polypeptide comprises an amino acid sequence of SEQ ID NO: 75.

In some embodiments, a MeV-H fused to the affinity binding polypeptide further comprises a targeting moiety. In some embodiments, the MeV-H comprises an amino acid sequence as provided for herein. In some embodiments, the targeting moiety is fused directly to the MeV-H protein. In some embodiments, the targeting moiety is fused indirectly to the MeV-H protein via, for example, a peptide linker as provided for herein. In some embodiments, the affinity binding polypeptide is located on the N-terminus of the MeV-H:targeting moiety fusion construct. In some embodiments, the affinity binding polypeptide is located on the C-terminus of the MeV-H:targeting moiety fusion construct. In some embodiments, the affinity binding polypeptide is located between the MeV-H protein and the targeting moiety. In some embodiments, the affinity binding polypeptide is located within the MeV-H protein as provided for herein. In some embodiments, the affinity binding polypeptide is located within the targeting moiety as provided for herein. In some embodiments, the MeV-H fused to the affinity binding polypeptide further comprising a targeting moiety comprises an amino acid sequence having at least 70% identity to SE ID NO: 76:

(SEQ ID NO: 76)
MGSRIVINREHLMIDRPYVLLAVLFVMFLSLIGLLAIAGIRLHRAAIYTA
EIHKSLSTNLDVTNSIEHQVKDVLTPLFKIIGDEVGLRTPQRFTDLVKFI
SDKIKFLNPDREYDFRDLTWCINPPERIKLDYDQYCADVAAEELMNALVN
STLLETRTTNQFLAVSKGNCSGPTTIRGQFSNMSLSLLDLYLGRGYNVSS
IVTMTSQGMYGGTYLVEKPNLSSKRSELSQLSMYRVFEVGVIRNPGLGAP
VFHMTNYLEQPVSNDLSNCMVALGELKLAALCHGEDSITIPYQGSGKGVS
FQLVKLGVWKSPTDMQSWVPLSTDDPVIDRLYLSSHRGVIADNQAKWAVP
TTRTDDKLRMETCFQQACKGKIQALCENPEWAPLKDNRIPSYGVLSVDLS
LTVELKIKIASGFGPLITHGSGMDLYKSNHNNVYWLTIPPMKNLALGVIN
TLEWIPRFKVSPALFNVPIKEAGEDCHAPTYLPAEVDGDVKLSSNLVILP
GQDLQYVLATYDTSAVEHAVVYYVYSPSRLSSYFYPFRLPIKGVPIELQV
ECFTWDQKLWCRHFCVLADSESGGHITHSGMVGMGVSCTVTREDGTNRRG
GDIGGGGSGGGGSGGGGSGGGGSQVQLQQPGAELVKPGASVKLSCKASGY
PFTSYWIHWVKQRPGRGLEWLGRIDPNSGDTKYNEKFKNKATLTVDKSST
TAYMQLSSLTSEDSAVYYCARSPYYSNDNSMDYWGQGTSVTVSSGGGGSG
GGGSGGGGSGGGGSDILLTQSPAILSVSPGERVSFSCRASQSIGTSIHWY
QQRTNDSPRLLIKYASESISGIPSRFSGSGSGTDFTLSINSVESEDIADY
YCQQSNSWPTTFGGGTKLEIKRGGSWSHPQFEK

or is substantially similar to SEQ ID NO: 76. (Bold and underlined sequence represents the affinity binding polypeptide, bolded sequence no underline represents linker peptide, underlined sequence represents the targeting moiety). In some embodiments, the MeV-H fused to the affinity binding polypeptide further comprising a targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 76. In some embodiments, the MeV-H fused to the affinity binding polypeptide further comprising a targeting moiety comprises an amino acid sequence of SEQ ID NO: 76.

In some embodiments, affinity binding peptides as provided for herein are fused to MeV-H. In some embodiments, MeV-F comprises an amino acid sequence having at least 70% identity to SEQ ID NO 77:

(SEQ ID NO: 77)
MGLKVNVSAIFMAVLLTLQTPTGQIHWGNLSKIGVVGIGSASYKVMTRSS
HQSLVIKLMPNITLLNNCTRVEIAEYRRLLRTVLEPIRDALNAMTQNIRP
VQSVASSRRHKRFAGVVLAGAALGVATAAQITAGIALHQSMLNSQAIDNL
RASLETTNQAIEAIRQAGQEMILAVQGVQDYINNELIPSMNQLSCDLIGQ
KLGLKLLRYYTEILSLFGPSLRDPISAEISIQALSYALGGDINKVLEKLG
YSGGDLLGILESRGIKARITHVDTESYLIVLSIAYPTLSEIKGVIVHRLE
GVSYNIGSQEWYTTVPKYVATQGYLISNFDESSCTFMPEGTVCSQNALYP
MSPLLQECLRGSTKSCARTLVSGSFGNRFILSQGNLIANCASILCKCYTT
GTIINQDPDKILTYIAADHCPVVEVNGVTIQVGSRRYPDAVYLHRIDLGP
PILLERLDVGTNLGNAIAKLEDAKELLESSDQILRSMKGLSSTCIVYILI
AVCLGGLIGIPALICCCRGR

or is substantially similar to SEQ ID NO: 77, or is an active fragment of SEQ ID NO: 77. In some embodiments, the MeV-F comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 77. In some embodiments, the MeV-F comprises an amino acid sequence of SEQ ID NO: 77.

In some embodiments, the MeV-F protein comprises a truncated cytoplasmic portion. In some embodiments, the truncated cytoplasmic portion of the F protein comprises at least 1 positively charged amino acid residue and no more than 9 consecutive amino acid residues as counted from the N-terminal end of the cytoplasmic portion of the F protein. In some embodiments, the truncated cytoplasmic portion of the measles F protein comprises a sequence of SEQ ID NO: 77.

In some embodiments, the affinity binding polypeptide is fused at the N-terminus of SEQ ID NO: 77, or a variant thereof as provided for herein. In some embodiments, the affinity binding polypeptide is fused at the C-terminus of SEQ ID NO: 77, or a variant thereof as provided for herein. In some embodiments, the one or more affinity binding polypeptides are fused at any location within or at the termini's of SEQ ID NO: 77, or a variant thereof as provided for herein, provided that the inclusion does not substantially negatively affect the purpose of the glycoprotein of SEQ ID NO: 77.

In some embodiments, the MeV-F fused to the affinity binding polypeptide comprises an amino acid sequence having at least 70% identity to SEQ ID NO: 78:

(SEQ ID NO: 78)
MGLKVNVSAIFMAVLLTLQTPTGWSHPQFEKGGSQIHWGNLSKIGVVGIG
SASYKVMTRSSHQSLVIKLMPNITLLNNCTRVEIAEYRRLLRTVLEPIRD
ALNAMTQNIRPVQSVASSRRHKRFAGVVLAGAALGVATAAQITAGIALHQ
SMLNSQAIDNLRASLETTNQAIEAIRQAGQEMILAVQGVQDYINNELIPS
MNQLSCDLIGQKLGLKLLRYYTEILSLFGPSLRDPISAEISIQALSYALG
GDINKVLEKLGYSGGDLLGILESRGIKARITHVDTESYFIVLSIAYPTLS
EIKGVIVHRLEGVSYNIGSQEWYTTVPKYVATQGYLISNFDESSCTFMPE
GTVCSQNALYPMSPLLQECLRGSTKSCARTLVSGSFGNRFILSQGNLIAN
CASILCKCYTTGTIINQDPDKILTYIAADHCPVVEVNGVTIQVGSRRYPD
AVYLHRIDLGPPISLERLDVGTNLGNAIAKLEDAKELLESSDQILRSMKG
LSSTSIVYILIAVCLGGLIGIPALICCCRGR

or is substantially similar to SEQ ID NO: 78. (Bold and underlined sequence represents the affinity binding polypeptide, bolded sequence no underline represents linker peptide, italic sequence represents the MeV-F signal peptide). The sequence of SEQ ID NO: 78 contains the MeV-F signal peptide MGLKVNVSAIFMAVLLTLQTPTG (SEQ ID NO: 169). Upon processing, the signal peptide may be cleaved leaving the sequence of SEQ ID NO: 170 (See Table 6). Thus, while embodiments may refer to further modifications of, mutations of, or percent identity to SEQ ID NO: 78, it is to be understood that the same modifications, mutations, and limitations to percent identity also apply to SEQ ID NO: 170. In some embodiments, the MeV-F fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 78. In some embodiments, the MeV-F fused to the affinity binding polypeptide comprises an amino acid sequence of SEQ ID NO: 78.

In some embodiments, affinity binding peptides as provided for herein are fused to SVCV-G. In some embodiments, SVCV-G comprises an amino acid sequence having at least 70% identity to SEQ ID NO 79:

(SEQ ID NO: 79)
MSIISYIAFLLLIDSNLGIPIFVPSGRNISWQPVIQPFDYQCPIHGNLPN
TMGLSATKLTIKSPSVFSTDKVSGWICHAAEWKTTCDYRWYGPQYITHSI
HPISPTIDECRRIIQRIASGTDEDLGFPPQSCGWASVTTVSNTNYRVVPH
SVHLEPYGGHWIDHEFNGGECREKVCEMKGNHSIWITEETVQHECAKHIE
EVEGIMYGNVPRGDVMYANNFIIDRHHRVYRFGGSCQMKFCNKDGIKFAR
GDWVEKTAGTLTTIHDNVPKCVDGTLVSGHRPGLDLIDTVFNLENVVEYT
LCEGTKRKINKQEKLTSVDLSYLAPRIGGFGSVFRVRNGTLERGSTTYIR
IEVEGPIVDSLNGTDPRTNASRVFWDDWELDGNIYQGFNGVYKGKDGKIH
IPLNMIESGIIDDELQHAFQADIIPHPHYDDDEIREDDIFFDNTGENGNP
VDAVVEWVSGWGTSLKFFGMTLVALILIFLLIRCCVACTYLMKRSKRPAT
ESHEMRSLV

or is substantially similar to SEQ ID NO: 79, or is an active fragment of SEQ ID NO: 79. In some embodiments, the SVCV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 79. In some embodiments, the SVCV-G comprises an amino acid sequence of SEQ ID NO: 79.

In some embodiments, SVCV-G is provided without a leader sequence. In some embodiments, SVCV-G without a leader sequence comprises an amino acid sequence having at least 70% identity to SEQ ID NO 80:

(SEQ ID NO: 80)
IPIFVPSGRNISWQPVIQPFDYQCPIHGNLPNTMGLSATKLTIKSPSVF
STDKVSGWICHAAEWKITCDYRWYGPQYITHSIHPISPTIDECRRIIQR
IASGTDEDLGFPPQSCGWASVTTVSNINYRVVPHSVHLEPYGGHWIDHE
FNGGECREKVCEMKGNHSIWITEETVQHECAKHIEEVEGIMYGNVPRGD
VMYANNFIIDRHHRVYRFGGSCQMKFCNKDGIKFARGDWVEKTAGTLTT
IHDNVPKCVDGTLVSGHRPGLDLIDTVFNLENVVEYTLCEGTKRKINKQ
EKLTSVDLSYLAPRIGGFGSVFRVRNGTLERGSTTYIRIEVEGPIVDSL
NGTDPRTNASRVFWDDWELDGNIYQGFNGVYKGKDGKIHIPLNMIESGI
IDDELQHAFQADIIPHPHYDDDEIREDDIFFDNTGENGNPVDAVVEWVS
GWGTSLKFFGMTLVALILIFLLIRCCVACTYLMKRSKRPATESHEMRSL
V

or is substantially similar to SEQ ID NO: 80, or is an active fragment of SEQ ID NO: 80. In some embodiments, the SVCV-G comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 80. In some embodiments, the SVCV-G comprises an amino acid sequence of SEQ ID NO: 80.

In some embodiments, the affinity binding polypeptide is fused at the N-terminus of SEQ ID NO: 79 or SEQ ID NO: 80, or a variant thereof as provided for herein. In some embodiments, the affinity binding polypeptide is fused at the C-terminus of SEQ ID NO: 79 or SEQ ID NO: 80, or a variant thereof as provided for herein. In some embodiments, the one or more affinity binding polypeptides are fused at any location within or at the termini's of SEQ ID NO: 79 or SEQ ID NO: 80, or a variant thereof as provided for herein, provided that the inclusion does not substantially negatively affect the purpose of the glycoprotein of SEQ ID NO: 79 or SEQ ID NO: 80.

In some embodiments, the SVCV-G fused to the affinity binding polypeptide comprises an amino acid sequence having at least 70% identity to SEQ ID NO: 81:

(SEQ ID NO: 81)
MSIISYIAFLLLIDSNLGWSHPQFEKGGSIPIFVPSGRNISWQPVIQPF
DYQCPIHGNLPNTMGLSATKLTIKSPSVFSTDKVSGWICHAAEWKTTCD
YRWYGPQYITHSIHPISPTIDECRRIIQRIASGTDEDLGFPPQSCGWAS
VTTVSNTNYRVVPHSVHLEPYGGHWIDHEFNGGECREKVCEMKGNHSIW
ITEETVQHECAKHIEEVEGIMYGNVPRGDVMYANNFIIDRHHRVYRFGG
SCQMKFCNKDGIKFARGDWVEKTAGTLTTIHDNVPKCVDGTLVSGHRPG
LDLIDTVFNLENVVEYTLCEGTKRKINKQEKLTSVDLSYLAPRIGGFGS
VFRVRNGTLERGSTTYIRIEVEGPIVDSLNGTDPRTNASRVFWDDWELD
GNIYQGFNGVYKGKDGKIHIPLNMIESGIIDDELQHAFQADIIPHPHYD
DDEIREDDIFFDNTGENGNPVDAVVEWVSGWGTSLKFFGMTLVALILIF
LLIRCCVACTYLMKRSKRPATESHEMRSLV

or is substantially similar to SEQ ID NO: 81. (Bold and underlined sequence represents the affinity binding polypeptide, bolded sequence no underline represents linker peptide, italic sequence represents the SVCV-G signal peptide). The sequence of SEQ ID NO: 81 contains the SVCV-G signal peptide MSIISYIAFLLLIDSNLG (SEQ ID NO: 171). Upon processing, the signal peptide may be cleaved leaving the sequence of SEQ ID NO: 172 (See Table 6).

Thus, while embodiments may refer to further modifications of, mutations of, or percent identity to SEQ ID NO: 81, it is to be understood that the same modifications, mutations, and limitations to percent identity also apply to SEQ ID NO: 172. In some embodiments, the SVCV-G fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 81. In some embodiments, the SVCV-G fused to the affinity binding polypeptide comprises an amino acid sequence of SEQ ID NO: 81.

In some embodiments, the pseudotyped viral-like particle or viral vector does not further comprise a second viral glycoprotein within the viral envelope.

In some embodiments, the pseudotyped viral-like particle or viral vector further comprises a second viral glycoprotein within the viral envelope. In some embodiments, the second viral glycoprotein is selected from the group including, but not limited to, HIV glycoprotein gp120, SIV glycoprotein gp120, EbV glycoprotein, NiV attachment protein (NiV-G), NiV fusion protein (NiV-F), MeV attachment protein (MeV-H), MeV fusion protein (MeV-F), VSV glycoprotein (VSV-G), SVCV glycoprotein (SVCV-G), or any variant thereof, or any combination thereof. In some embodiments, the second viral glycoprotein is HIV glycoprotein gp120, or any variant thereof. In some embodiments, the second viral glycoprotein is SIV glycoprotein gp120, or any variant thereof. In some embodiments, the second viral glycoprotein is EbV glycoprotein or any variant thereof. In some embodiments, the second viral glycoprotein is NiV-G, or any variant thereof. In some embodiments, the second viral glycoprotein is NiV-F, or any variant thereof. In some embodiments, the second viral glycoprotein is MeV-H, or any variant thereof. In some embodiments, the second viral glycoprotein is MeV-F, or any variant thereof. In some embodiments, the second viral glycoprotein is VSV-G, or any variant thereof. In some embodiments, the second viral glycoprotein is SVCV-G, or any variant thereof. One of skill in the art will recognize that any of the above recited glycoproteins may contain conservative amino acid substitutions that do not drastically change the function or the properties of the glycoproteins. Such conservative amino acid substitutions are as provided for herein. Further, one of skill in the art will recognize that certain amino acid substitutions, repetitions, or deletions may enhance the function or the properties of the glycoproteins. Accordingly, in some embodiments, the second viral glycoprotein is substantially similar to HIV glycoprotein gp120. In some embodiments, the second viral glycoprotein is substantially similar to SIV glycoprotein gp120. In some embodiments, the second viral glycoprotein is substantially similar to EbV glycoprotein. In some embodiments, the second viral glycoprotein is substantially similar to NiV-G. In some embodiments, the second viral glycoprotein is substantially similar to NiV-F. In some embodiments, the second viral glycoprotein is substantially similar to MeV-H. In some embodiments, the second viral glycoprotein is substantially similar to MeV-F. In some embodiments, the second viral glycoprotein is substantially similar to VSV-G. In some embodiments, the second viral glycoprotein is substantially similar to SVCV-G. In any embodiment, a substantially similar sequence is as provided for herein.

In some embodiments, the second viral glycoprotein does not comprise an affinity binding polypeptide.

In some embodiments, the second viral glycoprotein comprises at least a first, at least a second, at least a third, at least a fourth, or at least a fifth affinity binding polypeptide sequence. In some embodiments, the second viral glycoprotein comprises at least a first affinity binding polypeptide. In some embodiments, the identity of the at least a first affinity binding polypeptide is an affinity binding polypeptide as provided for herein. In some embodiments, the second viral glycoprotein comprises at least a second affinity binding polypeptide. In some embodiments, the identity of the at least a second affinity binding polypeptide is an affinity binding polypeptide as provided for herein. In some embodiments, the second viral glycoprotein comprises at least a third affinity binding polypeptide. In some embodiments, the identity of the at least a third affinity binding polypeptide is an affinity binding polypeptide as provided for herein. In some embodiments, the second viral glycoprotein comprises at least a fourth affinity binding polypeptide. In some embodiments, the identity of the at least a fourth affinity binding polypeptide is an affinity binding polypeptide as provided for herein. In some embodiments, the second viral glycoprotein comprises at least a fifth affinity binding polypeptide. In some embodiments, the identity of the at least a fifth affinity binding polypeptide is an affinity binding polypeptide as provided for herein. The number of affinity binding polypeptides fused to the glycoprotein is only limited in that the fusion constructs should not negatively affect the function or purpose of the glycoprotein. Thus, in some embodiments, the glycoprotein further comprises at least one affinity binding polypeptide. In some embodiments, the glycoprotein further comprises at least two affinity binding polypeptides. In some embodiments, the glycoprotein further comprises at least three affinity binding polypeptides. In some embodiments, the glycoprotein further comprises at least four affinity binding polypeptides. In some embodiments, the glycoprotein further comprises at least five affinity binding polypeptides. In some embodiments, the glycoprotein further comprises up to five affinity binding polypeptides. In some embodiments, the glycoprotein further comprises up to ten affinity binding polypeptides. In some embodiments, the glycoprotein further comprises up to twenty affinity binding polypeptides.

The at least a first affinity binding polypeptide of the second viral glycoprotein can be inserted at any position in the second glycoprotein that does not substantially negatively affect the function or purpose of the second glycoprotein. Accordingly, in some embodiments, the at least a first affinity binding polypeptide is located at the N-terminus, at the C-terminus, or within the second glycoprotein. In some embodiments, the at least a first affinity binding polypeptide is located at the N-terminus of the second glycoprotein. In some embodiments, the at least a first affinity binding polypeptide is located at the C-terminus of the second glycoprotein. In some embodiments, the at least a first affinity binding polypeptide is located within the second glycoprotein. In some embodiments, the at least a first affinity binding polypeptide is fused directly to the second glycoprotein. In some embodiments, the at least a first affinity binding polypeptide is fused indirectly to the second glycoprotein via, for example, a peptide linker as provided for herein. In embodiments where the at least a first affinity binding polypeptide of the second glycoprotein is located within the second glycoprotein, the affinity binding polypeptide may be fused to the glycoprotein directly, fused directly at the N-terminus of the affinity binding polypeptide and fused indirectly at the C-terminus of the affinity binding polypeptide via, for example, a peptide linker as provided for herein, fused indirectly at the N-terminus of the affinity binding polypeptide via, for example, a peptide linker as provided for herein and fused directly at the C-terminus of the affinity binding polypeptide, or fused indirectly at both the N and C terminus of the affinity binding polypeptide via, for example, a polypeptide linker as provided for herein.

The at least a second affinity binding polypeptide of the second viral glycoprotein can be inserted at any position in the second glycoprotein that does not affect the function or purpose of the glycoprotein. Further, the position of the at least a second affinity binding polypeptide is independent of the position of the at least a first affinity binding polypeptide, so long as the combination of first and second affinity binding polypeptide does not negatively affect the function or purpose of the second glycoprotein. Accordingly, in some embodiments, the at least a second affinity binding polypeptide is located at the N-terminus, at the C-terminus, or within the second glycoprotein. In some embodiments, the at least a second affinity binding polypeptide is located at the N-terminus of the second glycoprotein. In some embodiments, the at least a second affinity binding polypeptide is located at the C-terminus of the second glycoprotein. In some embodiments, the at least a second affinity binding polypeptide is located within the second glycoprotein. In some embodiments, the at least a second affinity binding polypeptide is fused directly to the second glycoprotein. In some embodiments, the at least a second affinity binding polypeptide is fused indirectly to the second glycoprotein via, for example, a peptide linker as provided for herein. In embodiments where the at least a second affinity binding polypeptide of the second viral glycoprotein is located within the second glycoprotein, the affinity binding polypeptide may be fused to the glycoprotein directly, fused directly at the N-terminus of the affinity binding polypeptide and fused indirectly at the C-terminus of the affinity binding polypeptide via, for example, a peptide linker as provided for herein, fused indirectly at the N-terminus of the affinity binding polypeptide via, for example, a peptide linker as provided for herein and fused directly at the C-terminus of the affinity binding polypeptide, or fused indirectly at both the N and C terminus of the affinity binding polypeptide via, for example, a polypeptide linker as provided for herein.

In some embodiments, the position of the at least a third affinity binding polypeptide of the second viral glycoprotein is as provided for herein for the at least a first and the at least a second affinity binding polypeptide of the second viral glycoprotein. In some embodiments, the position of the at least a fourth affinity binding polypeptide of the second viral glycoprotein is as provided for herein for the at least a first and the at least a second affinity binding polypeptide of the second viral glycoprotein. In some embodiments, the position of the at least a fifth affinity binding polypeptide of the second viral glycoprotein is as provided for herein for the at least a first and the at least a second affinity binding polypeptide of the second viral glycoprotein.

In some embodiments, the second viral glycoprotein comprises an amino acid sequence selected from the group including, but not limited to, SEQ ID NO: 25, SEQ ID NO: 29, SEQ ID NO: 30, SEQ ID NO: 33, SEQ ID NO: 34, SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 66. SEQ ID NO: 67, SEQ ID NO: 74, SEQ ID NO: 77, SEQ ID NO: 79, or SEQ ID NO: 80, or comprises an amino acid sequence that is substantially similar to an amino acid sequence selected from the group including, but not limited to, SEQ ID NO: 25, SEQ ID NO: 29, SEQ ID NO: 30, SEQ ID NO: 33, SEQ ID NO: 34, SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 66. SEQ ID NO: 67, SEQ ID NO: 74, SEQ ID NO: 77, SEQ ID NO: 79, or SEQ ID NO: 80. In some embodiments, the second viral glycoprotein comprises an amino acid sequence selected from the group including, but not limited to, SEQ ID NO: 26, SEQ ID NO: 27, SEQ ID NO: 28, SEQ ID NO: 31, SEQ ID NO: 32, SEQ ID NO: 35, SEQ ID NO: 68, SEQ ID NO: 69, SEQ ID NO: 70, SEQ ID NO: 71, SEQ ID NO: 72, SEQ ID NO: 73, SEQ ID NO: 75, SEQ ID NO: 76, SEQ ID NO: 78, SEQ ID NO: 81, SEQ ID NO: 132, SEQ ID NO: 133, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 166, SEQ ID NO: 167, SEQ ID NO: 168, SEQ ID NO: 170, SEQ ID NO: 172, SEQ ID NO: 188, SEQ ID NO: 189, SEQ ID NO: 190, or SEQ ID NO: 191, or comprises an amino acid sequence that is substantially similar to an amino acid sequence selected from the group including, but not limited to, SEQ ID NO: 26, SEQ ID NO: 27, SEQ ID NO: 28, SEQ ID NO: 31, SEQ ID NO: 32, SEQ ID NO: 35, SEQ ID NO: 68, SEQ ID NO: 69, SEQ ID NO: 70, SEQ ID NO: 71, SEQ ID NO: 72, SEQ ID NO: 73, SEQ ID NO: 75, SEQ ID NO: 76, SEQ ID NO: 78, SEQ ID NO: 81, SEQ ID NO: 132, SEQ ID NO: 133, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 166, SEQ ID NO: 167, SEQ ID NO: 168, SEQ ID NO: 170, SEQ ID NO: 172, SEQ ID NO: 188, SEQ ID NO: 189, SEQ ID NO: 190, or SEQ ID NO: 191.

In some embodiments, the targeting moiety of the recombinant viral-like particle or viral vector comprises a targeting moiety as provided for herein. In some embodiments, the targeting moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof, as provided for herein. In some embodiments, the targeting moiety binds to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsgl); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-la); Carbonic anhydrase IX (CA1X); Carcinoembryonic antigen (CEA); CCCTC-Binding Factor (Zinc FingerProtein)-Like (BORIS or Brother of the Regulator of Imprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECL1); Cyclin B1; Cytochrome P450 IB I (CYPIB 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLV1-Tax; Human papilloma virus E6 (HPV E6); Human papilloma virus E7 (HPV E7); Human Telomerase reverse transcriptase (hTERT); IgE; IL13Ra2; IL1 1Ra; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-I (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-I or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAPI; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member IA (XAGE1) as provided for herein.

In some embodiments, the targeting moiety is selected from the group consisting of Stem Cell Factor protein (SCF, KIT-ligand, KL, or steel factor) or a moiety that binds to cKit (CD117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1, as provided for herein.

In some embodiments, the targeting moiety binds CD7 as provided for herein.

In some embodiments, the targeting moiety binds CD8 as provided for herein.

In some embodiments, the targeting moiety does not comprise an affinity binding polypeptide. In some embodiments, the targeting moiety comprises an amino acid sequence of SEQ ID NO: 101, SEQ ID NO: 102, SEQ ID NO: 119, or SEQ ID NO: 120, or any variant thereof as provided for herein.

In some embodiments, the at least a first affinity binding polypeptide of the pseudotyped viral-like particle or viral vector is not fused to the glycoprotein of the pseudotyped viral-like particle or viral vector. In some embodiments, the at least a first affinity binding polypeptide of the pseudotyped viral-like particle or viral vector is fused to the targeting moiety of the pseudotyped viral-like particle or viral vector.

In some embodiments, a pseudotyped viral-like particle or viral vector is provided comprising: an envelope comprising a recombinant virus glycoprotein, a targeting moiety for binding to a target cell, and at least a first affinity binding polypeptide, wherein the affinity binding polypeptide is fused to the glycoprotein, the targeting moiety, or any combination thereof; and a nucleic acid molecule encoding a heterologous molecule of interest, wherein the at least a first affinity binding polypeptide is fused to the targeting moiety. In some embodiments, the virus glycoprotein is derived from a virus from the group consisting of Human Immunodeficiency Virus (HIV), Simian Immunodeficiency Virus (SIV), Ebola virus (EbV), Nipah Virus (NiV), Measles virus (MeV), Vesicular stomatitis virus (VSV), Spring viremia of carp virus (SVCV) or a combination thereof. In some embodiments, the virus glycoprotein is HIV glycoprotein gp120, SIV glycoprotein gp120, EbV glycoprotein, NiV-G, NiV-F, MeV-H, MeV-F, VSV-G, SVCV-G, any variant thereof, or any combination thereof. In some embodiments, the viral-like particle is a retroviral-like particle or retroviral vector. In some embodiments, the retroviral-like particle is a lentivirus based viral particle or viral vector. In some embodiments, the at least a first affinity binding polypeptide is an affinity tag selected from the group consisting of a polyhistidine tag, a polyarginine tag, a FLAG tag, a streptavidin tag, a calmodulin binding peptide, or a variant or combination thereof. In some embodiments, the streptavidin tag is selected from the group consisting of a streptavidin-binding peptide, a streptavidin binding tag, a strep-tag II, a twin-strep tag, or a variant or combination thereof. In some embodiments, the at least a first affinity binding polypeptide is a strep-tag II polypeptide sequence.

In some embodiments, the identity of the at least a first affinity binding polypeptide is as provided for herein. The at least a first affinity binding polypeptide can be inserted at any position in the targeting moiety that does not substantially negatively affect the function or purpose of the targeting moiety. Accordingly, in some embodiments, the at least a first affinity binding polypeptide is located at the N-terminus, at the C-terminus, or within the targeting moiety. In some embodiments, the at least a first affinity binding polypeptide is located at the N-terminus of the targeting moiety. In some embodiments, the at least a first affinity binding polypeptide is located at the C-terminus of the targeting moiety. In some embodiments, the at least a first affinity binding polypeptide is located within the targeting moiety. In some embodiments, the at least a first affinity binding polypeptide is fused directly to the targeting moiety. In some embodiments, the at least a first affinity binding polypeptide is fused indirectly to the targeting moiety via, for example, a peptide linker as provided for herein. In embodiments where the at least a first affinity binding polypeptide is located within the targeting moiety, the affinity binding polypeptide may be fused to the targeting moiety directly, fused directly at the N-terminus of the affinity binding polypeptide and fused indirectly at the C-terminus of the affinity binding polypeptide via, for example, a peptide linker as provided for herein, fused indirectly at the N-terminus of the affinity binding polypeptide via, for example, a peptide linker as provided for herein and fused directly at the C-terminus of the affinity binding polypeptide, or fused indirectly at both the N and C terminus of the affinity binding polypeptide via, for example, a polypeptide linker as provided for herein.

In some embodiments, the pseudotyped viral-like particle or viral vector further comprises at least a second affinity binding polypeptide. In some embodiments, both the at least a first and the at least a second affinity binding polypeptides are fused to the targeting moiety of the pseudotyped viral-like particle or viral vector. In some embodiments, the identity of the at least a second affinity binding polypeptide is as provided for herein. The at least a second affinity binding polypeptide can be inserted at any position in the targeting moiety that does not substantially negatively affect the function or purpose of the targeting moiety. Further, the position of the at least a second affinity binding polypeptide is independent of the position of the at least a first affinity binding polypeptide, so long as the combination of first and second affinity binding polypeptide does not substantially negatively affect the function or purpose of the targeting moiety. Accordingly, in some embodiments, the at least a second affinity binding polypeptide is located at the N-terminus, at the C-terminus, or within the targeting moiety. In some embodiments, the at least a second affinity binding polypeptide is located at the N-terminus of the targeting moiety. In some embodiments, the at least a second affinity binding polypeptide is located at the C-terminus of the targeting moiety. In some embodiments, the at least a second affinity binding polypeptide is located within the targeting moiety. In some embodiments, the at least a second affinity binding polypeptide is fused directly to the targeting moiety. In some embodiments, the at least a second affinity binding polypeptide is fused indirectly to the targeting moiety via, for example, a peptide linker as provided for herein. In embodiments where the at least a second affinity binding polypeptide is located within the targeting moiety, the affinity binding polypeptide may be fused to the targeting moiety directly, fused directly at the N-terminus of the affinity binding polypeptide and fused indirectly at the C-terminus of the affinity binding polypeptide via, for example, a peptide linker as provided for herein, fused indirectly at the N-terminus of the affinity binding polypeptide via, for example, a peptide linker as provided for herein and fused directly at the C-terminus of the affinity binding polypeptide, or fused indirectly at both the N and C terminus of the affinity binding polypeptide via, for example, a polypeptide linker as provided for herein.

In some embodiments, the pseudotyped viral-like particle or viral vector further comprises at least a third, at least a fourth, or at least a fifth affinity binding polypeptide. In some embodiments, the pseudotyped viral-like particle or viral vector further comprises at least a third affinity binding polypeptide. In some embodiments, the identity of the at least a third affinity binding polypeptide is as provided for herein. In some embodiments, the pseudotyped viral-like particle or viral vector further comprises at least a fourth affinity binding polypeptide.

In some embodiments, the identity of the at least a fourth affinity binding polypeptide is as provided for herein. In some embodiments, the pseudotyped viral-like particle or viral vector further comprises at least a fifth affinity binding polypeptide. In some embodiments, the identity of the at least a fifth affinity binding polypeptide is as provided for herein. In some embodiments, each additional affinity binding polypeptide is fused to the targeting moiety of the pseudotyped viral-like particle or viral vector. In some embodiments, the position of the at least a third affinity binding polypeptide on or within the targeting moiety is as provided for herein for the at least a first and the at least a second affinity binding polypeptide fused to the targeting moiety. In some embodiments, the position of the at least a fourth affinity binding polypeptide on or within the targeting moiety is as provided for herein for the at least a first and the at least a second affinity binding polypeptide fused to the targeting moiety. In some embodiments, the position of the at least a fifth affinity binding polypeptide on or within the targeting moiety is as provided for herein for the at least a first and the at least a second affinity binding polypeptide fused to the targeting moiety.

The number of affinity binding polypeptides fused to the targeting moiety is only limited in that the fusion constructs should not negatively affect the function or purpose of the targeting moiety. Thus, in some embodiments, the targeting moiety further comprises at least one affinity binding polypeptide. In some embodiments, the targeting moiety further comprises at least two affinity binding polypeptides. In some embodiments, the targeting moiety further comprises at least three affinity binding polypeptides. In some embodiments, the targeting moiety further comprises at least four affinity binding polypeptides. In some embodiments, the targeting moiety further comprises at least five affinity binding polypeptides. In some embodiments, the targeting moiety further comprises up to five affinity binding polypeptides. In some embodiments, the targeting moiety further comprises up to ten affinity binding polypeptides. In some embodiments, the targeting moiety further comprises up to twenty affinity binding polypeptides.

In some embodiments, the targeting moiety is selected from the group including, but not limited to, an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof. In some embodiments, the targeting protein is an scFv. In some embodiments, the targeting moiety is fused to the viral glycoprotein. In some embodiments the targeting moiety is fused directly to the viral glycoprotein. In some embodiments, the targeting moiety is fused indirectly to the viral glycoprotein via, for example, a peptide linker as provided for herein. In some embodiments, the targeting moiety is not fused to the viral glycoprotein.

In some embodiments, the targeting moiety is selected from the group consisting of Stem Cell Factor protein (SCF, KIT-ligand, KL, or steel factor) or a moiety that binds to cKit (CD117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1.

In some embodiments, the targeting moiety is selected to bind to a particular target protein. The targeting moiety may be selected to bind to any desired protein or peptide target.

In some embodiments, the targeting moiety binds to a target selected from the group including, but not limited to, CD7, CD8, cKit (CD 117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsgl); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-la); Carbonic anhydrase IX (CA1X); Carcinoembryonic antigen (CEA); CCCTC-Binding Factor (Zinc Finger Protein)-Like (BORIS or Brother of the Regulator of Imprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLII; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudin18.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECL1); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLV1-Tax; Human papilloma virus E6 (HPV E6); Human papilloma virus E7 (HPV E7); Human Telomerase reverse transcriptase (hTERT); IgE; IL13Ra2; IL1 1Ra; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-I (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAPI; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); X Antigen Family Member iA (XAGE1).

In some embodiments, the targeting moiety (a polypeptide) can bind to CD7. In some embodiments, the polypeptide binds to CD7. In some embodiments, the polypeptide that binds to CD7 is an antibody which binds to non-human primate CD7. In some embodiments, the polypeptide that binds to CD7 is an antibody which binds to human CD7. The sequence of human CD7 (UniProtKB P09564) is as follows (SEQ ID NO: 82):

(SEQ ID NO: 82)
MAGPPRLLLLPLLLALARGLPGALAAQEVQQSPHCTTVPVGASVNITCS
TSGGLRGIYLRQLGPQPQDIIYYEDGVVPTTDRRFRGRIDFSGSQDNLT
ITMHRLQLSDTGTYTCQAITEVNVYGSGTLVLVTEEQSQGWHRCSDAPP
RASALPAPPTGSALPDPQTASALPDPPAASALPAALAVISFLLGLGLGV
ACVLARTQIKKLCSWRDKNSAACVVYEDMSHSRCNTLSSPNQYQ

In some embodiments, the CD7 antibody comprises a Fc region. The Fc region can be linked to the heavy or light chain of the antibody. The Fc region may be fused directly to the heavy or light chain of the antibody or may be fused indirectly to the heavy or light chain of the antibody via, for example, a peptide linker as provided for herein. In some embodiments, the Fc region is an IgG Fc. In some embodiments, the IgG is selected from IgG1, IgG2, IgG3, or 1gG4. In some embodiments, the IgG fc is IgG1 Fc. In some embodiments, the antibody comprises an Fc constant region of SEQ ID NO: 83 as set forth below:

(SEQ ID NO: 83)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLIVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, the IgG fc is IgG2 Fc. In some embodiments, the antibody comprises an Fc constant region of SEQ ID NO: 84 as set forth below:

(SEQ ID NO: 84)
STKGPSVFPLAPCSRSTSESTAALGCLVKDYFPEPVTVSWNSGALTSGV
HTFPAVLQSSGLYSLSSVVTVPSSSLGTQTYTCNVDHKPSNTKVDKTVE
RKCCVECPPCPAPPVAGPSVFLFPPKPKDTLMISRTPEVTCVVVDVSHE
DPEVQFNWYVDGVEVHNAKIKPREEQFNSTFRVVSVLTVVHQDWLNGKE
YKCKVSNKGLPAPIEKTISKTKGQPREPQVYTLPPSREEMTKNQVSLTC
LVKGFYPSDIAVEWESNGQPENNYKITPPMLDSDGSFFLYSKLTVDKSR
WQQGNVFSCSVMHEALHNHYTQKSLSLSPGK

In some embodiments, the IgG fc is IgG4 Fc. In some embodiments, the antibody comprises an Fc constant region of SEQ ID NO: 85 as set forth below:

(SEQ ID NO: 85)
STKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVE
SKYGPPCPSCPAPEFLGGPSVFLFPPKPKDTLMISRTPEVTCVVVDVSQ
EDPEVQFNWYVDGVEVHNAKTKPREEQFNSTYRVVSVLTVLHQDWLNGK
EYKCKVSNKGLPSSIEKTISKAKGQPREPQVYTLPPSQEEMTKNQVSLT
CLVKGFYPSDIAVEWESNGQPENNYKTTPPVLDSDGSFFLYSRLTVDKS
RWQEGNVFSCSVMHEALHNHYTQKSLSLSLGK

In some embodiments, the IgG Fc is a variant of an IgG1 Fe protein (SEQ ID NO: 83). In some embodiments, the variant IgG1 Fe protein comprises one or more of the mutations that corresponds to those selected from the group consisting of: L234A, L235A, N297A, P329G, I253A, H310A, and H435A of SEQ ID NO: 83. Any of the mutations L234A, L235A, N297A, P329G, I253A, H310A, and H435A of SEQ ID NO: 83 may be present or absent and the mutations may be combined in any combination. In some embodiments, the variant IgG1 Fc protein comprises a mutation that corresponds to L234A and L235A of SEQ ID NO: 83. In some embodiments, the variant IgG1 Fc protein comprises a mutation that corresponds to N297A of SEQ ID NO: 83. In some embodiments, the variant IgG1 Fe protein comprises a mutation that corresponds to P329G of SEQ ID NO: 83. In some embodiments, the variant IgG1 Fe protein comprises a mutation that corresponds to L234A, L235A, N297A, and P329G of SEQ ID N: 83. In some embodiments, the variant IgG1 Fe protein comprises a mutation that corresponds to I253A of SEQ ID NO: 83. In some embodiments, the variant IgG1 Fc protein comprises a mutation that corresponds to H310A of SEQ ID NO: 83. In some embodiments, the variant IgG1 Fe protein comprises a mutation that corresponds to H435A of SEQ ID NO: 83. In some embodiments, the variant IgG1 Fe protein comprises a mutation that corresponds to I253A, H310A, and H435A of SEQ ID NO: 83.

In some embodiments, the IgG Fe is a variant of an IgG2 Fe protein (SEQ ID NO: 84). In some embodiments, the variant IgG2 Fc protein comprises one or more mutations selected from the group consisting of: N297A, P329G, I253A, H310A, and H435A as those position correspond to SEQ ID NO: 84. Any of the mutations N297A, P329G, 1253A, H310A, and H435A of SEQ ID NO: 84 may be present or absent and the mutations may be combined in any combination. In some embodiments, the variant IgG2 Fe protein comprises a mutation that corresponds to N297A of SEQ ID NO: 84. In some embodiments, the variant IgG2 Fe protein comprises a mutation that corresponds to P329G of SEQ ID NO: 84. In some embodiments, the variant IgG2 Fe protein comprises a mutation that corresponds to N297A and P329G of SEQ ID NO: 84. In some embodiments, the variant IgG2 Fe protein comprises a mutation that corresponds to I253A of SEQ ID NO: 84. In some embodiments, the variant IgG2 Fe protein comprises a mutation that corresponds to H310A of SEQ ID NO: 84. In some embodiments, the variant IgG2 Fe protein comprises a mutation that corresponds to H435A of SEQ ID NO: 84. In some embodiments, the variant IgG2 Fc protein comprises a mutation that corresponds to I253A, H310A, and H435A of SEQ ID NO: 84.

In some embodiments, the IgG Fe protein is a variant of an IgG4 Fe protein (SEQ ID NO: 85). In some embodiments, the variant IgG4 Fe protein comprises one or more mutations selected from the group consisting of: S228P, L235E, N297A, P329G, I253A, H310A, and H435A as those positions correspond to SEQ ID NO: 85. Any of the mutations S228P, L235E, N297A, P329G, I253A, H310A, and H435A of SEQ ID NO: 85 may be present or absent and the mutations may be combined in any combination. In some embodiments, the variant IgG4 Fe protein comprises a mutation that corresponds to S228P of SEQ ID NO: 85. In some embodiments, the variant IgG4 Fc protein comprises a mutation that corresponds to L235E of SEQ ID NO: 85. In some embodiments, the variant IgG4 Fc protein comprises a mutation that corresponds to N297A of SEQ ID NO: 85. In some embodiments, the variant IgG4 Fc protein comprises a mutation that corresponds to P329G of SEQ ID NO: 85. In some embodiments, the variant IgG4 Fc protein comprises a mutation that corresponds to S228P, L235E, N297A, and P329G of SEQ ID NO: 85. In some embodiments, the variant IgG4 Fc protein comprises a mutation that corresponds to I253A of SEQ ID NO: 85. In some embodiments, the variant IgG4 Fe protein comprises a mutation that corresponds to H310A of SEQ ID NO: 85. In some embodiments, the variant IgG4 Fc protein comprises a mutation that corresponds to H435A of SEQ ID NO: 85. In some embodiments, the variant IgG4 Fc protein comprises a mutation that corresponds to I253A, H310A, and H435A of SEQ ID NO: 85.

In some embodiments, the targeting moiety binds to CD7 and comprises an HCDR1, HCDR2, HCDR3, LCDR1, LCDR2, LCDR3, VH, and VL as recited in Table 2 and Table 3 below:

TABLE 2
CD7 targeting moiety CD7AB1 CDR sequences

| Numbering | | | | | | |
|---|---|---|---|---|---|---|
| System | HCDR1 | HCDR2 | HCDR3 | LCDR1 | LCDR2 | LCDR3 |
| Chothia | GYPFTSY | DPNSGD (SEQ | SPYYSNDNSM | RASQSIGTSIH | YASESIS | QQSNSWPTT |
| | (SEQ ID | ID NO: 87) | DY (SEQ ID | (SEQ ID NO: | (SEQ ID | (SEQ ID |
| | NO: 86) | | NO: 88) | 89) | NO: 90) | NO: 91) |
| Kabat | SYWIH | RIDPNSGDTKY | SEQ ID NO: | SEQ ID NO: | SEQ ID | SEQ ID |
| | (SEQ ID | NEKFKN (SEQ | 88 | 89 | NO: 90 | NO: 91 |
| | NO: 92) | ID NO: 93) | | | | |
| IMGT | GYPFTSYW | IDPNSGDT | ARSPYYSNDN | QSIGTS (SEQ | YA | SEQ ID |
| | (SEQ ID | (SEQ ID NO: | SMDY (SEQ | ID NO: 97) | | NO: 91 |
| | NO: 94) | 95) | ID NO: 96) | | | |

TABLE 3
CD7 targeting moiety CD7AB1 Variable Regions

| SEQ ID NO: | AB ID NO: | Region | Sequence |
|---|---|---|---|
| 98 | CD7AB1 | VH | QVQLQQPGAELVKPGASVKLSCKASGYPFTSYWIHWVKQRPGRGLEWL |
| | | | GRIDPNSGDTKYNEKFKNKATLTVDKSSTTAYMQLSSLTSEDSAVYYC |
| | | | ARSPYYSNDNSMDYWGQGTSVTVSS |
| 99 | CD7AB1 | VL | DILLTQSPAILSVSPGERVSFSCRASQSIGTSIHWYQQRINDSPRLLI |
| | | | KYASESISGIPSRFSGSGSGTDFTLSINSVESEDIADYYCQQSNSWPT |
| | | | TFGGGTKLEIKR |

The VH and the VL sequences can be in any format, including, but not limited to an scFv format where the VH and VL regions are linked with a peptide linker. Examples of peptide linkers that can be used to link various peptides provided for herein include, but are not limited to: (GGGGS)ₙ (SEQ ID NO: 4), wherein each n is independently 1-4. In some embodiments, n is 1. In some embodiments, n is 2. In some embodiments, n is 3. In some embodiments, n is 4. In some embodiments, the variable regions are not linked with a peptide linker. In some embodiments, the polypeptide comprises SEQ ID NO: 98 and SEQ ID NO: 99. In some embodiments, the targeting moiety comprises a linked peptide represented by a formula of V_{L}-Z-V_{H}, wherein Z is a peptide linker. In some embodiments, the targeting moiety comprises a heavy chain variable region as set forth in SEQ ID NO: 98 linked via a linker sequence GGGGSGGGGSGGGGSGGGGS (SEQ ID NO: 100) to a light chain variable region as set forth in SEQ ID NO: 99. In some embodiments, a targeting moiety comprising a V_{L} linked via a peptide linker to a VH has the sequence as set forth below:

(SEQ ID NO: 101)
DILLTQSPAILSVSPGERVSFSCRASQSIGTSIHWYQQRINDSPRLLIK
YASESISGIPSRFSGSGSGTDFTLSINSVESEDIADYYCQQSNSWPTTF
GGGTKLEIKRGGGGSGGGGSGGGGSGGGGSQVQLQQPGAELVKPGASVK
LSCKASGYPFTSYWIHWVKQRPGRGLEWLGRIDPNSGDTKYNEKFKNKA
TLTVDKSSTTAYMQLSSLTSEDSAVYYCARSPYYSNDNSMDYWGQGTSV
TVSS

In some embodiments, the targeting moiety comprises a linked peptide represented by a formula of V_{H}-Z-V_{L}, wherein Z is a peptide linker. In some embodiments, the targeting moiety comprises a heavy chain variable region as set forth in SEQ ID NO: 98 linked via a linker sequence GGGGSGGGGSGGGGSGGGGS (SEQ ID NO: 100) to a light chain variable region as set forth in SEQ ID NO: 99. In some embodiments, a targeting moiety comprising a VH linked via a peptide linker to a VL has the sequence as set forth below:

(SEQ ID NO: 102)
QVQLQQPGAELVKPGASVKLSCKASGYPFTSYWIHWVKQRPGRGLEWLG
RIDPNSGDTKYNEKFKNKATLTVDKSSTTAYMQLSSLTSEDSAVYYCAR
SPYYSNDNSMDYWGQGTSVTVSSGGGGSGGGGSGGGGSGGGGSDILLTQ
SPAILSVSPGERVSFSCRASQSIGTSIHWYQQRINDSPRLLIKYASESI
SGIPSRFSGSGSGTDFTLSINSVESEDIADYYCQQSNSWPTTFGGGTKL
EIKR

In some embodiments, the targeting moiety (a polypeptide) can bind to CD8.

In some embodiments, the polypeptide binds to CD8. In some embodiments, the polypeptide binds to CD8-alpha. In some embodiments, the polypeptide binds to CD8-beta. In some embodiments, the polypeptide binds to CD8 heterodimer. In some embodiments, the CD8 heterodimer comprises CD8-alpha and CD8-beta subunits. In some embodiments, the polypeptide binds to CD8-alpha homodimer. In some embodiments, the polypeptide that binds to CD8 is an antibody which binds to non-human primate CD8. In some embodiments, the antibody that binds to non-human primate CD8 is an antibody which binds to non-human primate CD8-alpha. In some embodiments, the antibody that binds to non-human primate CD8 is an antibody which binds to non-human primate CD8-beta. In some embodiments, the antibody that binds to non-human primate CD8 is an antibody which binds to non-human primate CD8-alpha homodimer. In some embodiments, the antibody that binds to non-human primate CD8 is an antibody which binds to non-human primate CD8 heterodimer. In some embodiments, the polypeptide that binds to CD8 is an antibody which binds to human CD8. In some embodiments, the antibody that binds to human CD8 is an antibody which binds to human CD8-alpha. In some embodiments, the antibody that binds to human CD8 is an antibody which binds to human CD8-beta. In some embodiments, the antibody that binds to human CD8 is an antibody which binds to human CD8-alpha homodimer. In some embodiments, the antibody that binds to human CD8 is an antibody which binds to human CD8 heterodimer. The sequence of human CD8-alpha (UniProtKB Q8TAW8) is as follows (SEQ ID NO: 103):

(SEQ ID NO: 103)
MALPVTALLLPLALLLHAARPSQFRVSPLDRIWNLGETVELKCQVLLS
NPTSGCSWLFQPRGAAASPTFLLYLSQNKPKAAEGLDTQRFSGKRLGD
TFVLTLSDFRRENEGCYFCSALSNSIMYFSHFVPVFLPAKPTTTPAPR
PPTPAPTIASQPLSLRPEACRPAAGGAVHTRGLDFACDIYIWAPLAGT
CGVLLLSLVITLYCNHRNRRRVCKCPRPVVKSGDKPSLSARYV

The sequence of human CD8-beta (UniProtKB Q8TD28) is as follows (SEQ ID NO: 104):

(SEQ ID NO: 104)
MRPRLWLLLAAQLTVLHGNSVLQQTPAYIKVQTNKMVMLSCEAKISLS
NMRIYWLRQRQAPSSDSHHEFLALWDSAKGTIHGEEVEQEKIAVFRDA
SRFILNLTSVKPEDSGIYFCMIVGSPELTFGKGTQLSVVDFLPTTAQP
TKKSTLKKRVCRLPRPETQKGPLCSPITLGLLVAGVLVLLVSLGVAIH
LCCRRRRARLRFMKQLYK

In some embodiments, the CD8 antibody comprises a Fc region. The Fc region can be linked to the heavy or light chain of the antibody. The Fc region may be fused directly to the heavy or light chain of the antibody or may be fused indirectly to the heavy or light chain of the antibody via, for example, a peptide linker as provided for herein. In some embodiments, the Fc region is an IgG Fe as provided for herein. In some embodiments, the IgG is selected from IgG1, IgG2, IgG3, or IgG4. In some embodiments, the IgG fc is IgG1 Fc as provided for herein. In some embodiments, the antibody comprises an Fe constant region of SEQ ID NO: 83. In some embodiments, the IgG fc is IgG2 Fc as provided for herein. In some embodiments, the antibody comprises an Fc constant region of SEQ ID NO: 84. In some embodiments, the IgG fc is IgG4 Fc as provided for herein. In some embodiments, the antibody comprises an Fc constant region of SEQ ID NO: 85.

In some embodiments, the targeting moiety binds to CD8 and comprises an HCDR1, HCDR2, HCDR3, LCDR1, LCDR2, LCDR3, VH, and VL as recited in Table 4 and Table 5 below:

TABLE 4
CD8 targeting moiety CD8AB1 CDR sequences

| Numbering | | | | | | |
|---|---|---|---|---|---|---|
| System | HCDR1 | HCDR2 | HCDR3 | LCDR1 | LCDR2 | LCDR3 |
| Chothia | RYTFTDY | YPYNGG (SEQ | DHRYNEGVSF | RASESVDGFGN | LASNLES | QQNNEDPYT |
| | (SEQ ID | ID NO: 106) | DY (SEQ ID | SFMN (SEQ | (SEQ ID | (SEQ ID |
| | NO: 105) | | NO: 107) | ID NO: 108) | NO: 109) | NO: 110) |
| Kabat | DYNLH | FIYPYNGGTGY | SEQ ID NO: | SEQ ID NO: | SEQ ID | SEQ ID |
| | (SEQ ID | NQKFKN (SEQ | 107 | 108 | NO: 109 | NO: 110 |
| | NO: 111) | ID NO: 112) | | | | |
| IMGT | RYTFTDYN | IYPYNGGT | ARDHRYNEGV | ESVDGFGNSF | LA | SEQ ID |
| | (SEQ ID | (SEQ ID NO: | SFDY (SEQ | (SEQ ID NO: | | NO: 110 |
| | NO: 113) | 114) | ID NO: | 116) | | |
| | | | 115) | | | |

TABLE 5
CD8 targeting moiety CD8AB1 Variable Regions

| SEQ ID NO: | AB ID NO: | Region | Sequence |
|---|---|---|---|
| 117 | CD7AB1 | VH | EVQLQQSGPELVKPGASVKISCKASRYTFTDYNLHWVKLSHEKSLEWI |
| | | | GFIYPYNGGTGYNQKFKNKAKLTVDYSSSTAYMELRSLTSVDAAVYYC |
| | | | ARDHRYNEGVSFDYWGQGTTLTVSS |
| 118 | CD7AB1 | VL | NIVLTQSPASLAVSLGQRATISCRASESVDGFGNSFMNWYQQKPGQSP |
| | | | KLLIYLASNLESGVPARFSGSGSRTDFTLTIDPVEADDAATYYCQQNN |
| | | | EDPYTFGGGTKLEIKR |

The VH and the VL sequences can be in any format, including, but not limited to an scFv format where the VH and VL regions are linked with a peptide linker. Examples of peptide linkers that can be used to link various peptides provided for herein include, but are not limited to: (GGGGS)ₙ (SEQ ID NO: 4), wherein each n is independently 1-4. In some embodiments, n is 1. In some embodiments, n is 2. In some embodiments, n is 3. In some embodiments, n is 4. In some embodiments, the variable regions are not linked with a peptide linker. In some embodiments, the polypeptide comprises SEQ ID NO: 117 and SEQ ID NO: 118.

In some embodiments, the targeting moiety comprises a linked peptide represented by a formula of V_{L}-Z-V_{H}, wherein Z is a peptide linker. In some embodiments, the targeting moiety comprises a heavy chain variable region as set forth in SEQ ID NO: 117 linked via a linker sequence GGGGSGGGGSGGGGSGGGGS (SEQ ID NO: 100) to a light chain variable region as set forth in SEQ ID NO: 118. In some embodiments, a targeting moiety comprising a V_{L} linked via a peptide linker to a V_{H} has the sequence as set forth below:

(SEQ ID NO: 119)
NIVLTQSPASLAVSLGQRATISCRASESVDGFGNSFMNWYQQKPGQSPK
LLIYLASNLESGVPARFSGSGSRTDFTLTIDPVEADDAATYYCQQNNED
PYTFGGGTKLEIKRGGGGSGGGGSGGGGSGGGGSEVQLQQSGPELVKPG
ASVKISCKASRYTFTDYNLHWVKLSHEKSLEWIGFIYPYNGGTGYNQKF
KNKAKLTVDYSSSTAYMELRSLTSVDAAVYYCARDHRYNEGVSFDYWGQ
GTTLTVSS

In some embodiments, the targeting moiety comprises a linked peptide represented by a formula of V_{H}-Z-V_{L}, wherein Z is a peptide linker. In some embodiments, the targeting moiety comprises a heavy chain variable region as set forth in SEQ ID NO: 117 linked via a linker sequence GGGGSGGGGSGGGGSGGGGS (SEQ ID NO: 100) to a light chain variable region as set forth in SEQ ID NO: 118. In some embodiments, a targeting moiety comprising a V_{H} linked via a peptide linker to a V_{L} has the sequence as set forth below:

(SEQ ID NO: 120)
EVQLQQSGPELVKPGASVKISCKASRYTFTDYNLHWVKLSHEKSLEWIGF
IYPYNGGTGYNQKFKNKAKLTVDYSSSTAYMELRSLTSVDAAVYYCARDH
RYNEGVSFDYWGQGTTLTVSSGGGGSGGGGSGGGGSGGGGSNIVLTQSPA
SLAVSLGQRATISCRASESVDGFGNSFMNWYQQKPGQSPKLLIYLASNLE
SGVPARFSGSGSRIDFTLTIDPVEADDAATYYCQQNNEDPYTEGGGTKLE
IKR

In some embodiments, the targeting moiety comprises an amino acid sequence having at least 70% identity to SEQ ID NO: 101, or is substantially similar to SEQ ID NO: 101, or is an active fragment of SEQ ID NO: 101. In some embodiments, the targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 101. In some embodiments, the targeting moiety comprises an amino acid sequence of SEQ ID NO: 101.

In some embodiments, the targeting moiety comprises an amino acid sequence having at least 70% identity to SEQ ID NO: 102, or is substantially similar to SEQ ID NO: 102, or is an active fragment of SEQ ID NO: 102. In some embodiments, the targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 102. In some embodiments, the targeting moiety comprises an amino acid sequence of SEQ ID NO: 102.

In some embodiments, the targeting moiety comprises an amino acid sequence having at least 70% identity to SEQ ID NO: 119, or is substantially similar to SEQ ID NO: 119, or is an active fragment of SEQ ID NO: 119. In some embodiments, the targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 119. In some embodiments, the targeting moiety comprises an amino acid sequence of SEQ ID NO: 119.

In some embodiments, the targeting moiety comprises an amino acid sequence having at least 70% identity to SEQ ID NO: 120, or is substantially similar to SEQ ID NO: 120, or is an active fragment of SEQ ID NO: 120. In some embodiments, the targeting moiety comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 120. In some embodiments, the targeting moiety comprises an amino acid sequence of SEQ ID NO: 120.As provided for herein, the affinity binding polypeptides may be located at the N-terminus, at the C-terminus, or within the targeting moiety. In some embodiments, the at least a first affinity binding polypeptide is located at the N-terminus of the targeting moiety. In some embodiments, the at least a first affinity binging polypeptide is located in the linker domain between the V_{H} and V_{L} regions of a targeting moiety as provided for herein. In some embodiments, the at least a first affinity binding polypeptide is located in the linker domain between the V_{H} and the Fc domain. In some embodiments, the at least a first affinity binding polypeptide is located in the linker domain between the V_{L} and the Fc domain.

In some embodiments, a targeting moiety as provided for herein may be flanked by one or more dipeptides as provided for herein. In some embodiments, the dipeptides may be present at the N-terminus, C-terminus, or both the N-terminus and C-terminus of the targeting moiety. Examples of such dipeptides include, but are not limited to, SA, AS, and TG. In any of the embodiments provided herein, the dipeptides may be present or absent.

In some embodiments, the affinity binding polypeptide fused to the targeting moiety comprises an amino acid sequence having at least 70% identity to SEQ ID NO 121:

(SEQ ID NO: 121)
METDTLLLWVLLLWVPGSTGDWSHPQFEKGGSSAQVQLQQPGAELVKPGA
SVKLSCKASGYPFTSYWIHWVKQRPGRGLEWLGRIDPNSGDTKYNEKFKN
KATLTVDKSSTTAYMQLSSLTSEDSAVYYCARSPYYSNDNSMDYWGQGTS
VTVSSGGGGSGGGGSGGGGSGGGGSDILLTQSPAILSVSPGERVSFSCRA
SQSIGTSIHWYQQRTNDSPRLLIKYASESISGIPSRFSGSGSGTDFTLSI
NSVESEDIADYYCQQSNSWPTTFGGGTKLEIKRASGGGGSGGGGSGGGGS
EPKSCDKTHTCPPCPAPEAAGGPSVFLFPPKPKDTLMASRTPEVTCVVVD
VSHEDPEVKFNWYVDGVEVHNAKTKPREEQYASTYRVVSVLTVLAQDWLN
GKEYKCKVSNKALGAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNAYTQKSLSLSPGKKIEVMYPPPYLDNEKSNG
TIIHVKGKHLCPSPLFPGPSKPFWVLVVVGGVLACYSLLVTVAFIIFWVR
SKRSRLLHSDYMNRVRQGYS

or is substantially similar to SEQ ID NO: 121, or is an active fragment of SEQ ID NO: 121. (Bold and underlined sequence represents the affinity binding polypeptide, bolded sequence no underline represents linker peptide, italic sequence represents leader peptide, underlined sequence represents targeting moiety). The sequence of SEQ ID NO: 121 contains the leader peptide METDTLLLWVLLLWVPGSTGD (SEQ ID NO: 173). Upon processing, the leader peptide may be cleaved leaving the sequence of SEQ ID NO: 174 (See Table 6). Thus, while embodiments may refer to further modifications of, mutations of, or percent identity to SEQ ID NO: 121, it is to be understood that the same modifications, mutations, and limitations to percent identity also apply to SEQ ID NO: 174. In some embodiments, the sequence of SEQ ID NO: 121 further comprises an amino acid deletion at position 34, 35, 296, 297, or a combination thereof as compared to SEQ ID NO: 121. In some embodiments, the deletion at position 34 comprises a S34 deletion. In some embodiments, the deletion at position 35 comprises a A35 deletion. In some embodiments, the deletion at position 296 comprises a A296 deletion. In some embodiments, the deletion at position 297 comprises a S297 deletion. In some embodiments, the affinity binding polypeptide fused to the targeting moiety comprising amino acid deletions at positions S34, A35, A296, and S297 as compared to SEQ ID NO: 121 comprises the amino acid sequence of SEQ ID NO: 175 (See Table 6). In some embodiments, the affinity binding polypeptide fused to the targeting moiety comprising amino acid deletions at positions S34, A35, A296, and S297 as compared to SEQ ID NO: 121 and further comprising removal of the leader peptide comprises the amino acid sequence of SEQ ID NO: 176 (See Table 6). In some embodiments, the targeting moiety fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 121. In some embodiments, the targeting moiety fused to the affinity binding polypeptide comprises an amino acid sequence of SEQ ID NO: 121.

In some embodiments, the affinity binding polypeptide is attached to the viral surface through an IgG Fc as provided for herein. In some embodiments, the affinity binding polypeptide is attached to the viral surface through a flexible stalk. In some embodiments, the flexible stalk comprises a flexible peptide linker. In some embodiments, the flexible peptide linker is any flexible peptide linker. In some embodiments, the flexible peptide linker is as provided for herein. In some embodiments, the flexible peptide linker is selected from the group of flexible linkers including, but not limited to, (GGGGA)n (SEQ ID NO: 122), (GGGGS)ₙ (SEQ ID NO: 4), GSAGSAAGSGEF (SEQ ID NO: 123), KESGSVSSEQLAQFRSLD (SEQ ID NO: 124), EGKSSGSGSESKST (SEQ ID NO: 125), or any combination thereof, wherein each n is, independently, an integer selected from 1-4. In some embodiments, each n is, independently, an integer selected from 1-4, 1-5, 1-6, 1-7, 1-8, 1-9, or 1-10. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, each n is, independently, greater than 10. In some embodiments, the flexible peptide linker is (GGGGA)ₙ (SEQ ID NO: 122) and n is 1. In some embodiments, the flexible peptide linker is (GGGGA)ₙ (SEQ ID NO: 122) and n is 2. In some embodiments, the flexible peptide linker is (GGGGA)ₙ (SEQ ID NO: 122) and n is 3. In some embodiments, the flexible peptide linker is (GGGGA)ₙ (SEQ ID NO: 122) and n is 4. In some embodiments, the flexible peptide linker is (GGGGA)ₙ (SEQ ID NO: 122) and n is 5. In some embodiments, the flexible peptide linker is (GGGGA)ₙ (SEQ ID NO: 122) and n is 6. In some embodiments, the flexible peptide linker is (GGGGA)ₙ (SEQ ID NO: 122) and n is 7. In some embodiments, the flexible peptide linker is (GGGGA)ₙ (SEQ ID NO: 122) and n is 8. In some embodiments, the flexible peptide linker is (GGGGA)ₙ (SEQ ID NO: 122) and n is 9. In some embodiments, the flexible peptide linker is (GGGGA)ₙ (SEQ ID NO: 122) and n is 10. In some embodiments, the flexible peptide linker is (GGGGA)ₙ (SEQ ID NO: 122) and n is greater than 10. In some embodiments, the flexible peptide linker is (GGGGS)ₙ(SEQ ID NO: 4) and n is 1. In some embodiments, the flexible peptide linker is (GGGGS)ₙ (SEQ ID NO: 4) and n is 2. In some embodiments, the flexible peptide linker is (GGGGS)ₙ (SEQ ID NO: 4) and n is 3. In some embodiments, the flexible peptide linker is (GGGGS)ₙ (SEQ ID NO: 4) and n is 4. In some embodiments, the flexible peptide linker is (GGGGS)ₙ (SEQ ID NO: 4) and n is 5. In some embodiments, the flexible peptide linker is (GGGGS)ₙ (SEQ ID NO: 4) and n is 6. In some embodiments, the flexible peptide linker is (GGGGS)ₙ (SEQ ID NO: 4) and n is 7. In some embodiments, the flexible peptide linker is (GGGGS)ₙ (SEQ ID NO: 4) and n is 8. In some embodiments, the flexible peptide linker is (GGGGS)ₙ (SEQ ID NO: 4) and n is 9. In some embodiments, the flexible peptide linker is (GGGGS)ₙ (SEQ ID NO: 4) and n is 10. In some embodiments, the flexible peptide linker is (GGGGS)ₙ (SEQ ID NO: 4) and n is greater than 10. In some embodiments, the flexible peptide linker is GSAGSAAGSGEF (SEQ ID NO: 123). In some embodiments, the flexible peptide linker is KESGSVSSEQLAQFRSLD (SEQ ID NO: 124). In some embodiments, the flexible peptide linker is EGKSSGSGSESKST (SEQ ID NO: 125).

As provided for herein, the affinity binding polypeptides may be located at the N-terminus, at the C-terminus, or within the targeting moiety. In some embodiments, the at least a first affinity binding polypeptide is located at the N-terminus of the targeting moiety. In some embodiments, the at least a first affinity binging polypeptide is located in the linker domain between the V_{H} and V_{L} regions of a targeting moiety as provided for herein. In some embodiments, the at least a first affinity binding polypeptide is located between the V_{H} and the flexible stalk. In some embodiments, the at least a first affinity binding polypeptide is located between the V_{L} and the flexible stalk.

In some embodiments, the affinity binding polypeptide fused to the targeting moiety comprises an amino acid sequence having at least 70% identity to SEQ ID NO 126:

(SEQ ID NO: 126)
MALPVTALLLPLALLLHAARPWSHPQFEKGGSGSDIQVQLQQPGAELVKP
GASVKLSCKASGYPFTSYWIHWVKQRPGRGLEWLGRIDPNSGDTKYNEKF
KNKATLTVDKSSTTAYMQLSSLTSEDSAVYYCARSPYYSNDNSMDYWGQG
TSVTVSSGGGGSGGGGSGGGGSGGGGSDILLTQSPAILSVSPGERVSFSC
RASQSIGTSIHWYQQRTNDSPRLLIKYASESISGIPSRFSGSGSGTDFTL
SINSVESEDIADYYCQQSNSWPTTFGGGTKLEIKRVNGGGGSGGGGSGGG
GSGGGGSAVHTRGLDFACDIYIWAPLAGTCGVLLLSLVITLYCNHRNRRR
VNRVRQGYS

or is substantially similar to SEQ ID NO: 126, or is an active fragment of SEQ ID NO: 126. (Bold and underlined sequence represents the affinity binding polypeptide, bolded sequence no underline represents linker peptide, italic sequence represents leader peptide, underlined sequence represents targeting moiety). The sequence of SEQ ID NO: 126 contains the leader peptide MALPVTALLLPLALLLHAARP (SEQ ID NO: 177). Upon processing, the leader peptide may be cleaved leaving the sequence of SEQ ID NO: 178 (See Table 6). Thus, while embodiments may refer to further modifications of, mutations of, or percent identity to SEQ ID NO: 126, it is to be understood that the same modifications, mutations, and limitations to percent identity also apply to SEQ ID NO: 178. In some embodiments, the targeting moiety fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 126. In some embodiments, the targeting moiety fused to the affinity binding polypeptide comprises an amino acid sequence of SEQ ID NO: 126.

In some embodiments, the affinity binding polypeptide fused to the targeting moiety comprises an amino acid sequence having at least 70% identity to SEQ ID NO 129:

(SEQ ID NO: 129)
METDTLLLWVLLLWVPGSTGDWSHPQFEKGGGGSGGGGSSAQVQLQQPGA
ELVKPGASVKLSCKASGYPFTSYWIHWVKQRPGRGLEWLGRIDPNSGDTK
YNEKFKNKATLTVDKSSTTAYMQLSSLISEDSAVYYCARSPYYSNDNSMD
YWGQGTSVTVSSGGGGSGGGGSGGGGSGGGGSDILLTQSPAILSVSPGER
VSFSCRASQSIGTSIHWYQQRTNDSPRLLIKYASESISGIPSRFSGSGSG
TDFTLSINSVESEDIADYYCQQSNSWPTTFGGGTKLEIKRASGGGGSGGG
GSGGGGSEPKSCDKTHTCPPCPAPEAAGGPSVFLFPPKPKDTLMASRTPE
VTCVVVDVSHEDPEVKFNWYVDGVEVHNAKTKPREEQYASTYRVVSVLTV
LAQDWLNGKEYKCKVSNKALGAPIEKTISKAKGQPREPQVYTLPPSRDEL
TKNQVSLTCLVKGFYPSDIAVEWESNGQPENNYKTTPPVLDSDGSFFLYS
KLTVDKSRWQQGNVFSCSVMHEALHNAYTQKSLSLSPGKKIEVMYPPPYL
DNEKSNGTIIHVKGKHLCPSPLFPGPSKPFWVLVVVGGVLACYSLLVTVA
FIIFWVRSKRSRLLHSDYMNRVRQGYS

or is substantially similar to SEQ ID NO: 129, or is an active fragment of SEQ ID NO: 129. (Bold and underlined sequence represents the affinity binding polypeptide, bolded sequence no underline represents linker peptide, italic sequence represents leader peptide, underlined sequence represents targeting moiety). The sequence of SEQ ID NO: 129 contains the leader peptide METDTLLLWVLLLWVPGSTGD (SEQ ID NO: 173). Upon processing, the leader peptide may be cleaved leaving the sequence of SEQ ID NO: 179 (See Table 6). Thus, while embodiments may refer to further modifications of, mutations of, or percent identity to SEQ ID NO: 129, it is to be understood that the same modifications, mutations, and limitations to percent identity also apply to SEQ ID NO: 179. In some embodiments, the sequence of SEQ ID NO: 129 further comprises an amino acid deletion at position 40, 41, 291, 292, or a combination thereof as compared to SEQ ID NO: 129. In some embodiments, the deletion at position 40 comprises a S40 deletion. In some embodiments, the deletion at position 41 comprises a A41 deletion. In some embodiments, the deletion at position 291 comprises a A291 deletion. In some embodiments, the deletion at position 292 comprises a S292 deletion. In some embodiments, the affinity binding polypeptide fused to the targeting moiety comprising amino acid deletions at positions 541, A42, A291, and S292 as compared to SEQ ID NO: 129 comprises the amino acid sequence of SEQ ID NO: 180 (See Table 6). In some embodiments, the affinity binding polypeptide fused to the targeting moiety comprising amino acid deletions at positions 541, A42, A291, and S292 as compared to SEQ ID NO: 129 and further comprising removal of the leader peptide comprises the amino acid sequence of SEQ ID NO: 181 (See Table 6). In some embodiments, the targeting moiety fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 129. In some embodiments, the targeting moiety fused to the affinity binding polypeptide comprises an amino acid sequence of SEQ ID NO: 129.

In some embodiments, the affinity binding polypeptide fused to the targeting moiety comprises an amino acid sequence having at least 70% identity to SEQ ID NO 130:

(SEQ ID NO: 130)
METDTLLLWVLLLWVPGSTGDSAQVQLQQPGAELVKPGASVKLSCKASGY
PFTSYWIHWVKQRPGRGLEWLGRIDPNSGDTKYNEKFKNKATLTVDKSST
TAYMQLSSLTSEDSAVYYCARSPYYSNDNSMDYWGQGTSVTVSSGGGGSG
GGGSWSHPQFEKGGGGSGGGGSDILLTQSPAILSVSPGERVSFSCRASQS
IGTSIHWYQQRTNDSPRLLIKYASESISGIPSRFSGSGSGTDFTLSINSV
ESEDIADYYCQQSNSWPTTFGGGTKLEIKRASGGGGSGGGGSGGGGSEPK
SCDKTHTCPPCPAPEAAGGPSVFLFPPKPKDTLMASRTPEVTCVVVDVSH
EDPEVKFNWYVDGVEVHNAKTKPREEQYASTYRVVSVLTVLAQDWLNGKE
YKCKVSNKALGAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNAYTQKSLSLSPGKKIEVMYPPPYLDNEKSNGTII
HVKGKHLCPSPLFPGPSKPFWVLVVVGGVLACYSLLVTVAFIIFWVRSKR
SRLLHSDYMNRVRQGYS

or is substantially similar to SEQ ID NO: 130, or is an active fragment of SEQ ID NO: 130. (Bold and underlined sequence represents the affinity binding polypeptide, bolded sequence no underline represents linker peptide, italic sequence represents leader peptide, underlined sequence represents targeting moiety). The sequence of SEQ ID NO: 130 contains the leader peptide METDTLLLWVLLLWVPGSTGD (SEQ ID NO: 173). Upon processing, the leader peptide may be cleaved leaving the sequence of SEQ ID NO: 182 (See Table 6). Thus, while embodiments may refer to further modifications of, mutations of, or percent identity to SEQ ID NO: 130, it is to be understood that the same modifications, mutations, and limitations to percent identity also apply to SEQ ID NO: 182. In some embodiments, the sequence of SEQ ID NO: 130 further comprises an amino acid deletion at position 22, 23, 281, 282, or a combination thereof as compared to SEQ ID NO: 130. In some embodiments, the deletion at position 22 comprises a S22 deletion. In some embodiments, the deletion at position 23 comprises a A23 deletion. In some embodiments, the deletion at position 281 comprises a A281 deletion. In some embodiments, the deletion at position 282 comprises a S282 deletion.

In some embodiments, the affinity binding polypeptide fused to the targeting moiety comprising amino acid deletions at positions S22, A23, A281, and S282 as compared to SEQ ID NO: 130 comprises the amino acid sequence of SEQ ID NO: 183 (See Table 6). In some embodiments, the affinity binding polypeptide fused to the targeting moiety comprising amino acid deletions at positions S22, A23, A281, and S292 as compared to SEQ ID NO: 130 and further comprising removal of the leader peptide comprises the amino acid sequence of SEQ ID NO: 184 (See Table 6). In some embodiments, the targeting moiety fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 130. In some embodiments, the targeting moiety fused to the affinity binding polypeptide comprises an amino acid sequence of SEQ ID NO: 130.

In some embodiments, the affinity binding polypeptide fused to the targeting moiety comprises an amino acid sequence having at least 70% identity to SEQ ID NO 131:

(SEQ ID NO: 131)
METDTLLLWVLLLWVPGSTGDSAQVQLQQPGAELVKPGASVKLSCKASGY
PFTSYWIHWVKQRPGRGLEWLGRIDPNSGDTKYNEKFKNKATLTVDKSST
TAYMQLSSLTSEDSAVYYCARSPYYSNDNSMDYWGQGTSVTVSSGGGGSG
GGGSGGGGSGGGGSDILLTQSPAILSVSPGERVSFSCRASQSIGTSIHWY
QQRTNDSPRLLIKYASESISGIPSRFSGSGSGTDFTLSINSVESEDIADY
YCQQSNSWPTTFGGGTKLEIKRASGGGGSGGGGSWSHPQFEKGGGGSEPK
SCDKTHTCPPCPAPEAAGGPSVFLFPPKPKDTLMASRTPEVTCVVVDVSH
EDPEVKFNWYVDGVEVHNAKTKPREEQYASTYRVVSVLTVLAQDWLNGKE
YKCKVSNKALGAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNAYTQKSLSLSPGKKIEVMYPPPYLDNEKSNGIII
HVKGKHLCPSPLFPGPSKPFWVLVVVGGVLACYSLLVTVAFIIFWVRSKR
SRLLHSDYMNRVRQGYS

or is substantially similar to SEQ ID NO: 131, or is an active fragment of SEQ ID NO: 131. (Bold and underlined sequence represents the affinity binding polypeptide, bolded sequence no underline represents linker peptide, italic sequence represents leader peptide, underlined sequence represents targeting moiety). The sequence of SEQ ID NO: 131 contains the leader peptide METDTLLLWVLLLWVPGSTGD (SEQ ID NO: 173). Upon processing, the leader peptide may be cleaved leaving the sequence of SEQ ID NO: 185 (See Table 6). Thus, while embodiments may refer to further modifications of, mutations of, or percent identity to SEQ ID NO: 131, it is to be understood that the same modifications, mutations, and limitations to percent identity also apply to SEQ ID NO: 185. In some embodiments, the sequence of SEQ ID NO: 131 further comprises an amino acid deletion at position 22, 23, 273, 274, or a combination thereof as compared to SEQ ID NO: 131. In some embodiments, the deletion at position 22 comprises a S22 deletion. In some embodiments, the deletion at position 23 comprises a A23 deletion. In some embodiments, the deletion at position 273 comprises a A273 deletion. In some embodiments, the deletion at position 274 comprises a S274 deletion.

In some embodiments, the affinity binding polypeptide fused to the targeting moiety comprising amino acid deletions at positions S22, A23, A273, and S274 as compared to SEQ ID NO: 131 comprises the amino acid sequence of SEQ ID NO: 186 (See Table 6). In some embodiments, the affinity binding polypeptide fused to the targeting moiety comprising amino acid deletions at positions S22, A23, A273, and S274 as compared to SEQ ID NO: 131 and further comprising removal of the leader peptide comprises the amino acid sequence of SEQ ID NO: 187 (See Table 6).In some embodiments, the targeting moiety fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 131. In some embodiments, the targeting moiety fused to the affinity binding polypeptide comprises an amino acid sequence of SEQ ID NO: 131.

In some embodiments, the pseudotyped viral-like particle or viral vector further comprises a second targeting moiety. In some embodiments, the second targeting moiety is any targeting moiety as provided for herein. In some embodiments, the second targeting moiety does not comprise as affinity binding polypeptide. In some embodiments, the second targeting moiety further comprises at least a first, at least a second, at least a third, at least a fourth, or at least a fifth affinity binding polypeptide sequence. In some embodiments, the second targeting moiety further comprises at least a first affinity binding polypeptide sequence. In some embodiments, the identity of the at least a first affinity binding polypeptide sequence is as provided for herein. The at least a first affinity binding polypeptide of the second targeting moiety can be inserted at any position in the second targeting moiety that does not substantially negatively affect the function or purpose of the targeting moiety. Accordingly, in some embodiments, the at least a first affinity binding polypeptide is located at the N-terminus, at the C-terminus, or within the second targeting moiety. In some embodiments, the at least a first affinity binding polypeptide is located at the N-terminus of the second targeting moiety. In some embodiments, the at least a first affinity binding polypeptide is located at the C-terminus of the second targeting moiety. In some embodiments, the at least a first affinity binding polypeptide is located within the second targeting moiety. In some embodiments, the at least a first affinity binding polypeptide is fused directly to the second targeting moiety. In some embodiments, the at least a first affinity binding polypeptide is fused indirectly to the second targeting moiety via, for example, a peptide linker as provided for herein. In embodiments where the at least a first affinity binding polypeptide of the second targeting moiety is located within the second targeting moiety, the affinity binding polypeptide may be fused to the targeting moiety directly, fused directly at the N-terminus of the affinity binding polypeptide and fused indirectly at the C-terminus of the affinity binding polypeptide via, for example, a peptide linker as provided for herein, fused indirectly at the N-terminus of the affinity binding polypeptide via, for example, a peptide linker as provided for herein and fused directly at the C-terminus of the affinity binding polypeptide, or fused indirectly at both the N and C terminus of the affinity binding polypeptide via, for example, a polypeptide linker as provided for herein.

In some embodiments, the second targeting moiety further comprises at least a second affinity binding polypeptide. In some embodiments, the identity of the at least a second affinity binding polypeptide is as provided for herein. The at least a second affinity binding polypeptide of the second targeting moiety can be inserted at any position in the second targeting moiety that does not affect the function or purpose of the glycoprotein. Further, the position of the at least a second affinity binding polypeptide is independent of the position of the at least a first affinity binding polypeptide, so long as the combination of first and second affinity binding polypeptide does not negatively affect the function or purpose of the second targeting moiety. Accordingly, in some embodiments, the at least a second affinity binding polypeptide is located at the N-terminus, at the C-terminus, or within the second targeting moiety. In some embodiments, the at least a second affinity binding polypeptide is located at the N-terminus of the second targeting moiety. In some embodiments, the at least a second affinity binding polypeptide is located at the C-terminus of the second targeting moiety. In some embodiments, the at least a second affinity binding polypeptide is located within the second targeting moiety. In some embodiments, the at least a second affinity binding polypeptide is fused directly to the second targeting moiety. In some embodiments, the at least a second affinity binding polypeptide is fused indirectly to the second targeting moiety via, for example, a peptide linker as provided for herein. In embodiments where the at least a second affinity binding polypeptide of the second targeting moiety is located within the targeting moiety, the affinity binding polypeptide may be fused to the targeting moiety directly, fused directly at the N-terminus of the affinity binding polypeptide and fused indirectly at the C-terminus of the affinity binding polypeptide via, for example, a peptide linker as provided for herein, fused indirectly at the N-terminus of the affinity binding polypeptide via, for example, a peptide linker as provided for herein and fused directly at the C-terminus of the affinity binding polypeptide, or fused indirectly at both the N and C terminus of the affinity binding polypeptide via, for example, a polypeptide linker as provided for herein. In some embodiments, the second targeting moiety further comprises at least a third, at least a fourth, or at least a fifth affinity binding polypeptide. In some embodiments, the second targeting moiety further comprises at least a third affinity binding polypeptide. In some embodiments, the identity of the at least a third affinity binding polypeptide is as provided for herein. In some embodiments, the second targeting moiety further comprises at least a fourth affinity binding polypeptide. In some embodiments, the identity of the at least a fourth affinity binding polypeptide is as provided for herein. In some embodiments, the second targeting moiety further comprises at least a fifth affinity binding polypeptide. In some embodiments, the identity of the at least a fifth affinity binding polypeptide is as provided for herein. In some embodiments, the position of the at least a third affinity binding polypeptide on or within the targeting moiety is as provided for herein for the at least a first and the at least a second affinity binding polypeptide fused to the second targeting moiety. In some embodiments, the position of the at least a fourth affinity binding polypeptide on or within the targeting moiety is as provided for herein for the at least a first and the at least a second affinity binding polypeptide fused to the second targeting moiety. In some embodiments, the position of the at least a fifth affinity binding polypeptide on or within the targeting moiety is as provided for herein for the at least a first and the at least a second affinity binding polypeptide fused to the second targeting moiety.

The number of affinity binding polypeptides fused to the second targeting moiety is only limited in that the fusion constructs should not negatively affect the function or purpose of the second targeting moiety. Thus, in some embodiments, the second targeting moiety further comprises at least one affinity binding polypeptide. In some embodiments, the second targeting moiety further comprises at least two affinity binding polypeptides. In some embodiments, the second targeting moiety further comprises at least three affinity binding polypeptides. In some embodiments, the second targeting moiety further comprises at least four affinity binding polypeptides. In some embodiments, the second targeting moiety further comprises at least five affinity binding polypeptides. In some embodiments, the second targeting moiety further comprises up to five affinity binding polypeptides. In some embodiments, the second targeting moiety further comprises up to ten affinity binding polypeptides. In some embodiments, the second targeting moiety further comprises up to twenty affinity binding polypeptides.

In some embodiments, the affinity binding polypeptide fused to the second targeting moiety comprises an amino acid sequence having at least 70% identity to SEQ ID NO: 121, SEQ ID NO: 174, SEQ ID NO: 175, or SEQ ID NO: 176, or is substantially similar to SEQ ID NO: 121, SEQ ID NO: 174, SEQ ID NO: 175, or SEQ ID NO: 176, or is an active fragment of SEQ ID NO: 121, SEQ ID NO: 174, SEQ ID NO: 175, or SEQ ID NO: 176. In some embodiments, the second targeting moiety fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 121, SEQ ID NO: 174, SEQ ID NO: 175, or SEQ ID NO: 176. In some embodiments, the second targeting moiety fused to the affinity binding polypeptide comprises an amino acid sequence of SEQ ID NO: 121. In some embodiments, the second targeting moiety fused to the affinity binding polypeptide comprises an amino acid sequence of SEQ ID NO: 174. In some embodiments, the second targeting moiety fused to the affinity binding polypeptide comprises an amino acid sequence of SEQ ID NO: 175. In some embodiments, the second targeting moiety fused to the affinity binding polypeptide comprises an amino acid sequence of SEQ ID NO: 176.

In some embodiments, the affinity binding polypeptide fused to the second targeting moiety comprises an amino acid sequence having at least 70% identity to SEQ ID NO: 126 or SEQ ID NO: 178, or is substantially similar to SEQ ID NO: 126 or SEQ ID NO: 178, or is an active fragment of SEQ ID NO: 126 or SEQ ID NO: 178. In some embodiments, the second targeting moiety fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 126 or SEQ ID NO: 178. In some embodiments, the second targeting moiety fused to the affinity binding polypeptide comprises an amino acid sequence of SEQ ID NO: 126. In some embodiments, the second targeting moiety fused to the affinity binding polypeptide comprises an amino acid sequence of SEQ ID NO: 178.

In some embodiments, the affinity binding polypeptide fused to the second targeting moiety comprises an amino acid sequence having at least 70% identity to SEQ ID NO: 129, SEQ ID NO: 179, SEQ ID NO: 180, or SEQ ID NO: 181, or is substantially similar to SEQ ID NO: 129, SEQ ID NO: 179, SEQ ID NO: 180, or SEQ ID NO: 181, or is an active fragment of SEQ ID NO: 129, SEQ ID NO: 179, SEQ ID NO: 180, or SEQ ID NO: 181. In some embodiments, the second targeting moiety fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 129, SEQ ID NO: 179, SEQ ID NO: 180, or SEQ ID NO: 181. In some embodiments, the second targeting moiety fused to the affinity binding polypeptide comprises an amino acid sequence of SEQ ID NO: 129. In some embodiments, the second targeting moiety fused to the affinity binding polypeptide comprises an amino acid sequence of SEQ ID NO: 179. In some embodiments, the second targeting moiety fused to the affinity binding polypeptide comprises an amino acid sequence of SEQ ID NO: 180. In some embodiments, the second targeting moiety fused to the affinity binding polypeptide comprises an amino acid sequence of SEQ ID NO: 181.

In some embodiments, the affinity binding polypeptide fused to the second targeting moiety comprises an amino acid sequence having at least 70% identity to SEQ ID NO: 130, SEQ ID NO: 182, SEQ ID NO: 183, or SEQ ID NO: 184, or is substantially similar to SEQ ID NO: 130, SEQ ID NO: 182, SEQ ID NO: 183, or SEQ ID NO: 184, or is an active fragment of SEQ ID NO: 130, SEQ ID NO: 182, SEQ ID NO: 183, or SEQ ID NO: 184. In some embodiments, the second targeting moiety fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 130, SEQ ID NO: 182, SEQ ID NO: 183, or SEQ ID NO: 184. In some embodiments, the second targeting moiety fused to the affinity binding polypeptide comprises an amino acid sequence of SEQ ID NO: 130. In some embodiments, the second targeting moiety fused to the affinity binding polypeptide comprises an amino acid sequence of SEQ ID NO: 182. In some embodiments, the second targeting moiety fused to the affinity binding polypeptide comprises an amino acid sequence of SEQ ID NO: 183. In some embodiments, the second targeting moiety fused to the affinity binding polypeptide comprises an amino acid sequence of SEQ ID NO: 184.

In some embodiments, the affinity binding polypeptide fused to the second targeting moiety comprises an amino acid sequence having at least 70% identity to SEQ ID NO: 131, SEQ ID NO: 185, SEQ ID NO: 186, or SEQ ID NO: 187, or is substantially similar to SEQ ID NO: 131, SEQ ID NO: 185, SEQ ID NO: 186, or SEQ ID NO: 187, or is an active fragment of SEQ ID NO: 131, SEQ ID NO: 185, SEQ ID NO: 186, or SEQ ID NO: 187. In some embodiments, the second targeting moiety fused to the affinity binding polypeptide comprises an amino acid sequence having least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 131, SEQ ID NO: 185, SEQ ID NO: 186, or SEQ ID NO: 187. In some embodiments, the second targeting moiety fused to the affinity binding polypeptide comprises an amino acid sequence of SEQ ID NO: 131. In some embodiments, the second targeting moiety fused to the affinity binding polypeptide comprises an amino acid sequence of SEQ ID NO: 185. In some embodiments, the second targeting moiety fused to the affinity binding polypeptide comprises an amino acid sequence of SEQ ID NO: 186. In some embodiments, the second targeting moiety fused to the affinity binding polypeptide comprises an amino acid sequence of SEQ ID NO: 187.

In some embodiments, the affinity binding polypeptides as provided for herein are located only on the virus glycoprotein, or on a combination of more than one viral glycoproteins as provided for herein. In some embodiments, the affinity binding polypeptides as provided for herein are located only on the targeting moiety, or on a combination of more than one targeting moieties as provided for herein. In some embodiments, the affinity binding polypeptides as provided for herein are located on both the virus glycoprotein and the targeting moiety.

In some embodiments, a pseudotyped viral-like particle or viral vector is provided. In some embodiments, the pseudotyped viral-like particle or viral vector comprises an engineered enveloped comprising a recombinant virus glycoprotein, an engineered targeting moiety for binding to a target cell, and at least a first affinity binding polypeptide, wherein the at least a first affinity binding polypeptide is fused to the glycoprotein, and the glycoprotein comprises an amino acid sequence of SEQ ID NO: 25, SEQ ID NO: 29, SEQ ID NO: 30, SEQ ID NO: 33, SEQ ID NO: 34, SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 66, SEQ ID NO: 67, SEQ ID NO: 74; SEQ ID NO: 77, SEQ ID NO: 79, or SEQ ID NO: 80. In some embodiments, the pseudotyped viral-like particle or viral vector further comprises a nucleic acid molecule encoding a polypeptide of interest. In some embodiments, the pseudotyped viral-like particle or viral vector is a lentivirus.

In some embodiments, the virus glycoprotein is an Ebola virus glycoprotein and comprises the amino acid sequence of SEQ ID NO: 25, or a variant thereof as provided for herein.

In some embodiments, the virus glycoprotein is a Nipah G (NiV-G) protein and comprises the amino acid sequence of SEQ ID NO: 29 or SEQ ID NO: 30 or a variant thereof as provided for herein. In some embodiments, the NiV-G protein comprises the amino acid sequence of SEQ ID NO: 29 or a variant thereof as provided for herein. In some embodiments, the NiV-G protein comprises the amino acid sequence of SEQ ID NO: 30 or a variant thereof as provided for herein.

In some embodiments, the virus glycoprotein is a Nipah F (NiV-F) protein and comprises the amino acid sequence of SEQ ID NO: 33 or SEQ ID NO: 34 or a variant thereof as provided for herein. In some embodiments, the NiV-F protein comprises the amino acid sequence of SEQ ID NO: 33 or a variant thereof as provided for herein. In some embodiments, the NiV-F protein comprises the amino acid sequence of SEQ ID NO: 34 or a variant thereof as provided for herein.

In some embodiments, the virus glycoprotein is a VSV-G protein and comprises the amino acid sequence of SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 66, or SEQ ID NO: 67, or a variant thereof as provided for herein. In some embodiments, the VSV-G protein comprises the amino acid sequence of SEQ ID NO: 36, or a variant thereof as provided for herein. In some embodiments, the VSV-G protein comprises the amino acid sequence of SEQ ID NO: 37, or a variant thereof as provided for herein. In some embodiments, the VSV-G protein comprises the amino acid sequence of SEQ ID NO: 38, or a variant thereof as provided for herein. In some embodiments, the VSV-G protein comprises the amino acid sequence of SEQ ID NO: 39, or a variant thereof as provided for herein. In some embodiments, the VSV-G protein comprises the amino acid sequence of SEQ ID NO: 40, or a variant thereof as provided for herein. In some embodiments, the VSV-G protein comprises the amino acid sequence of SEQ ID NO: 41, or a variant thereof as provided for herein. In some embodiments, the VSV-G protein comprises the amino acid sequence of SEQ ID NO: 42, or a variant thereof as provided for herein. In some embodiments, the VSV-G protein comprises the amino acid sequence of SEQ ID NO: 43, or a variant thereof as provided for herein. In some embodiments, the VSV-G protein comprises the amino acid sequence of SEQ ID NO: 44, or a variant thereof as provided for herein. In some embodiments, the VSV-G protein comprises the amino acid sequence of SEQ ID NO: 45, or a variant thereof as provided for herein. In some embodiments, the VSV-G protein comprises the amino acid sequence of SEQ ID NO: 46, or a variant thereof as provided for herein. In some embodiments, the VSV-G protein comprises the amino acid sequence of SEQ ID NO: 47, or a variant thereof as provided for herein. In some embodiments, the VSV-G protein comprises the amino acid sequence of SEQ ID NO: 49, or a variant thereof as provided for herein. In some embodiments, the VSV-G protein comprises the amino acid sequence of SEQ ID NO: 50, or a variant thereof as provided for herein. In some embodiments, the VSV-G protein comprises the amino acid sequence of SEQ ID NO: 52, or a variant thereof as provided for herein. In some embodiments, the VSV-G protein comprises the amino acid sequence of SEQ ID NO: 53, or a variant thereof as provided for herein. In some embodiments, the VSV-G protein comprises the amino acid sequence of SEQ ID NO: 55, or a variant thereof as provided for herein. In some embodiments, the VSV-G protein comprises the amino acid sequence of SEQ ID NO: 56, or a variant thereof as provided for herein. In some embodiments, the VSV-G protein comprises the amino acid sequence of SEQ ID NO: 58, or a variant thereof as provided for herein. In some embodiments, the VSV-G protein comprises the amino acid sequence of SEQ ID NO: 59, or a variant thereof as provided for herein. In some embodiments, the VSV-G protein comprises the amino acid sequence of SEQ ID NO: 61, or a variant thereof as provided for herein. In some embodiments, the VSV-G protein comprises the amino acid sequence of SEQ ID NO: 62, or a variant thereof as provided for herein. In some embodiments, the VSV-G protein comprises the amino acid sequence of SEQ ID NO: 64, or a variant thereof as provided for herein. In some embodiments, the VSV-G protein comprises the amino acid sequence of SEQ ID NO: 65, or a variant thereof as provided for herein. In some embodiments, the VSV-G protein comprises the amino acid sequence of SEQ ID NO: 66, or a variant thereof as provided for herein. In some embodiments, the VSV-G protein comprises the amino acid sequence of SEQ ID NO: 67, or a variant thereof as provided for herein.

In some embodiments, the virus glycoprotein is a measles virus MeV-H protein and comprises the amino acid sequence of SEQ ID NO: 74, or a variant thereof as provided for herein.

In some embodiments, the virus glycoprotein is a measles virus MeV-F protein and comprises the amino acid sequence of SEQ ID NO: 77, or a variant thereof as provided for herein.

In some embodiments, the virus glycoprotein is a SVCV-G protein and comprises the amino acid sequence of SEQ ID NO: 79 or SEQ ID NO: 80, or a variant thereof as provided for herein. In some embodiments, the SVCV-G protein comprises the amino acid sequence of SEQ ID NO: 79, or a variant thereof as provided for herein. In some embodiments, the SVCV-G protein comprises the amino acid sequence of SEQ ID NO: 80, or a variant thereof as provided for herein.

In some embodiments, the identity of the at least a first affinity binding polypeptide is as provided for herein. In some embodiments, the at least a first affinity binding polypeptide is located at the N-terminus, at the C-terminus, or within the glycoprotein. In some embodiments, the location of the at least a first affinity binding polypeptide with respect to the glycoprotein is as provided for herein.

In some embodiments, a pseudotyped viral-like particle or viral vector is provided. In some embodiments, the pseudotyped viral-like particle or viral vector comprises an engineered enveloped comprising a recombinant virus glycoprotein, an engineered targeting moiety for binding to a target cell, and at least a first affinity binding polypeptide, wherein the at least a first affinity binding polypeptide is fused to the glycoprotein. In some embodiments, the affinity binding polypeptide fused to the glycoprotein comprises a sequence selected from the group including, but not limited to, SEQ ID NO: 26, SEQ ID NO: 27, SEQ ID NO: 28, SEQ ID NO: 31, SEQ ID NO: 32, SEQ ID NO: 35, SEQ ID NO: 68, SEQ ID NO: 69, SEQ ID NO: 70, SEQ ID NO: 71, SEQ ID NO: 72, SEQ ID NO: 73, SEQ ID NO: 75, SEQ ID NO: 76, SEQ ID NO: 78, SEQ ID NO: 81, SEQ ID NO: 132, SEQ ID NO: 133, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 147, SEQ IF NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 166, SEQ ID NO: 167, SEQ ID NO: 168, SEQ ID NO: 170, or SEQ ID NO: 172, or any variants thereof as provided for herein. In some embodiments, the pseudotyped viral-like particle or viral vector further comprises a nucleic acid molecule encoding a polypeptide of interest. In some embodiments, the pseudotyped viral-like particle or viral vector is a lentivirus.

In some embodiments, the virus glycoprotein is an Ebola virus glycoprotein and the affinity binding polypeptide fused to the Ebola virus glycoprotein comprises the amino acid sequence of SEQ ID NO: 26, SEQ ID NO: 27, SEQ ID NO: 28, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 147, SEQ IF NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 188, SEQ ID NO: 189, SEQ ID NO: 190, or SEQ ID NO: 191, or any variant thereof as provided for herein. In some embodiments, the affinity binding polypeptide fused to the Ebola virus glycoprotein comprises the amino acid sequence of SEQ ID NO: 26, or a variant thereof as provided for herein. In some embodiments, the affinity binding polypeptide fused to the Ebola virus glycoprotein comprises the amino acid sequence of SEQ ID NO: 27, or a variant thereof as provided for herein. In some embodiments, the affinity binding polypeptide fused to the Ebola virus glycoprotein comprises the amino acid sequence of SEQ ID NO: 28, or a variant thereof as provided for herein. In some embodiments, the affinity binding polypeptide fused to the Ebola virus glycoprotein comprises the amino acid sequence of SEQ ID NO: 135, or a variant thereof as provided for herein. In some embodiments, the affinity binding polypeptide fused to the Ebola virus glycoprotein comprises the amino acid sequence of SEQ ID NO: 136, or a variant thereof as provided for herein. In some embodiments, the affinity binding polypeptide fused to the Ebola virus glycoprotein comprises the amino acid sequence of SEQ ID NO: 137, or a variant thereof as provided for herein. In some embodiments, the affinity binding polypeptide fused to the Ebola virus glycoprotein comprises the amino acid sequence of SEQ ID NO: 138, or a variant thereof as provided for herein. In some embodiments, the affinity binding polypeptide fused to the Ebola virus glycoprotein comprises the amino acid sequence of SEQ ID NO: 139, or a variant thereof as provided for herein. In some embodiments, the affinity binding polypeptide fused to the Ebola virus glycoprotein comprises the amino acid sequence of SEQ ID NO: 140, or a variant thereof as provided for herein. In some embodiments, the affinity binding polypeptide fused to the Ebola virus glycoprotein comprises the amino acid sequence of SEQ ID NO: 141, or a variant thereof as provided for herein. In some embodiments, the affinity binding polypeptide fused to the Ebola virus glycoprotein comprises the amino acid sequence of SEQ ID NO: 142, or a variant thereof as provided for herein. In some embodiments, the affinity binding polypeptide fused to the Ebola virus glycoprotein comprises the amino acid sequence of SEQ ID NO: 143, or a variant thereof as provided for herein. In some embodiments, the affinity binding polypeptide fused to the Ebola virus glycoprotein comprises the amino acid sequence of SEQ ID NO: 144, or a variant thereof as provided for herein. In some embodiments, the affinity binding polypeptide fused to the Ebola virus glycoprotein comprises the amino acid sequence of SEQ ID NO: 145, or a variant thereof as provided for herein. In some embodiments, the affinity binding polypeptide fused to the Ebola virus glycoprotein comprises the amino acid sequence of SEQ ID NO: 147, or a variant thereof as provided for herein. In some embodiments, the affinity binding polypeptide fused to the Ebola virus glycoprotein comprises the amino acid sequence of SEQ ID NO: 148, or a variant thereof as provided for herein. In some embodiments, the affinity binding polypeptide fused to the Ebola virus glycoprotein comprises the amino acid sequence of SEQ ID NO: 149, or a variant thereof as provided for herein. In some embodiments, the affinity binding polypeptide fused to the Ebola virus glycoprotein comprises the amino acid sequence of SEQ ID NO: 150, or a variant thereof as provided for herein. In some embodiments, the affinity binding polypeptide fused to the Ebola virus glycoprotein comprises the amino acid sequence of SEQ ID NO: 151, or a variant thereof as provided for herein. In some embodiments, the affinity binding polypeptide fused to the Ebola virus glycoprotein comprises the amino acid sequence of SEQ ID NO: 152, or a variant thereof as provided for herein. In some embodiments, the affinity binding polypeptide fused to the Ebola virus glycoprotein comprises the amino acid sequence of SEQ ID NO: 153, or a variant thereof as provided for herein. In some embodiments, the affinity binding polypeptide fused to the Ebola virus glycoprotein comprises the amino acid sequence of SEQ ID NO: 154, or a variant thereof as provided for herein. In some embodiments, the affinity binding polypeptide fused to the Ebola virus glycoprotein comprises the amino acid sequence of SEQ ID NO: 155, or a variant thereof as provided for herein. In some embodiments, the affinity binding polypeptide fused to the Ebola virus glycoprotein comprises the amino acid sequence of SEQ ID NO: 156, or a variant thereof as provided for herein. In some embodiments, the affinity binding polypeptide fused to the Ebola virus glycoprotein comprises the amino acid sequence of SEQ ID NO: 157, or a variant thereof as provided for herein. In some embodiments, the affinity binding polypeptide fused to the Ebola virus glycoprotein comprises the amino acid sequence of SEQ ID NO: 158, or a variant thereof as provided for herein. In some embodiments, the affinity binding polypeptide fused to the Ebola virus glycoprotein comprises the amino acid sequence of SEQ ID NO: 159, or a variant thereof as provided for herein. In some embodiments, the affinity binding polypeptide fused to the Ebola virus glycoprotein comprises the amino acid sequence of SEQ ID NO: 160, or a variant thereof as provided for herein. In some embodiments, the affinity binding polypeptide fused to the Ebola virus glycoprotein comprises the amino acid sequence of SEQ ID NO: 161, or a variant thereof as provided for herein. In some embodiments, the affinity binding polypeptide fused to the Ebola virus glycoprotein comprises the amino acid sequence of SEQ ID NO: 162, or a variant thereof as provided for herein. In some embodiments, the affinity binding polypeptide fused to the Ebola virus glycoprotein comprises the amino acid sequence of SEQ ID NO: 163, or a variant thereof as provided for herein. In some embodiments, the affinity binding polypeptide fused to the Ebola virus glycoprotein comprises the amino acid sequence of SEQ ID NO: 164, or a variant thereof as provided for herein. In some embodiments, the affinity binding polypeptide fused to the Ebola virus glycoprotein comprises the amino acid sequence of SEQ ID NO: 188, or a variant thereof as provided for herein. In some embodiments, the affinity binding polypeptide fused to the Ebola virus glycoprotein comprises the amino acid sequence of SEQ ID NO: 189, or a variant thereof as provided for herein. In some embodiments, the affinity binding polypeptide fused to the Ebola virus glycoprotein comprises the amino acid sequence of SEQ ID NO: 190, or a variant thereof as provided for herein. In some embodiments, the affinity binding polypeptide fused to the Ebola virus glycoprotein comprises the amino acid sequence of SEQ ID NO: 191, or a variant thereof as provided for herein.

In some embodiments, the virus glycoprotein is a Nipah G (NiV-G) protein and the affinity binding polypeptide fused to the NiV-G protein comprises the amino acid sequence of SEQ ID NO: 31, SEQ ID NO: 32, or any variant thereof as provided for herein. In some embodiments, the affinity binding polypeptide fused to the NiV-G protein comprises the amino acid sequence of SEQ ID NO: 31, or a variant thereof as provided for herein. In some embodiments, the affinity binding polypeptide fused to the NiV-G protein comprises the amino acid sequence of SEQ ID NO: 32, or a variant thereof as provided for herein.

In some embodiments, the virus glycoprotein is a Nipah F (NiV-F) protein and the affinity binding polypeptide fused to the NiV-F protein comprises the amino acid sequence of SEQ ID NO: 35 or SEQ ID NO: 166, or any variant thereof as provided for herein. In some embodiments, the affinity binding polypeptide fused to the NiV-F protein comprises the amino acid sequence of SEQ ID NO: 35, or any variant thereof as provided for herein. In some embodiments, the affinity binding polypeptide fused to the NiV-F protein comprises the amino acid sequence of SEQ ID NO: 166, or any variant thereof as provided for herein

In some embodiments, the virus glycoprotein is a VSV-G protein and the affinity binding polypeptide fused to the VSV-G protein comprises the amino acid sequence of SEQ ID NO: 68, SEQ ID NO: 69, SEQ ID NO: 70, SEQ ID NO: 71, SEQ ID NO: 72, SEQ ID NO: 73, SEQ ID NO: 132, SEQ ID NO: 133, SEQ ID NO: 167, or SEQ ID NO: 168, or any variant thereof as provided for herein. In some embodiments, the affinity binding polypeptide fused to the VSV-G protein comprises the amino acid sequence of SEQ ID NO: 68, or a variant as provided for herein. In some embodiments, the affinity binding polypeptide fused to the VSV-G protein comprises the amino acid sequence of SEQ ID NO: 69, or a variant as provided for herein. In some embodiments, the affinity binding polypeptide fused to the VSV-G protein comprises the amino acid sequence of SEQ ID NO: 70, or a variant as provided for herein. In some embodiments, the affinity binding polypeptide fused to the VSV-G protein comprises the amino acid sequence of SEQ ID NO: 71, or a variant as provided for herein. In some embodiments, the affinity binding polypeptide fused to the VSV-G protein comprises the amino acid sequence of SEQ ID NO: 72, or a variant as provided for herein. In some embodiments, the affinity binding polypeptide fused to the VSV-G protein comprises the amino acid sequence of SEQ ID NO: 73, or a variant as provided for herein. In some embodiments, the affinity binding polypeptide fused to the VSV-G protein comprises the amino acid sequence of SEQ ID NO: 132, or a variant as provided for herein. In some embodiments, the affinity binding polypeptide fused to the VSV-G protein comprises the amino acid sequence of SEQ ID NO: 133, or a variant as provided for herein. In some embodiments, the affinity binding polypeptide fused to the VSV-G protein comprises the amino acid sequence of SEQ ID NO: 167, or a variant as provided for herein. In some embodiments, the affinity binding polypeptide fused to the VSV-G protein comprises the amino acid sequence of SEQ ID NO: 168, or a variant as provided for herein.

In some embodiments, the virus glycoprotein is a measles virus MeV-H protein and the affinity binding polypeptide fused to the MeV-H protein comprises the amino acid sequence of SEQ ID NO: 75 or SEQ ID NO: 76, or any variant thereof as provided for herein. In some embodiments, the affinity binding polypeptide fused to the MeV-H protein comprises the amino acid sequence of SEQ ID NO: 75, or a variant thereof as provided for herein. In some embodiments, the affinity binding polypeptide fused to the MeV-H protein comprises the amino acid sequence of SEQ ID NO: 76, or a variant thereof as provided for herein.

In some embodiments, the virus glycoprotein is a measles virus MeV-F protein and the affinity binding polypeptide fused to the MeV-F protein comprises the amino acid sequence of SEQ ID NO: 78 or SEQ ID NO: 170, or any variant thereof as provided for herein. In some embodiments, the affinity binding polypeptide fused to the MeV-F protein comprises the amino acid sequence of SEQ ID NO: 78, or a variant thereof as provided for herein. In some embodiments, the affinity binding polypeptide fused to the MeV-F protein comprises the amino acid sequence of SEQ ID NO: 170, or a variant thereof as provided for herein.

In some embodiments, the virus glycoprotein is a SVCV-G protein and the affinity binding polypeptide fused to the SVCV-G protein comprises the amino acid sequence of SEQ ID NO: 81 or SEQ ID NO: 172, or any variant thereof as provided for herein. In some embodiments, the affinity binding polypeptide fused to the SVCV-G protein comprises the amino acid sequence of SEQ ID NO: 81, or any variant thereof as provided for herein. In some embodiments, the affinity binding polypeptide fused to the SVCV-G protein comprises the amino acid sequence of SEQ ID NO: 172, or any variant thereof as provided for herein.

In some embodiments, the glycoprotein may comprise at least a second, at least a third, at least a fourth, or at least a fifth affinity binding polypeptide. In some embodiments, the identity of the at least a second, at least a third, at least a fourth, or at least a fifth affinity binding polypeptide is as provided for herein. In some embodiments, the location of the at least a second, at least a third, at least a fourth, or at least a fifth affinity binding polypeptide with respect to the glycoprotein is as provided for herein. In some embodiments, the glycoprotein comprises more than one affinity binding polypeptides as provided for herein.

In some embodiments, the pseudotyped viral-like particle or viral vector further comprises a second viral glycoprotein that does not comprise an affinity binding polypeptide.

In some embodiments, the identity of the second glycoprotein is identical to the first glycoprotein. In some embodiments, the first glycoprotein and the second glycoprotein comprise unique viral glycoproteins.

In some embodiments, a pseudotyped viral-like particle or viral vector is provided. In some embodiments, the pseudotyped viral-like particle or viral vector comprises an engineered envelope comprising a recombinant virus glycoprotein, an engineered targeting moiety for binding to a target cell, and at least a first affinity binding polypeptide, wherein the targeting moiety is an scFv comprising an amino acid sequence of SEQ ID NO: 101, SEQ ID NO: 102, SEQ ID NO: 119, SEQ ID NO: 120, or any variant thereof as provided for herein, and the at least a first affinity binding polypeptide is fused to the targeting moiety. In some embodiments, the pseudotyped viral-like particle or viral vector further comprises a nucleic acid molecule encoding a polypeptide of interest. In some embodiments, the pseudotyped viral-like particle or viral vector is a lentivirus. In some embodiments, the scFv is fused to the virus glycoprotein.

In some embodiments, the scFv is fused directly to the virus glycoprotein. In some embodiments, the scFv is fused indirectly to the virus glycoprotein via, for example, a peptide linker as provided for herein. In some embodiments, the scFv is not fused to the virus glycoprotein.

In some embodiments, the identity of the at least a first affinity binding polypeptide is as provided for herein. In some embodiments, the at least a first affinity binding polypeptide is located at the N-terminus, at the C-terminus, or within the scFv. In some embodiments, the location of the at least a first affinity binding polypeptide with respect to the scFv is as provided for herein.

In some embodiments, a pseudotyped viral-like particle or viral vector is provided. In some embodiments, the pseudotyped viral-like particle or viral vector comprises an engineered envelope comprising a recombinant virus glycoprotein, an engineered targeting moiety for binding to a target cell, and at least a first affinity binding polypeptide, wherein the targeting moiety is an scFv and the at least a first affinity binding polypeptide fused to the targeting moiety. In some embodiments, the at least a first affinity binding polypeptide fused to the targeting moiety comprises an amino acid sequence of SEQ ID NO: 121, SEQ ID NO: 126, SEQ ID NO: 129, SEQ ID NO: 130, SEQ ID NO: 131, SEQ ID NO: 174, SEQ ID NO: 175, SEQ ID NO: 176, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, or SEQ ID NO: 187, or any variant as provided for herein. In some embodiments, the at least a first affinity binding polypeptide fused to the targeting moiety comprises an amino acid sequence of SEQ ID NO: 121. In some embodiments, the at least a first affinity binding polypeptide fused to the targeting moiety comprises an amino acid sequence of SEQ ID NO: 126. In some embodiments, the at least a first affinity binding polypeptide fused to the targeting moiety comprises an amino acid sequence of SEQ ID NO: 129. In some embodiments, the at least a first affinity binding polypeptide fused to the targeting moiety comprises an amino acid sequence of SEQ ID NO: 130. In some embodiments, the at least a first affinity binding polypeptide fused to the targeting moiety comprises an amino acid sequence of SEQ ID NO: 131. In some embodiments, the at least a first affinity binding polypeptide fused to the targeting moiety comprises an amino acid sequence of SEQ ID NO: 174. In some embodiments, the at least a first affinity binding polypeptide fused to the targeting moiety comprises an amino acid sequence of SEQ ID NO: 175. In some embodiments, the at least a first affinity binding polypeptide fused to the targeting moiety comprises an amino acid sequence of SEQ ID NO: 176. In some embodiments, the at least a first affinity binding polypeptide fused to the targeting moiety comprises an amino acid sequence of SEQ ID NO: 178. In some embodiments, the at least a first affinity binding polypeptide fused to the targeting moiety comprises an amino acid sequence of SEQ ID NO: 179. In some embodiments, the at least a first affinity binding polypeptide fused to the targeting moiety comprises an amino acid sequence of SEQ ID NO: 180. In some embodiments, the at least a first affinity binding polypeptide fused to the targeting moiety comprises an amino acid sequence of SEQ ID NO: 181. In some embodiments, the at least a first affinity binding polypeptide fused to the targeting moiety comprises an amino acid sequence of SEQ ID NO: 182. In some embodiments, the at least a first affinity binding polypeptide fused to the targeting moiety comprises an amino acid sequence of SEQ ID NO: 183. In some embodiments, the at least a first affinity binding polypeptide fused to the targeting moiety comprises an amino acid sequence of SEQ ID NO: 184. In some embodiments, the at least a first affinity binding polypeptide fused to the targeting moiety comprises an amino acid sequence of SEQ ID NO: 185. In some embodiments, the at least a first affinity binding polypeptide fused to the targeting moiety comprises an amino acid sequence of SEQ ID NO: 186. In some embodiments, the at least a first affinity binding polypeptide fused to the targeting moiety comprises an amino acid sequence of SEQ ID NO: 187. In some embodiments, the pseudotyped viral-like particle or viral vector further comprises a nucleic acid molecule encoding a polypeptide of interest. In some embodiments, the pseudotyped viral-like particle or viral vector is a lentivirus. In some embodiments, the scFv is fused to the virus glycoprotein.

In some embodiments, the scFv is fused directly to the virus glycoprotein. In some embodiments, the scFv is fused indirectly to the virus glycoprotein via, for example, a peptide linker as provided for herein. In some embodiments, the scFv is not fused to the virus glycoprotein.

In some embodiments, the scFv comprises at least a second affinity binding polypeptide. In some embodiments, the identity of the at least a second affinity binding polypeptide is as provided for herein. In some embodiments, the at least a second affinity binding polypeptide is located at the N-terminus, at the C-terminus, or within the scFv. In some embodiments, the location of the at least a second affinity binding polypeptide with respect to the scFv is as provided for herein.

In some embodiments, the scFv comprises at least a third, at least a fourth, or at least a fifth affinity binding polypeptides. In some embodiments, the scFv comprises at least a third affinity binding polypeptide. In some embodiments, the identity of the at least a third affinity binding polypeptide is as provided for herein. In some embodiments, the at least a third affinity binding polypeptide is located at the N-terminus, at the C-terminus, or within the scFv. In some embodiments, the location of the at least a third affinity binding polypeptide with respect to the scFv is as provided for herein. In some embodiments, the scFv comprises at least a fourth affinity binding polypeptide. In some embodiments, the identity of the at least a fourth affinity binding polypeptide is as provided for herein. In some embodiments, the at least a fourth affinity binding polypeptide is located at the N-terminus, at the C-terminus, or within the scFv. In some embodiments, the location of the at least a fourth affinity binding polypeptide with respect to the scFv is as provided for herein. In some embodiments, the scFv comprises at least a fifth affinity binding polypeptide. In some embodiments, the identity of the at least a fifth affinity binding polypeptide is as provided for herein. In some embodiments, the at least a fifth affinity binding polypeptide is located at the N-terminus, at the C-terminus, or within the scFv. In some embodiments, the location of the at least a fifth affinity binding polypeptide with respect to the scFv is as provided for herein. In some embodiments, each additional affinity binding polypeptide is located, independently, at the N-terminus, at the C-terminus, or within the scFv.

It is to be understood that in any of the embodiments provided for herein, the affinity binding polypeptides may further comprise a protease cleavage recognition site, such that the affinity binding polypeptide may be removed from the pseudotyped viral-like particle or viral vector. Non-limiting examples of protease recognition sites that may be used for such purposes are the tobacco etch virus (TEV) protease recognition sequences ENLYFQG (SEQ ID NO: 127) and ENLYFQS (SEQ ID NO: 128). Other protease cleavage recognition sequences are known in the art and are within the scope of the present application.

TABLE 6

| | SEQ ID NOs absent | |
|---|---|---|
| | affinity binding | SEQ ID NOs with affinity |
| Construct | polypeptides | binding polypeptides |
| Ebola Virus | 25 | 26, 27, 28, 135, 136, 137, |
| Glycoprotein | | 138, 139, 140, 141, 142, 143, |
| | | 144, 145, 147, 148, 149, 150, |
| | | 151, 152, 153, 154, 155, 156, |
| | | 157, 158, 159, 160, 161, 162, |
| | | 163, 164, 188, 189, 190, 191 |
| NiV-G | 29, 30 | 31, 32 |
| NiV-F | 33, 34 | 35, 166 |
| VSV-G | 36, 37, 39, 40, 41, 42, 43, 44, | 68, 69, 70, 71, 72, 73, 132, |
| | 45, 46, 47, 49, 50, 52, 53, 55, | 133, 167, 168, |
| | 56, 58, 59, 61, 62, 64, 65, 66, | |
| | 67 | |
| MeV-H | 74 | 75, 76 |
| MeV-F | 77 | 78, 170 |
| SVCV-G | 79, 80 | 81, 172 |
| Targeting | 101, 102, 119, 120 | 121, 126, 129, 130, 131, 174, |
| moiety | | 175, 176, 178, 179, 180, 181, |
| | | 182, 183, 184, 185, 186, 187 |

In some embodiments, a viral particle as provided for herein comprises a glycoprotein and a targeting moiety as provided for in Table 6. In some embodiments, the glycoprotein comprises an amino acid sequence corresponding to a SEQ ID NO absent an affinity binding polypeptide as provided for in Table 6, and the targeting moiety comprises an amino acid sequence corresponding to a SEQ ID NO with an affinity binding polypeptide as provided for in Table 6. In some embodiments, the glycoprotein comprises an amino acid sequence having at least 70% identity to a SEQ ID NO absent an affinity binding polypeptide as provided for in Table 6, and the targeting moiety comprises an amino acid sequence having at least 70% identity to a SEQ ID NO with an affinity binding polypeptide as provided for in Table 6. In some embodiments, the glycoprotein comprises an amino acid sequence having at least 80% identity to a SEQ ID NO absent an affinity binding polypeptide as provided for in Table 6, and the targeting moiety comprises an amino acid sequence having at least 80% identity to a SEQ ID NO with an affinity binding polypeptide as provided for in Table 6. In some embodiments, the glycoprotein comprises an amino acid sequence having at least 85% identity to a SEQ ID NO absent an affinity binding polypeptide as provided for in Table 6, and the targeting moiety comprises an amino acid sequence having at least 85% identity to a SEQ ID NO with an affinity binding polypeptide as provided for in Table 6. In some embodiments, the glycoprotein comprises an amino acid sequence having at least 90% identity to a SEQ ID NO absent an affinity binding polypeptide as provided for in Table 6, and the targeting moiety comprises an amino acid sequence having at least 90% identity to a SEQ ID NO with an affinity binding polypeptide as provided for in Table 6. In some embodiments, the glycoprotein comprises an amino acid sequence having at least 95% identity to a SEQ ID NO absent an affinity binding polypeptide as provided for in Table 6, and the targeting moiety comprises an amino acid sequence having at least 95% identity to a SEQ ID NO with an affinity binding polypeptide as provided for in Table 6. In some embodiments, the glycoprotein comprises an amino acid sequence having at least 98% identity to a SEQ ID NO absent an affinity binding polypeptide as provided for in Table 6, and the targeting moiety comprises an amino acid sequence having at least 98% identity to a SEQ ID NO with an affinity binding polypeptide as provided for in Table 6. In some embodiments, the glycoprotein comprises an amino acid sequence having at least 100% identity to a SEQ ID NO absent an affinity binding polypeptide as provided for in Table 6, and the targeting moiety comprises an amino acid sequence having at least 100% identity to a SEQ ID NO with an affinity binding polypeptide as provided for in Table 6. In some embodiments, the glycoprotein comprising an amino acid sequence corresponding to a SEQ ID NO absent an affinity binding polypeptide as provided for in Table 6 comprises an amino acid sequence of SEQ ID NO: 25, SEQ ID NO: 29, SEQ ID NO: 30, SEQ ID NO: 33, SEQ ID NO: 34, SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 57, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 66, SEQ ID NO: 67, 74, SEQ ID NO: 77, SEQ ID NO: 79, or SEQ ID NO: 80. In some embodiments, the targeting moiety comprises an amino acid sequence corresponding to a SEQ ID NO with an affinity binding polypeptide as provided for in Table 6 comprises an amino acid sequence of SEQ ID NO: 121, SEQ ID NO: 126, SEQ ID NO: 129, SEQ ID NO: 130, SEQ ID NO: 131, SEQ ID NO: 174, SEQ ID NO: 175, SEQ ID NO: 176, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, or SEQ ID NO: 187.

In some embodiments, the glycoprotein comprises an amino acid sequence corresponding to a SEQ ID NO with an affinity binding polypeptide as provided for in Table 6, and the targeting moiety comprises an amino acid sequence corresponding to a SEQ ID NO absent an affinity binding polypeptide as provided for in Table 6. In some embodiments, the glycoprotein comprises an amino acid sequence having at least 70% identity to a SEQ ID NO with an affinity binding polypeptide as provided for in Table 6, and the targeting moiety comprises an amino acid sequence having at least 70% identity to a SEQ ID NO absent an affinity binding polypeptide as provided for in Table 6. In some embodiments, the glycoprotein comprises an amino acid sequence having at least 80% identity to a SEQ ID NO with an affinity binding polypeptide as provided for in Table 6, and the targeting moiety comprises an amino acid sequence having at least 80% identity to a SEQ ID NO absent an affinity binding polypeptide as provided for in Table 6. In some embodiments, the glycoprotein comprises an amino acid sequence having at least 85% identity to a SEQ ID NO with an affinity binding polypeptide as provided for in Table 6, and the targeting moiety comprises an amino acid sequence having at least 85% identity to a SEQ ID NO absent an affinity binding polypeptide as provided for in Table 6. In some embodiments, the glycoprotein comprises an amino acid sequence having at least 90% identity to a SEQ ID NO with an affinity binding polypeptide as provided for in Table 6, and the targeting moiety comprises an amino acid sequence having at least 90% identity to a SEQ ID NO absent an affinity binding polypeptide as provided for in Table 6. In some embodiments, the glycoprotein comprises an amino acid sequence having at least 95% identity to a SEQ ID NO with an affinity binding polypeptide as provided for in Table 6, and the targeting moiety comprises an amino acid sequence having at least 95% identity to a SEQ ID NO absent an affinity binding polypeptide as provided for in Table 6. In some embodiments, the glycoprotein comprises an amino acid sequence having at least 98% identity to a SEQ ID NO with an affinity binding polypeptide as provided for in Table 6, and the targeting moiety comprises an amino acid sequence having at least 98% identity to a SEQ ID NO absent an affinity binding polypeptide as provided for in Table 6. In some embodiments, the glycoprotein comprises an amino acid sequence having at least 100% identity to a SEQ ID NO with an affinity binding polypeptide as provided for in Table 6, and the targeting moiety comprises an amino acid sequence having at least 100% identity to a SEQ ID NO absent an affinity binding polypeptide as provided for in Table 6. In some embodiments, the glycoprotein comprising an amino acid sequence corresponding to a SEQ ID NO with an affinity binding polypeptide as provided for in Table 6 comprises an amino acid sequence of SEQ ID NO: 26. SEQ ID NO: 27. SEQ ID NO: 28, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 144, SEQ ID NO: 145, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 188, SEQ ID NO: 189, SEQ ID NO: 190, SEQ ID NO: 191, SEQ ID NO: 31, SEQ ID NO: 32, SEQ ID NO: 35, SEQ ID NO: 166, SEQ ID NO: 68, SEQ ID NO: 69, SEQ ID NO: 70, SEQ ID NO: 71, SEQ ID NO: 72, SEQ ID NO: 73, SEQ ID NO: 132, SEQ ID NO: 133, SEQ ID NO: 167, SEQ ID NO: 168, SEQ ID NO: 75, SEQ ID NO: 76, SEQ ID NO: 78, SEQ ID NO: 170, SEQ ID NO: 81, or SEQ ID NO: 172. In some embodiments, the targeting moiety comprising an amino acid sequence corresponding to a SEQ ID NO absent an affinity binding polypeptide as provided for in Table 6 comprises an amino acid sequence of SEQ ID NO: 101, SEQ ID NO: 102, SEQ ID NO: 119, or SEQ ID NO: 120.

In some embodiments, the glycoprotein comprises an amino acid sequence corresponding to a SEQ ID NO with an affinity binding polypeptide as provided for in Table 6, and the targeting moiety comprises an amino acid sequence corresponding to a SEQ ID NO with an affinity binding polypeptide as provided for in Table 6. In some embodiments, the glycoprotein comprises an amino acid sequence having at least 70% identity to a SEQ ID NO with an affinity binding polypeptide as provided for in Table 6, and the targeting moiety comprises an amino acid sequence having at least 70% identity to a SEQ ID NO with an affinity binding polypeptide as provided for in Table 6. In some embodiments, the glycoprotein comprises an amino acid sequence having at least 80% identity to a SEQ ID NO with an affinity binding polypeptide as provided for in Table 6, and the targeting moiety comprises an amino acid sequence having at least 80% identity to a SEQ ID NO with an affinity binding polypeptide as provided for in Table 6. In some embodiments, the glycoprotein comprises an amino acid sequence having at least 85% identity to a SEQ ID NO with an affinity binding polypeptide as provided for in Table 6, and the targeting moiety comprises an amino acid sequence having at least 85% identity to a SEQ ID NO with an affinity binding polypeptide as provided for in Table 6. In some embodiments, the glycoprotein comprises an amino acid sequence having at least 90% identity to a SEQ ID NO with an affinity binding polypeptide as provided for in Table 6, and the targeting moiety comprises an amino acid sequence having at least 90% identity to a SEQ ID NO with an affinity binding polypeptide as provided for in Table 6. In some embodiments, the glycoprotein comprises an amino acid sequence having at least 95% identity to a SEQ ID NO with an affinity binding polypeptide as provided for in Table 6, and the targeting moiety comprises an amino acid sequence having at least 95% identity to a SEQ ID NO with an affinity binding polypeptide as provided for in Table 6. In some embodiments, the glycoprotein comprises an amino acid sequence having at least 98% identity to a SEQ ID NO with an affinity binding polypeptide as provided for in Table 6, and the targeting moiety comprises an amino acid sequence having at least 98% identity to a SEQ ID NO with an affinity binding polypeptide as provided for in Table 6. In some embodiments, the glycoprotein comprises an amino acid sequence having at least 100% identity to a SEQ ID NO with an affinity binding polypeptide as provided for in Table 6, and the targeting moiety comprises an amino acid sequence having at least 100% identity to a SEQ ID NO with an affinity binding polypeptide as provided for in Table 6. In some embodiments, the glycoprotein comprising an amino acid sequence corresponding to a SEQ ID NO with an affinity binding polypeptide as provided for in Table 6 comprises an amino acid sequence of SEQ ID NO: 26. SEQ ID NO: 27. SEQ ID NO: 28, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 144, SEQ ID NO: 145, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 188, SEQ ID NO: 189, SEQ ID NO: 190, SEQ ID NO: 191, SEQ ID NO: 31, SEQ ID NO: 32, SEQ ID NO: 35, SEQ ID NO: 166, SEQ ID NO: 68, SEQ ID NO: 69, SEQ ID NO: 70, SEQ ID NO: 71, SEQ ID NO: 72, SEQ ID NO: 73, SEQ ID NO: 132, SEQ ID NO: 133, SEQ ID NO: 167, SEQ ID NO: 168, SEQ ID NO: 75, SEQ ID NO: 76, SEQ ID NO: 78, SEQ ID NO: 170, SEQ ID NO: 81, or SEQ ID NO: 172. In some embodiments, the targeting moiety comprises an amino acid sequence corresponding to a SEQ ID NO with an affinity binding polypeptide as provided for in Table 6 comprises an amino acid sequence of SEQ ID NO: 121, SEQ ID NO: 126, SEQ ID NO: 129, SEQ ID NO: 130, SEQ ID NO: 131, SEQ ID NO: 174, SEQ ID NO: 175, SEQ ID NO: 176, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, or SEQ ID NO: 187.

### Polypeptide and Nucleic Acid Molecules

In some embodiments, a polypeptide molecule is provided. In some embodiments, the polypeptide molecule comprises a virus glycoprotein. In some embodiments, the virus glycoprotein further comprises at least a first affinity binding polypeptide sequence. In some embodiments, the identity of the at least a first affinity binding polypeptide is as provided for herein. In some embodiments, the at least a first affinity binding polypeptide is any polypeptide sequence that will bind to a binder. For example, the affinity binding polypeptide may comprise of GFP and the binder may comprise of an anti-GFP antibody. In some embodiments, the affinity binding polypeptide may bind to a non-proteinaceous binder. For example, the affinity binding polypeptide may be a polyhistidine sequence and the binder may be a nickel resin. Accordingly, in some embodiments, the at least a first affinity binding polypeptide is an affinity tag selected from the group including, but not limited to a polyhistidine tag, a polyarginine tag, a FLAG tag, a streptavidin tag, a calmodulin binding peptide, or any variant thereof, or any combination thereof. In some embodiments, the at least a first affinity binding polypeptide is a polyhistidine tag. In some embodiments, the at least a first affinity binding polypeptide is a polyarginine tag. In some embodiments, the at least a first affinity binding polypeptide is a FLAG tag. In some embodiments, the at least a first affinity binding polypeptide is a streptavidin tag. In some embodiments, the at least a first affinity binding polypeptide is a calmodulin binding peptide. In some embodiments, the at least a first affinity binding polypeptide is any variation of a recited affinity binding polypeptide. Such variations may include one or more amino acid substitutions, deletions, or additions in order to alter the binding affinity of the at least a first affinity binding polypeptide for the corresponding binder.

Any such known variation is within the scope of the present application.

In some embodiments, the at least a first affinity binding polypeptide is a streptavidin tag. In some embodiments, the streptavidin tag is selected from the group consisting of a streptavidin-binding peptide, a streptavidin binding tag, a strep-tag II, a twin-strep tag, or any variation thereof, or any combination thereof. In some embodiments, the streptavidin tag consists of a streptavidin-binding peptide. In some embodiments, the streptavidin tag consists of a streptavidin binding tag. In some embodiments, the streptavidin tag consists of a strep-tag II. In some embodiments, the streptavidin tag consists of a twin-strep tag. In some embodiments, the streptavidin tag comprises the formula of Formula I:

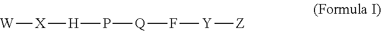

wherein X can be any amino acid and Y and Z can either both be G, or Y is E and Z is R or K. In some embodiments, X is A. In some embodiments, X is R. In some embodiments, X is N. In some embodiments, X is D. In some embodiments, X is C. In some embodiments, X is Q. In some embodiments, X is E. In some embodiments, X is G. In some embodiments, X is H. In some embodiments, X is I. In some embodiments, X is L. In some embodiments, X is K. In some embodiments, X is M. In some embodiments, X is F. In some embodiments, X is P. In some embodiments, X is S. In some embodiments, X is T. In some embodiments, X is W. In some embodiments, X is Y. In some embodiments, X is V. In some embodiments, Y is G and Z is G. In some embodiments, Y is E and Z is R. In some embodiments, Y is E and Z is K. In some embodiments, the streptavidin tag of Formula I is given by the sequence WSHPQFEK (SEQ ID NO: 1). In some embodiments, the sequence of strep-tag II is the sequence of SEQ ID NO: 1.

In some embodiments, the streptavidin tag comprises the formula of Formula II:

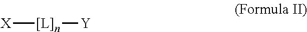

wherein X is a sequence of Formula I, Y is a sequence of Formula I, L is a linker peptide sequence, and n is an integer from 1 to 10. In some embodiments, X and Y comprise the same sequence. In some embodiments, X and Y comprises unique sequences of the formula of Formula I. In some embodiment, n is 1. In some embodiment, n is 2. In some embodiment, n is 3. In some embodiment, n is 4. In some embodiment, n is 5. In some embodiment, n is 6. In some embodiment, n is 7. In some embodiment, n is 8. In some embodiment, n is 9. In some embodiment, n is 10. It is to be understood that the portion of Formula II represented by [L]ᵤ is not meant to indicate that the linker L is to be repeated n times, but rather, when n is greater than 1, each instance of L may be the same or a unique peptide linker sequence. Thus, for example, in embodiments where n is 3, one could envisage the formula of Formula II to be written thusly:

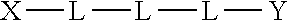

wherein each L, may comprise the same or a unique peptide linker sequence. For clarity, one could also envisage the formula of Formula II to be written thusly when n is 3:

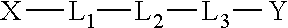

wherein each L₁, L₂, and L₃ may comprise the same or a unique peptide linker sequence. This clarifying representation of the formula of Formula II holds true for each value of n. In some embodiments, each L is the same peptide linker sequence. In some embodiments, each L is, individually, a unique linker peptide sequence. In some embodiments, the linker is a flexible peptide linker. In some embodiments, the linker is a non-cleavable peptide linker. In some embodiments the linker is a cleavable peptide linker. In some embodiments, the linker is a peptide linker as provided for herein. In some embodiments, the linker is given by the sequence GGGS (SEQ ID NO: 22). In some embodiments, the linker is given by the sequence GGSA (SEQ ID NO: 23). It is to be understood, for example, that when n is 3 or greater, the identity of the linker can be identical or unique between any two or more linkers. Thus, for example, when n is 3, L₁, L₂, and L₃ can all be the same; L₁, L₂, and L₃ can all be different; L₁ and L₂ can be the same while L₃ is different; L₁ and L₃ can be the same while L₂ is different; or L₂ and L₃ can be the same while L₁ is different. Such optionality likewise holds true for embodiments when n is 4 or greater. In some embodiments, X is given by SEQ ID NO 1, Y is given by Formula I, n is an integer between 1 and 10 and each L is, independently, a linker as provided for herein. In some embodiments, X is given by Formula I, Y is given by SEQ ID NO: 1, n is an integer between 1 and 10, and each L is, independently, a linker as provided for herein. In some embodiments, X is given by SEQ ID NO: 1, Y is given by SEQ ID NO: 1, n is 3, L₁ is given by SEQ ID NO: 22, L₂ is given by SEQ ID NO: 22, and L₃ is given by SEQ ID NO: 23. In some embodiments, the streptavidin tag of Formula II comprises the sequence WSHPQFEKGGGSGGGSGGSAWSHPQFEK (SEQ ID NO: 24). In some embodiments, the sequence of the twin-strep tag is the sequence of SEQ ID NO: 24.

In some embodiments, the at least a first affinity binding polypeptide is fused to the glycoprotein. The at least a first affinity binding polypeptide can be inserted at any position in the glycoprotein. Accordingly, in some embodiments, the at least a first affinity binding polypeptide is located at the N-terminus, at the C-terminus, or within the glycoprotein. In some embodiments, the at least a first affinity binding polypeptide is located at the N-terminus of the glycoprotein. In some embodiments, the at least a first affinity binding polypeptide is located at the C-terminus of the glycoprotein. In some embodiments, the at least a first affinity binding polypeptide is located within the glycoprotein. In some embodiments, the at least a first affinity binding polypeptide is fused directly to the glycoprotein. In some embodiments, the at least a first affinity binding polypeptide is fused indirectly to the glycoprotein via, for example, a peptide linker as provided for herein. In embodiments where the at least a first affinity binding polypeptide is located within the glycoprotein, the affinity binding polypeptide may be fused to the glycoprotein directly, fused directly at the N-terminus of the affinity binding polypeptide and fused indirectly at the C-terminus of the affinity binding polypeptide via, for example, a peptide linker as provided for herein, fused indirectly at the N-terminus of the affinity binding polypeptide via, for example, a peptide linker as provided for herein and fused directly at the C-terminus of the affinity binding polypeptide, or fused indirectly at both the N and C terminus of the affinity binding polypeptide via, for example, a polypeptide linker as provided for herein.

In some embodiments, the polypeptide further comprises at least a second affinity binding polypeptide fused to the glycoprotein. The at least a second affinity binding polypeptide can be inserted at any position in the glycoprotein. Further, the position of the at least a second affinity binding polypeptide is independent of the position of the at least a first affinity binding polypeptide. Accordingly, in some embodiments, the at least a second affinity binding polypeptide is located at the N-terminus, at the C-terminus, or within the glycoprotein. In some embodiments, the at least a second affinity binding polypeptide is located at the N-terminus of the glycoprotein. In some embodiments, the at least a second affinity binding polypeptide is located at the C-terminus of the glycoprotein. In some embodiments, the at least a second affinity binding polypeptide is located within the glycoprotein. In some embodiments, the at least a second affinity binding polypeptide is fused directly to the glycoprotein. In some embodiments, the at least a second affinity binding polypeptide is fused indirectly to the glycoprotein via, for example, a peptide linker as provided for herein. In embodiments where the at least a second affinity binding polypeptide is located within the glycoprotein, the affinity binding polypeptide may be fused to the glycoprotein directly, fused directly at the N-terminus of the affinity binding polypeptide and fused indirectly at the C-terminus of the affinity binding polypeptide via, for example, a peptide linker as provided for herein, fused indirectly at the N-terminus of the affinity binding polypeptide via, for example, a peptide linker as provided for herein and fused directly at the C-terminus of the affinity binding polypeptide, or fused indirectly at both the N and C terminus of the affinity binding polypeptide via, for example, a polypeptide linker as provided for herein.

In some embodiments, the polypeptide further comprises at least a third, at least a fourth, or at least a fifth affinity binding polypeptide fused to the glycoprotein. In some embodiments, the polypeptide comprises at least a third affinity binding polypeptide fused to the glycoprotein. In some embodiments, the position of the at least a third affinity binding polypeptide is as provided for herein for the at least a first and the at least a second affinity binding polypeptide. In some embodiments, the polypeptide comprises at least a fourth affinity binding polypeptide fused to the glycoprotein. In some embodiments, the position of the at least a fourth affinity binding polypeptide is as provided for herein for the at least a first and the at least a second affinity binding polypeptide. In some embodiments, the polypeptide comprises at least a fifth affinity binding polypeptide fused to the glycoprotein. In some embodiments, the position of the at least a fifth affinity binding polypeptide is as provided for herein for the at least a first and the at least a second affinity binding polypeptide. The number of affinity binding polypeptides fused to the glycoprotein is only limited in that the fusion constructs should not negatively affect the function or purpose of the glycoprotein. Thus, in some embodiments, the polypeptide further comprises at least one affinity binding polypeptide. In some embodiments, the polypeptide further comprises at least two affinity binding polypeptides. In some embodiments, the polypeptide further comprises at least three affinity binding polypeptides. In some embodiments, the polypeptide further comprises at least four affinity binding polypeptides. In some embodiments, the polypeptide further comprises at least five affinity binding polypeptides. In some embodiments, the polypeptide further comprises up to five affinity binding polypeptides. In some embodiments, the polypeptide further comprises up to ten affinity binding polypeptides. In some embodiments, the polypeptide further comprises up to twenty affinity binding polypeptides.

In some embodiments, the polypeptide further comprises a targeting moiety. In some embodiments, the targeting moiety is fused directly to the glycoprotein. In some embodiments, the targeting moiety is fused indirectly to the glycoprotein via, for example, a peptide linker sequence as provided for herein. In some embodiments, the targeting moiety is not fused to the glycoprotein.

In some embodiments, the sequence of the viral glycoprotein is as provided for herein. In some embodiments, the sequence of the affinity binding polypeptide fused to the glycoprotein is as provided for herein. In some embodiments, the targeting moiety binds a target as provided for herein. In some embodiments, the sequence of the targeting moiety is as provided for herein.

In some embodiments, a polypeptide molecule is provided. In some embodiments, the polypeptide molecule comprises a targeting moiety. In some embodiments, the targeting moiety further comprises at least a first affinity binding polypeptide sequence. In some embodiments, the identity of the at least a first affinity binding polypeptide is as provided for herein. In some embodiments, the at least a first affinity binding polypeptide is any polypeptide sequence that will bind to a binder. For example, the affinity binding polypeptide may comprise of GFP and the binder may comprise of an anti-GFP antibody. In some embodiments, the affinity binding polypeptide may bind to a non-proteinaceous binder. For example, the affinity binding polypeptide may be a polyhistidine sequence and the binder may be a nickel resin. Accordingly, in some embodiments, the at least a first affinity binding polypeptide is an affinity tag selected from the group including, but not limited to a polyhistidine tag, a polyarginine tag, a FLAG tag, a streptavidin tag, a calmodulin binding peptide, or any variant thereof, or any combination thereof. In some embodiments, the at least a first affinity binding polypeptide is a polyhistidine tag. In some embodiments, the at least a first affinity binding polypeptide is a polyarginine tag. In some embodiments, the at least a first affinity binding polypeptide is a FLAG tag. In some embodiments, the at least a first affinity binding polypeptide is a streptavidin tag. In some embodiments, the at least a first affinity binding polypeptide is a calmodulin binding peptide. In some embodiments, the at least a first affinity binding polypeptide is any variation of a recited affinity binding polypeptide. Such variations may include one or more amino acid substitutions, deletions, or additions in order to alter the binding affinity of the at least a first affinity binding polypeptide for the corresponding binder. Any such known variation is within the scope of the present application.

In some embodiments, the at least a first affinity binding polypeptide is a streptavidin tag. In some embodiments, the streptavidin tag is selected from the group consisting of a streptavidin-binding peptide, a streptavidin binding tag, a strep-tag II, a twin-strep tag, or any variation thereof, or any combination thereof. In some embodiments, the streptavidin tag consists of a streptavidin-binding peptide. In some embodiments, the streptavidin tag consists of a streptavidin binding tag. In some embodiments, the streptavidin tag consists of a strep-tag II. In some embodiments, the streptavidin tag consists of a twin-strep tag. In some embodiments, the streptavidin tag comprises the formula of Formula I:

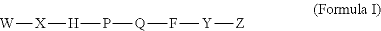

wherein X can be any amino acid and Y and Z can either both be G, or Y is E and Z is R or K. In some embodiments, X is A. In some embodiments, X is R. In some embodiments, X is N. In some embodiments, X is D. In some embodiments, X is C. In some embodiments, X is Q. In some embodiments, X is E. In some embodiments, X is G. In some embodiments, X is H. In some embodiments, X is I. In some embodiments, X is L. In some embodiments, X is K. In some embodiments, X is M. In some embodiments, X is F. In some embodiments, X is P. In some embodiments, X is S. In some embodiments, X is T. In some embodiments, X is W. In some embodiments, X is Y. In some embodiments, X is V. In some embodiments, Y is G and Z is G. In some embodiments, Y is E and Z is R. In some embodiments, Y is E and Z is K. In some embodiments, the streptavidin tag of Formula I is given by the sequence WSHPQFEK (SEQ ID NO: 1). In some embodiments, the sequence of strep-tag II is the sequence of SEQ ID NO: 1.

In some embodiments, the streptavidin tag comprises the formula of Formula II:

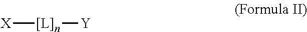

wherein X is a sequence of Formula I, Y is a sequence of Formula I, L is a linker peptide sequence, and n is an integer from 1 to 10. In some embodiments, X and Y comprise the same sequence. In some embodiments, X and Y comprises unique sequences of the formula of Formula I. In some embodiment, n is 1. In some embodiment, n is 2. In some embodiment, n is 3. In some embodiment, n is 4. In some embodiment, n is 5. In some embodiment, n is 6. In some embodiment, n is 7. In some embodiment, n is 8. In some embodiment, n is 9. In some embodiment, n is 10. It is to be understood that the portion of Formula II represented by [L]n is not meant to indicate that the linker L is to be repeated n times, but rather, when n is greater than 1, each instance of L may be the same or a unique peptide linker sequence. Thus, for example, in embodiments where n is 3, one could envisage the formula of Formula II to be written thusly:

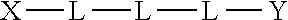

wherein each L, may comprise the same or a unique peptide linker sequence. For clarity, one could also envisage the formula of Formula II to be written thusly when n is 3:

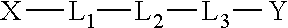

wherein each L₁, L₂, and L₃ may comprise the same or a unique peptide linker sequence. This clarifying representation of the formula of Formula II holds true for each value of n. In some embodiments, each L is the same peptide linker sequence. In some embodiments, each L is, individually, a unique linker peptide sequence. In some embodiments, the linker is a flexible peptide linker. In some embodiments, the linker is a non-cleavable peptide linker. In some embodiments the linker is a cleavable peptide linker. In some embodiments, the linker is a peptide linker as provided for herein. In some embodiments, the linker is given by the sequence GGGS (SEQ ID NO: 22). In some embodiments, the linker is given by the sequence GGSA (SEQ ID NO: 23). It is to be understood, for example, that when n is 3 or greater, the identity of the linker can be identical or unique between any two or more linkers. Thus, for example, when n is 3, L₁, L₂, and L₃ can all be the same; L₁, L₂, and L₃ can all be different; L₁ and L₂ can be the same while L₃ is different; L₁ and L₃ can be the same while L₂ is different; or L₂ and L₃ can be the same while L₁ is different. Such optionality likewise holds true for embodiments when n is 4 or greater. In some embodiments, X is given by SEQ ID NO 1, Y is given by Formula I, n is an integer between 1 and 10 and each L is, independently, a linker as provided for herein. In some embodiments, X is given by Formula I, Y is given by SEQ ID NO: 1, n is an integer between 1 and 10, and each L is, independently, a linker as provided for herein. In some embodiments, X is given by SEQ ID NO: 1, Y is given by SEQ ID NO: 1, n is 3, L₁ is given by SEQ ID NO: 22, L₂ is given by SEQ ID NO: 22, and L₃ is given by SEQ ID NO: 23. In some embodiments, the streptavidin tag of Formula II comprises the sequence WSHPQFEKGGGSGGGSGGSAWSHPQFEK (SEQ ID NO: 24). In some embodiments, the sequence of the twin-strep tag is the sequence of SEQ ID NO: 24.

In some embodiments, the at least a first affinity binding polypeptide is fused to the targeting moiety. The at least a first affinity binding polypeptide can be inserted at any position in the targeting moiety. Accordingly, in some embodiments, the at least a first affinity binding polypeptide is located at the N-terminus, at the C-terminus, or within the targeting moiety. In some embodiments, the at least a first affinity binding polypeptide is located at the N-terminus of the targeting moiety. In some embodiments, the at least a first affinity binding polypeptide is located at the C-terminus of the targeting moiety. In some embodiments, the at least a first affinity binding polypeptide is located within the targeting moiety. In some embodiments, the at least a first affinity binding polypeptide is fused directly to the targeting moiety. In some embodiments, the at least a first affinity binding polypeptide is fused indirectly to the targeting moiety via, for example, a peptide linker as provided for herein. In embodiments where the at least a first affinity binding polypeptide is located within the targeting moiety, the affinity binding polypeptide may be fused to the targeting moiety directly, fused directly at the N-terminus of the affinity binding polypeptide and fused indirectly at the C-terminus of the affinity binding polypeptide via, for example, a peptide linker as provided for herein, fused indirectly at the N-terminus of the affinity binding polypeptide via, for example, a peptide linker as provided for herein and fused directly at the C-terminus of the affinity binding polypeptide, or fused indirectly at both the N and C terminus of the affinity binding polypeptide via, for example, a polypeptide linker as provided for herein.

In some embodiments, the polypeptide further comprises at least a second affinity binding polypeptide fused to the targeting moiety. The at least a second affinity binding polypeptide can be inserted at any position in the targeting moiety. Further, the position of the at least a second affinity binding polypeptide is independent of the position of the at least a first affinity binding polypeptide. Accordingly, in some embodiments, the at least a second affinity binding polypeptide is located at the N-terminus, at the C-terminus, or within the targeting moiety. In some embodiments, the at least a second affinity binding polypeptide is located at the N-terminus of the targeting moiety. In some embodiments, the at least a second affinity binding polypeptide is located at the C-terminus of the targeting moiety. In some embodiments, the at least a second affinity binding polypeptide is located within the targeting moiety. In some embodiments, the at least a second affinity binding polypeptide is fused directly to the targeting moiety. In some embodiments, the at least a second affinity binding polypeptide is fused indirectly to the targeting moiety via, for example, a peptide linker as provided for herein. In embodiments where the at least a second affinity binding polypeptide is located within the targeting moiety, the affinity binding polypeptide may be fused to the targeting moiety directly, fused directly at the N-terminus of the affinity binding polypeptide and fused indirectly at the C-terminus of the affinity binding polypeptide via, for example, a peptide linker as provided for herein, fused indirectly at the N-terminus of the affinity binding polypeptide via, for example, a peptide linker as provided for herein and fused directly at the C-terminus of the affinity binding polypeptide, or fused indirectly at both the N and C terminus of the affinity binding polypeptide via, for example, a polypeptide linker as provided for herein.

In some embodiments, the polypeptide further comprises at least a third, at least a fourth, or at least a fifth affinity binding polypeptide fused to the targeting moiety. In some embodiments, the polypeptide comprises at least a third affinity binding polypeptide fused to the targeting moiety. In some embodiments, the position of the at least a third affinity binding polypeptide is as provided for herein for the at least a first and the at least a second affinity binding polypeptide. In some embodiments, the polypeptide comprises at least a fourth affinity binding polypeptide fused to the targeting moiety. In some embodiments, the position of the at least a fourth affinity binding polypeptide is as provided for herein for the at least a first and the at least a second affinity binding polypeptide. In some embodiments, the polypeptide comprises at least a fifth affinity binding polypeptide fused to the targeting moiety. In some embodiments, the position of the at least a fifth affinity binding polypeptide is as provided for herein for the at least a first and the at least a second affinity binding polypeptide. The number of affinity binding polypeptides fused to the targeting moiety is only limited in that the fusion constructs should not negatively affect the function or purpose of the targeting moiety. Thus, in some embodiments, the polypeptide further comprises at least one affinity binding polypeptide. In some embodiments, the polypeptide further comprises at least two affinity binding polypeptides. In some embodiments, the polypeptide further comprises at least three affinity binding polypeptides. In some embodiments, the polypeptide further comprises at least four affinity binding polypeptides. In some embodiments, the polypeptide further comprises at least five affinity binding polypeptides. In some embodiments, the polypeptide further comprises up to five affinity binding polypeptides. In some embodiments, the polypeptide further comprises up to ten affinity binding polypeptides. In some embodiments, the polypeptide further comprises up to twenty affinity binding polypeptides.

In some embodiments, the targeting moiety of the polypeptide is selected from the group including, but not limited to, an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof. In some embodiments, the targeting moiety is an scFv.

In some embodiments, the polypeptide further comprises a virus glycoprotein. In some embodiments, the virus glycoprotein is fused directly to the targeting moiety. In some embodiments, the virus glycoprotein is fused indirectly to the targeting moiety via, for example, a peptide linker sequence as provided for herein. In some embodiments, the virus glycoprotein is not fused to the targeting moiety.

In some embodiments, the sequence of the affinity binding polypeptide fused to the glycoprotein is as provided for herein. In some embodiments, the targeting moiety binds a target as provided for herein. In some embodiments, the sequence of the targeting moiety is as provided for herein. In some embodiments, the sequence of the virus glycoprotein is as provided for herein

In some embodiments, a nucleic acid molecule is provided. In some embodiments, the nucleic acid molecule encodes for a polypeptide as provided for herein. In some embodiments, the nucleic acid molecule encodes for a virus glycoprotein. In some embodiments, the identity of the glycoprotein is as provided for herein. In some embodiments, the nucleic acid molecule further encodes for at least a first, at least a second, at least a third, at least a fourth, or at least a fifth affinity binding polypeptides. In some embodiments, the identity of the affinity binding polypeptides is as provided for herein. In some embodiments, the nucleic acid molecule encodes for a glycoprotein:affinity binding polypeptide fusion construct, wherein the at least a first, at least a second, at least a third, at least a fourth, or at least a fifth affinity binding polypeptides are located at the N-terminus of the glycoprotein, at the C-terminus of the glycoprotein, within the glycoprotein, or any combination thereof. In embodiments wherein there are more than one affinity binding polypeptides fused or linked to the glycoprotein, the location of each affinity binding polypeptide is independent of the location of the other affinity binding polypeptides. The number of affinity binding polypeptides encoded is only limited in that the fusion constructs should not negatively affect the function or purpose of the glycoprotein. Thus, in some embodiments, the nucleic acid molecule encodes for a glycoprotein that further comprises at least one affinity binding polypeptide. In some embodiments, the nucleic acid molecule encodes for a glycoprotein that further comprises at least two affinity binding polypeptides. In some embodiments, the nucleic acid molecule encodes for a glycoprotein that further comprises at least three affinity binding polypeptides. In some embodiments, the nucleic acid molecule encodes for a glycoprotein that further comprises at least four affinity binding polypeptides. In some embodiments, the nucleic acid molecule encodes for a glycoprotein that further comprises at least five affinity binding polypeptides. In some embodiments, the nucleic acid molecule encodes for a glycoprotein that further comprises up to five affinity binding polypeptides. In some embodiments, the nucleic acid molecule encodes for a glycoprotein that further comprises up to ten affinity binding polypeptides. In some embodiments, the nucleic acid molecule encodes for a glycoprotein that further comprises up to twenty affinity binding polypeptides. In some embodiments, the nucleic acid molecule further encodes for a targeting moiety. In some embodiments, the targeting moiety is fused or linked to the glycoprotein as provided for herein. In some embodiments, the targeting moiety is not fused or linked to the glycoprotein.

In some embodiments, the nucleic acid molecule encodes for a targeting moiety. In some embodiments, the identity of the targeting moiety is as provided for herein. In some embodiments, the nucleic acid molecule further encodes for at least a first, at least a second, at least a third, at least a fourth, or at least a fifth affinity binding polypeptides. In some embodiments, the identity of the affinity binding polypeptides is as provided for herein. In some embodiments, the nucleic acid molecule encodes for a targeting moiety:affinity binding polypeptide fusion construct, wherein the at least a first, at least a second, at least a third, at least a fourth, or at least a fifth affinity binding polypeptides are located at the N-terminus of the targeting moiety, at the C-terminus of the targeting moiety, within the targeting moiety, or any combination thereof. In embodiments wherein there are more than one affinity binding polypeptides fused or linked to the targeting moiety, the location of each affinity binding polypeptide is independent of the location of the other affinity binding polypeptides. The number of affinity binding polypeptides encoded is only limited in that the fusion constructs should not negatively affect the function or purpose of the targeting moiety. Thus, in some embodiments, the nucleic acid molecule encodes for a targeting moiety that further comprises at least one affinity binding polypeptide. In some embodiments, the nucleic acid molecule encodes for a targeting moiety that further comprises at least two affinity binding polypeptides.

In some embodiments, the nucleic acid molecule encodes for a targeting moiety that further comprises at least three affinity binding polypeptides. In some embodiments, the nucleic acid molecule encodes for a targeting moiety that further comprises at least four affinity binding polypeptides. In some embodiments, the nucleic acid molecule encodes for a targeting moiety that further comprises at least five affinity binding polypeptides. In some embodiments, the nucleic acid molecule encodes for a targeting moiety that further comprises up to five affinity binding polypeptides. In some embodiments, the nucleic acid molecule encodes for a targeting moiety that further comprises up to ten affinity binding polypeptides. In some embodiments, the nucleic acid molecule encodes for a targeting moiety that further comprises up to twenty affinity binding polypeptides. In some embodiments, the nucleic acid molecule further encodes for a glycoprotein. In some embodiments, the glycoprotein is fused or linked to the targeting moiety as provided for herein. In some embodiments, the glycoprotein is not fused or linked to the targeting moiety.

### Polypeptides of Interest

As provided for herein, the pseudotyped viral-like particle or viral vector as provided for herein can also comprise a nucleic acid molecule encoding a heterologous molecule of interest. As used herein, “heterologous molecule of interest” is meant to refer to any product that may be encoded by a nucleic acid molecule. As non-limiting examples, “heterologous molecule of interest” may refer to an siRNA, an shRNA, a peptide, a polypeptide, a protein, a viral payload, a viral genome, or a combination thereof. In some embodiments, the polypeptide is a chimeric antigen receptor (“CAR”). In some embodiments, the heterologous molecule of interest is an siRNA, an shRNA, a non-coding RNA (e.g. a guide RNA for a CRISPR system), a peptide, a polypeptide, a protein, a viral payload, a viral genome, a chimeric antigen receptor (“CAR”), or a combination thereof. In some embodiments, the heterologous molecule of interest is a CAR.

A “chimeric antigen receptor” or “CAR” as used herein refers to an antigen-binding domain that is fused, directly, or indirectly (e.g. via a hinge or transmembrane domain to an intracellular signaling domain capable of activating or stimulating an immune cell. Most commonly, the CAR's extracellular binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries). In various embodiments, this scFv is fused to a transmembrane domain and then to an intracellular signaling domain. However, the antigen binding domain can be any molecule that can bind to the to target on the cell. For example, the antigen binding domain of a CAR can be an antibody, a scFv antibody, an antigen binding domain, an ankyrin repeat (e.g. DARPIN), a VHH domain antibody, a nanobody, single domain antibody, a FN3 domain, or any combination thereof. In some embodiments, a CAR includes those that solely provide CD3ζ signals upon antigen binding. In some embodiments, the CAR includes those that provide both costimulation (e.g. CD28 or CD137) and activation (CD3ζ). In some embodiments, the CARs include those that provide multiple costimulation (e.g. CD28 and CD137) and activation (CD3ζ). In various embodiments, the CAR is selected to have high affinity or avidity for the antigen. In some embodiments, the antigen-binding domain binds to CD20. In some embodiments, the antigen-binding domain comprises a CD20 antibody, or fragment thereof. In some embodiments, antibody fragments are as provided for herein, such as but not limited to a scFv antibody, an antigen binding domain, an ankyrin repeat (e.g. DARPIN), a VHH domain antibody, a nanobody, single domain antibody, a FN3 domain, or any combination thereof.

In some embodiments, the antigen-binding domain of the CAR comprises a V_{H} domain, a V_{L} domain, or a V_{H} and a V_{L} domain. In some embodiments, the V_{H} domain comprises an amino acid sequence having at least 75%, 80%, 85%, 85%, 87%, 88%, 89%, 90%, 91%, 92%, 93%, 94%, 95%, 96%, 97%, 98%, 99%, or 100% identity to SEQ ID NO: 192, or any value or range in-between.

(SEQ ID NO: 192)
EVQLVESGGGLVQPGRSLRLSCAASGFTFNDYAMHWVRQAPGKGLEWVST
ISWNSGSIGYADSVKGRFTISRDNAKKSLYLQMNSLRAEDTALYYCAKDI
QYGNYYYGMDVWGQGTTVTVSS

In some embodiments, the V_{H} domain comprises an amino acid sequence having at least 90% identity to SEQ ID NO: 192. In some embodiments, the V_{H} domain comprises an amino acid sequence having at least 95% identity to SEQ ID NO: 192. In some embodiments, the V_{H} domain comprises an amino acid sequence having at least 98% identity to SEQ ID NO: 192. In some embodiments, the V_{H} domain comprises an amino acid sequence having at least 99% identity to SEQ ID NO: 192. In some embodiments, the V_{H} domain comprises an amino acid sequence having the sequence of SEQ ID NO: 192.

In some embodiments, the V_{L} domain comprises an amino acid sequence having at least 75%, 80%, 85%, 85%, 87%, 88%, 89%, 90%, 91%, 92%, 93%, 94%, 95%, 96%, 97%, 98%, 99%, or 100% identity to SEQ ID NO: 193, or any value or range in-between.

(SEQ ID NO: 193)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPITFGQ
GTRLEIK

In some embodiments, the V_{L} domain comprises an amino acid sequence having at least 90% identity to SEQ ID NO: 193. In some embodiments, the V_{L} domain comprises an amino acid sequence having at least 95% identity to SEQ ID NO: 193. In some embodiments, the V_{L} domain comprises an amino acid sequence having at least 98% identity to SEQ ID NO: 193. In some embodiments, the V_{L} domain comprises an amino acid sequence having at least 99% identity to SEQ ID NO: 193. In some embodiments, the V_{L} domain comprises an amino acid sequence having the sequence of SEQ ID NO: 193.

In some embodiments, the antigen-binding domain of the CAR comprises a V_{H} domain and a V_{L} domain. In some embodiments, the V_{H} and V_{L} domain are not linked by a linker peptide. In some embodiments, the V_{H} and V_{L} domain are linked by a linker peptide, such as those as provided for herein, including but not limited to: (GGGGS)ᵤ(SEQ ID NO: 4), wherein each n is independently 1-5. In some embodiment n is 1. In some embodiment n is 2. In some embodiment n is 3. In some embodiment n is 4. In some embodiment n is 5.

In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 192, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 193. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 192, and comprises a V_{L} having at least 90% identity to SEQ ID NO: 193. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 192, and comprises a V_{L} having at least 95% identity to SEQ ID NO: 193. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 192, and comprises a V_{L} having at least 98% identity to SEQ ID NO: 193. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 192, and comprises a V_{L} having at least 99% identity to SEQ ID NO: 193. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 192, and comprises a V_{L} having the sequence of SEQ ID NO: 193. In some embodiments, the antigen-binding domain of the CAR comprising a V_{H} domain and a V_{L} domain comprises a V_{H} domain having at least 90% identity to SEQ ID NO: 192, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 193. In some embodiments, the antigen-binding domain of the CAR comprising a V_{H} domain and a V_{L} domain comprises a V_{H} domain having at least 95% identity to SEQ ID NO: 192, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 193. In some embodiments, the antigen-binding domain of the CAR comprising a V_{H} domain and a V_{L} domain comprises a V_{H} domain having at least 98% identity to SEQ ID NO: 192, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 193. In some embodiments, the antigen-binding domain of the CAR comprising a V_{H} domain and a V_{L} domain comprises a V_{H} domain having at least 99% identity to SEQ ID NO: 192, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 193. In some embodiments, the antigen-binding domain of the CAR comprising a V_{H} domain and a V_{L} domain comprises a V_{H} domain having an amino acid sequence of SEQ ID NO: 192, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 193. In some embodiments, the antigen-binding domain of the CAR comprising a V_{H} domain and a V_{L} domain comprises a V_{H} domain having at least 90% identity to SEQ ID NO: 192, and comprises a V_{L} having at least 90% identity to SEQ ID NO: 193. In some embodiments, the antigen-binding domain of the CAR comprising a V_{H} domain and a V_{L} domain comprises a V_{H} domain having at least 95% identity to SEQ ID NO: 192, and comprises a V_{L} having at least 90% identity to SEQ ID NO: 193. In some embodiments, the antigen-binding domain of the CAR comprising a V_{H} domain and a V_{L} domain comprises a V_{H} domain having at least 90% identity to SEQ ID NO: 192, and comprises a V_{L} having at least 95% identity to SEQ ID NO: 193. In some embodiments, the antigen-binding domain of the CAR comprising a V_{H} domain and a V_{L} domain comprises a V_{H} domain having at least 95% identity to SEQ ID NO: 192, and comprises a V_{L} having at least 95% identity to SEQ ID NO: 193. In some embodiments, the antigen-binding domain of the CAR comprising a V_{H} domain and a V_{L} domain comprises a V_{H} domain having at least 98% identity to SEQ ID NO: 192, and comprises a V_{L} having at least 98% identity to SEQ ID NO: 193. In some embodiments, the antigen-binding domain of the CAR comprising a V_{H} domain and a V_{L} domain comprises a V_{H} domain having at least 99% identity to SEQ ID NO: 192, and comprises a V_{L} having at least 99% identity to SEQ ID NO: 193. In some embodiments, the antigen-binding domain of the CAR comprising a V_{H} domain and a V_{L} domain comprises a V_{H} domain having an amino acid sequence of SEQ ID NO: 192, and comprises a V_{L} having an amino acid sequence of SEQ ID NO: 193.

In some embodiments, the antigen-binding domain of the CAR comprises a formula of V_{H}-Z-V_{L}, wherein V_{H} is a heavy chain variable region comprising the amino acid sequence of SEQ ID NO: 192, Z is a linker comprising the amino acid sequence GGGGSGGGGSGGGGS (SEQ ID NO: 3), and V_{L} is a light chain variable region comprising the amino acid sequence of SEQ ID NO: 193. In some embodiments, the antigen-binding domain of the CAR comprising a formula of V_{H}-Z-V_{L} has an amino acid sequence as set forth below:

(SEQ ID NO: 194)
EVQLVESGGGLVQPGRSLRLSCAASGFTFNDYAMHWVRQAPGKGLEWVST
ISWNSGSIGYADSVKGRFTISRDNAKKSLYLQMNSLRAEDTALYYCAKDI
QYGNYYYGMDVWGQGTTVTVSSGGGGSGGGGSGGGGSEIVLTQSPATLSL
SPGERATLSCRASQSVSSYLAWYQQKPGQAPRLLIYDASNRATGIPARFS
GSGSGTDFTLTISSLEPEDFAVYYCQQRSNWPITFGQGTRLEIK

In some embodiments, the antigen-binding domain of the CAR comprises an amino acid sequence having at least 85%, 86%, 87%, 88%, 89%, 90%, 91%, 92%, 93%, 94%, 95%, 96%, 97%, 98%, 99%, or 100% identity to a sequence of SEQ ID NO: 194. In some embodiments, the antigen-binding domain of the CAR comprises an amino acid sequence having at least 90% identity to a sequence of SEQ ID NO: 194. In some embodiments, the antigen-binding domain of the CAR comprises an amino acid sequence having at least 95% identity to a sequence of SEQ ID NO: 194. In some embodiments, the antigen-binding domain of the CAR comprises an amino acid sequence having at least 98% identity to a sequence of SEQ ID NO: 194. In some embodiments, the antigen-binding domain of the CAR comprises an amino acid sequence having at least 99% identity to a sequence of SEQ ID NO: 194. In some embodiments, the antigen-binding domain of the CAR comprises the amino acid sequence of SEQ ID NO: 194.

In some embodiments, the antigen-binding domain of the CAR comprises a formula of V_{L}-Z-V_{H}, wherein V_{L} is a light chain variable region comprising the amino acid sequence of SEQ ID NO: 193, Z is a linker comprising the amino acid sequence GGGGSGGGGSGGGGS (SEQ ID NO: 3), and V_{H} is a light chain variable region comprising the amino acid sequence of SEQ ID NO: 192. In some embodiments, the antigen-binding domain of the CAR comprising a formula of V_{L}-Z-V_{H} has an amino acid sequence as set forth below:

(SEQ ID NO: 195)
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPITFGQ
GTRLEIKGGGGSGGGGSGGGGSEVQLVESGGGLVQPGRSLRLSCAASGFT
FNDYAMHWVRQAPGKGLEWVSTISWNSGSIGYADSVKGRFTISRDNAKKS
LYLQMNSLRAEDTALYYCAKDIQYGNYYYGMDVWGQGTTVTVSS

In some embodiments, the antigen-binding domain of the CAR comprises an amino acid sequence having at least 85%, 86%, 87%, 88%, 89%, 90%, 91%, 92%, 93%, 94%, 95%, 96%, 97%, 98%, 99%, or 100% identity to a sequence of SEQ ID NO: 195. In some embodiments, the antigen-binding domain of the CAR comprises an amino acid sequence having at least 90% identity to a sequence of SEQ ID NO: 195. In some embodiments, the antigen-binding domain of the CAR comprises an amino acid sequence having at least 95% identity to a sequence of SEQ ID NO: 195. In some embodiments, the antigen-binding domain of the CAR comprises an amino acid sequence having at least 98% identity to a sequence of SEQ ID NO: 195. In some embodiments, the antigen-binding domain of the CAR comprises an amino acid sequence having at least 99% identity to a sequence of SEQ ID NO: 195. In some embodiments, the antigen-binding domain of the CAR comprises the amino acid sequence of SEQ ID NO: 195.

In some embodiments, the antigen-binding domain of the CAR comprises a V_{H} domain, a V_{L} domain, or a V_{H} and a V_{L} domain. In some embodiments, the V_{H} domain comprises an amino acid sequence having at least 75%, 80%, 85%, 85%, 87%, 88%, 89%, 90%, 91%, 92%, 93%, 94%, 95%, 96%, 97%, 98%, 99%, or 100% identity to SEQ ID NO: 196, or any value or range in-between.

(SEQ ID NO: 196)
DIVLTQSPAILSASPGEKVTMTCRASSSVNYMDWYQKKPGSSPKPWIYAT
SNLASGVPARFSGSGSGTSYSLTISRVEAEDAATYYCQQWSENPPTFGGG
TKLEIKGSTS

In some embodiments, the V_{H} domain comprises an amino acid sequence having at least 90% identity to SEQ ID NO: 196. In some embodiments, the V_{H} domain comprises an amino acid sequence having at least 95% identity to SEQ ID NO: 196. In some embodiments, the V_{H} domain comprises an amino acid sequence having at least 98% identity to SEQ ID NO: 196. In some embodiments, the V_{H} domain comprises an amino acid sequence having at least 99% identity to SEQ ID NO: 196. In some embodiments, the V_{H} domain comprises an amino acid sequence having the sequence of SEQ ID NO: 196.

In some embodiments, the V_{L} domain comprises an amino acid sequence having at least 75%, 80%, 85%, 85%, 87%, 88%, 89%, 90%, 91%, 92%, 93%, 94%, 95%, 96%, 97%, 98%, 99%, or 100% identity to SEQ ID NO: 197, or an value or range in-between.

(SEQ ID NO: 197)
EVQLQQSGAELVKPGASVKMSCKASGYTFTSYNMHWVKQTPGQGLEWIGA
IYPGNGDTSYNQKFKGKATLTADKSSSTAYMQLSSLTSEDSADYYCARSN
YYGSSYWFFDVWGAGTTVTVSS

In some embodiments, the V_{L} domain comprises an amino acid sequence having at least 90% identity to SEQ ID NO: 197. In some embodiments, the V_{L} domain comprises an amino acid sequence having at least 95% identity to SEQ ID NO: 197. In some embodiments, the V_{L} domain comprises an amino acid sequence having at least 98% identity to SEQ ID NO: 197. In some embodiments, the V_{L} domain comprises an amino acid sequence having at least 99% identity to SEQ ID NO: 197. In some embodiments, the V_{L} domain comprises an amino acid sequence having the sequence of SEQ ID NO: 197.

In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 196, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 197. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 196, and comprises a V_{L} having at least 90% identity to SEQ ID NO: 197. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 196, and comprises a V_{L} having at least 95% identity to SEQ ID NO: 197. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 196, and comprises a V_{L}, having at least 98% identity to SEQ ID NO: 197. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 196, and comprises a V_{L} having at least 99% identity to SEQ ID NO: 197. In some embodiments, the antigen-binding domain of the CAR comprising a V_{H} domain and a V_{L} domain comprises a V_{H} domain having at least 75%, 80%, 85%, 85%, 87%, 88%, 89%, 90%, 91%, 92%, 93%, 94%, 95%, 96%, 97%, 98%, 99%, or 100% identity to SEQ ID NO: 196, and comprises a V_{L} having the sequence of SEQ ID NO: 197. In some embodiments, the antigen-binding domain of the CAR comprising a V_{H} domain and a V_{L} domain comprises a V_{H} domain having at least 90% identity to SEQ ID NO: 196, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 197. In some embodiments, the antigen-binding domain of the CAR comprising a V_{H} domain and a V_{L} domain comprises a V_{H} domain having at least 95% identity to SEQ ID NO: 196, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 197. In some embodiments, the antigen-binding domain of the CAR comprising a V_{H} domain and a V_{L} domain comprises a V_{H} domain having at least 98% identity to SEQ ID NO: 196, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 197. In some embodiments, the antigen-binding domain of the CAR comprising a V_{H} domain and a V_{L} domain comprises a V_{H} domain having at least 99% identity to SEQ ID NO: 196, and comprises a V_{L} having at least 75%, 80%, 85%, 85%, 87%, 88%, 89%, 90%, 91%, 92%,93%,94%,95%,96%,97%,98%, 99%, or 100% identity to SEQ ID NO: 197. In some embodiments, the antigen-binding domain of the CAR comprising a V_{H} domain and a V_{L} domain comprises a V_{H} domain having an amino acid sequence of SEQ ID NO: 196, and comprises a V_{L} having at least 75%, 80%, 85%, 85%, 87%, 88%, 89%, 90%, 91%, 92%, 93%, 94%, 95%, 96%, 97%, 98%, 99%, or 100% identity to SEQ ID NO: 197. In some embodiments, the antigen-binding domain of the CAR comprising a V_{H} domain and a V_{L} domain comprises a V_{H} domain having at least 90% identity to SEQ ID NO: 196, and comprises a V_{L} having at least 90% identity to SEQ ID NO: 197. In some embodiments, the antigen-binding domain of the CAR comprising a V_{H} domain and a V_{L} domain comprises a V_{H} domain having at least 95% identity to SEQ ID NO: 196, and comprises a V_{L} having at least 90% identity to SEQ ID NO: 197. In some embodiments, the antigen-binding domain of the CAR comprising a V_{H} domain and a ViL domain comprises a V_{H} domain having at least 90% identity to SEQ ID NO: 196, and comprises a V_{L} having at least 95% identity to SEQ ID NO: 197. In some embodiments, the antigen-binding domain of the CAR comprising a V_{H} domain and a V_{L} domain comprises a V_{H} domain having at least 95% identity to SEQ ID NO: 196, and comprises a V_{L} having at least 95% identity to SEQ ID NO: 197. In some embodiments, the antigen-binding domain of the CAR comprising a V_{H} domain and a V_{L} domain comprises a V_{H} domain having at least 98% identity to SEQ ID NO: 196, and comprises a V_{L} having at least 98% identity to SEQ ID NO: 197. In some embodiments, the antigen-binding domain of the CAR comprising a V_{H} domain and a V_{L} domain comprises a V_{H} domain having at least 99% identity to SEQ ID NO: 196, and comprises a V_{L} having at least 99% identity to SEQ ID NO: 197. In some embodiments, the antigen-binding domain of the CAR comprising a Vr domain and a V_{L} domain comprises a V_{H} domain having an amino acid sequence of SEQ ID NO: 196, and comprises a V_{L} having an amino acid sequence of SEQ ID NO: 197.

In some embodiments, the antigen-binding domain of the CAR comprises a formula of V_{H}-Z-V_{L}, wherein V_{H} is a heavy chain variable region comprising the amino acid sequence of SEQ ID NO: 196, Z is a linker comprising the amino acid sequence GGGGSGGGGSGGGGS (SEQ ID NO: 3), and V_{L} is a light chain variable region comprising the amino acid sequence of SEQ ID NO: 197. In some embodiments, the antigen-binding domain of the CAR comprising a formula of V_{H}-Z-V_{L} has an amino acid sequence as set forth below:

(SEQ ID NO: 198)
DIVLTQSPAILSASPGEKVTMTCRASSSVNYMDWYQKKPGSSPKPWIYAT
SNLASGVPARFSGSGSGTSYSLTISRVEAEDAATYYCQQWSFNPPTFGGG
TKLEIKGSTSGGGGSGGGGSGGGGSSEVQLQQSGAELVKPGASVKMSCKA
SGYTFTSYNMHWVKQTPGQGLEWIGAIYPGNGDTSYNQKFKGKATLIADK
SSSTAYMQLSSLTSEDSADYYCARSNYYGSSYWFFDVWGAGTTVTVSS

In some embodiments, the antigen-binding domain of the CAR comprises an amino acid sequence having at least 85%, 86%, 87%, 88%, 89%, 90%, 91%, 92%, 93%, 94%, 95%, 96%, 97%, 98%, 99%, or 100% identity to a sequence of SEQ ID NO: 198. In some embodiments, the antigen-binding domain of the CAR comprises an amino acid sequence having at least 90% identity to a sequence of SEQ ID NO: 198. In some embodiments, the antigen-binding domain of the CAR comprises an amino acid sequence having at least 95% identity to a sequence of SEQ ID NO: 198. In some embodiments, the antigen-binding domain of the CAR comprises an amino acid sequence having at least 98% identity to a sequence of SEQ ID NO: 198. In some embodiments, the antigen-binding domain of the CAR comprises an amino acid sequence having at least 99% identity to a sequence of SEQ ID NO: 198. In some embodiments, the antigen-binding domain of the CAR comprises the amino acid sequence of SEQ ID NO: 198.

In some embodiments, the antigen-binding domain of the CAR comprises a formula of V_{L}-Z-V_{H}, wherein V_{L} is a light chain variable region comprising the amino acid sequence of SEQ ID NO: 197, Z is a linker comprising the amino acid sequence GGGGSGGGGSGGGGS (SEQ ID NO: 3), and V_{H} is a light chain variable region comprising the amino acid sequence of SEQ ID NO: 196. In some embodiments, the antigen-binding domain of the CAR comprising a formula of V_{L}-Z-V_{H} has an amino acid sequence as set forth below:

(SEQ ID NO: 199)
SEVQLQQSGAELVKPGASVKMSCKASGYTFTSYNMHWVKQTPGQGLEWIG
AIYPGNGDTSYNQKFKGKATLTADKSSSTAYMQLSSLTSEDSADYYCARS
NYYGSSYWFFDVWGAGTTVTVSSGGGGSGGGGSGGGGSDIVLTQSPAILS
ASPGEKVTMTCRASSSVNYMDWYQKKPGSSPKPWIYATSNLASGVPARFS
GSGSGTSYSLTISRVEAEDAATYYCQQWSFNPPTFGGGTKLEIKGSTS

In some embodiments, the antigen-binding domain of the CAR comprises an amino acid sequence having at least 85%, 86%, 87%, 88%, 89%, 90%, 91%, 92%, 93%, 94%, 95%, 96%, 97%, 98%, 99%, or 100% identity to a sequence of SEQ ID NO: 199. In some embodiments, the antigen-binding domain of the CAR comprises an amino acid sequence having at least 90% identity to a sequence of SEQ ID NO: 199. In some embodiments, the antigen-binding domain of the CAR comprises an amino acid sequence having at least 95% identity to a sequence of SEQ ID NO: 199. In some embodiments, the antigen-binding domain of the CAR comprises an amino acid sequence having at least 98% identity to a sequence of SEQ ID NO: 199. In some embodiments, the antigen-binding domain of the CAR comprises an amino acid sequence having at least 99% identity to a sequence of SEQ ID NO: 199. In some embodiments, the antigen-binding domain of the CAR comprises the amino acid sequence of SEQ ID NO: 199.

In some embodiments, the antigen-binding domain of the CAR comprises rituximab, ocrelizumab, obinutuzumab, ofatumumab, ibritumomab tiuxetan, tositumomab, or ublituximab. In some embodiment, the antigen-binding domain comprises rituximab. In some embodiment, the antigen-binding domain comprises ofatumumab. In some embodiments, the CAR comprises the 4-1BB domain as well. These are merely illustrative in nature and are not limiting to the present embodiments and any chimeric antigen receptor can be delivered in conjunction with the viral particles and vectors provided for herein. These are non-limiting examples of CARs and any CAR construct could be encoded for by the nucleic acid molecule.

In some embodiments, the pseudotyped viral particle further comprises a heterologous nucleic acid molecule encoding a cargo of interest. The nucleic acid molecule may be useful for modulating the expression of a target gene. In some embodiments, the cargo can be used to modulate the activity of a cell or express a protein that is trafficked to the surface of the target cell. Therefore, in some embodiments, the nucleic acid may comprise an siRNA or an shRNA. The nucleic acid may also encode for a cargo of interest. Therefore, in some embodiments, the cargo of interest may comprise a polypeptide or portion thereof, a protein or portion thereof, a chimeric antigen receptor or portion thereof, or a tumor antigen or a portion thereof. In some embodiments, the cargo of interest is an antibody that is produced by the virus, which can then be secreted by the cell that is infected with the virus. The term “protein” can refer to any polypeptide that carries a native function in a cellular environment. Therefore, in some embodiments, the protein encoded by the nucleic acid cargo of interest may comprise an enzyme, a nuclear receptor, a transporter, a ribosomal protein, a membrane bound protein, a cytoplasmic protein, a G-protein coupled receptor, a voltage gated ion channel, a secretory protein, a mitochondria protein, a cytokine, a chimeric antigen receptor, a tumor antigen, or a portion or chimeric species thereof.

### Methods of Viral Production and Purification

The present application also contemplates methods of producing and purifying the pseudotyped viruses described herein. In some embodiments, a method of making a pseudotyped viral-like particle or viral vector is provided. In some embodiments, the pseudotyped viral-like particle or viral vector is as provided for herein. In some embodiments, the method comprises transfecting or transducing a packaging cell line with a nucleic acid molecule as provided for herein under conditions sufficient to produce the pseudotyped viral like particle or viral vector. In some embodiments, the method further comprises isolating the pseudotyped viral-like particle or viral vector.

In some embodiments, a method of purifying a pseudotyped viral-like particle or viral vector is provided. In some embodiments, the pseudotyped viral-like particle or viral vector is as provided for herein. In some embodiments, the method comprises transfecting or transducing a packaging cell line with a nucleic acid molecule; culturing the packaging cell line under conditions sufficient to produce the pseudotypd viral like particle or viral vector; isolating the pseudotyped viral-like particle or viral vector; and purifying the pseudotyped viral-like particle or viral vector. In some embodiments, the nucleic acid molecule is as provided for herein. In some embodiments, the nucleic acid molecule encodes for a recombinant virus glycoprotein, an engineered targeting moiety for binding to a target cell, and at least a first affinity binding polypeptide, wherein the affinity binding polypeptide is fused to the glycoprotein, the targeting moiety, or any combination thereof. In some embodiments the affinity binding polypeptide is fused to the glycoprotein as provided for herein. In some embodiments, the affinity binding polypeptide is fused to the targeting moiety as provided for herein. In some embodiments, the affinity binding polypeptides are fused to the glycoprotein and to the targeting moiety as provided for herein. In some embodiments, the identity of the glycoprotein is as provided for herein. In some embodiments, the identity of the targeting moiety is as provided for herein. In some embodiments, the identity of the affinity binding polypeptides is as provided for herein. In some embodiments, the purifying step further comprises purification of the pseudotyped viral-like particle or viral vector via use of the affinity binding polypeptide included in the glycoprotein, the targeting moiety, or both.

In some embodiments, the isolating step comprises collecting the packaging cell line supernatant containing the pseudotyped viral-like particle or viral vector. The viral-like particle or viral vector may be isolated from the packaging cell line via any known method of viral isolation during viral production. Such methods are within the scope of the present application.

In some embodiments, the purifying step further comprises incubating the isolated pseudotyped viral-like particle or viral vector with a stationary phase; washing the stationary phase with an appropriate wash buffer; and eluting the pseudotyped viral-like particle or viral vector, thereby purifying the pseudotyped viral-like particle or viral vector. In some embodiments, the stationary phase comprises an interacting molecule which interacts with the affinity binding polypeptide of the glycoprotein, the targeting moiety, or both. Stationary phases for use in protein or virus purification are well known in the art and any such stationary phase may be utilized provided that it pairs with the affinity binding polypeptide as provided for herein. As a non-limiting examples, the affinity binding polypeptide may be an antibody, and the stationary phase may consist of anchored target proteins to said antibody. In another non-limiting example, the affinity binding polypeptide may be a polyhistidine tag, and the stationary phase may comprise an antibody recognizing polyhistidine or the stationary phase may comprise nickel. In another non-limiting example, the affinity binding polypeptide may be a streptavidin tag selected from the group including but not limited to a streptavidin-binding peptide, a streptavidin binding tag, a strep-tag II, a twin-strep tag, or any variation thereof, and the stationary phase may comprise an antibody recognizing streptavidin or the stationary phase may comprise a small molecule or other compound that binds streptavidin. In some embodiments, the stationary phase comprises biotin. In some embodiments, the stationary phase comprises Strep-Tactin®. Other stationary phase:affinity binding polypeptide combinations are known in the art and such combinations are within the scope of the present application. Further, the application of the stationary phase may be any such application known in the art to promote the purification of proteins and/or viruses. For example, the stationary phase may consist of a column, a filter, a spin column, magnetic beads, a label free device, or the like. In some embodiments, the stationary phase comprises a column. In some embodiments, the stationary phase comprises a filter. In some embodiments, the stationary phase comprises a spin column. In some embodiments, the stationary phase comprises magnetic beads. In some embodiments, the stationary phase comprises a label free device.

In some embodiments, the stationary phase comprises a column. In some embodiments, the affinity binding polypeptide is a streptavidin tag selected from the group including but not limited to a streptavidin-binding peptide, a streptavidin binding tag, a strep-tag II, a twin-strep tag, or any variation thereof as provided for herein; and the stationary phase comprises an antibody recognizing streptavidin, a small molecule or other compound that binds streptavidin, biotin, Strep-Tactin®, or the like. In some embodiments, the isolated pseudotyped viral-like particle or viral vector is incubated with the stationary phase, resulting in bound isolated pseudotyped viral-like particle or viral vector. In some embodiments, the bound isolated pseudotyped viral-like particle or viral vector is washed with an appropriate wash buffer, resulting in a washed, bound isolated pseudotyped viral-like particle or viral vector. In some embodiments, the washed, bound isolated pseudotyped viral-like particle or viral vector is eluted from the stationary phase, thereby purifying the pseudotyped viral-like particle or viral vector.

In some embodiments, the stationary phase comprises magnetic beads. In some embodiments, the affinity binding polypeptide is a streptavidin tag selected from the group including but not limited to a streptavidin-binding peptide, a streptavidin binding tag, a strep-tag II, a twin-strep tag, or any variation thereof as provided for herein; and the stationary phase comprises an antibody recognizing streptavidin, a small molecule or other compound that binds streptavidin, biotin, Strep-Tactin®, or the like. In some embodiments, the isolated pseudotyped viral-like particle or viral vector is incubated with the stationary phase, resulting in bound isolated pseudotyped viral-like particle or viral vector. In some embodiments, the bound isolated pseudotyped viral-like particle or viral vector is washed with an appropriate wash buffer, resulting in a washed, bound isolated pseudotyped viral-like particle or viral vector. In some embodiments, the washed, bound isolated pseudotyped viral-like particle or viral vector is eluted from the stationary phase, thereby purifying the pseudotyped viral-like particle or viral vector.

In some embodiments, the stationary phase comprises a label free device. In some embodiments, the label free device comprises biosensors coated with the stationary phase, wherein when the isolated pseudotyped viral-like particle or viral vector is incubated with the stationary phase coated biosensor, resulting in bound isolated pseudotyped viral-like particle or viral vector. In some embodiments, the bound isolated pseudotyped viral-like particle or viral vector is washed with an appropriate wash buffer, resulting in a washed, bound isolated pseudotyped viral-like particle or viral vector. In some embodiments, the washed, bound isolated pseudotyped viral-like particle or viral vector is eluted from the stationary phase coated biosensor, thereby purifying the pseudotyped viral-like particle or viral vector. The label free device may be any appropriate label free device. In some embodiments, the label free device is an Octet® Label-free device.

In embodiments where the affinity binding polypeptide in located on the N or C-terminus or the glycoprotein, the targeting moiety, or both, the affinity binding polypeptide may further comprise a protease cleavage recognition site. In such embodiments, the purification method may further comprise treating the eluted pseudotyped viral-like particle or viral vector with an appropriate protease, thereby cleaving the affinity binding polypeptide from the glycoprotein, the targeting moiety, or both. In some embodiments, the method may further comprise applying the pseudotyped viral-like particle or viral vector, protease, and cleaved affinity binding polypeptide solution to an appropriate stationary phase to purify the pseudotyped viral-like particle or viral vector from the protease and cleaved affinity binding polypeptide.

In any of the embodiments provided for herein, the method may further comprise preparing the purified pseudotyped viral-like particle or viral vector into a drug product or pharmaceutical composition. In any of the embodiments provided for herein, the method may further comprise concentrating the purified pseudotyped viral-like particle or viral vector, resulting in a concentrated purified pseudotyped viral-like particle or viral vector. In any of the embodiments provided for herein, the method may further comprise preparing the concentrated purified pseudotyped viral-like particle or viral vector into a drug product or pharmaceutical composition.

### Enumerated Embodiments

In some embodiments, the following embodiments are provided:
1. A pseudotyped viral-like particle or viral vector comprising:
   an envelope comprising a recombinant virus glycoprotein, a targeting moiety for binding to a target cell, and at least a first affinity binding polypeptide, wherein the affinity binding polypeptide is fused to the glycoprotein, the targeting moiety, or any combination thereof; and
   a nucleic acid molecule encoding a heterologous molecule of interest.
2. The pseudotyped viral-like particle or viral vector of embodiment 1, wherein the virus glycoprotein is derived from a virus from the group consisting of Human Immunodeficiency Virus (HIV), Simian Immunodeficiency Virus (SIV), Ebola virus (EbV), Nipah Virus (NiV), Measles virus (MeV), Vesicular stomatitis virus (VSV), Spring viremia of carp virus (SVCV) or a combination thereof.
3. The pseudotyped viral-like particle or viral vector of embodiment I or 2, wherein the virus glycoprotein is HIV glycoprotein gp120, SIV glycoprotein gp120, EbV glycoprotein, NiV-G, NiV-F, MeV-H, MeV-F, VSV-G, SVCV-G, any variant thereof, or any combination thereof.
4. The pseudotyped viral-like particle or viral vector of any one of embodiments 1-3, wherein the viral-like particle is a retroviral-like particle or retroviral vector.
5. The pseudotyped viral-like particle or viral vector of embodiment 4, wherein the retroviral-like particle is a lentivirus based viral particle or viral vector.
6. The pseudotyped viral-like particle or viral vector of any one of embodiments 1-5, wherein the at least a first affinity binding polypeptide is an affinity tag selected from the group consisting of a polyhistidine tag, a polyarginine tag, a FLAG tag, a streptavidin tag, a calmodulin binding peptide, or a variant or combination thereof.
7. The pseudotyped viral-like particle or viral vector of embodiment 6, wherein the streptavidin tag is selected from the group consisting of a streptavidin-binding peptide, a streptavidin binding tag, a strep-tag II, a twin-strep tag, a tag of Formula I, a tag of Formula II, or a variant or combination thereof.
8. The pseudotyped viral-like particle or viral vector of embodiments 1-7, wherein the at least a first affinity binding polypeptide is a strep-tag II polypeptide sequence.
9. The pseudotyped viral-like particle or viral vector of any one of embodiments 1-8, wherein the at least a first affinity binding polypeptide is fused to the glycoprotein.
10. The pseudotyped viral-like particle or viral vector of embodiment 9, wherein the at least a first affinity binding polypeptide is located at the N-terminus, at the C-terminus, or within the glycoprotein.
11. The pseudotyped viral-like particle or viral vector of embodiments 9 or 10, wherein the pseudotyped viral-like particle or viral vector comprises at least a second affinity binding polypeptide.
12. The pseudotyped viral-like particle or viral vector of embodiment 11, wherein the at least a second affinity binding polypeptide is located at the N-terminus, at the C-terminus, or within the glycoprotein.
13. The pseudotyped viral-like particle or viral vector of any one of embodiments 9-12, wherein the pseudotyped viral-like particle or viral vector comprises at least a third, at least a fourth, or at least a fifth affinity binding polypeptide.
14. The pseudotyped viral-like particle or viral vector of embodiment 13, wherein each additional affinity binding polypeptide is located, independently, at the N-terminus, at the C-terminus, or within the glycoprotein.
15. The pseudotyped viral-like particle or viral vector of any one of embodiments 9-14, wherein the glycoprotein is Ebola glycoprotein.
15a. The pseudotyped viral-like particle or viral vector of any one of embodiments 9-15, wherein the at least a first affinity binding polypeptide is fused to the N-terminus of the Ebola glycoprotein.
15b. The pseudotyped viral-like particle or viral vector of any one of embodiments 9-15, wherein the at least a first affinity binding polypeptide replaces or is within the MLD domain of the Ebola glycoprotein.
16. The pseudotyped viral-like particle or viral vector of embodiment 15, wherein the Ebola glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 26, SEQ ID NO: 27, SEQ ID NO: 28, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 188, SEQ ID NO: 189, SEQ ID NO: 190, or SEQ ID NO: 191.
17. The pseudotyped viral-like particle or viral vector of embodiment 15, wherein the Ebola glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 26 or SEQ ID NO: 147.
18. The pseudotyped viral-like particle or viral vector of embodiment 15, wherein the Ebola glycoprotein fused to the affinity binding polypeptide comprises an amino acid sequence having at least 90% identity to SEQ ID NO: 26 or SEQ ID NO: 147.
19. The pseudotyped viral-like particle or viral vector of embodiment 15, wherein the Ebola glycoprotein fused to the affinity binding polypeptide comprises an amino acid sequence having at least 95% identity to SEQ ID NO: 26 or SEQ ID NO: 147.
20. The pseudotyped viral-like particle or viral vector of embodiment 15, wherein the Ebola glycoprotein fused to the affinity binding polypeptide comprises an amino acid sequence having at least 98% identity to SEQ ID NO: 26 or SEQ ID NO: 147.
21. The pseudotyped viral-like particle or viral vector of embodiment 15, wherein the Ebola glycoprotein fused to the affinity binding polypeptide comprises an amino acid of SEQ ID NO: 26.
22. The pseudotyped viral-like particle or viral vector of embodiment 15, wherein the Ebola glycoprotein fused to the affinity binding polypeptide comprises an amino acid of SEQ ID NO: 147.
23. The pseudotyped viral-like particle or viral vector of embodiment 15, wherein the Ebola glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 27 or SEQ ID NO: 148.
24. The pseudotyped viral-like particle or viral vector of embodiment 15, wherein the Ebola glycoprotein fused to the affinity binding polypeptide comprises an amino acid sequence having at least 90% identity to SEQ ID NO: 27 or SEQ ID NO: 148.
25. The pseudotyped viral-like particle or viral vector of embodiment 15, wherein the Ebola glycoprotein fused to the affinity binding polypeptide comprises an amino acid sequence having at least 95% identity to SEQ ID NO: 27 or SEQ ID NO: 148.
26. The pseudotyped viral-like particle or viral vector of embodiment 15, wherein the Ebola glycoprotein fused to the affinity binding polypeptide comprises an amino acid sequence having at least 98% identity to SEQ ID NO: 27 or SEQ ID NO: 148.
27. The pseudotyped viral-like particle or viral vector of embodiment 15, wherein the Ebola glycoprotein fused to the affinity binding polypeptide comprises an amino acid of SEQ ID NO: 27.
28. The pseudotyped viral-like particle or viral vector of embodiment 15, wherein the Ebola glycoprotein fused to the affinity binding polypeptide comprises an amino acid of SEQ ID NO: 148.
29. The pseudotyped viral-like particle or viral vector of embodiment 15, wherein the Ebola glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 28, SEQ ID NO: 137, SEQ ID NO: 151, or SEQ ID NO: 156.
30. The pseudotyped viral-like particle or viral vector of embodiment 15, wherein the Ebola glycoprotein fused to the affinity binding polypeptide comprises an amino acid sequence having at least 90% identity to SEQ ID NO: 28, SEQ ID NO: 137, SEQ ID NO: 151, or SEQ ID NO: 156.
31. The pseudotyped viral-like particle or viral vector of embodiment 15, wherein the Ebola glycoprotein fused to the affinity binding polypeptide comprises an amino acid sequence having at least 95% identity to SEQ ID NO: 28, SEQ ID NO: 137, SEQ ID NO: 151, or SEQ ID NO: 156.
32. The pseudotyped viral-like particle or viral vector of embodiment 15, wherein the Ebola glycoprotein fused to the affinity binding polypeptide comprises an amino acid sequence having at least 98% identity to SEQ ID NO: 28, SEQ ID NO: 137, SEQ ID NO: 151, or SEQ ID NO: 156.
33. The pseudotyped viral-like particle or viral vector of embodiment 15, wherein the Ebola glycoprotein fused to the affinity binding polypeptide comprises an amino acid of SEQ ID NO: 28.
34. The pseudotyped viral-like particle or viral vector of embodiment 15, wherein the Ebola glycoprotein fused to the affinity binding polypeptide comprises an amino acid of SEQ ID NO: 137.
35. The pseudotyped viral-like particle or viral vector of embodiment 15, wherein the Ebola glycoprotein fused to the affinity binding polypeptide comprises an amino acid of SEQ ID NO: 151.
36. The pseudotyped viral-like particle or viral vector of embodiment 15, wherein the Ebola glycoprotein fused to the affinity binding polypeptide comprises an amino acid of SEQ ID NO: 156.
37. The pseudotyped viral-like particle or viral vector of embodiment 15, wherein the Ebola glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 135, SEQ ID NO: 149, SEQ ID NO: 152, or SEQ ID NO: 153.
38. The pseudotyped viral-like particle or viral vector of embodiment 15, wherein the Ebola glycoprotein fused to the affinity binding polypeptide comprises an amino acid sequence having at least 90% identity to SEQ ID NO: 135, SEQ ID NO: 149, SEQ ID NO: 152, or SEQ ID NO: 153.
39. The pseudotyped viral-like particle or viral vector of embodiment 15, wherein the Ebola glycoprotein fused to the affinity binding polypeptide comprises an amino acid sequence having at least 95% identity to SEQ ID NO: 135, SEQ ID NO: 149, SEQ ID NO: 152, or SEQ ID NO: 153.
40. The pseudotyped viral-like particle or viral vector of embodiment 15, wherein the Ebola glycoprotein fused to the affinity binding polypeptide comprises an amino acid sequence having at least 98% identity to SEQ ID NO: 135, SEQ ID NO: 149, SEQ ID NO: 152, or SEQ ID NO: 153.
41. The pseudotyped viral-like particle or viral vector of embodiment 15, wherein the Ebola glycoprotein fused to the affinity binding polypeptide comprises an amino acid of SEQ ID NO: 135.
42. The pseudotyped viral-like particle or viral vector of embodiment 15, wherein the Ebola glycoprotein fused to the affinity binding polypeptide comprises an amino acid of SEQ ID NO: 149.
43. The pseudotyped viral-like particle or viral vector of embodiment 15, wherein the Ebola glycoprotein fused to the affinity binding polypeptide comprises an amino acid of SEQ ID NO: 152.
44. The pseudotyped viral-like particle or viral vector of embodiment 15, wherein the Ebola glycoprotein fused to the affinity binding polypeptide comprises an amino acid of SEQ ID NO: 153.
45. The pseudotyped viral-like particle or viral vector of embodiment 15, wherein the Ebola glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 136, SEQ ID NO: 150, SEQ ID NO: 154, or SEQ ID NO: 155.
46. The pseudotyped viral-like particle or viral vector of embodiment 15, wherein the Ebola glycoprotein fused to the affinity binding polypeptide comprises an amino acid sequence having at least 90% identity to SEQ ID NO: 136, SEQ ID NO: 150, SEQ ID NO: 154, or SEQ ID NO: 155.
47. The pseudotyped viral-like particle or viral vector of embodiment 15, wherein the Ebola glycoprotein fused to the affinity binding polypeptide comprises an amino acid sequence having at least 95% identity to SEQ ID NO: 136, SEQ ID NO: 150, SEQ ID NO: 154, or SEQ ID NO: 155.
48. The pseudotyped viral-like particle or viral vector of embodiment 15, wherein the Ebola glycoprotein fused to the affinity binding polypeptide comprises an amino acid sequence having at least 98% identity to SEQ ID NO: 136, SEQ ID NO: 150, SEQ ID NO: 154, or SEQ ID NO: 155.
49. The pseudotyped viral-like particle or viral vector of embodiment 15, wherein the Ebola glycoprotein fused to the affinity binding polypeptide comprises an amino acid of SEQ ID NO: 136.
50. The pseudotyped viral-like particle or viral vector of embodiment 15, wherein the Ebola glycoprotein fused to the affinity binding polypeptide comprises an amino acid of SEQ ID NO: 150.
51. The pseudotyped viral-like particle or viral vector of embodiment 15, wherein the Ebola glycoprotein fused to the affinity binding polypeptide comprises an amino acid of SEQ ID NO: 154.
52. The pseudotyped viral-like particle or viral vector of embodiment 15, wherein the Ebola glycoprotein fused to the affinity binding polypeptide comprises an amino acid of SEQ ID NO: 155.
53. The pseudotyped viral-like particle or viral vector of embodiment 15, wherein the Ebola glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 138, SEQ ID NO: 139, SEQ ID NO: 157, or SEQ ID NO: 158.
54. The pseudotyped viral-like particle or viral vector of embodiment 15, wherein the Ebola glycoprotein fused to the affinity binding polypeptide comprises an amino acid sequence having at least 90% identity to SEQ ID NO: 138, SEQ ID NO: 139, SEQ ID NO: 157, or SEQ ID NO: 158.
55. The pseudotyped viral-like particle or viral vector of embodiment 15, wherein the Ebola glycoprotein fused to the affinity binding polypeptide comprises an amino acid sequence having at least 95% identity to SEQ ID NO: 138, SEQ ID NO: 139, SEQ ID NO: 157, or SEQ ID NO: 158.
56. The pseudotyped viral-like particle or viral vector of embodiment 15, wherein the Ebola glycoprotein fused to the affinity binding polypeptide comprises an amino acid sequence having at least 98% identity to SEQ ID NO: 138, SEQ ID NO: 139, SEQ ID NO: 157, or SEQ ID NO: 158.
57. The pseudotyped viral-like particle or viral vector of embodiment 15, wherein the Ebola glycoprotein fused to the affinity binding polypeptide comprises an amino acid of SEQ ID NO: 138.
58. The pseudotyped viral-like particle or viral vector of embodiment 15, wherein the Ebola glycoprotein fused to the affinity binding polypeptide comprises an amino acid of SEQ ID NO: 139.
59. The pseudotyped viral-like particle or viral vector of embodiment 15, wherein the Ebola glycoprotein fused to the affinity binding polypeptide comprises an amino acid of SEQ ID NO: 157.
60. The pseudotyped viral-like particle or viral vector of embodiment 15, wherein the Ebola glycoprotein fused to the affinity binding polypeptide comprises an amino acid of SEQ ID NO: 158.
61. The pseudotyped viral-like particle or viral vector of embodiment 15, wherein the Ebola glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 140, SEQ ID NO: 141, SEQ ID NO: 159, or SEQ ID NO: 160.
62. The pseudotyped viral-like particle or viral vector of embodiment 15, wherein the Ebola glycoprotein fused to the affinity binding polypeptide comprises an amino acid sequence having at least 90% identity to SEQ ID NO: 140, SEQ ID NO: 141, SEQ ID NO: 159, or SEQ ID NO: 160.
63. The pseudotyped viral-like particle or viral vector of embodiment 15, wherein the Ebola glycoprotein fused to the affinity binding polypeptide comprises an amino acid sequence having at least 95% identity to SEQ ID NO: 140, SEQ ID NO: 141, SEQ ID NO: 159, or SEQ ID NO: 160.
64. The pseudotyped viral-like particle or viral vector of embodiment 15, wherein the Ebola glycoprotein fused to the affinity binding polypeptide comprises an amino acid sequence having at least 98% identity to SEQ ID NO: 140, SEQ ID NO: 141, SEQ ID NO: 159, or SEQ ID NO: 160.
65. The pseudotyped viral-like particle or viral vector of embodiment 15, wherein the Ebola glycoprotein fused to the affinity binding polypeptide comprises an amino acid of SEQ ID NO: 140.
66. The pseudotyped viral-like particle or viral vector of embodiment 15, wherein the Ebola glycoprotein fused to the affinity binding polypeptide comprises an amino acid of SEQ ID NO: 141.
67. The pseudotyped viral-like particle or viral vector of embodiment 15, wherein the Ebola glycoprotein fused to the affinity binding polypeptide comprises an amino acid of SEQ ID NO: 159.
68. The pseudotyped viral-like particle or viral vector of embodiment 15, wherein the Ebola glycoprotein fused to the affinity binding polypeptide comprises an amino acid of SEQ ID NO: 160.
69. The pseudotyped viral-like particle or viral vector of embodiment 15, wherein the Ebola glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 142, SEQ ID NO: 143, SEQ ID NO: 161, or SEQ ID NO: 162.
70. The pseudotyped viral-like particle or viral vector of embodiment 15, wherein the Ebola glycoprotein fused to the affinity binding polypeptide comprises an amino acid sequence having at least 90% identity to SEQ ID NO: 142, SEQ ID NO: 143, SEQ ID NO: 161, or SEQ ID NO: 162.
71. The pseudotyped viral-like particle or viral vector of embodiment 15, wherein the Ebola glycoprotein fused to the affinity binding polypeptide comprises an amino acid sequence having at least 95% identity to SEQ ID NO: 142, SEQ ID NO: 143, SEQ ID NO: 161, or SEQ ID NO: 162.
72. The pseudotyped viral-like particle or viral vector of embodiment 15, wherein the Ebola glycoprotein fused to the affinity binding polypeptide comprises an amino acid sequence having at least 98% identity to SEQ ID NO: 142, SEQ ID NO: 143, SEQ ID NO: 161, or SEQ ID NO: 162.
73. The pseudotyped viral-like particle or viral vector of embodiment 15, wherein the Ebola glycoprotein fused to the affinity binding polypeptide comprises an amino acid of SEQ ID NO: 142.
74. The pseudotyped viral-like particle or viral vector of embodiment 15, wherein the Ebola glycoprotein fused to the affinity binding polypeptide comprises an amino acid of SEQ ID NO: 143.
75. The pseudotyped viral-like particle or viral vector of embodiment 15, wherein the Ebola glycoprotein fused to the affinity binding polypeptide comprises an amino acid of SEQ ID NO: 161.
76. The pseudotyped viral-like particle or viral vector of embodiment 15, wherein the Ebola glycoprotein fused to the affinity binding polypeptide comprises an amino acid of SEQ ID NO: 162.
77. The pseudotyped viral-like particle or viral vector of embodiment 15, wherein the Ebola glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 144, SEQ ID NO: 145, SEQ ID NO: 163, or SEQ ID NO: 164.
78. The pseudotyped viral-like particle or viral vector of embodiment 15, wherein the Ebola glycoprotein fused to the affinity binding polypeptide comprises an amino acid sequence having at least 90% identity to SEQ ID NO: 144, SEQ ID NO: 145, SEQ ID NO: 163, or SEQ ID NO: 164.
79. The pseudotyped viral-like particle or viral vector of embodiment 15, wherein the Ebola glycoprotein fused to the affinity binding polypeptide comprises an amino acid sequence having at least 95% identity to SEQ ID NO: 144, SEQ ID NO: 145, SEQ ID NO: 163, or SEQ ID NO: 164.
80. The pseudotyped viral-like particle or viral vector of embodiment 15, wherein the Ebola glycoprotein fused to the affinity binding polypeptide comprises an amino acid sequence having at least 98% identity to SEQ ID NO: 144, SEQ ID NO: 145, SEQ ID NO: 163, or SEQ ID NO: 164.
81. The pseudotyped viral-like particle or viral vector of embodiment 15, wherein the Ebola glycoprotein fused to the affinity binding polypeptide comprises an amino acid of SEQ ID NO: 144.
82. The pseudotyped viral-like particle or viral vector of embodiment 15, wherein the Ebola glycoprotein fused to the affinity binding polypeptide comprises an amino acid of SEQ ID NO: 145.
83. The pseudotyped viral-like particle or viral vector of embodiment 15, wherein the Ebola glycoprotein fused to the affinity binding polypeptide comprises an amino acid of SEQ ID NO: 163.
84. The pseudotyped viral-like particle or viral vector of embodiment 15, wherein the Ebola glycoprotein fused to the affinity binding polypeptide comprises an amino acid of SEQ ID NO: 164.
85. The pseudotyped viral-like particle or viral vector of embodiment 15, wherein the Ebola glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 188, SEQ ID NO: 189, SEQ ID NO: 190, or SEQ ID NO: 191.
86. The pseudotyped viral-like particle or viral vector of embodiment 15, wherein the Ebola glycoprotein fused to the affinity binding polypeptide comprises an amino acid sequence having at least 90% identity to SEQ ID NO: 188, SEQ ID NO: 189, SEQ ID NO: 190, or SEQ ID NO: 191.
87. The pseudotyped viral-like particle or viral vector of embodiment 15, wherein the Ebola glycoprotein fused to the affinity binding polypeptide comprises an amino acid sequence having at least 95% identity to SEQ ID NO: 188, SEQ ID NO: 189, SEQ ID NO: 190, or SEQ ID NO: 191.
88. The pseudotyped viral-like particle or viral vector of embodiment 15, wherein the Ebola glycoprotein fused to the affinity binding polypeptide comprises an amino acid sequence having at least 98% identity to SEQ ID NO: 188, SEQ ID NO: 189, SEQ ID NO: 190, or SEQ ID NO: 191.
89. The pseudotyped viral-like particle or viral vector of embodiment 15, wherein the Ebola glycoprotein fused to the affinity binding polypeptide comprises an amino acid of SEQ ID NO: 188.
90. The pseudotyped viral-like particle or viral vector of embodiment 15, wherein the Ebola glycoprotein fused to the affinity binding polypeptide comprises an amino acid of SEQ ID NO: 189.
91. The pseudotyped viral-like particle or viral vector of embodiment 15, wherein the Ebola glycoprotein fused to the affinity binding polypeptide comprises an amino acid of SEQ ID NO: 190.
92. The pseudotyped viral-like particle or viral vector of embodiment 15, wherein the Ebola glycoprotein fused to the affinity binding polypeptide comprises an amino acid of SEQ ID NO: 191.
93. The pseudotyped viral-like particle or viral vector of any one of embodiments 9-14, wherein the glycoprotein is NiV-G glycoprotein.
94. The pseudotyped viral-like particle or viral vector of embodiment 93, wherein the NiV-G glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 31 or SEQ ID NO: 32.
95. The pseudotyped viral-like particle or viral vector of embodiment 93, wherein the NiV-G glycoprotein fused to the affinity binding polypeptide comprises an amino acid sequence having at least 90% identity to SEQ ID NO: 31.
96. The pseudotyped viral-like particle or viral vector of embodiment 93, wherein the NiV-G glycoprotein fused to the affinity binding polypeptide comprises an amino acid sequence having at least 95% identity to SEQ ID NO: 31.
97. The pseudotyped viral-like particle or viral vector of embodiment 93, wherein the NiV-G glycoprotein fused to the affinity binding polypeptide comprises an amino acid sequence having at least 98% identity to SEQ ID NO: 31.
98. The pseudotyped viral-like particle or viral vector of embodiment 93, wherein the NiV-G glycoprotein fused to the affinity binding polypeptide comprises an amino acid sequence of SEQ ID NO: 31.
99. The pseudotyped viral-like particle or viral vector of embodiment 93, wherein the NiV-G glycoprotein fused to the affinity binding polypeptide comprises an amino acid sequence having at least 90% identity to SEQ ID NO: 32.
100. The pseudotyped viral-like particle or viral vector of embodiment 93, wherein the NiV-G glycoprotein fused to the affinity binding polypeptide comprises an amino acid sequence having at least 95% identity to SEQ ID NO: 32.
101. The pseudotyped viral-like particle or viral vector of embodiment 93, wherein the NiV-G glycoprotein fused to the affinity binding polypeptide comprises an amino acid sequence having at least 98% identity to SEQ ID NO: 32.
102. The pseudotyped viral-like particle or viral vector of embodiment 93, wherein the NiV-G glycoprotein fused to the affinity binding polypeptide comprises an amino acid sequence of SEQ ID NO: 32.
103. The pseudotyped viral-like particle or viral vector of any one of embodiments 9-14, wherein the glycoprotein is NiV-F glycoprotein.
104. The pseudotyped viral-like particle or viral vector of embodiment 103, wherein the NiV-F glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 35 or SEQ ID NO: 166.
105. The pseudotyped viral-like particle or viral vector of embodiment 103, wherein the NiV-F glycoprotein fused to the affinity binding polypeptide comprises an amino acid sequence having at least 90% identity to SEQ ID NO: 35 or SEQ ID NO: 166.
106. The pseudotyped viral-like particle or viral vector of embodiment 103, wherein the NiV-F glycoprotein fused to the affinity binding polypeptide comprises an amino acid sequence having at least 95% identity to SEQ ID NO: 35 or SEQ ID NO: 166.
107. The pseudotyped viral-like particle or viral vector of embodiment 103, wherein the NiV-F glycoprotein fused to the affinity binding polypeptide comprises an amino acid sequence having at least 98% identity to SEQ ID NO: 35 or SEQ ID NO: 166.
108. The pseudotyped viral-like particle or viral vector of embodiment 103, wherein the NiV-F glycoprotein fused to the affinity binding polypeptide comprises an amino acid sequence of SEQ ID NO: 35.
109. The pseudotyped viral-like particle or viral vector of embodiment 103, wherein the NiV-F glycoprotein fused to the affinity binding polypeptide comprises an amino acid sequence of SEQ ID NO: 166.
110. The pseudotyped viral-like particle or viral vector of any one of embodiments 9-14, wherein the glycoprotein is VSV-G glycoprotein.
110a. The pseudotyped viral-like particle or viral vector of embodiment 110, wherein the VSV-G glycoprotein comprises an amino acid sequence having at least 85%, 86%, 87%, 88%, 89%, 90%, 91%, 92%, 93%, 94%, 95%, 96%, 97%, 98%, or 99% identity to SEQ ID NO: 37 and comprises a mutation at position 182 as compared to SEQ ID NO: 37.
110b. The pseudotyped viral-like particle or viral vector of embodiment 110, wherein the VSV-G glycoprotein comprises an amino acid sequence of SEQ ID NO: 39, SEQ ID NO: 40, SEQ ID NO: 41, SEQ ID NO: 64, or SEQ ID NO: 65.
111. The pseudotyped viral-like particle or viral vector of embodiment 110, wherein the VSV-G glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 68, SEQ ID NO: 69, SEQ ID NO: 70, SEQ ID NO: 71, SEQ ID NO: 72, SEQ ID NO: 73, SEQ ID NO: 132, SEQ ID NO: 133, SEQ ID NO: 167, or SEQ ID NO: 168.
112. The pseudotyped viral-like particle or viral vector of embodiment 110, wherein the VSV-G glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 68 or SEQ ID NO: 69.
113. The pseudotyped viral-like particle or viral vector of embodiment 110, wherein the VSV-G glycoprotein fused to the affinity binding polypeptide comprises an amino acid sequence having at least 90% identity to SEQ ID NO: 68 or SEQ ID NO: 69.
114. The pseudotyped viral-like particle or viral vector of embodiment 110, wherein the VSV-G glycoprotein fused to the affinity binding polypeptide comprises an amino acid sequence having at least 95% identity to SEQ ID NO: 68 or SEQ ID NO: 69.
115. The pseudotyped viral-like particle or viral vector of embodiment 110, wherein the VSV-G glycoprotein fused to the affinity binding polypeptide comprises an amino acid sequence having at least 98% identity to SEQ ID NO: 68 or SEQ ID NO: 69.
116. The pseudotyped viral-like particle or viral vector of embodiment 110, wherein the VSV-G glycoprotein fused to the affinity binding polypeptide comprises an amino acid sequence of SEQ ID NO: 68.
117. The pseudotyped viral-like particle or viral vector of embodiment 110, wherein the VSV-G glycoprotein fused to the affinity binding polypeptide comprises an amino acid sequence of SEQ ID NO: 69.
118. The pseudotyped viral-like particle or viral vector of embodiment 110, wherein the VSV-G glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 70.
119. The pseudotyped viral-like particle or viral vector of embodiment 110, wherein the VSV-G glycoprotein fused to the affinity binding polypeptide comprises an amino acid sequence having at least 90% identity to SEQ ID NO: 70.
120. The pseudotyped viral-like particle or viral vector of embodiment 110, wherein the VSV-G glycoprotein fused to the affinity binding polypeptide comprises an amino acid sequence having at least 95% identity to SEQ ID NO: 70.
121. The pseudotyped viral-like particle or viral vector of embodiment 110, wherein the VSV-G glycoprotein fused to the affinity binding polypeptide comprises an amino acid sequence having at least 98% identity to SEQ ID NO: 70.
122. The pseudotyped viral-like particle or viral vector of embodiment 110, wherein the VSV-G glycoprotein fused to the affinity binding polypeptide comprises an amino acid sequence of SEQ ID NO: 70.
123. The pseudotyped viral-like particle or viral vector of embodiment 110, wherein the VSV-G glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 71.
124. The pseudotyped viral-like particle or viral vector of embodiment 110, wherein the VSV-G glycoprotein fused to the affinity binding polypeptide comprises an amino acid sequence having at least 90% identity to SEQ ID NO: 71.
125. The pseudotyped viral-like particle or viral vector of embodiment 110, wherein the VSV-G glycoprotein fused to the affinity binding polypeptide comprises an amino acid sequence having at least 95% identity to SEQ ID NO: 71.
126. The pseudotyped viral-like particle or viral vector of embodiment 110, wherein the VSV-G glycoprotein fused to the affinity binding polypeptide comprises an amino acid sequence having at least 98% identity to SEQ ID NO: 71.
127. The pseudotyped viral-like particle or viral vector of embodiment 110, wherein the VSV-G glycoprotein fused to the affinity binding polypeptide comprises an amino acid sequence of SEQ ID NO: 71.
128. The pseudotyped viral-like particle or viral vector of embodiment 110, wherein the VSV-G glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 72.
129. The pseudotyped viral-like particle or viral vector of embodiment 110, wherein the VSV-G glycoprotein fused to the affinity binding polypeptide comprises an amino acid sequence having at least 90% identity to SEQ ID NO: 72.
130. The pseudotyped viral-like particle or viral vector of embodiment 110, wherein the VSV-G glycoprotein fused to the affinity binding polypeptide comprises an amino acid sequence having at least 95% identity to SEQ ID NO: 72.
131. The pseudotyped viral-like particle or viral vector of embodiment 110, wherein the VSV-G glycoprotein fused to the affinity binding polypeptide comprises an amino acid sequence having at least 98% identity to SEQ ID NO: 72.
132. The pseudotyped viral-like particle or viral vector of embodiment 110, wherein the VSV-G glycoprotein fused to the affinity binding polypeptide comprises an amino acid sequence of SEQ ID NO: 72.
133. The pseudotyped viral-like particle or viral vector of embodiment 110, wherein the VSV-G glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 73.
134. The pseudotyped viral-like particle or viral vector of embodiment 110, wherein the VSV-G glycoprotein fused to the affinity binding polypeptide comprises an amino acid sequence having at least 90% identity to SEQ ID NO: 73.
135. The pseudotyped viral-like particle or viral vector of embodiment 110, wherein the VSV-G glycoprotein fused to the affinity binding polypeptide comprises an amino acid sequence having at least 95% identity to SEQ ID NO: 73.
136. The pseudotyped viral-like particle or viral vector of embodiment 110, wherein the VSV-G glycoprotein fused to the affinity binding polypeptide comprises an amino acid sequence having at least 98% identity to SEQ ID NO: 73.
137. The pseudotyped viral-like particle or viral vector of embodiment 110, wherein the VSV-G glycoprotein fused to the affinity binding polypeptide comprises an amino acid sequence of SEQ ID NO: 73.
138. The pseudotyped viral-like particle or viral vector of embodiment 110, wherein the VSV-G glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 132 or SEQ ID NO: 167.
139. The pseudotyped viral-like particle or viral vector of embodiment 110, wherein the VSV-G glycoprotein fused to the affinity binding polypeptide comprises an amino acid sequence having at least 90% identity to SEQ ID NO: 132 or SEQ ID NO: 167.
140. The pseudotyped viral-like particle or viral vector of embodiment 110, wherein the VSV-G glycoprotein fused to the affinity binding polypeptide comprises an amino acid sequence having at least 95% identity to SEQ ID NO: 132 or SEQ ID NO: 167.
141. The pseudotyped viral-like particle or viral vector of embodiment 110, wherein the VSV-G glycoprotein fused to the affinity binding polypeptide comprises an amino acid sequence having at least 98% identity to SEQ ID NO: 132 or SEQ ID NO: 167.
142. The pseudotyped viral-like particle or viral vector of embodiment 110, wherein the VSV-G glycoprotein fused to the affinity binding polypeptide comprises an amino acid sequence of SEQ ID NO: 132.
143. The pseudotyped viral-like particle or viral vector of embodiment 110, wherein the VSV-G glycoprotein fused to the affinity binding polypeptide comprises an amino acid sequence of SEQ ID NO: 167.
144. The pseudotyped viral-like particle or viral vector of embodiment 110, wherein the VSV-G glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 133 or SEQ ID NO: 168.
145. The pseudotyped viral-like particle or viral vector of embodiment 110, wherein the VSV-G glycoprotein fused to the affinity binding polypeptide comprises an amino acid sequence having at least 90% identity to SEQ ID NO: 133 or SEQ ID NO: 168.
146. The pseudotyped viral-like particle or viral vector of embodiment 110, wherein the VSV-G glycoprotein fused to the affinity binding polypeptide comprises an amino acid sequence having at least 95% identity to SEQ ID NO: 133 or SEQ ID NO: 168.
147. The pseudotyped viral-like particle or viral vector of embodiment 110, wherein the VSV-G glycoprotein fused to the affinity binding polypeptide comprises an amino acid sequence having at least 98% identity to SEQ ID NO: 133 or SEQ ID NO: 168.
148. The pseudotyped viral-like particle or viral vector of embodiment 110, wherein the VSV-G glycoprotein fused to the affinity binding polypeptide comprises an amino acid sequence of SEQ ID NO: 133.
149. The pseudotyped viral-like particle or viral vector of embodiment 110, wherein the VSV-G glycoprotein fused to the affinity binding polypeptide comprises an amino acid sequence of SEQ ID NO: 168.
150. The pseudotyped viral-like particle or viral vector of any one of embodiments 9-14, wherein the glycoprotein is MeV-H glycoprotein.
151. The pseudotyped viral-like particle or viral vector of embodiment 150, wherein the MeV-H glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 75 or SEQ ID NO: 76.
152. The pseudotyped viral-like particle or viral vector of embodiment 150, wherein the MeV-H glycoprotein fused to the affinity binding polypeptide comprises an amino acid sequence having at least 90% identity to SEQ ID NO: 75.
153. The pseudotyped viral-like particle or viral vector of embodiment 150, wherein the MeV-H glycoprotein fused to the affinity binding polypeptide comprises an amino acid sequence having at least 95% identity to SEQ ID NO: 75.
154. The pseudotyped viral-like particle or viral vector of embodiment 150, wherein the MeV-H glycoprotein fused to the affinity binding polypeptide comprises an amino acid sequence having at least 98% identity to SEQ ID NO: 75.
155. The pseudotyped viral-like particle or viral vector of embodiment 150, wherein the MeV-H glycoprotein fused to the affinity binding polypeptide comprises an amino acid of SEQ ID NO: 75.
156. The pseudotyped viral-like particle or viral vector of embodiment 150, wherein the MeV-H glycoprotein fused to the affinity binding polypeptide comprises an amino acid sequence having at least 90% identity to SEQ ID NO: 76.
157. The pseudotyped viral-like particle or viral vector of embodiment 150, wherein the MeV-H glycoprotein fused to the affinity binding polypeptide comprises an amino acid sequence having at least 95% identity to SEQ ID NO: 76.
158. The pseudotyped viral-like particle or viral vector of embodiment 150, wherein the MeV-H glycoprotein fused to the affinity binding polypeptide comprises an amino acid sequence having at least 98% identity to SEQ ID NO: 76.
159. The pseudotyped viral-like particle or viral vector of embodiment 150, wherein the MeV-H glycoprotein fused to the affinity binding polypeptide comprises an amino acid of SEQ ID NO: 76.
160. The pseudotyped viral-like particle or viral vector of any one of embodiments 9-14, wherein the glycoprotein is MeV-F glycoprotein.
161. The pseudotyped viral-like particle or viral vector of embodiment 160, wherein the MeV-F glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 78 or SEQ ID NO: 170.
162. The pseudotyped viral-like particle or viral vector of embodiment 160, wherein the MeV-F glycoprotein fused to the affinity binding polypeptide comprises an amino acid sequence having at least 90% identity to SEQ ID NO: 78 or SEQ ID NO: 170.
163. The pseudotyped viral-like particle or viral vector of embodiment 160, wherein the MeV-F glycoprotein fused to the affinity binding polypeptide comprises an amino acid sequence having at least 95% identity to SEQ ID NO: 78 or SEQ ID NO: 170.
164. The pseudotyped viral-like particle or viral vector of embodiment 160, wherein the MeV-F glycoprotein fused to the affinity binding polypeptide comprises an amino acid sequence having at least 98% identity to SEQ ID NO: 78 or SEQ ID NO: 170.
165. The pseudotyped viral-like particle or viral vector of embodiment 160, wherein the MeV-F glycoprotein fused to the affinity binding polypeptide comprises an amino acid sequence of SEQ ID NO: 78.
166. The pseudotyped viral-like particle or viral vector of embodiment 160, wherein the MeV-F glycoprotein fused to the affinity binding polypeptide comprises an amino acid sequence of SEQ ID NO: 170.
167. The pseudotyped viral-like particle or viral vector of any one of embodiments 9-14, wherein the glycoprotein is SVCV-G glycoprotein.
168. The pseudotyped viral-like particle or viral vector of embodiment 167, wherein the SVCV-G glycoprotein fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 81 or SEQ ID NO: 172.
169. The pseudotyped viral-like particle or viral vector of embodiment 167, wherein the SVCV-G glycoprotein fused to the affinity binding polypeptide comprises an amino acid sequence having at least 90% identity to SEQ ID NO: 81 or SEQ ID NO: 172.
170. The pseudotyped viral-like particle or viral vector of embodiment 167, wherein the SVCV-G glycoprotein fused to the affinity binding polypeptide comprises an amino acid sequence having at least 95% identity to SEQ ID NO: 81 or SEQ ID NO: 172.
171. The pseudotyped viral-like particle or viral vector of embodiment 167, wherein the SVCV-G glycoprotein fused to the affinity binding polypeptide comprises an amino acid sequence having at least 98% identity to SEQ ID NO: 81 or SEQ ID NO: 172.
172. The pseudotyped viral-like particle or viral vector of embodiment 167, wherein the SVCV-G glycoprotein fused to the affinity binding polypeptide comprises an amino acid sequence of SEQ ID NO: 81.
173. The pseudotyped viral-like particle or viral vector of embodiment 167, wherein the SVCV-G glycoprotein fused to the affinity binding polypeptide comprises an amino acid sequence of SEQ ID NO: 172.
174. The pseudotyped viral-like particle or viral vector of any one of embodiments 1-173, wherein the engineered envelope comprises a second viral glycoprotein.
175. The pseudotyped viral-like particle or viral vector of embodiment 174, wherein the second viral glycoprotein is HIV glycoprotein gp120, SIV glycoprotein gp120, EbV glycoprotein, NiV-G, NiV-F, MeV-H, MeV-F, VSV-G, SVCV-G, any variant thereof, or any combination thereof.
176. The pseudotyped viral-like particle or viral vector of embodiments 174 or 175, wherein the second viral glycoprotein does not comprise an affinity binding polypeptide.
177. The pseudotyped viral-like particle or viral vector of embodiments 174 or 175, wherein the second viral glycoprotein comprises at least a first, at least a second, at least a third, at least a fourth, or at least a fifth affinity binding polypeptide sequence.
178. The pseudotyped viral-like particle or viral vector of embodiment 177, wherein each affinity binding polypeptide is located, independently, at the N-terminus, at the C-terminus, or within the second glycoprotein.
179. The pseudotyped viral-like particle or viral vector of any one of embodiments 1-178, wherein the targeting moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.
180. The pseudotyped viral-like particle or viral vector of any one of embodiments 1-179, wherein the targeting moiety is an scFv.
181. The pseudotyped viral-like particle or viral vector of any one of embodiments 1-180, wherein the targeting moiety is fused to the viral glycoprotein via a linker, such as a peptide linker.
182. The pseudotyped viral-like particle or viral vector of any one of embodiments 1-180, wherein the targeting moiety is not fused to the viral glycoprotein.
183. The pseudotyped viral-like particle or viral vector of any one of embodiments 1-182, wherein the targeting moiety binds to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsgl); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-la); Carbonic anhydrase IX (CA1X); Carcinoembryonic antigen (CEA); CCCTC-Binding Factor (Zinc Finger Protein)-Like (BORIS or Brother of the Regulator of Imprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudin18.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-I (CLL-1 or CLECL1); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLV1-Tax; Human papilloma virus E6 (HPV E6); Human papilloma virus E7 (HPV E7); Human Telomerase reverse transcriptase (hTERT); IgE; IL13Ra2; IL1 1Ra; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-I (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAPI; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member 1A (XAGE1).
184. The pseudotyped viral-like particle or viral vector of any one of embodiments 1-182, wherein the targeting moiety is selected from the group consisting of Stem Cell Factor protein (SCF, KIT-ligand, KL, or steel factor) or a moiety that binds to cKit (CD 117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1.
185. The pseudotyped viral-like particle or viral vector of embodiment 183 or embodiment 184, wherein the targeting moiety binds to CD7.
186. The pseudotyped viral-like particle or viral vector of embodiment 185, wherein the targeting moiety comprises an HCDR1 having the amino acid sequence of SEQ ID NO: 86, an HCDR2 having the amino acid sequence of SEQ ID NO: 87; an HCDR3 having the amino acid sequence of SEQ ID NO: 88; an LCDR1 having the amino acid sequence of SEQ ID NO: 89; an LCDR2 having the amino acid sequence of SEQ ID NO: 90; and an LCDR3 having the amino acid sequence of SEQ ID NO: 91; wherein the targeting moiety comprises an amino acid sequence having at least 85% identity to SEQ ID NO: 101 or SEQ ID NO: 102.
187. The pseudotyped viral-like particle or viral vector of embodiment 186, wherein the targeting moiety comprises an amino acid sequence having at least 85% identity to SEQ ID NO: 101.
188. The pseudotyped viral-like particle or viral vector of embodiment 186, wherein the targeting moiety comprises an amino acid sequence having at least 85% identity to SEQ ID NO: 102.
189. The pseudotyped viral-like particle or viral vector of embodiment 183 or 184, wherein the targeting moiety binds to CD8.
190. The pseudotyped viral-like particle or viral vector of embodiment 189, wherein the targeting moiety comprises an HCDR1 having the amino acid sequence of SEQ ID NO: 105, an HCDR2 having the amino acid sequence of SEQ ID NO: 106; an HCDR3 having the amino acid sequence of SEQ ID NO: 107; an LCDR1 having the amino acid sequence of SEQ ID NO: 108; an LCDR2 having the amino acid sequence of SEQ ID NO: 109; and an LCDR3 having the amino acid sequence of SEQ ID NO: 110; wherein the targeting moiety comprises an amino acid sequence having at least 85% identity to SEQ ID NO: 119 or SEQ ID NO: 120.
191. The pseudotyped viral-like particle or viral vector of embodiment 190, wherein the targeting moiety comprises an amino acid sequence having at least 85% identity to SEQ ID NO: 119.
192. The pseudotyped viral-like particle or viral vector of embodiment 190, wherein the targeting moiety comprises an amino acid sequence having at least 85% identity to SEQ ID NO: 120.
193. The pseudotyped viral-like particle or viral vector of any one of embodiments 1-8, wherein the at least a first affinity binding polypeptide is fused to the targeting moiety.
194. The pseudotyped viral-like particle or viral vector of embodiment 193, wherein the targeting moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.
195. The pseudotyped viral-like particle or viral vector of embodiment 193 or embodiment
194, wherein the targeting moiety is an scFv.
196. The pseudotyped viral-like particle or viral vector of any one of embodiments 193-195, wherein the targeting moiety binds to CD7, CD8, cKit (CD117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsgl); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-la); Carbonic anhydrase IX (CA1X); Carcinoembryonic antigen (CEA); CCCTC-Binding Factor (Zinc Finger Protein)-Like (BORIS or Brother of the Regulator of Imprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECL1); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLV1-Tax; Human papilloma virus E6 (HPV E6); Human papilloma virus E7 (HPV E7); Human Telomerase reverse transcriptase (hTERT); IgE; IL13Ra2; IL1 lRa; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-I (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAP1; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member 1A (XAGE1).
197. The pseudotyped viral-like particle or viral vector of any one of embodiments 193-195, wherein the targeting moiety is selected from the group consisting of Stem Cell Factor protein (SCF, KIT-ligand, KL, or steel factor) or a moiety that binds to cKit (CD 117), CD4, CD8, CD3, CD5, CD6, CD7, CD2, TCR alpha, TCR beta, TCR gamma, TCR delta, CD10, CD34, CD14, CD68, CCR7, CD62L, CD25, CCR2, CCR3, CCR4, CCR5, CCR6, CCR7, CXCR3, CD39, CD73, CTLA-4, GITR, LAG-3, LRRC32, Neurophili-1, and CX3CR1.
198. The pseudotyped viral-like particle or viral vector of embodiment 196 or embodiment 197, wherein the targeting moiety binds to CD7.
199. The pseudotyped viral-like particle or viral vector of embodiment 198, wherein the targeting moiety comprises an HCDR1 having the amino acid sequence of SEQ ID NO: 86, an HCDR2 having the amino acid sequence of SEQ ID NO: 87; an HCDR3 having the amino acid sequence of SEQ ID NO: 88; an LCDR1 having the amino acid sequence of SEQ ID NO: 89; an LCDR2 having the amino acid sequence of SEQ ID NO: 90; and an LCDR3 having the amino acid sequence of SEQ ID NO: 91; wherein the targeting moiety comprises an amino acid sequence having at least 85% identity to SEQ ID NO: 101 or SEQ ID NO: 102.
200. The pseudotyped viral-like particle or viral vector of embodiment 199, wherein the targeting moiety comprises an amino acid sequence having at least 90% identity to SEQ ID NO: 101.
201. The pseudotyped viral-like particle or viral vector of embodiment 199, wherein the targeting moiety comprises an amino acid sequence having at least 95% identity to SEQ ID NO: 101.
202. The pseudotyped viral-like particle or viral vector of embodiment 199, wherein the targeting moiety comprises an amino acid sequence having at least 98% identity to SEQ ID NO: 101.
203. The pseudotyped viral-like particle or viral vector of embodiment 199, wherein the targeting moiety comprises an amino acid sequence of SEQ ID NO: 101.
204. The pseudotyped viral-like particle or viral vector of embodiment 199, wherein the targeting moiety comprises an amino acid sequence having at least 90% identity to SEQ ID NO: 102.
205. The pseudotyped viral-like particle or viral vector of embodiment 199, wherein the targeting moiety comprises an amino acid sequence having at least 95% identity to SEQ ID NO: 102.
206. The pseudotyped viral-like particle or viral vector of embodiment 199, wherein the targeting moiety comprises an amino acid sequence having at least 98% identity to SEQ ID NO: 102.
207. The pseudotyped viral-like particle or viral vector of embodiment 199, wherein the targeting moiety comprises an amino acid sequence of SEQ ID NO: 102.
208. The pseudotyped viral-like particle or viral vector of embodiment 196 or embodiment 197, wherein the targeting moiety binds to CD8.
209. The pseudotyped viral-like particle or viral vector of embodiment 208, wherein the targeting moiety comprises an HCDR1 having the amino acid sequence of SEQ ID NO: 105, an HCDR2 having the amino acid sequence of SEQ ID NO: 106; an HCDR3 having the amino acid sequence of SEQ ID NO: 107; an LCDR1 having the amino acid sequence of SEQ ID NO: 108; an LCDR2 having the amino acid sequence of SEQ ID NO: 109; and an LCDR3 having the amino acid sequence of SEQ ID NO: 110; wherein the targeting moiety comprises an amino acid sequence having at least 85% identity to SEQ ID NO: 119 or SEQ ID NO: 120.
210. The pseudotyped viral-like particle or viral vector of embodiment 209, wherein the targeting moiety comprises an amino acid sequence having at least 90% identity to SEQ ID NO: 119.
211. The pseudotyped viral-like particle or viral vector of embodiment 209, wherein the targeting moiety comprises an amino acid sequence having at least 95% identity to SEQ ID NO: 119.
212. The pseudotyped viral-like particle or viral vector of embodiment 209, wherein the targeting moiety comprises an amino acid sequence having at least 98% identity to SEQ ID NO: 119.
213. The pseudotyped viral-like particle or viral vector of embodiment 209, wherein the targeting moiety comprises an amino acid sequence of SEQ ID NO: 119.
214. The pseudotyped viral-like particle or viral vector of embodiment 209, wherein the targeting moiety comprises an amino acid sequence having at least 90% identity to SEQ ID NO: 120.
215. The pseudotyped viral-like particle or viral vector of embodiment 209, wherein the targeting moiety comprises an amino acid sequence having at least 95% identity to SEQ ID NO: 120.
216. The pseudotyped viral-like particle or viral vector of embodiment 209, wherein the targeting moiety comprises an amino acid sequence having at least 98% identity to SEQ ID NO: 120.
217. The pseudotyped viral-like particle or viral vector of embodiment 209, wherein the targeting moiety comprises an amino acid sequence of SEQ ID NO: 120.
218. The pseudotyped viral-like particle or viral vector of any one of embodiments 193-217, wherein the at least a first affinity binding polypeptide is located at the N-terminus, at the C-terminus, or within the targeting moiety.
219. The pseudotyped viral-like particle or viral vector of embodiment 218, wherein the targeting moiety fused to the affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 121, SEQ ID NO: 126, SEQ ID NO: 129, SEQ ID NO: 130, SEQ ID NO: 131, SEQ ID NO: 174, SEQ ID NO: 175, SEQ ID NO: 176, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, or SEQ ID NO: 187.
220. The pseudotyped viral-like particle or viral vector of embodiment 219, wherein the targeting moiety fused to the affinity binding polypeptide comprises an amino acid sequence having at least 90% identity to SEQ ID NO: 121, SEQ ID NO: 174, SEQ ID NO: 175, or SEQ ID NO: 176.
221. The pseudotyped viral-like particle or viral vector of embodiment 219, wherein the targeting moiety fused to the affinity binding polypeptide comprises an amino acid sequence having at least 95% identity to SEQ ID NO: 121, SEQ ID NO: 174, SEQ ID NO: 175, or SEQ ID NO: 176.
222. The pseudotyped viral-like particle or viral vector of embodiment 219, wherein the targeting moiety fused to the affinity binding polypeptide comprises an amino acid sequence having at least 98% identity to SEQ ID NO: 121, SEQ ID NO: 174, SEQ ID NO: 175, or SEQ ID NO: 176.
223. The pseudotyped viral-like particle or viral vector of embodiment 219, wherein the targeting moiety fused to the affinity binding polypeptide comprises an amino acid sequence of SEQ ID NO: 121.
224. The pseudotyped viral-like particle or viral vector of embodiment 219, wherein the targeting moiety fused to the affinity binding polypeptide comprises an amino acid sequence of SEQ ID NO: 174.
225. The pseudotyped viral-like particle or viral vector of embodiment 219, wherein the targeting moiety fused to the affinity binding polypeptide comprises an amino acid sequence of SEQ ID NO: 175.
226. The pseudotyped viral-like particle or viral vector of embodiment 219, wherein the targeting moiety fused to the affinity binding polypeptide comprises an amino acid sequence of SEQ ID NO: 176.
227. The pseudotyped viral-like particle or viral vector of embodiment 219, wherein the targeting moiety fused to the affinity binding polypeptide comprises an amino acid sequence having at least 90% identity to SEQ ID NO: 126 or SEQ ID NO: 178.
228. The pseudotyped viral-like particle or viral vector of embodiment 219, wherein the targeting moiety fused to the affinity binding polypeptide comprises an amino acid sequence having at least 95% identity to SEQ ID NO: 126 or SEQ ID NO: 178.
229. The pseudotyped viral-like particle or viral vector of embodiment 219, wherein the targeting moiety fused to the affinity binding polypeptide comprises an amino acid sequence having at least 98% identity to SEQ ID NO: 126 or SEQ ID NO: 178.
230. The pseudotyped viral-like particle or viral vector of embodiment 219, wherein the targeting moiety fused to the affinity binding polypeptide comprises an amino acid sequence of SEQ ID NO: 126.
231. The pseudotyped viral-like particle or viral vector of embodiment 219, wherein the targeting moiety fused to the affinity binding polypeptide comprises an amino acid sequence of SEQ ID NO: 178.
232. The pseudotyped viral-like particle or viral vector of embodiment 219, wherein the targeting moiety fused to the affinity binding polypeptide comprises an amino acid sequence having at least 90% identity to SEQ ID NO: 129, SEQ ID NO: 179, SEQ ID NO: 180, or SEQ ID NO: 181.
233. The pseudotyped viral-like particle or viral vector of embodiment 219, wherein the targeting moiety fused to the affinity binding polypeptide comprises an amino acid sequence having at least 95% identity to SEQ ID NO: 129, SEQ ID NO: 179, SEQ ID NO: 180, or SEQ ID NO: 181.
234. The pseudotyped viral-like particle or viral vector of embodiment 219, wherein the targeting moiety fused to the affinity binding polypeptide comprises an amino acid sequence having at least 98% identity to SEQ ID NO: 129, SEQ ID NO: 179, SEQ ID NO: 180, or SEQ ID NO: 181.
235. The pseudotyped viral-like particle or viral vector of embodiment 219, wherein the targeting moiety fused to the affinity binding polypeptide comprises an amino acid sequence of SEQ ID NO: 129.
236. The pseudotyped viral-like particle or viral vector of embodiment 219, wherein the targeting moiety fused to the affinity binding polypeptide comprises an amino acid sequence of SEQ ID NO: 179.
237. The pseudotyped viral-like particle or viral vector of embodiment 219, wherein the targeting moiety fused to the affinity binding polypeptide comprises an amino acid sequence of SEQ ID NO: 180.
238. The pseudotyped viral-like particle or viral vector of embodiment 219, wherein the targeting moiety fused to the affinity binding polypeptide comprises an amino acid sequence of SEQ ID NO: 181.
239. The pseudotyped viral-like particle or viral vector of embodiment 219, wherein the targeting moiety fused to the affinity binding polypeptide comprises an amino acid sequence having at least 90% identity to SEQ ID NO: 130, SEQ ID NO: 182, SEQ ID NO: 183, or SEQ ID NO: 184.
240. The pseudotyped viral-like particle or viral vector of embodiment 219, wherein the targeting moiety fused to the affinity binding polypeptide comprises an amino acid sequence having at least 95% identity to SEQ ID NO: 130, SEQ ID NO: 182, SEQ ID NO: 183, or SEQ ID NO: 184.
241. The pseudotyped viral-like particle or viral vector of embodiment 219, wherein the targeting moiety fused to the affinity binding polypeptide comprises an amino acid sequence having at least 98% identity to SEQ ID NO: 130, SEQ ID NO: 182, SEQ ID NO: 183, or SEQ ID NO: 184.
242. The pseudotyped viral-like particle or viral vector of embodiment 219, wherein the targeting moiety fused to the affinity binding polypeptide comprises an amino acid sequence of SEQ ID NO: 130.
243. The pseudotyped viral-like particle or viral vector of embodiment 219, wherein the targeting moiety fused to the affinity binding polypeptide comprises an amino acid sequence of SEQ ID NO: 182.
244. The pseudotyped viral-like particle or viral vector of embodiment 219, wherein the targeting moiety fused to the affinity binding polypeptide comprises an amino acid sequence of SEQ ID NO: 183.
245. The pseudotyped viral-like particle or viral vector of embodiment 219, wherein the targeting moiety fused to the affinity binding polypeptide comprises an amino acid sequence of SEQ ID NO: 184.
246. The pseudotyped viral-like particle or viral vector of embodiment 219, wherein the targeting moiety fused to the affinity binding polypeptide comprises an amino acid sequence having at least 90% identity to SEQ ID NO: 131, SEQ ID NO: 185, SEQ ID NO: 186, or SEQ ID NO: 187.
247. The pseudotyped viral-like particle or viral vector of embodiment 219, wherein the targeting moiety fused to the affinity binding polypeptide comprises an amino acid sequence having at least 95% identity to SEQ ID NO: 131, SEQ ID NO: 185, SEQ ID NO: 186, or SEQ ID NO: 187.
248. The pseudotyped viral-like particle or viral vector of embodiment 219, wherein the targeting moiety fused to the affinity binding polypeptide comprises an amino acid sequence having at least 98% identity to SEQ ID NO: 131, SEQ ID NO: 185, SEQ ID NO: 186, or SEQ ID NO: 187.
249. The pseudotyped viral-like particle or viral vector of embodiment 219, wherein the targeting moiety fused to the affinity binding polypeptide comprises an amino acid sequence of SEQ ID NO: 131.
250. The pseudotyped viral-like particle or viral vector of embodiment 219, wherein the targeting moiety fused to the affinity binding polypeptide comprises an amino acid sequence of SEQ ID NO: 185.
251. The pseudotyped viral-like particle or viral vector of embodiment 219, wherein the targeting moiety fused to the affinity binding polypeptide comprises an amino acid sequence of SEQ ID NO: 186.
252. The pseudotyped viral-like particle or viral vector of embodiment 219, wherein the targeting moiety fused to the affinity binding polypeptide comprises an amino acid sequence of SEQ ID NO: 187.
253. The pseudotyped viral-like particle or viral vector of any one of embodiments 193-252, wherein the pseudotyped viral-like particle or viral vector further comprises at least a second affinity binding polypeptide.
254. The pseudotyped viral-like particle or viral vector of embodiment 253, wherein the at least a second affinity binding polypeptide is located at the N-terminus, at the C-terminus, or within the targeting moiety.
255. The pseudotyped viral-like particle or viral vector of any one of embodiments 193-254, wherein the pseudotyped viral-like particle or viral vector further comprises at least a third, at least a fourth, or at least a fifth affinity binding polypeptide.
256. The pseudotyped viral-like particle or viral vector of embodiment 255, wherein each additional affinity binding polypeptide is located, independently, at the N-terminus, at the C-terminus, or within the targeting moiety.
257. The pseudotyped viral-like particle or viral vector of any one of embodiments 193-256, wherein the pseudotyped viral-like particle or viral vector further comprises a second targeting moiety.
258. The pseudotyped viral-like particle or viral vector of embodiment 257, wherein the second targeting moiety does not comprise an affinity binding polypeptide.
259. The pseudotyped viral-like particle or viral vector of embodiment 257, wherein the second targeting moiety further comprises at least a first, at least a second, at least a third, at least a fourth, or at least a fifth affinity binding polypeptide sequence.
260. The pseudotyped viral-like particle or viral vector of embodiment 259, wherein each affinity binding polypeptide is located, independently, at the N-terminus, at the C-terminus, or within the second targeting moiety.
261. The pseudotyped viral-like particle or viral vector of any one of embodiments 193-260, wherein the virus glycoprotein comprises an amino acid sequence as set forth in SEQ ID NO: 25, SEQ ID NO: 29, SEQ ID NO: 30, SEQ ID NO: 33, SEQ ID NO: 34, SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 66, SEQ ID NO: 67, SEQ ID NO: 74; SEQ ID NO: 77, SEQ ID NO: 79, or SEQ ID NO: 80.
262. A pseudotyped viral-like particle or viral vector comprising:
   an engineered envelope comprising a recombinant virus glycoprotein, an engineered targeting moiety for binding to a target cell, and at least a first affinity binding polypeptide, wherein the at least a first affinity binding polypeptide is fused to the glycoprotein, and the glycoprotein comprises an amino acid sequence as set forth in SEQ ID NO: 25, SEQ ID NO: 29, SEQ ID NO: 30, SEQ ID NO: 33, SEQ ID NO: 34, SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 66, SEQ ID NO: 67, SEQ ID NO: 74; SEQ ID NO: 77, SEQ ID NO: 79, or SEQ ID NO: 80; and
   a nucleic acid encoding a heterologous molecule of interest.
263. A pseudotyped viral-like particle or viral vector comprising:
   an engineered envelope comprising a recombinant virus glycoprotein, an engineered targeting moiety for binding to a target cell, and at least a first affinity binding polypeptide, wherein the at least a first affinity binding polypeptide is fused to the glycoprotein, and the at least a first affinity binding polypeptide fused to the glycoprotein comprises an amino acid sequence as set forth in SEQ ID NO: 26, SEQ ID NO: 27, SEQ ID NO: 28, SEQ ID NO: 31, SEQ ID NO: 32, SEQ ID NO: 35; SEQ ID NO: 68, SEQ ID NO: 69, SEQ ID NO: 70, SEQ ID NO: 71, SEQ ID NO: 72, SEQ ID NO: 73, SEQ ID NO: 75, SEQ ID NO: 76, SEQ ID NO: 78, SEQ ID NO: 81, SEQ ID NO: 132, SEQ ID NO: 133, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 166, SEQ ID NO: 167, SEQ ID NO: 168, SEQ ID NO: 170, SEQ ID NO: 172, SEQ ID NO: 188, SEQ ID NO: 189, SEQ ID NO: 190, or SEQ ID NO: 191; and
   a nucleic acid encoding a heterologous molecule of interest.
264. The pseudotyped viral-like particle or viral vector of embodiment 260 or embodiment 261, wherein the viral-like particle or viral vector is a lentivirus.
265. The pseudotyped viral-like particle or viral vector of any one of embodiments 260-262, wherein the pseudotyped viral-like particle or viral vector further comprises a glycoprotein that does not contain an affinity binding polypeptide.
266. A pseudotyped viral-like particle or viral vector comprising:
   an engineered envelope comprising a recombinant virus glycoprotein, an engineered targeting moiety for binding to a target cell, and at least a first affinity binding polypeptide, wherein the targeting moiety is an scFv comprising an amino acid sequence of SEQ ID NO: 101, SEQ ID NO: 102, SEQ ID NO: 119 or SEQ ID NO: 120, and the at least a first affinity binding polypeptide is fused to the targeting moiety; and
   a nucleic acid encoding a heterologous molecule of interest.
267. A pseudotyped viral-like particle or viral vector comprising:
   an engineered envelope comprising a recombinant virus glycoprotein, an engineered targeting moiety for binding to a target cell, and at least a first affinity binding polypeptide, wherein the targeting moiety is an scFv, and wherein the at least a first affinity binding polypeptide is fused to the targeting moiety and comprises an amino acid sequence of SEQ ID NO: 121, SEQ ID NO: 126, SEQ ID NO: 129, SEQ ID NO: 130, SEQ ID NO: 131, SEQ ID NO: 174, SEQ ID NO: 175, SEQ ID NO: 176, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, or SEQ ID NO: 187; and
   a nucleic acid encoding a heterologous molecule of interest.
268. The pseudotyped viral-like particle or viral vector of embodiment 264 or embodiment 265, wherein the viral-like particle or viral vector is a lentivirus.
269. The pseudotyped viral-like particle or viral vector of any one of embodiments 264-266, wherein the scFv is fused to the virus glycoprotein.
270. The pseudotyped viral-like particle or viral vector of any one of embodiments 264-266, wherein the scFv is not fused to the virus glycoprotein.
271. A pseudotyped viral-like particle or viral vector comprising:
   an engineered envelope comprising a recombinant virus glycoprotein, an engineered targeting moiety for binding to a target cell, and at least two affinity binding polypeptides,
   wherein the recombinant virus glycoprotein is fused to a first affinity binding polypeptide and comprises an amino acid sequence of SEQ ID NO: 26, SEQ ID NO: 27, SEQ ID NO: 28, SEQ ID NO: 31, SEQ ID NO: 32, SEQ ID NO: 35; SEQ ID NO: 68, SEQ ID NO: 69, SEQ ID NO: 70, SEQ ID NO: 71, SEQ ID NO: 72, SEQ ID NO: 73, SEQ ID NO: 75, SEQ ID NO: 76, SEQ ID NO: 78, SEQ ID NO: 81, SEQ ID NO: 132, SEQ ID NO: 133, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 166, SEQ ID NO: 167, SEQ ID NO: 168, SEQ ID NO: 170, SEQ ID NO: 172, SEQ ID NO: 188, SEQ ID NO: 189, SEQ ID NO: 190, or SEQ ID NO: 191,
   wherein the targeting moiety is an scFv,
   wherein the targeting moiety is fused to a second affinity binding polypeptide and comprises an amino acid sequence of SEQ ID NO: 121, SEQ ID NO: 126, SEQ ID NO: 129, SEQ ID NO: 130, SEQ ID NO: 131, SEQ ID NO: 174, SEQ ID NO: 175, SEQ ID NO: 176, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, or SEQ ID NO: 187; and
   a nucleic acid encoding a heterologous molecule of interest.
272. The pseudotyped viral-like particle or viral vector of embodiment 271, wherein the viral-like particle or viral vector is a lentivirus.
273. The pseudotyped viral-like particle or viral vector of any one of embodiments 1-272, wherein the heterologous molecule of interest is an siRNA, an shRNA, a non-coding RNA (e.g. a guide RNA for a CRISPR system), a peptide, a polypeptide, a protein, a viral payload, a viral genome, or a combination thereof.
274. The pseudotyped viral-like particle or viral vector of any one of embodiments 1-273, wherein the heterologous molecule of interest is a chimeric antigen receptor (“CAR”).
275. A polypeptide molecule comprising a virus glycoprotein, wherein the glycoprotein further comprises at least a first affinity binding polypeptide sequence.
276. The polypeptide of embodiment 275, wherein the at least a first affinity binding polypeptide is an affinity tag selected from the group consisting of a polyhistidine tag, a polyarginine tag, a FLAG tag, a streptavidin tag, a calmodulin binding peptide, or a variant or combination thereof.
277. The polypeptide of embodiment 276, wherein the streptavidin tag is selected from the group consisting of a streptavidin-binding peptide, a streptavidin binding tag, a strep-tag II, a twin-strep tag, or a variant or combination thereof.
278. The polypeptide of embodiments 276 or 277, wherein the at least a first affinity binding polypeptide is a strep-tag II polypeptide sequence.
279. The polypeptide of any one of embodiments embodiment 275-278, wherein the at least a first affinity binding polypeptide sequence is located at the N-terminus, at the C-terminus, or within the glycoprotein.
280. The polypeptide of any one of embodiments 275-279, wherein the polypeptide further comprises at least a second, at least a third, at least a fourth, or at least a fifth affinity binding polypeptide sequence.
281. The polypeptide of embodiment 280, wherein each additional affinity binding polypeptide sequence is located, independently, at the N-terminus, at the C-terminus, or within the glycoprotein.
282. The polypeptide of any one of embodiments 275-281, wherein the polypeptide further comprises a targeting moiety.
283. The polypeptide of embodiment 282, wherein the targeting moiety is not fused or linked to the glycoprotein.
284. The polypeptide of embodiment 282, wherein the targeting moiety is fused or linked to the glycoprotein via a peptide linker sequence.
285. A polypeptide molecule comprising a targeting moiety, wherein the targeting moiety further comprises at least a first affinity binding polypeptide sequence.
286. The polypeptide of embodiment 285, wherein the at least a first affinity binding polypeptide is an affinity tag selected from the group consisting of a polyhistidine tag, a polyarginine tag, a FLAG tag, a streptavidin tag, a calmodulin binding peptide, or a variant or combination thereof.
287. The polypeptide of embodiment 286, wherein the streptavidin tag is selected from the group consisting of a streptavidin-binding peptide, a streptavidin binding tag, a strep-tag II, a twin-strep tag, or a variant or combination thereof.
288. The polypeptide of any one of embodiments 285-287, wherein the at least a first affinity binding polypeptide is a strep-tag II polypeptide sequence
289. The polypeptide of any one of embodiments embodiment 285-288, wherein the at least a first affinity binding polypeptide sequence is located at the N-terminus, at the C-terminus, or within the targeting moiety.
290. The polypeptide of any one of embodiments 285-289, wherein the polypeptide further comprises at least a second, at least a third, at least a fourth, or at least a fifth affinity binding polypeptide sequence.
291. The polypeptide of embodiment 290, wherein each additional affinity binding polypeptide sequence is located, independently, at the N-terminus, at the C-terminus, or within the targeting moiety.
292. The polypeptide of any one of embodiments 285-291, wherein the targeting moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.
293. The polypeptide of any one of embodiments 285-292, wherein the targeting moiety is an scFv.
294. The polypeptide of any one of embodiments 285-193, wherein the polypeptide further comprises a virus glycoprotein.
295. The polypeptide of embodiment 294, wherein the virus glycoprotein is not fused or linked to the targeting moiety.
296. The polypeptide of embodiment 294, wherein the virus glycoprotein is fused or linked to the targeting moiety via a peptide linker sequence.
297. A nucleic acid molecule encoding for the polypeptide of any one of embodiments 275-296.
298. A method of making the pseudotyped viral-like particle or viral vector of any one of embodiments 1-274, the method comprising transfecting or transducing a packaging cell line with the nucleic acid molecule of embodiment 297 under conditions sufficient to produce the pseudotyped viral like particle or viral vector of any one of embodiments 1-274.
299. The method of embodiment 298, further comprising isolating the pseudotyped viral-like particle or viral vector.
300. A method of purifying a pseudotyped viral-like particle or viral vector, the method comprising transfecting or transducing a packaging cell line with a nucleic acid molecule encoding for a recombinant virus glycoprotein, an engineered targeting moiety for binding to a target cell, and at least a first affinity binding polypeptide, wherein the affinity binding polypeptide is fused to the glycoprotein, the targeting moiety, or any combination thereof; culturing the packaging cell line under conditions sufficient to produce the pseudotyped viral-like particle or viral vector; isolating the pseudotyped viral-like particle or viral vector; and purifying the pseudotyped viral-like particle or viral vector via use of the affinity binding polypeptide included in the glycoprotein, the targeting moiety, or both.
301. The method of embodiment 300, wherein isolating the pseudotyped viral-like particle or viral vector comprises collecting the packaging cell line supernatant containing the pseudotyped viral-like particle or viral vector.
302. The method of embodiments 300 or 301, wherein purifying the pseudotyped viral-like particle or viral vector comprises incubating the isolated pseudotyped viral-like particle or viral vector with a stationary phase comprising an interacting molecule which interacts with the affinity binding polypeptide of the glycoprotein, the targeting moiety, or both; washing the stationary phase with an appropriate wash buffer; and eluting the pseudotyped viral-like particle or viral vector, thereby purifying the pseudotyped viral-like particle or viral vector.
303. The method of any one of embodiments 300-302, wherein the pseudotyped viral-like particle or viral vector comprises the pseudotyped viral-like particle or viral vector of any one of embodiments 1-274.

### Additional Sequences

The following table provides sequences for those SEQ ID Nos recited above that are not included in the text above. The following table is only intended to supplement the provided description above and is not intended to connote any embodiments preference.

TABLE 7
Additional glycoprotein and binder constructs with affinity binding peptide(s)

| SEQ | Glycoprotein | |
|---|---|---|
| ID NO | or binder | Sequence |
| 137 | Ebola | MGVTGILQLPRDRFWSHPQFEKGGSKRTSFFLWVIILFQRTFSIPLGVIHNST |
| | glycoprotein | LQVSDVDKLVCRDKLSSTNQLRSVGLNLEGNGVATDVPSATKRWGFRSGVPPK |
| | | VVNYEAGEWAENCYNLEIKKPDGSECLPAAPDGIRGFPRCRYVHKVSGTGPCA |
| | | GDFAFHKEGAFFLYDRLASTVIYRGTTFAEGVVAFLILPQAKKDFFSSHPLRE |
| | | PVNATEDPSSGYYSTTIRYQATGFGTNETEYLFEVDNLTYVQLESRFTPQFLL |
| | | QLNETIYTSGKRSNTTGKLIWKVNPEIDTTIGEWAFWETKKNLTRKIRSEEGG |
| | | GGSGGGGSGGGGSQVQLQQPGAELVKPGASVKLSCKASGYPFTSYWIHWVKQR |
| | | PGRGLEWLGRIDPNSGDTKYNEKFKNKATLTVDKSSTTAYMQLSSLISEDSAV |
| | | YYCARSPYYSNDNSMDYWGQGISVTVSSGGGGSGGGGSGGGGSGGGGSDILLT |
| | | QSPAILSVSPGERVSFSCRASQSIGTSIHWYQQRTNDSPRLLIKYASESISGI |
| | | PSRFSGSGSGTDFTLSINSVESEDIADYYCQQSNSWPTTFGGGTKLEIKRGGG |
| | | GSGGGGSGGGGSNTIAGVAGLITGGRRTRREAIVNAQPKCNPNLHYWTTQDEG |
| | | AAIGLAWIPYFGPAAEGIYIEGLMHNQDGLICGLRQLANETTQALQLFLRATT |
| | | ELRTFSILNRKAIDFLLQRWGGTCHILGPDCCIEPHDWTKNITDKIDQIIHDF |
| | | VDKTLPDQGDNDNWWTGWRQWIPAGIGVTGVIIAVIALFCICKFVF |
| 139 | Ebola | MGVTGILQLPRDRFKRTSFFLWVIILFQRTFSIPLGVIHNSTLQVSDVDKLVC |
| | glycoprotein | RDKLSSTNQLRSVGLNLEGNGVATDVPSATKRWGFRSGVPPKVVNYEAGEWAE |
| | | NCYNLEIKKPDGSECLPAAPDGIRGFPRCRYVHKVSGTGPCAGDFAFHKEGAF |
| | | FLYDRLASTVIYRGTTFAEGVVAFLILPQAKKDFFSSHPLREPVNATEDPSSG |
| | | YYSTTIRYQATGFGTNETEYLFEVDNLTYVQLESRFTPQFLLQLNETIYTSGK |
| | | RSNTTGKLIWKVNPEIDTTIGEWAFWETKKNLTRKIRSEEGGGGSGGGGSGGG |
| | | GSQVQLQQPGAELVKPGASVKLSCKASGYPFTSYWIHWVKQRPGRGLEWLGRI |
| | | DPNSGDTKYNEKFKNKATLTVDKSSTTAYMQLSSLTSEDSAVYYCARSPYYSN |
| | | DNSMDYWGQGTSVTVSSGGGGSGGGGSGGGGSGGGGSDILLTQSPAILSVSPG |
| | | ERVSFSCRASQSIGTSIHWYQQRTNDSPRLLIKYASESISGIPSRFSGSGSGT |
| | | DFTLSINSVESEDIADYYCQQSNSWPTTFGGGTKLEIKRGGGGSGGGGSGGGG |
| | | SWSHPQFEKKESGSVSSEQLAQFRSLDWSHPQFEKNTIAGVAGLITGGRRTRR |
| | | EAIVNAQPKCNPNLHYWTTQDEGAAIGLAWIPYFGPAAEGIYIEGLMHNQDGL |
| | | ICGLRQLANETTQALQLFLRATTELRTFSILNRKAIDFLLQRWGGTCHILGPD |
| | | CCIEPHDWTKNITDKIDQIIHDFVDKTLPDQGDNDNWWTGWRQWIPAGIGVTG |
| | | VIIAVIALFCICKFVF |
| 141 | Ebola | MGVTGILQLPRDRFKRTSFFLWVIILFQRTFSIPLGVIHNSTLQVSDVDKLVC |
| | glycoprotein | RDKLSSTNQLRSVGLNLEGNGVATDVPSATKRWGFRSGVPPKVVNYEAGEWAE |
| | | NCYNLEIKKPDGSECLPAAPDGIRGFPRCRYVHKVSGTGPCAGDFAFHKEGAF |
| | | FLYDRLASTVIYRGTTFAEGVVAFLILPQAKKDFFSSHPLREPVNATEDPSSG |
| | | YYSTTIRYQATGFGTNETEYLFEVDNLTYVQLESRFTPQFLLQLNETIYTSGK |
| | | RSNTTGKLIWKVNPEIDTTIGEWAFWETKKNLTRKIRSEEGGGGSGGGGSGGG |
| | | GSQVQLQQPGAELVKPGASVKLSCKASGYPFTSYWIHWVKQRPGRGLEWLGRI |
| | | DPNSGDTKYNEKFKNKATLTVDKSSTTAYMQLSSLTSEDSAVYYCARSPYYSN |
| | | DNSMDYWGQGTSVTVSSGGGGSGGGGSGGGGSGGGGSDILLTQSPAILSVSPG |
| | | ERVSFSCRASQSIGTSIHWYQQRTNDSPRLLIKYASESISGIPSRFSGSGSGT |
| | | DFTLSINSVESEDIADYYCQQSNSWPTTFGGGTKLEIKRGGGGSGGGGSGGGG |
| | | SWSHPQFEKNTIAGVAGLITGGRRTRREAIVNAQPKCNPNLHYWTTQDEGAAI |
| | | GLAWIPYFGPAAEGIYIEGLMHNQDGLICGLRQLANETTQALQLFLRATTELR |
| | | TFSILNRKAIDFLLQRWGGTCHILGPDCCIEPHDWTKNITDKIDQIIHDFVDK |
| | | TLPDQGDNDNWWTGWRQWIPAGIGVTGVIIAVIALFCICKFVF |
| 143 | Ebola | MGVTGILQLPRDRFKRTSFFLWVIILFQRTFSIPLGVIHNSTLQVSDVDKLVC |
| | glycoprotein | RDKLSSTNQLRSVGLNLEGNGVATDVPSATKRWGFRSGVPPKVVNYEAGEWAE |
| | | NCYNLEIKKPDGSECLPAAPDGIRGFPRCRYVHKVSGTGPCAGDFAFHKEGAF |
| | | FLYDRLASTVIYRGTTFAEGVVAFLILPQAKKDFFSSHPLREPVNATEDPSSG |
| | | YYSTTIRYQATGFGTNETEYLFEVDNLTYVQLESRFTPQFLLQLNETIYTSGK |
| | | RSNTTGKLIWKVNPEIDTTIGEWAFWETKKNLTRKIRSEEWSHPQFEKKESGS |
| | | VSSEQLAQFRSLDWSHPQFEKGGGGSGGGGSGGGGSQVQLQQPGAELVKPGAS |
| | | VKLSCKASGYPFTSYWIHWVKQRPGRGLEWLGRIDPNSGDTKYNEKFKNKATL |
| | | TVDKSSTTAYMQLSSLTSEDSAVYYCARSPYYSNDNSMDYWGQGTSVTVSSGG |
| | | GGSGGGGSGGGGSGGGGSDILLTQSPAILSVSPGERVSFSCRASQSIGTSIHW |
| | | YQQRTNDSPRLLIKYASESISGIPSRFSGSGSGTDFTLSINSVESEDIADYYC |
| | | QQSNSWPTTFGGGTKLEIKRGGGGSGGGGSGGGGSNTIAGVAGLITGGRRTRR |
| | | EAIVNAQPKCNPNLHYWTTQDEGAAIGLAWIPYFGPAAEGIYIEGLMHNQDGL |
| | | ICGLRQLANETTQALQLFLRATTELRTFSILNRKAIDFLLQRWGGTCHILGPD |
| | | CCIEPHDWTKNITDKIDQIIHDFVDKTLPDQGDNDNWWTGWRQWIPAGIGVTG |
| | | VIIAVIALFCICKFVF |
| 145 | Ebola | MGVTGILQLPRDRFKRISFFLWVIILFQRTFSIPLGVIHNSTLQVSDVDKLVC |
| | glycoprotein | RDKLSSTNQLRSVGLNLEGNGVATDVPSATKRWGFRSGVPPKVVNYEAGEWAE |
| | | NCYNLEIKKPDGSECLPAAPDGIRGFPRCRYVHKVSGTGPCAGDFAFHKEGAF |
| | | FLYDRLASTVIYRGTTFAEGVVAFLILPQAKKDFFSSHPLREPVNATEDPSSG |
| | | YYSTTIRYQATGFGTNETEYLFEVDNLTYVQLESRFTPQFLLQLNETIYTSGK |
| | | RSNTTGKLIWKVNPEIDTTIGEWAFWETKKNLTRKIRSEEWSHPQFEKGGGGS |
| | | GGGGSGGGGSQVQLQQPGAELVKPGASVKLSCKASGYPFTSYWIHWVKQRPGR |
| | | GLEWLGRIDPNSGDTKYNEKFKNKATLTVDKSSTTAYMQLSSLTSEDSAVYYC |
| | | ARSPYYSNDNSMDYWGQGTSVTVSSGGGGSGGGGSGGGGSGGGGSDILLTQSP |
| | | AILSVSPGERVSFSCRASQSIGTSIHWYQQRTNDSPRLLIKYASESISGIPSR |
| | | FSGSGSGTDFTLSINSVESEDIADYYCQQSNSWPTTFGGGTKLEIKRGGGGSG |
| | | GGGSGGGGSNTIAGVAGLITGGRRTRREAIVNAQPKCNPNLHYWTTQDEGAAI |
| | | GLAWIPYFGPAAEGIYIEGLMHNQDGLICGLRQLANETTQALQLFLRATTELR |
| | | TFSILNRKAIDFLLQRWGGTCHILGPDCCIEPHDWTKNITDKIDQIIHDFVDK |
| | | TLPDQGDNDNWWTGWRQWIPAGIGVTGVIIAVIALFCICKFVF |
| 147 | Ebola | WSHPQFEKGGSKRTSFFLWVIILFQRTFSIPLGVIHNSTLQVSDVDKLVCRDK |
| | glycoprotein | LSSTNQLRSVGLNLEGNGVATDVPSATKRWGFRSGVPPKVVNYEAGEWAENCY |
| | | NLEIKKPDGSECLPAAPDGIRGFPRCRYVHKVSGTGPCAGDFAFHKEGAFFLY |
| | | DRLASTVIYRGTTFAEGVVAFLILPQAKKDFFSSHPLREPVNATEDPSSGYYS |
| | | TTIRYQATGFGTNETEYLFEVDNLTYVQLESRFTPQFLLQLNETIYTSGKRSN |
| | | TTGKLIWKVNPEIDTTIGEWAFWETKKNLTRKIRSEELSFTVVSNGAKNISGQ |
| | | SPARTSSDPGINTTTEDHKIMASENSSAMVQVHSQGREAAVSHLTTLATISTS |
| | | PQSLTTKPGPDNSTHNTPVYKLDISEATQVEQHHRRTDNDSTASDTPSATTAA |
| | | GPPKAENTNTSKSTDFLDPATTTSPQNHSETAGNNNTHHQDIGEESASSGKLG |
| | | LITNTIAGVAGLITGGRRTRREAIVNAQPKCNPNLHYWTTQDEGAAIGLAWIP |
| | | YFGPAAEGIYIEGLMHNQDGLICGLRQLANETTQALQLFLRATTELRTFSILN |
| | | RKAIDFLLQRWGGTCHILGPDCCIEPHDWTKNITDKIDQIIHDFVDKTLPDQG |
| | | DNDNWWTGWRQWIPAGIGVTGVIIAVIALFCICKFVF |
| 148 | Ebola | WSHPQFEKGGSKRTSFFLWVIILFQRTFSIPLGVIHNSTLQVSDVDKLVCRDK |
| | glycoprotein | LSSTNQLRSVGLNLEGNGVATDVPSATKRWGFRSGVPPKVVNYEAGEWAENCY |
| | | NLEIKKPDGSECLPAAPDGIRGFPRCRYVHKVSGTGPCAGDFAFHKEGAFFLY |
| | | DRLASTVIYRGTTFAEGVVAFLILPQAKKDFFSSHPLREPVNATEDPSSGYYS |
| | | TTIRYQATGFGTNETEYLFEVDNLTYVQLESRFTPQFLLQLNETIYTSGKRSN |
| | | TTGKLIWKVNPEIDTTIGEWAFWETKKNLTRKIRSEESANTIAGVAGLITGGR |
| | | RTRREAIVNAQPKCNPNLHYWTTQDEGAAIGLAWIPYFGPAAEGIYIEGLMHN |
| | | QDGLICGLRQLANETTQALQLFLRATTELRTFSILNRKAIDFLLQRWGGTCHI |
| | | LGPDCCIEPHDWTKNITDKIDQIIHDFVDKTLPDQGDNDNWWTGWRQWIPAGI |
| | | GVTGVIIAVIALFCICKFVF |
| 149 | Ebola | KRTSFFLWVIILFQRTFSIPLGVIHNSTLQVSDVDKLVCRDKLSSTNQLRSVG |
| | glycoprotein | LNLEGNGVATDVPSATKRWGFRSGVPPKVVNYEAGEWAENCYNLEIKKPDGSE |
| | | CLPAAPDGIRGFPRCRYVHKVSGTGPCAGDFAFHKEGAFFLYDRLASTVIYRG |
| | | TTFAEGVVAFLILPQAKKDFFSSHPLREPVNATEDPSSGYYSTTIRYQATGFG |
| | | TNETEYLFEVDNLTYVQLESRFTPQFLLQLNETIYTSGKRSNTTGKLIWKVNP |
| | | EIDTTIGEWAFWETKKNLTRKIRSEESWSHPQFEKANTIAGVAGLITGGRRTR |
| | | REAIVNAQPKCNPNLHYWTTQDEGAAIGLAWIPYFGPAAEGIYIEGLMHNQDG |
| | | LICGLRQLANETTQALQLFLRATTELRTFSILNRKAIDFLLQRWGGTCHILGP |
| | | DCCIEPHDWTKNITDKIDQIIHDFVDKTLPDQGDNDNWWTGWRQWIPAGIGVT |
| | | GVIIAVIALFCICKFVF |
| 150 | Ebola | KRTSFFLWVIILFQRTFSIPLGVIHNSTLQVSDVDKLVCRDKLSSTNQLRSVG |
| | glycoprotein | LNLEGNGVATDVPSATKRWGFRSGVPPKVVNYEAGEWAENCYNLEIKKPDGSE |
| | | CLPAAPDGIRGFPRCRYVHKVSGTGPCAGDFAFHKEGAFFLYDRLASTVIYRG |
| | | TTFAEGVVAFLILPQAKKDFFSSHPLREPVNATEDPSSGYYSTTIRYQATGFG |
| | | TNETEYLFEVDNLTYVQLESRFTPQFLLQLNETIYTSGKRSNITGKLIWKVNP |
| | | EIDTTIGEWAFWETKKNLTRKIRSEESWSHPQFEKKESGSVSSEQLAQFRSLD |
| | | WSHPQFEKANTIAGVAGLITGGRRTRREAIVNAQPKCNPNLHYWTTQDEGAAI |
| | | GLAWIPYFGPAAEGIYIEGLMHNQDGLICGLRQLANETTQALQLFLRATTELR |
| | | TFSILNRKAIDFLLQRWGGTCHILGPDCCIEPHDWTKNITDKIDQIIHDFVDK |
| | | TLPDQGDNDNWWTGWRQWIPAGIGVTGVIIAVIALFCICKFVF |
| 151 | Ebola | WSHPQFEKGGSKRTSFFLWVIILFQRTFSIPLGVIHNSTLQVSDVDKLVCRDK |
| | glycoprotein | LSSTNQLRSVGLNLEGNGVATDVPSATKRWGFRSGVPPKVVNYEAGEWAENCY |
| | | NLEIKKPDGSECLPAAPDGIRGFPRCRYVHKVSGTGPCAGDFAFHKEGAFFLY |
| | | DRLASTVIYRGTTFAEGVVAFLILPQAKKDFFSSHPLREPVNATEDPSSGYYS |
| | | TTIRYQATGFGTNETEYLFEVDNLTYVQLESRFTPQFLLQLNETIYTSGKRSN |
| | | TTGKLIWKVNPEIDTTIGEWAFWEIKKNLTRKIRSEESAGGGGSGGGGSGGGG |
| | | SASQVQLQQPGAELVKPGASVKLSCKASGYPFTSYWIHWVKQRPGRGLEWLGR |
| | | IDPNSGDTKYNEKFKNKATLTVDKSSTTAYMQLSSLTSEDSAVYYCARSPYYS |
| | | NDNSMDYWGQGTSVTVSSGGGGSGGGGSGGGGSGGGGSDILLTQSPAILSVSP |
| | | GERVSFSCRASQSIGTSIHWYQQRTNDSPRLLIKYASESISGIPSRFSGSGSG |
| | | TDFTLSINSVESEDIADYYCQQSNSWPTTFGGGTKLEIKRTGGGGGSGGGGSG |
| | | GGGSSANTIAGVAGLITGGRRTRREAIVNAQPKCNPNLHYWTTQDEGAAIGLA |
| | | WIPYFGPAAEGIYIEGLMHNQDGLICGLRQLANETTQALQLFLRATTELRTFS |
| | | ILNRKAIDFLLQRWGGTCHILGPDCCIEPHDWTKNITDKIDQIIHDFVDKTLP |
| | | DQGDNDNWWTGWRQWIPAGIGVTGVIIAVIALFCICKFVF |
| 152 | Ebola | MGVTGILQLPRDRFKRTSFFLWVIILFQRTFSIPLGVIHNSTLQVSDVDKLVC |
| | glycoprotein | RDKLSSTNQLRSVGLNLEGNGVATDVPSATKRWGFRSGVPPKVVNYEAGEWAE |
| | | NCYNLEIKKPDGSECLPAAPDGIRGFPRCRYVHKVSGTGPCAGDFAFHKEGAF |
| | | FLYDRLASTVIYRGTTFAEGVVAFLILPQAKKDFFSSHPLREPVNATEDPSSG |
| | | YYSTTIRYQATGFGTNETEYLFEVDNLTYVQLESRFTPQFLLQLNETIYTSGK |
| | | RSNTTGKLIWKVNPEIDTTIGEWAFWETKKNLTRKIRSEEWSHPQFEKNTIAG |
| | | VAGLITGGRRTRREAIVNAQPKCNPNLHYWTTQDEGAAIGLAWIPYFGPAAEG |
| | | IYIEGLMHNQDGLICGLRQLANETTQALQLFLRATTELRTFSILNRKAIDFLL |
| | | QRWGGTCHILGPDCCIEPHDWTKNITDKIDQIIHDFVDKTLPDQGDNDNWWTG |
| | | WRQWIPAGIGVTGVIIAVIALFCICKFVF* |
| 153 | Ebola | KRTSFFLWVIILFQRTFSIPLGVIHNSTLQVSDVDKLVCRDKLSSTNQLRSVG |
| | glycoprotein | LNLEGNGVATDVPSATKRWGFRSGVPPKVVNYEAGEWAENCYNLEIKKPDGSE |
| | | CLPAAPDGIRGFPRCRYVHKVSGTGPCAGDFAFHKEGAFFLYDRLASTVIYRG |
| | | TTFAEGVVAFLILPQAKKDFFSSHPLREPVNATEDPSSGYYSTTIRYQATGFG |
| | | TNETEYLFEVDNLTYVQLESRFTPQFLLQLNETIYTSGKRSNTTGKLIWKVNP |
| | | EIDTTIGEWAFWETKKNLTRKIRSEEWSHPQFEKNTIAGVAGLITGGRRTRRE |
| | | AIVNAQPKCNPNLHYWTIQDEGAAIGLAWIPYFGPAAEGIYIEGLMHNQDGLI |
| | | CGLRQLANETTQALQLFLRATTELRTFSILNRKAIDFLLQRWGGTCHILGPDC |
| | | CIEPHDWTKNITDKIDQIIHDFVDKTLPDQGDNDNWWTGWRQWIPAGIGVTGV |
| | | IIAVIALFCICKFVF |
| 154 | Ebola | MGVTGILQLPRDRFKRTSFFLWVIILFQRTFSIPLGVIHNSTLQVSDVDKLVC |
| | glycoprotein | RDKLSSTNQLRSVGLNLEGNGVATDVPSATKRWGFRSGVPPKVVNYEAGEWAE |
| | | NCYNLEIKKPDGSECLPAAPDGIRGFPRCRYVHKVSGTGPCAGDFAFHKEGAF |
| | | FLYDRLASTVIYRGTTFAEGVVAFLILPQAKKDFFSSHPLREPVNATEDPSSG |
| | | YYSTTIRYQATGFGTNETEYLFEVDNLTYVQLESRFTPQFLLQLNETIYTSGK |
| | | RSNTTGKLIWKVNPEIDTTIGEWAFWETKKNLTRKIRSEEWSHPQFEKKESGS |
| | | VSSEQLAQFRSLDWSHPQFEKNTIAGVAGLITGGRRTRREAIVNAQPKCNPNL |
| | | HYWTTQDEGAAIGLAWIPYFGPAAEGIYIEGLMHNQDGLICGLRQLANETTQA |
| | | LQLFLRATTELRTFSILNRKAIDFLLQRWGGTCHILGPDCCIEPHDWTKNITD |
| | | KIDQIIHDFVDKILPDQGDNDNWWIGWRQWIPAGIGVIGVIIAVIALFCICKF |
| | | VF* |
| 155 | Ebola | KRTSFFLWVIILFQRTFSIPLGVIHNSTLQVSDVDKLVCRDKLSSTNQLRSVG |
| | glycoprotein | LNLEGNGVATDVPSATKRWGFRSGVPPKVVNYEAGEWAENCYNLEIKKPDGSE |
| | | CLPAAPDGIRGFPRCRYVHKVSGTGPCAGDFAFHKEGAFFLYDRLASTVIYRG |
| | | TTFAEGVVAFLILPQAKKDFFSSHPLREPVNATEDPSSGYYSTTIRYQATGFG |
| | | TNETEYLFEVDNLTYVQLESRFTPQFLLQLNETIYTSGKRSNTTGKLIWKVNP |
| | | EIDTTIGEWAFWETKKNLTRKIRSEEWSHPQFEKKESGSVSSEQLAQFRSLDW |
| | | SHPQFEKNTIAGVAGLITGGRRTRREAIVNAQPKCNPNLHYWTTQDEGAAIGL |
| | | AWIPYFGPAAEGIYIEGLMHNQDGLICGLRQLANETTQALQLFLRATTELRTF |
| | | SILNRKAIDFLLQRWGGTCHILGPDCCIEPHDWTKNITDKIDQIIHDFVDKTL |
| | | PDQGDNDNWWIGWRQWIPAGIGVTGVIIAVIALFCICKFVF |
| 156 | Ebola | WSHPQFEKGGSKRTSFFLWVIILFQRTFSIPLGVIHNSTLQVSDVDKLVCRDK |
| | glycoprotein | LSSTNQLRSVGLNLEGNGVATDVPSATKRWGFRSGVPPKVVNYEAGEWAENCY |
| | | NLEIKKPDGSECLPAAPDGIRGFPRCRYVHKVSGTGPCAGDFAFHKEGAFFLY |
| | | DRLASTVIYRGTTFAEGVVAFLILPQAKKDFFSSHPLREPVNATEDPSSGYYS |
| | | TTIRYQATGFGTNETEYLFEVDNLTYVQLESRFTPQFLLQLNETIYTSGKRSN |
| | | TTGKLIWKVNPEIDTTIGEWAFWETKKNLTRKIRSEEGGGGSGGGGSGGGGSQ |
| | | VQLQQPGAELVKPGASVKLSCKASGYPFTSYWIHWVKQRPGRGLEWLGRIDPN |
| | | SGDTKYNEKFKNKATLTVDKSSTTAYMQLSSLTSEDSAVYYCARSPYYSNDNS |
| | | MDYWGQGTSVTVSSGGGGSGGGGSGGGGSGGGGSDILLTQSPAILSVSPGERV |
| | | SFSCRASQSIGTSIHWYQQRTNDSPRLLIKYASESISGIPSRFSGSGSGTDFT |
| | | LSINSVESEDIADYYCQQSNSWPTTFGGGTKLEIKRGGGGSGGGGSGGGGSNT |
| | | IAGVAGLITGGRRTRREAIVNAQPKCNPNLHYWTTQDEGAAIGLAWIPYFGPA |
| | | AEGIYIEGLMHNQDGLICGLRQLANETTQALQLFLRATTELRTFSILNRKAID |
| | | FLLQRWGGTCHILGPDCCIEPHDWTKNITDKIDQIIHDFVDKTLPDQGDNDNW |
| | | WTGWRQWIPAGIGVTGVIIAVIALFCICKFVF |
| 157 | Ebola | KRTSFFLWVIILFQRTFSIPLGVIHNSTLQVSDVDKLVCRDKLSSTNQLRSVG |
| | glycoprotein | LNLEGNGVATDVPSATKRWGFRSGVPPKVVNYEAGEWAENCYNLEIKKPDGSE |
| | | CLPAAPDGIRGFPRCRYVHKVSGTGPCAGDFAFHKEGAFFLYDRLASTVIYRG |
| | | TTFAEGVVAFLILPQAKKDFFSSHPLREPVNATEDPSSGYYSTTIRYQATGFG |
| | | TNETEYLFEVDNLTYVQLESRFTPQFLLQLNETIYTSGKRSNTTGKLIWKVNP |
| | | EIDTTIGEWAFWETKKNLTRKIRSEESAGGGGSGGGGSGGGGSASQVQLQQPG |
| | | AELVKPGASVKLSCKASGYPFTSYWIHWVKQRPGRGLEWLGRIDPNSGDTKYN |
| | | EKFKNKATLTVDKSSTTAYMQLSSLTSEDSAVYYCARSPYYSNDNSMDYWGQG |
| | | TSVTVSSGGGGSGGGGSGGGGSGGGGSDILLTQSPAILSVSPGERVSFSCRAS |
| | | QSIGTSIHWYQQRTNDSPRLLIKYASESISGIPSRFSGSGSGTDFTLSINSVE |
| | | SEDIADYYCQQSNSWPTTFGGGTKLEIKRTGGGGGSGGGGSGGGGSWSHPQFE |
| | | KKESGSVSSEQLAQFRSLDWSHPQFEKSANTIAGVAGLITGGRRTRREAIVNA |
| | | QPKCNPNLHYWTTQDEGAAIGLAWIPYFGPAAEGIYIEGLMHNQDGLICGLRQ |
| | | LANETTQALQLFLRATTELRTFSILNRKAIDFLLQRWGGTCHILGPDCCIEPH |
| | | DWTKNITDKIDQIIHDFVDKTLPDQGDNDNWWTGWRQWIPAGIGVTGVIIAVI |
| | | ALFCICKFVF |
| 158 | Ebola | KRTSFFLWVIILFQRTFSIPLGVIHNSTLQVSDVDKLVCRDKLSSTNQLRSVG |
| | glycoprotein | LNLEGNGVATDVPSATKRWGFRSGVPPKVVNYEAGEWAENCYNLEIKKPDGSE |
| | | CLPAAPDGIRGFPRCRYVHKVSGTGPCAGDFAFHKEGAFFLYDRLASTVIYRG |
| | | TTFAEGVVAFLILPQAKKDFFSSHPLREPVNATEDPSSGYYSTTIRYQATGFG |
| | | TNETEYLFEVDNLTYVQLESRFTPQFLLQLNETIYTSGKRSNTTGKLIWKVNP |
| | | EIDTTIGEWAFWETKKNLTRKIRSEEGGGGSGGGGSGGGGSQVQLQQPGAELV |
| | | KPGASVKLSCKASGYPFTSYWIHWVKQRPGRGLEWLGRIDPNSGDTKYNEKFK |
| | | NKATLTVDKSSTTAYMQLSSLTSEDSAVYYCARSPYYSNDNSMDYWGQGTSVT |
| | | VSSGGGGSGGGGSGGGGSGGGGSDILLTQSPAILSVSPGERVSFSCRASQSIG |
| | | TSIHWYQQRTNDSPRLLIKYASESISGIPSRFSGSGSGTDFTLSINSVESEDI |
| | | ADYYCQQSNSWPTTFGGGTKLEIKRGGGGSGGGGSGGGGSWSHPQFEKKESGS |
| | | VSSEQLAQFRSLDWSHPQFEKNTIAGVAGLITGGRRTRREAIVNAQPKCNPNL |
| | | HYWTTQDEGAAIGLAWIPYFGPAAEGIYIEGLMHNQDGLICGLRQLANETTQA |
| | | LQLFLRATTELRIFSILNRKAIDFLLQRWGGTCHILGPDCCIEPHDWTKNITD |
| | | KIDQIIHDFVDKTLPDQGDNDNWWIGWRQWIPAGIGVTGVIIAVIALFCICKF |
| | | VF |
| 159 | Ebola | KRTSFFLWVIILFQRTFSIPLGVIHNSTLQVSDVDKLVCRDKLSSTNQLRSVG |
| | glycoprotein | LNLEGNGVATDVPSATKRWGFRSGVPPKVVNYEAGEWAENCYNLEIKKPDGSE |
| | | CLPAAPDGIRGFPRCRYVHKVSGTGPCAGDFAFHKEGAFFLYDRLASTVIYRG |
| | | TTFAEGVVAFLILPQAKKDFFSSHPLREPVNATEDPSSGYYSTTIRYQATGFG |
| | | TNETEYLFEVDNLTYVQLESRFTPQFLLQLNETIYTSGKRSNTTGKLIWKVNP |
| | | EIDTTIGEWAFWETKKNLTRKIRSEESAGGGGSGGGGSGGGGSASQVQLQQPG |
| | | AELVKPGASVKLSCKASGYPFTSYWIHWVKQRPGRGLEWLGRIDPNSGDTKYN |
| | | EKFKNKATLTVDKSSTTAYMQLSSLTSEDSAVYYCARSPYYSNDNSMDYWGQG |
| | | TSVTVSSGGGGSGGGGSGGGGSGGGGSDILLTQSPAILSVSPGERVSFSCRAS |
| | | QSIGTSIHWYQQRTNDSPRLLIKYASESISGIPSRFSGSGSGTDFTLSINSVE |
| | | SEDIADYYCQQSNSWPTTFGGGTKLEIKRTGGGGGSGGGGSGGGGSWSHPQFE |
| | | KSANTIAGVAGLITGGRRTRREAIVNAQPKCNPNLHYWTTQDEGAAIGLAWIP |
| | | YFGPAAEGIYIEGLMHNQDGLICGLRQLANETTQALQLFLRATTELRIFSILN |
| | | RKAIDFLLQRWGGTCHILGPDCCIEPHDWTKNITDKIDQIIHDFVDKTLPDQG |
| | | DNDNWWIGWRQWIPAGIGVTGVIIAVIALFCICKFVF* |
| 160 | Ebola | KRTSFFLWVIILFQRTFSIPLGVIHNSTLQVSDVDKLVCRDKLSSTNQLRSVG |
| | glycoprotein | LNLEGNGVATDVPSATKRWGFRSGVPPKVVNYEAGEWAENCYNLEIKKPDGSE |
| | | CLPAAPDGIRGFPRCRYVHKVSGTGPCAGDFAFHKEGAFFLYDRLASTVIYRG |
| | | TTFAEGVVAFLILPQAKKDFFSSHPLREPVNATEDPSSGYYSTTIRYQATGFG |
| | | TNETEYLFEVDNLTYVQLESRFTPQFLLQLNETIYTSGKRSNTTGKLIWKVNP |
| | | EIDTTIGEWAFWETKKNLTRKIRSEEGGGGSGGGGSGGGGSQVQLQQPGAELV |
| | | KPGASVKLSCKASGYPFTSYWIHWVKQRPGRGLEWLGRIDPNSGDTKYNEKFK |
| | | NKATLTVDKSSTTAYMQLSSLTSEDSAVYYCARSPYYSNDNSMDYWGQGTSVT |
| | | VSSGGGGSGGGGSGGGGSGGGGSDILLTQSPAILSVSPGERVSFSCRASQSIG |
| | | TSIHWYQQRTNDSPRLLIKYASESISGIPSRFSGSGSGTDFTLSINSVESEDI |
| | | ADYYCQQSNSWPTTFGGGTKLEIKRGGGGSGGGGSGGGGSWSHPQFEKNTIAG |
| | | VAGLITGGRRTRREAIVNAQPKCNPNLHYWTTQDEGAAIGLAWIPYFGPAAEG |
| | | IYIEGLMHNQDGLICGLRQLANETTQALQLFLRATTELRTFSILNRKAIDFLL |
| | | QRWGGTCHILGPDCCIEPHDWTKNITDKIDQIIHDFVDKTLPDQGDNDNWWTG |
| | | WRQWIPAGIGVTGVIIAVIALFCICKFVF |
| 161 | Ebola | KRTSFFLWVIILFQRIFSIPLGVIHNSTLQVSDVDKLVCRDKLSSTNQLRSVG |
| | glycoprotein | LNLEGNGVATDVPSATKRWGFRSGVPPKVVNYEAGEWAENCYNLEIKKPDGSE |
| | | CLPAAPDGIRGFPRCRYVHKVSGTGPCAGDFAFHKEGAFFLYDRLASTVIYRG |
| | | TTFAEGVVAFLILPQAKKDFFSSHPLREPVNATEDPSSGYYSTTIRYQATGFG |
| | | TNETEYLFEVDNLTYVQLESRFTPQFLLQLNETIYTSGKRSNTTGKLIWKVNP |
| | | EIDTTIGEWAFWETKKNLTRKIRSEESAWSHPQFEKKESGSVSSEQLAQFRSL |
| | | DWSHPQFEKGGGGSGGGGSGGGGSASQVQLQQPGAELVKPGASVKLSCKASGY |
| | | PFTSYWIHWVKQRPGRGLEWLGRIDPNSGDTKYNEKFKNKATLTVDKSSTTAY |
| | | MQLSSLTSEDSAVYYCARSPYYSNDNSMDYWGQGTSVTVSSGGGGSGGGGSGG |
| | | GGSGGGGSDILLIQSPAILSVSPGERVSFSCRASQSIGTSIHWYQQRTNDSPR |
| | | LLIKYASESISGIPSRFSGSGSGTDFTLSINSVESEDIADYYCQQSNSWPTTF |
| | | GGGTKLEIKRTGGGGGSGGGGSGGGGSSANTIAGVAGLITGGRRTRREAIVNA |
| | | QPKCNPNLHYWTTQDEGAAIGLAWIPYFGPAAEGIYIEGLMHNQDGLICGLRQ |
| | | LANETTQALQLFLRATTELRTFSILNRKAIDFLLQRWGGTCHILGPDCCIEPH |
| | | DWTKNITDKIDQIIHDFVDKTLPDQGDNDNWWTGWRQWIPAGIGVTGVIIAVI |
| | | ALFCICKFVF* |
| 162 | Ebola | KRTSFFLWVIILFQRTFSIPLGVIHNSTLQVSDVDKLVCRDKLSSTNQLRSVG |
| | glycoprotein | LNLEGNGVATDVPSATKRWGFRSGVPPKVVNYEAGEWAENCYNLEIKKPDGSE |
| | | CLPAAPDGIRGFPRCRYVHKVSGTGPCAGDFAFHKEGAFFLYDRLASTVIYRG |
| | | TTFAEGVVAFLILPQAKKDFFSSHPLREPVNATEDPSSGYYSTTIRYQATGFG |
| | | TNETEYLFEVDNLTYVQLESRFTPQFLLQLNETIYTSGKRSNTTGKLIWKVNP |
| | | EIDTTIGEWAFWETKKNLTRKIRSEEWSHPQFEKKESGSVSSEQLAQFRSLDW |
| | | SHPQFEKGGGGSGGGGSGGGGSQVQLQQPGAELVKPGASVKLSCKASGYPFTS |
| | | YWIHWVKQRPGRGLEWLGRIDPNSGDTKYNEKFKNKATLTVDKSSTTAYMQLS |
| | | SLTSEDSAVYYCARSPYYSNDNSMDYWGQGTSVTVSSGGGGSGGGGSGGGGSG |
| | | GGGSDILLTQSPAILSVSPGERVSFSCRASQSIGTSIHWYQQRTNDSPRLLIK |
| | | YASESISGIPSRFSGSGSGTDFTLSINSVESEDIADYYCQQSNSWPTTFGGGT |
| | | KLEIKRGGGGSGGGGSGGGGSNTIAGVAGLITGGRRTRREAIVNAQPKCNPNL |
| | | HYWTTQDEGAAIGLAWIPYFGPAAEGIYIEGLMHNQDGLICGLRQLANETTQA |
| | | LQLFLRATTELRTFSILNRKAIDFLLQRWGGTCHILGPDCCIEPHDWTKNITD |
| | | KIDQIIHDFVDKTLPDQGDNDNWWIGWRQWIPAGIGVTGVIIAVIALFCICKF |
| | | VF |
| 163 | Ebola | KRTSFFLWVIILFQRTFSIPLGVIHNSTLQVSDVDKLVCRDKLSSTNQLRSVG |
| | glycoprotein | LNLEGNGVATDVPSATKRWGFRSGVPPKVVNYEAGEWAENCYNLEIKKPDGSE |
| | | CLPAAPDGIRGFPRCRYVHKVSGTGPCAGDFAFHKEGAFFLYDRLASTVIYRG |
| | | TTFAEGVVAFLILPQAKKDFFSSHPLREPVNATEDPSSGYYSTTIRYQATGFG |
| | | TNETEYLFEVDNLTYVQLESRFTPQFLLQLNETIYTSGKRSNTTGKLIWKVNP |
| | | EIDTTIGEWAFWETKKNLTRKIRSEESAWSHPQFEKGGGGSGGGGSGGGGSAS |
| | | QVQLQQPGAELVKPGASVKLSCKASGYPFTSYWIHWVKQRPGRGLEWLGRIDP |
| | | NSGDTKYNEKFKNKATLTVDKSSTTAYMQLSSLTSEDSAVYYCARSPYYSNDN |
| | | SMDYWGQGTSVTVSSGGGGSGGGGSGGGGSGGGGSDILLTQSPAILSVSPGER |
| | | VSFSCRASQSIGTSIHWYQQRTNDSPRLLIKYASESISGIPSRFSGSGSGTDF |
| | | TLSINSVESEDIADYYCQQSNSWPTTFGGGTKLEIKRTGGGGGSGGGGSGGGG |
| | | SSANTIAGVAGLITGGRRTRREAIVNAQPKCNPNLHYWTTQDEGAAIGLAWIP |
| | | YFGPAAEGIYIEGLMHNQDGLICGLRQLANETTQALQLFLRATTELRTFSILN |
| | | RKAIDFLLQRWGGTCHILGPDCCIEPHDWTKNITDKIDQIIHDFVDKTLPDQG |
| | | DNDNWWTGWRQWIPAGIGVTGVIIAVIALFCICKFVF* |
| 164 | Ebola | KRTSFFLWVIILFQRTFSIPLGVIHNSTLQVSDVDKLVCRDKLSSTNQLRSVG |
| | glycoprotein | LNLEGNGVATDVPSATKRWGFRSGVPPKVVNYEAGEWAENCYNLEIKKPDGSE |
| | | CLPAAPDGIRGFPRCRYVHKVSGTGPCAGDFAFHKEGAFFLYDRLASTVIYRG |
| | | TTFAEGVVAFLILPQAKKDFFSSHPLREPVNATEDPSSGYYSTTIRYQATGFG |
| | | TNETEYLFEVDNLTYVQLESRFTPQFLLQLNETIYTSGKRSNTTGKLIWKVNP |
| | | EIDTTIGEWAFWETKKNLTRKIRSEEWSHPQFEKGGGGSGGGGSGGGGSQVQL |
| | | QQPGAELVKPGASVKLSCKASGYPFTSYWIHWVKQRPGRGLEWLGRIDPNSGD |
| | | TKYNEKFKNKATLTVDKSSTTAYMQLSSLTSEDSAVYYCARSPYYSNDNSMDY |
| | | WGQGTSVTVSSGGGGSGGGGSGGGGSGGGGSDILLTQSPAILSVSPGERVSFS |
| | | CRASQSIGTSIHWYQQRTNDSPRLLIKYASESISGIPSRFSGSGSGTDFTLSI |
| | | NSVESEDIADYYCQQSNSWPTTFGGGTKLEIKRGGGGSGGGGSGGGGSNTIAG |
| | | VAGLITGGRRTRREAIVNAQPKCNPNLHYWTTQDEGAAIGLAWIPYFGPAAEG |
| | | IYIEGLMHNQDGLICGLRQLANETTQALQLFLRATTELRIFSILNRKAIDFLL |
| | | QRWGGTCHILGPDCCIEPHDWTKNITDKIDQIIHDFVDKTLPDQGDNDNWWTG |
| | | WRQWIPAGIGVTGVIIAVIALFCICKFVF |
| 189 | Ebola | KRTSFFLWVIILFQRTFSIPLGVIHNSTLQVSDVDKLVCRDKLSSTNQLRSVG |
| | glycoprotein | LNLEGNGVATDVPSATKRWGFRSGVPPKVVNYEAGEWAENCYNLEIKKPDGSE |
| | | CLPAAPDGIRGFPRCRYVHKVSGTGPCAGDFAFHKEGAFFLYDRLASTVIYRG |
| | | TTFAEGVVAFLILPQAKKDFFSSHPLREPVNATEDPSSGYYSTTIRYQATGFG |
| | | TNETEYLFEVDNLTYVQLESRFTPQFLLQLNETIYTSGKRSNTTGKLIWKVNP |
| | | EIDTTIGEWAFWETKKNLTRKIRSEESAWSHPQFEKGGGGSGGGGSGGGGSAS |
| | | QVQLQQPGAELVKPGASVKLSCKASGYPFTSYWIHWVKQRPGRGLEWLGRIDP |
| | | NSGDTKYNEKFKNKATLTVDKSSTTAYMQLSSLTSEDSAVYYCARSPYYSNDN |
| | | SMDYWGQGTSVTVSSGGGGSGGGGSGGGGSGGGGSDILLTQSPAILSVSPGER |
| | | VSFSCRASQSIGTSIHWYQQRTNDSPRLLIKYASESISGIPSRFSGSGSGTDF |
| | | TLSINSVESEDIADYYCQQSNSWPTTFGGGTKLEIKRTGGGGGSGGGGSGGGG |
| | | SWSHPQFEKSANTIAGVAGLITGGRRTRREAIVNAQPKCNPNLHYWTTQDEGA |
| | | AIGLAWIPYFGPAAEGIYIEGLMHNQDGLICGLRQLANETTQALQLFLRATTE |
| | | LRTFSILNRKAIDFLLQRWGGTCHILGPDCCIEPHDWTKNITDKIDQIIHDFV |
| | | DKTLPDQGDNDNWWTGWRQWIPAGIGVTGVIIAVIALFCICKFVF |
| 190 | Ebola | MGVTGILQLPRDRFKRTSFFLWVIILFQRTFSIPLGVIHNSTLQVSDVDKLVC |
| | glycoprotein | RDKLSSTNQLRSVGLNLEGNGVATDVPSATKRWGFRSGVPPKVVNYEAGEWAE |
| | | NCYNLEIKKPDGSECLPAAPDGIRGFPRCRYVHKVSGTGPCAGDFAFHKEGAF |
| | | FLYDRLASTVIYRGTTFAEGVVAFLILPQAKKDFFSSHPLREPVNATEDPSSG |
| | | YYSTTIRYQATGFGTNETEYLFEVDNLTYVQLESRFTPQFLLQLNETIYTSGK |
| | | RSNTTGKLIWKVNPEIDTTIGEWAFWETKKNLTRKIRSEEWSHPQFEKGGGGS |
| | | GGGGSGGGGSQVQLQQPGAELVKPGASVKLSCKASGYPFTSYWIHWVKQRPGR |
| | | GLEWLGRIDPNSGDTKYNEKFKNKATLTVDKSSTTAYMQLSSLTSEDSAVYYC |
| | | ARSPYYSNDNSMDYWGQGTSVTVSSGGGGSGGGGSGGGGSGGGGSDILLTQSP |
| | | AILSVSPGERVSFSCRASQSIGTSIHWYQQRTNDSPRLLIKYASESISGIPSR |
| | | FSGSGSGTDFTLSINSVESEDIADYYCQQSNSWPTTFGGGTKLEIKRGGGGSG |
| | | GGGSGGGGSWSHPQFEKNTIAGVAGLITGGRRTRREAIVNAQPKCNPNLHYWT |
| | | TQDEGAAIGLAWIPYFGPAAEGIYIEGLMHNQDGLICGLRQLANETTQALQLF |
| | | LRATTELRTFSILNRKAIDFLLQRWGGTCHILGPDCCIEPHDWTKNITDKIDQ |
| | | IIHDFVDKTLPDQGDNDNWWTGWRQWIPAGIGVTGVIIAVIALFCICKFVF |
| 191 | Ebola | KRTSFFLWVIILFQRTFSIPLGVIHNSTLQVSDVDKLVCRDKLSSTNQLRSVG |
| | glycoprotein | LNLEGNGVATDVPSATKRWGFRSGVPPKVVNYEAGEWAENCYNLEIKKPDGSE |
| | | CLPAAPDGIRGFPRCRYVHKVSGTGPCAGDFAFHKEGAFFLYDRLASTVIYRG |
| | | TTFAEGVVAFLILPQAKKDFFSSHPLREPVNATEDPSSGYYSTTIRYQATGFG |
| | | TNETEYLFEVDNLTYVQLESRFTPQFLLQLNETIYTSGKRSNTTGKLIWKVNP |
| | | EIDTTIGEWAFWETKKNLTRKIRSEEWSHPQFEKGGGGSGGGGSGGGGSQVQL |
| | | QQPGAELVKPGASVKLSCKASGYPFTSYWIHWVKQRPGRGLEWLGRIDPNSGD |
| | | TKYNEKFKNKATLTVDKSSTTAYMQLSSLTSEDSAVYYCARSPYYSNDNSMDY |
| | | WGQGTSVTVSSGGGGSGGGGSGGGGSGGGGSDILLTQSPAILSVSPGERVSFS |
| | | CRASQSIGTSIHWYQQRTNDSPRLLIKYASESISGIPSRFSGSGSGTDFTLSI |
| | | NSVESEDIADYYCQQSNSWPTTFGGGTKLEIKRGGGGSGGGGSGGGGSWSHPQ |
| | | FEKNTIAGVAGLITGGRRTRREAIVNAQPKCNPNLHYWTTQDEGAAIGLAWIP |
| | | YFGPAAEGIYIEGLMHNQDGLICGLRQLANETTQALQLFLRATTELRTFSILN |
| | | RKAIDFLLQRWGGTCHILGPDCCIEPHDWTKNITDKIDQIIHDFVDKTLPDQG |
| | | DNDNWWTGWRQWIPAGIGVTGVIIAVIALFCICKFVF |
| 166 | NiV-F | WSHPQFEKGGSILHYEKLSKIGLVKGVTRKYKIKSNPLTKDIVIKMIPNVSNM |
| | | SQCTGSVMENYKTRLNGILTPIKGALEIYKNNTHDLVGDVRLAGVIMAGVAIG |
| | | IATAAQITAGVALYEAMKNADNINKLKSSIESTNEAVVKLQETAEKTVYVLTA |
| | | LQDYINTNLVPTIDKISCKQTELSLDLALSKYLSDLLFVFGPNLQDPVSNSMT |
| | | IQAISQAFGGNYETLLRTLGYATEDFDDLLESDSITGQIIYVDLSSYYIIVRV |
| | | YFPILTEIQQAYIQELLPVSFNNDNSEWISIVPNFILVRNTLISNIEIGFCLI |
| | | TKRSVICNQDYATPMINNMRECLTGSTEKCPRELVVSSHVPRFALSNGVLFAN |
| | | CISVTCQCQTTGRAISQSGEQTLLMIDNTTCPTAVIGNVIISLGKYLGSVNYN |
| | | SEGIAIGPPVFTDKVDISSQISSMNQSLQQSKDYIKEAQRLLDTVNPSLISML |
| | | SMIILYVLSIASLCIGLITFISFIIVEKKRNT |
| 167 | VSV-G | KFSGGWSHPQFEKGGSTIVFPHNQKGNWKNVPSNYHYCPSSSDLNWHNDLIGT |
| | | ALQVKMPKSHKAIQADGWMCHASKWVTTCDFRWYGPKYITHSIRSFTPSVEQC |
| | | KESIEQTKQGTWLNPGFPPQSCGYATVTDAEAVIVQVTPHHVLVDEYTGEWVD |
| | | SQFINGKCSNYICPTVHNSTTWHSDYKVKGLCDSNLESMDITFFSEDGELSSL |
| | | GKEGTGFRSNYFAYENGGKACKMQYCKHWGVRLPSGVWFEMADKDLFAAARFP |
| | | ECPEGSSISAPSQTSVDVSLIQDVERILDYSLCQETWSKIRAGLPISPVDLSY |
| | | LAPKNPGTGPAFTIINGILKYFETRYIRVDIAAPILSRMVGMISGTTAERELW |
| | | DDWAPYEDVEIGPNGVLRTSSGYKFPLYMIGHGMLDSDLHLSSKAQVFEHPHI |
| | | QDAASQLPDDESLFFGDTGLSKNPIELVEGWFSSWKSSIASFFFIIGLIIGLF |
| | | LVLRVGIHLCIKLKHTKKRQIYTDIEMNRLGK |
| 168 | VGV-G | KFTIVFPSGGWSHPQFEKGGSHNQKGNWKNVPSNYHYCPSSSDLNWHNDLIGT |
| | | ALQVKMPKSHKAIQADGWMCHASKWVTTCDFRWYGPKYITHSIRSFTPSVEQC |
| | | KESIEQTKQGTWLNPGFPPQSCGYATVIDAEAVIVQVTPHHVLVDEYTGEWVD |
| | | SQFINGKCSNYICPTVHNSTTWHSDYKVKGLCDSNLESMDITFFSEDGELSSL |
| | | GKEGTGFRSNYFAYENGGKACKMQYCKHWGVRLPSGVWFEMADKDLFAAARFP |
| | | ECPEGSSISAPSQTSVDVSLIQDVERILDYSLCQETWSKIRAGLPISPVDLSY |
| | | LAPKNPGTGPAFTIINGTLKYFETRYIRVDIAAPILSRMVGMISGTTAERELW |
| | | DDWAPYEDVEIGPNGVLRTSSGYKFPLYMIGHGMLDSDLHLSSKAQVFEHPHI |
| | | QDAASQLPDDESLFFGDTGLSKNPIELVEGWFSSWKSSIASFFFIIGLIIGLF |
| | | LVLRVGIHLCIKLKHTKKRQIYTDIEMNRLGK |
| 170 | MeV-F | WSHPQFEKGGSQIHWGNLSKIGVVGIGSASYKVMTRSSHQSLVIKLMPNITLL |
| | | NNCTRVEIAEYRRLLRTVLEPIRDALNAMTQNIRPVQSVASSRRHKRFAGVVL |
| | | AGAALGVATAAQITAGIALHQSMLNSQAIDNLRASLETTNQAIEAIRQAGQEM |
| | | ILAVQGVQDYINNELIPSMNQLSCDLIGQKLGLKLLRYYTEILSLFGPSLRDP |
| | | ISAEISIQALSYALGGDINKVLEKLGYSGGDLLGILESRGIKARITHVDTESY |
| | | FIVLSIAYPTLSEIKGVIVHRLEGVSYNIGSQEWYTTVPKYVATQGYLISNFD |
| | | ESSCTFMPEGTVCSQNALYPMSPLLQECLRGSTKSCARTLVSGSFGNRFILSQ |
| | | GNLIANCASILCKCYTTGTIINQDPDKILTYIAADHCPVVEVNGVTIQVGSRR |
| | | YPDAVYLHRIDLGPPISLERLDVGTNLGNAIAKLEDAKELLESSDQILRSMKG |
| | | LSSTSIVYILIAVCLGGLIGIPALICCCRGR |
| 172 | SVCV-G | WSHPQFEKGGSIPIFVPSGRNISWQPVIQPFDYQCPIHGNLPNTMGLSATKLT |
| | | IKSPSVFSTDKVSGWICHAAEWKTTCDYRWYGPQYITHSIHPISPTIDECRRI |
| | | IQRIASGTDEDLGFPPQSCGWASVTTVSNTNYRVVPHSVHLEPYGGHWIDHEF |
| | | NGGECREKVCEMKGNHSIWITEETVQHECAKHIEEVEGIMYGNVPRGDVMYAN |
| | | NFIIDRHHRVYRFGGSCQMKFCNKDGIKFARGDWVEKTAGILITIHDNVPKCV |
| | | DGTLVSGHRPGLDLIDTVFNLENVVEYTLCEGTKRKINKQEKLISVDLSYLAP |
| | | RIGGFGSVFRVRNGTLERGSTTYIRIEVEGPIVDSLNGTDPRTNASRVFWDDW |
| | | ELDGNIYQGFNGVYKGKDGKIHIPLNMIESGIIDDELQHAFQADIIPHPHYDD |
| | | DEIREDDIFFDNTGENGNPVDAVVEWVSGWGTSLKFFGMTLVALILIFLLIRC |
| | | CVACTYLMKRSKRPATESHEMRSLV |
| 174 | Binder | WSHPQFEKGGSSAQVQLQQPGAELVKPGASVKLSCKASGYPFTSYWIHWVKQR |
| | | PGRGLEWLGRIDPNSGDTKYNEKFKNKATLTVDKSSTTAYMQLSSLTSEDSAV |
| | | YYCARSPYYSNDNSMDYWGQGTSVTVSSGGGGSGGGGSGGGGSGGGGSDILLT |
| | | QSPAILSVSPGERVSFSCRASQSIGTSIHWYQQRTNDSPRLLIKYASESISGI |
| | | PSRFSGSGSGTDFTLSINSVESEDIADYYCQQSNSWPTTFGGGTKLEIKRASG |
| | | GGGSGGGGSGGGGSEPKSCDKTHTCPPCPAPEAAGGPSVFLFPPKPKDTLMAS |
| | | RTPEVTCVVVDVSHEDPEVKFNWYVDGVEVHNAKTKPREEQYASTYRVVSVLT |
| | | VLAQDWLNGKEYKCKVSNKALGAPIEKTISKAKGQPREPQVYTLPPSRDELTK |
| | | NQVSLTCLVKGFYPSDIAVEWESNGQPENNYKTTPPVLDSDGSFFLYSKLTVD |
| | | KSRWQQGNVFSCSVMHEALHNAYTQKSLSLSPGKKIEVMYPPPYLDNEKSNGT |
| | | IIHVKGKHLCPSPLFPGPSKPFWVLVVVGGVLACYSLLVTVAFIIFWVRSKRS |
| | | RLLHSDYMNRVRQGYS |
| 175 | Binder | METDTLLLWVLLLWVPGSTGDWSHPQFEKGGSQVQLQQPGAELVKPGASVKLS |
| | | CKASGYPFTSYWIHWVKQRPGRGLEWLGRIDPNSGDTKYNEKFKNKATLTVDK |
| | | SSTTAYMQLSSLISEDSAVYYCARSPYYSNDNSMDYWGQGTSVTVSSGGGGSG |
| | | GGGSGGGGSGGGGSDILLTQSPAILSVSPGERVSFSCRASQSIGTSIHWYQQR |
| | | TNDSPRLLIKYASESISGIPSRFSGSGSGTDFTLSINSVESEDIADYYCQQSN |
| | | SWPTTFGGGTKLEIKRGGGGSGGGGSGGGGSEPKSCDKTHTCPPCPAPEAAGG |
| | | PSVFLFPPKPKDTLMASRTPEVTCVVVDVSHEDPEVKFNWYVDGVEVHNAKTK |
| | | PREEQYASTYRVVSVLTVLAQDWLNGKEYKCKVSNKALGAPIEKTISKAKGQP |
| | | REPQVYTLPPSRDELTKNQVSLTCLVKGFYPSDIAVEWESNGQPENNYKTTPP |
| | | VLDSDGSFFLYSKLTVDKSRWQQGNVFSCSVMHEALHNAYTQKSLSLSPGKKI |
| | | EVMYPPPYLDNEKSNGTIIHVKGKHLCPSPLFPGPSKPFWVLVVVGGVLACYS |
| | | LLVTVAFIIFWVRSKRSRLLHSDYMNRVRQGYS |
| 176 | Binder | WSHPQFEKGGSQVQLQQPGAELVKPGASVKLSCKASGYPFTSYWIHWVKQRPG |
| | | RGLEWLGRIDPNSGDTKYNEKFKNKATLTVDKSSTTAYMQLSSLTSEDSAVYY |
| | | CARSPYYSNDNSMDYWGQGTSVTVSSGGGGSGGGGSGGGGSGGGGSDILLTQS |
| | | PAILSVSPGERVSFSCRASQSIGTSIHWYQQRTNDSPRLLIKYASESISGIPS |
| | | RFSGSGSGTDFTLSINSVESEDIADYYCQQSNSWPTTFGGGTKLEIKRGGGGS |
| | | GGGGSGGGGSEPKSCDKTHTCPPCPAPEAAGGPSVFLFPPKPKDTLMASRTPE |
| | | VTCVVVDVSHEDPEVKFNWYVDGVEVHNAKTKPREEQYASTYRVVSVLTVLAQ |
| | | DWLNGKEYKCKVSNKALGAPIEKTISKAKGQPREPQVYTLPPSRDELTKNQVS |
| | | LTCLVKGFYPSDIAVEWESNGQPENNYKTTPPVLDSDGSFFLYSKLTVDKSRW |
| | | QQGNVFSCSVMHEALHNAYTQKSLSLSPGKKIEVMYPPPYLDNEKSNGTIIHV |
| | | KGKHLCPSPLFPGPSKPFWVLVVVGGVLACYSLLVTVAFIIFWVRSKRSRLLH |
| | | SDYMNRVRQGYS |
| 178 | Binder | WSHPQFEKGGSGSDIQVQLQQPGAELVKPGASVKLSCKASGYPFTSYWIHWVK |
| | | QRPGRGLEWLGRIDPNSGDTKYNEKFKNKATLTVDKSSTTAYMQLSSLTSEDS |
| | | AVYYCARSPYYSNDNSMDYWGQGTSVTVSSGGGGSGGGGSGGGGSGGGGSDIL |
| | | LTQSPAILSVSPGERVSFSCRASQSIGTSIHWYQQRTNDSPRLLIKYASESIS |
| | | GIPSRFSGSGSGTDFTLSINSVESEDIADYYCQQSNSWPTTFGGGTKLEIKRV |
| | | NGGGGSGGGGSGGGGSGGGGSAVHTRGLDFACDIYIWAPLAGTCGVLLLSLVI |
| | | TLYCNHRNRRRVNRVRQGYS |
| 179 | Binder | WSHPQFEKGGGGSGGGGSSAQVQLQQPGAELVKPGASVKLSCKASGYPFTSYW |
| | | IHWVKQRPGRGLEWLGRIDPNSGDTKYNEKFKNKATLTVDKSSTTAYMQLSSL |
| | | TSEDSAVYYCARSPYYSNDNSMDYWGQGTSVTVSSGGGGSGGGGSGGGGSGGG |
| | | GSDILLTQSPAILSVSPGERVSFSCRASQSIGTSIHWYQQRTNDSPRLLIKYA |
| | | SESISGIPSRFSGSGSGTDFTLSINSVESEDIADYYCQQSNSWPTTFGGGTKL |
| | | EIKRASGGGGSGGGGSGGGGSEPKSCDKTHTCPPCPAPEAAGGPSVFLFPPKP |
| | | KDTLMASRTPEVTCVVVDVSHEDPEVKFNWYVDGVEVHNAKIKPREEQYASTY |
| | | RVVSVLTVLAQDWLNGKEYKCKVSNKALGAPIEKTISKAKGQPREPQVYTLPP |
| | | SRDELTKNQVSLTCLVKGFYPSDIAVEWESNGQPENNYKTTPPVLDSDGSFFL |
| | | YSKLTVDKSRWQQGNVFSCSVMHEALHNAYTQKSLSLSPGKKIEVMYPPPYLD |
| | | NEKSNGTIIHVKGKHLCPSPLFPGPSKPFWVLVVVGGVLACYSLLVTVAFIIF |
| | | WVRSKRSRLLHSDYMNRVRQGYS |
| 180 | Binder | METDTLLLWVLLLWVPGSTGDWSHPQFEKGGGGSGGGGSQVQLQQPGAELVKP |
| | | GASVKLSCKASGYPFTSYWIHWVKQRPGRGLEWLGRIDPNSGDTKYNEKFKNK |
| | | ATLTVDKSSTTAYMQLSSLTSEDSAVYYCARSPYYSNDNSMDYWGQGTSVTVS |
| | | SGGGGSGGGGSGGGGSGGGGSDILLTQSPAILSVSPGERVSFSCRASQSIGTS |
| | | IHWYQQRTNDSPRLLIKYASESISGIPSRFSGSGSGTDFTLSINSVESEDIAD |
| | | YYCQQSNSWPTTFGGGTKLEIKRGGGGSGGGGSGGGGSEPKSCDKTHTCPPCP |
| | | APEAAGGPSVFLFPPKPKDTLMASRTPEVTCVVVDVSHEDPEVKFNWYVDGVE |
| | | VHNAKTKPREEQYASTYRVVSVLTVLAQDWLNGKEYKCKVSNKALGAPIEKTI |
| | | SKAKGQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNAYTQKSLS |
| | | LSPGKKIEVMYPPPYLDNEKSNGTIIHVKGKHLCPSPLFPGPSKPFWVLVVVG |
| | | GVLACYSLLVTVAFIIFWVRSKRSRLLHSDYMNRVRQGYS |
| 181 | Binder | WSHPQFEKGGGGSGGGGSQVQLQQPGAELVKPGASVKLSCKASGYPFTSYWIH |
| | | WVKQRPGRGLEWLGRIDPNSGDTKYNEKFKNKATLTVDKSSTTAYMQLSSLTS |
| | | EDSAVYYCARSPYYSNDNSMDYWGQGTSVTVSSGGGGSGGGGSGGGGSGGGGS |
| | | DILLTQSPAILSVSPGERVSFSCRASQSIGTSIHWYQQRTNDSPRLLIKYASE |
| | | SISGIPSRFSGSGSGTDFTLSINSVESEDIADYYCQQSNSWPTTFGGGTKLEI |
| | | KRGGGGSGGGGSGGGGSEPKSCDKTHTCPPCPAPEAAGGPSVFLFPPKPKDTL |
| | | MASRTPEVTCVVVDVSHEDPEVKFNWYVDGVEVHNAKTKPREEQYASTYRVVS |
| | | VLTVLAQDWLNGKEYKCKVSNKALGAPIEKTISKAKGQPREPQVYTLPPSRDE |
| | | LTKNQVSLTCLVKGFYPSDIAVEWESNGQPENNYKTTPPVLDSDGSFFLYSKL |
| | | TVDKSRWQQGNVFSCSVMHEALHNAYTQKSLSLSPGKKIEVMYPPPYLDNEKS |
| | | NGTIIHVKGKHLCPSPLFPGPSKPFWVLVVVGGVLACYSLLVTVAFIIFWVRS |
| | | KRSRLLHSDYMNRVRQGYS |
| 182 | Binder | SAQVQLQQPGAELVKPGASVKLSCKASGYPFTSYWIHWVKQRPGRGLEWLGRI |
| | | DPNSGDTKYNEKFKNKATLTVDKSSTTAYMQLSSLTSEDSAVYYCARSPYYSN |
| | | DNSMDYWGQGTSVTVSSGGGGSGGGGSWSHPQFEKGGGGSGGGGSDILLTQSP |
| | | AILSVSPGERVSFSCRASQSIGTSIHWYQQRTNDSPRLLIKYASESISGIPSR |
| | | FSGSGSGTDFTLSINSVESEDIADYYCQQSNSWPTTFGGGTKLEIKRASGGGG |
| | | SGGGGSGGGGSEPKSCDKTHTCPPCPAPEAAGGPSVFLFPPKPKDTLMASRTP |
| | | EVTCVVVDVSHEDPEVKFNWYVDGVEVHNAKTKPREEQYASTYRVVSVLTVLA |
| | | QDWLNGKEYKCKVSNKALGAPIEKTISKAKGQPREPQVYTLPPSRDELTKNQV |
| | | SLTCLVKGFYPSDIAVEWESNGQPENNYKTTPPVLDSDGSFFLYSKLTVDKSR |
| | | WQQGNVFSCSVMHEALHNAYTQKSLSLSPGKKIEVMYPPPYLDNEKSNGTIIH |
| | | VKGKHLCPSPLFPGPSKPFWVLVVVGGVLACYSLLVTVAFIIFWVRSKRSRLL |
| | | HSDYMNRVRQGYS |
| 183 | Binder | METDILLLWVLLLWVPGSTGDQVQLQQPGAELVKPGASVKLSCKASGYPFTSY |
| | | WIHWVKQRPGRGLEWLGRIDPNSGDTKYNEKFKNKATLTVDKSSTTAYMQLSS |
| | | LTSEDSAVYYCARSPYYSNDNSMDYWGQGTSVTVSSGGGGSGGGGSWSHPQFE |
| | | KGGGGSGGGGSDILLTQSPAILSVSPGERVSFSCRASQSIGTSIHWYQQRTND |
| | | SPRLLIKYASESISGIPSRFSGSGSGTDFTLSINSVESEDIADYYCQQSNSWP |
| | | TTFGGGTKLEIKRGGGGSGGGGSGGGGSEPKSCDKTHTCPPCPAPEAAGGPSV |
| | | FLFPPKPKDTLMASRIPEVTCVVVDVSHEDPEVKFNWYVDGVEVHNAKIKPRE |
| | | EQYASTYRVVSVLIVLAQDWLNGKEYKCKVSNKALGAPIEKTISKAKGQPREP |
| | | QVYTLPPSRDELTKNQVSLTCLVKGFYPSDIAVEWESNGQPENNYKTTPPVLD |
| | | SDGSFFLYSKLTVDKSRWQQGNVFSCSVMHEALHNAYTQKSLSLSPGKKIEVM |
| | | YPPPYLDNEKSNGTIIHVKGKHLCPSPLFPGPSKPFWVLVVVGGVLACYSLLV |
| | | TVAFIIFWVRSKRSRLLHSDYMNRVRQGYS |
| 184 | Binder | QVQLQQPGAELVKPGASVKLSCKASGYPFTSYWIHWVKQRPGRGLEWLGRIDP |
| | | NSGDTKYNEKFKNKATLTVDKSSTTAYMQLSSLTSEDSAVYYCARSPYYSNDN |
| | | SMDYWGQGTSVTVSSGGGGSGGGGSWSHPQFEKGGGGSGGGGSDILLTQSPAI |
| | | LSVSPGERVSFSCRASQSIGTSIHWYQQRTNDSPRLLIKYASESISGIPSRFS |
| | | GSGSGTDFTLSINSVESEDIADYYCQQSNSWPTTFGGGTKLEIKRGGGGSGGG |
| | | GSGGGGSEPKSCDKTHTCPPCPAPEAAGGPSVFLFPPKPKDTLMASRTPEVTC |
| | | VVVDVSHEDPEVKFNWYVDGVEVHNAKTKPREEQYASTYRVVSVLIVLAQDWL |
| | | NGKEYKCKVSNKALGAPIEKTISKAKGQPREPQVYTLPPSRDELTKNQVSLTC |
| | | LVKGFYPSDIAVEWESNGQPENNYKTTPPVLDSDGSFFLYSKLTVDKSRWQQG |
| | | NVFSCSVMHEALHNAYTQKSLSLSPGKKIEVMYPPPYLDNEKSNGTIIHVKGK |
| | | HLCPSPLFPGPSKPFWVLVVVGGVLACYSLLVTVAFIIFWVRSKRSRLLHSDY |
| | | MNRVRQGYS |
| 185 | binder | SAQVQLQQPGAELVKPGASVKLSCKASGYPFTSYWIHWVKQRPGRGLEWLGRI |
| | | DPNSGDTKYNEKFKNKATLTVDKSSTTAYMQLSSLTSEDSAVYYCARSPYYSN |
| | | DNSMDYWGQGTSVTVSSGGGGSGGGGSGGGGSGGGGSDILLTQSPAILSVSPG |
| | | ERVSFSCRASQSIGTSIHWYQQRTNDSPRLLIKYASESISGIPSRFSGSGSGT |
| | | DFTLSINSVESEDIADYYCQQSNSWPTTFGGGTKLEIKRASGGGGSGGGGSWS |
| | | HPQFEKGGGGSEPKSCDKTHTCPPCPAPEAAGGPSVFLFPPKPKDTLMASRTP |
| | | EVTCVVVDVSHEDPEVKFNWYVDGVEVHNAKTKPREEQYASTYRVVSVLTVLA |
| | | QDWLNGKEYKCKVSNKALGAPIEKTISKAKGQPREPQVYTLPPSRDELTKNQV |
| | | SLTCLVKGFYPSDIAVEWESNGQPENNYKTTPPVLDSDGSFFLYSKLTVDKSR |
| | | WQQGNVFSCSVMHEALHNAYTQKSLSLSPGKKIEVMYPPPYLDNEKSNGTIIH |
| | | VKGKHLCPSPLFPGPSKPFWVLVVVGGVLACYSLLVTVAFIIFWVRSKRSRLL |
| | | HSDYMNRVRQGYS |
| 186 | Binder | METDTLLLWVLLLWVPGSTGDQVQLQQPGAELVKPGASVKLSCKASGYPFTSY |
| | | WIHWVKQRPGRGLEWLGRIDPNSGDTKYNEKFKNKATLTVDKSSTTAYMQLSS |
| | | LTSEDSAVYYCARSPYYSNDNSMDYWGQGTSVTVSSGGGGSGGGGSGGGGSGG |
| | | GGSDILLTQSPAILSVSPGERVSFSCRASQSIGTSIHWYQQRTNDSPRLLIKY |
| | | ASESISGIPSRFSGSGSGTDFTLSINSVESEDIADYYCQQSNSWPTTFGGGTK |
| | | LEIKRGGGGSGGGGSWSHPQFEKGGGGSEPKSCDKTHTCPPCPAPEAAGGPSV |
| | | FLFPPKPKDTLMASRTPEVTCVVVDVSHEDPEVKFNWYVDGVEVHNAKTKPRE |
| | | EQYASTYRVVSVLTVLAQDWLNGKEYKCKVSNKALGAPIEKTISKAKGQPREP |
| | | QVYTLPPSRDELTKNQVSLTCLVKGFYPSDIAVEWESNGQPENNYKTTPPVLD |
| | | SDGSFFLYSKLTVDKSRWQQGNVFSCSVMHEALHNAYTQKSLSLSPGKKIEVM |
| | | YPPPYLDNEKSNGTIIHVKGKHLCPSPLFPGPSKPFWVLVVVGGVLACYSLLV |
| | | TVAFIIFWVRSKRSRLLHSDYMNRVRQGYS |
| 187 | Binder | QVQLQQPGAELVKPGASVKLSCKASGYPFTSYWIHWVKQRPGRGLEWLGRIDP |
| | | NSGDTKYNEKFKNKATLTVDKSSTTAYMQLSSLTSEDSAVYYCARSPYYSNDN |
| | | SMDYWGQGTSVTVSSGGGGSGGGGSGGGGSGGGGSDILLTQSPAILSVSPGER |
| | | VSFSCRASQSIGTSIHWYQQRTNDSPRLLIKYASESISGIPSRFSGSGSGTDF |
| | | TLSINSVESEDIADYYCQQSNSWPTTFGGGTKLEIKRGGGGSGGGGSWSHPQF |
| | | EKGGGGSEPKSCDKTHTCPPCPAPEAAGGPSVFLFPPKPKDTLMASRTPEVTC |
| | | VVVDVSHEDPEVKFNWYVDGVEVHNAKTKPREEQYASTYRVVSVLTVLAQDWL |
| | | NGKEYKCKVSNKALGAPIEKTISKAKGQPREPQVYTLPPSRDELTKNQVSLTC |
| | | LVKGFYPSDIAVEWESNGQPENNYKTTPPVLDSDGSFFLYSKLTVDKSRWQQG |
| | | NVFSCSVMHEALHNAYTQKSLSLSPGKKIEVMYPPPYLDNEKSNGTIIHVKGK |
| | | HLCPSPLFPGPSKPFWVLVVVGGVLACYSLLVTVAFIIFWVRSKRSRLLHSDY |
| | | MNRVRQGYS |

### EXAMPLES

The following examples are illustrative, but not limiting, of the compounds, compositions and methods described herein. Other suitable modifications and adaptations known to those skilled in the art are within the scope of the following embodiments.

### Example 1: Construction of VSV-G:Aflinity Binding Polypeptide Constructs

Pseudotyped lentiviral constructs harboring an affinity binding polypeptide in the viral glycoprotein were designed. A VSV-G protein was modified to contain a Strep Tag II polypeptide at various locations at or near the N-terminus of the VSG-G protein (SEQ ID NO: 70, SEQ ID NO: 71, SEQ ID NO: 72), or within the body of the VSV-G protein (SEQ ID NO: 73). The Strep Tag It polypeptide is as shown in SEQ ID NO: 1. Each construct was designed to have the Strep Tag 1I polypeptide fused either directly to the VSV-G polypeptide sequence (SEQ ID NO: 70, 71, 72, and 73) or to be linked via a short linker polypeptide (SEQ ID NO: 132, SEQ ID NO: 133, SEQ ID NO: 167, and SEQ ID NO: 168). The VSV-G construct represented by SEQ ID NO: 73 was structurally designed such that the affinity binding polypeptide would be solvent exposed and thus the affinity binding polypeptide would be accessible for affinity based purification.

### Example 2: Construction of Targeting Moiety:Affinity Binding Polypeptide Constructs

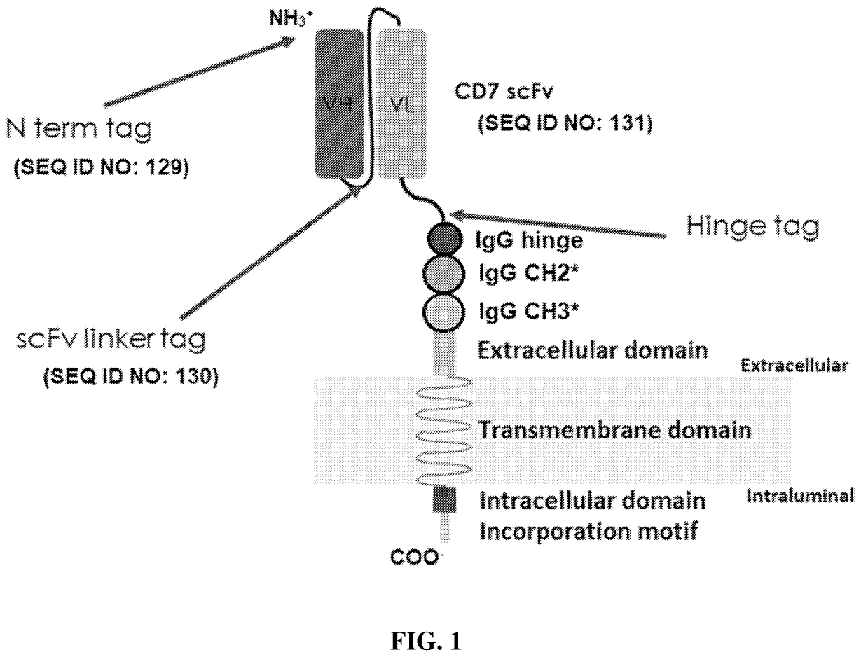

Pseudotyped lentiviral constructs harboring an affinity binding polypeptide in the viral targeting moiety were designed. A CD7 targeting moiety polypeptide as provided for herein was modified to contain a Strep Tag II polypeptide at the N-terminus of the targeting moiety (SEQ ID NO: 129, SEQ ID NO: 179, SEQ ID NO: 180, and SEQ ID NO: 181), within the scFv linker portion (SEQ ID NO: 130, SEQ ID NO: 182, SEQ ID NO: 183, and SEQ ID NO: 184), or within the IgG hinge region (SEQ ID NO: 131, SEQ ID NO: 185, SEQ ID NO: 186, and SEQ ID NO: 187) (FIG. 1). The Strep Tag II polypeptide is as shown in SEQ ID NO: 1. Each construct was designed to have the Strep Tag II polypeptide fused either directly to the targeting moiety or to be linked via a short linker polypeptide.

### Example 3: Assessment of Affinity Binding Polypeptide on Virus Function

To determine the effect of the affinity binding polypeptide on viral function, viral constructs harboring a Strep Tag II polypeptide in the VSV-G protein or in the targeting moiety were purified via traditional viral purification techniques and assessed. VSV-G proteins constructs included those of Example 1 above. Targeting moiety constructs included those of Example 2 above. The constructs were evaluated for their capability to deliver a CD20 CAR construct (CAR20) to SupT1 cells. SupT1 cells were seeded in 96-well tissue culture plates with 25,000 cells per well in RPMI+10% FBS culture media. Concentrated lentiviral suspensions for each construct were diluted in a 3-fold dilution series in culture media. Diluted viral suspensions were then added to SupT1 cells in respective wells. Culture media was replaced 6 hours later. The treated SupT1 cells were analyzed by flow cytometry on days 4 or 7 after transduction. On each day of analysis, cells were stained with a cell viability marker to identify live cells and an anti-CAR20 antibody to identify CAR20 positive cells.

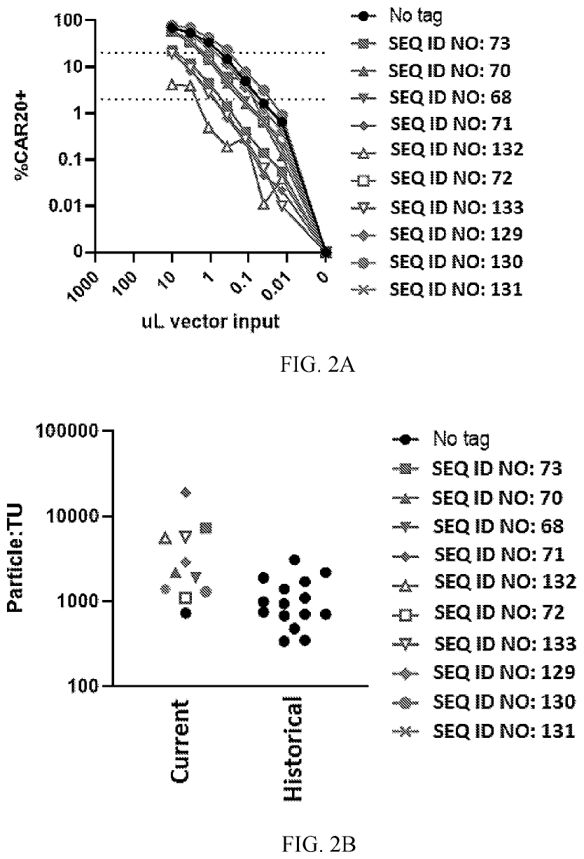

As shown in FIG. 2A and FIG. 2B, viral constructs harboring affinity binding polypeptides in the targeting moiety of the construct had a similar CAR20 transduction profile as compared to viral constructs having no affinity tag and had overlapping particle:infectivity (P:TU) ratios as compared to the untagged constructs. Viral constructs harboring affinity binding polypeptides in the glycoprotein were more variable. Constructs corresponding to SEQ ID NO: 70 and SEQ ID NO: 72 had a similar CAR20 transduction profile as compared to viral constructs having no affinity tag and had overlapping P:TU ratios as compared to the untagged constructs. The P:TU did not necessarily correspond to the Strep Tag II position. The Strep Tag II polypeptide position in SEQ ID NO: 71 is near the N-terminus of the VSV-G protein and only one amino acid shift as compared to SEQ ID NO: 70, and yet the P:TU ratios for these two constructs were not overlapping.

The data from the example shows that incorporation of an affinity binding polypeptide such as Strep Tag II into the targeting moiety or glycoprotein of a pseudotyped lentiviral construct results in functional viral particles.

### Example 4: Purification of Pseudotyped Lentiviral Constructs Harboring Affinity Binding Polypeptide Using Column Chromatography

Biotin in Buffer does not Affect Titer

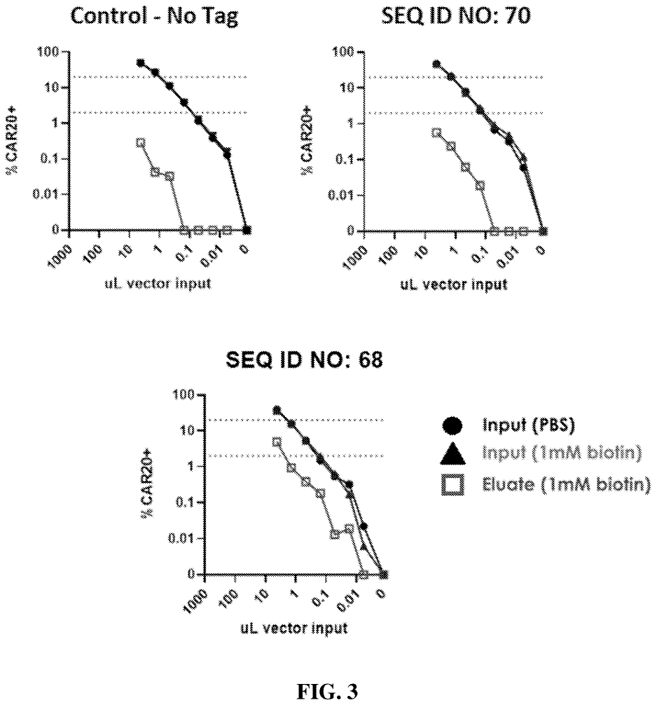

Prior to purification of viral constructs via the Strep Tag II polypeptide, it was assessed whether the presence of biotin in the elution buffer would affect viral transduction. Untagged pseudotyped virus and pseudotyped virus harboring a Strep Tag II polypeptide in the VSV-G protein either with or without a flexible linker were purified via traditional viral purification methods. Biotin (1 mM) was added to the buffer solution and the viral titer assessed. As shown in FIG. 3, the presence of 1 mM biotin did not affect the titer of each construct tested.

Functional Viral Particles Purified from Streptactin Column

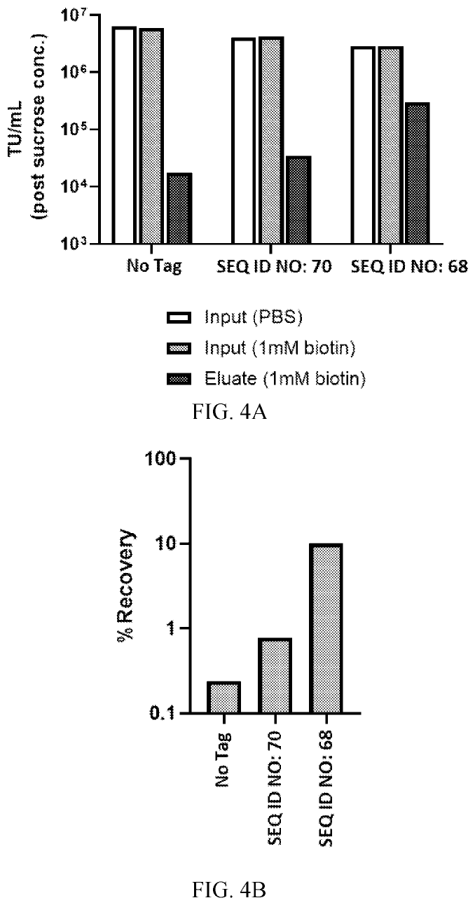

Pseudotyped lentiviral constructs harboring a Strep Tag II polypeptide in the viral glycoprotein were purified via a Strep-Tactin column. In brief, SupT1 cells were seeded in 96-well tissue culture plates with 25,000 cells per well with RPMI+10% FBS culture media. Concentrated lentiviral suspensions for each condition were diluted into three preparations: 1:23 in PBS, 1:23 in PBS with 1 mM biotin (hereafter, elution buffer), or 1:20 in PBS. The 1:20 PBS dilution for each condition was loaded to PBS-washed Strep-Tactin columns. Column-bound vector was washed with PBS then eluted with elution buffer into a clean container. The three preparations, (1) input vector in PBS, (2) input vector in elution buffer, (3) column-eluted vector in elution buffer, were each diluted in a 3-fold dilution series in culture media. Diluted vectors were then added to SupT1 cells in respective wells. Culture media was replaced 6 hours later. The treated SupT1 cells were analyzed by flow cytometry on days 4 and 7 after transduction following staining with a cell viability marker to identify live cells and an anti-CAR20 antibody to identify CAR20 positive cells. As shown in FIG. 4A and FIG. 4B, both viral constructs with a Strep Tag II polypeptide had higher recovery of functional virus off the Strep-Tactin column than an untagged control. The eluate of the untagged control generated a small fraction of CAR20+ cells, indicatin minor non-specific retention to the column prior to elution.

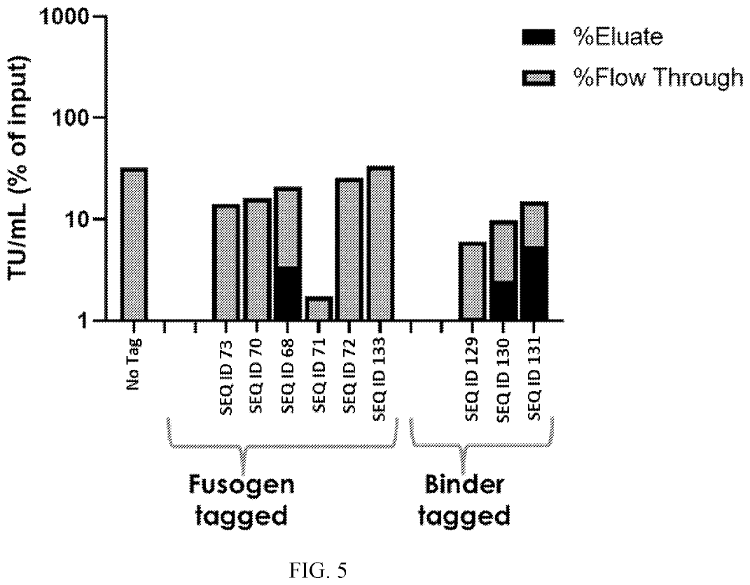

Alternate constructs of pseudotyped lentiviral constructs harboring a Strep Tag II polypeptide in the viral glycoprotein as well as those harboring a Strep Tag II polypeptide in the targeting moiety were also purified via a Strep-Tactin column. Post elution, purified viral product was tested for functional activity. As shown in FIG. 5, only one Strep Tag II: glycoprotein construct and only two Strep Tag II: targeting moiety constructs produced functional viral constructs in the eluate fraction. However, the sum of the flow through and eluate fractions did not equal the input, indicating that the viral constructs may be non-specifically sticking in the column and/or leaking from the column during wash steps. Nonetheless, inclusion of a Strep Tag II polypeptide in either a VSV-G protein or a targeting moiety as provided for herein resulted in functional viral purification.

The data from the present example illustrates that the required component of the elution buffer does not affect the viral titer and illustrates the proof of concept that functional virus may be recovered using the affinity binding polypeptide constructs provided for herein.

### Example 5: Purification of Pseudotyped Lentiviral Constructs Harboring Affinity Binding Polypeptide Using Magnetic Beads

The ability of pseudotyped lentiviral constructs harboring a Strep Tag II polypeptide in the viral glycoprotein as well as those harboring a Strep Tag II polypeptide in the targeting moiety to be purified using magnetic bead separation was examined. Viral constructs were prepared according to the methods provided for above. Post elution, purified viral product was tested for functional activity.

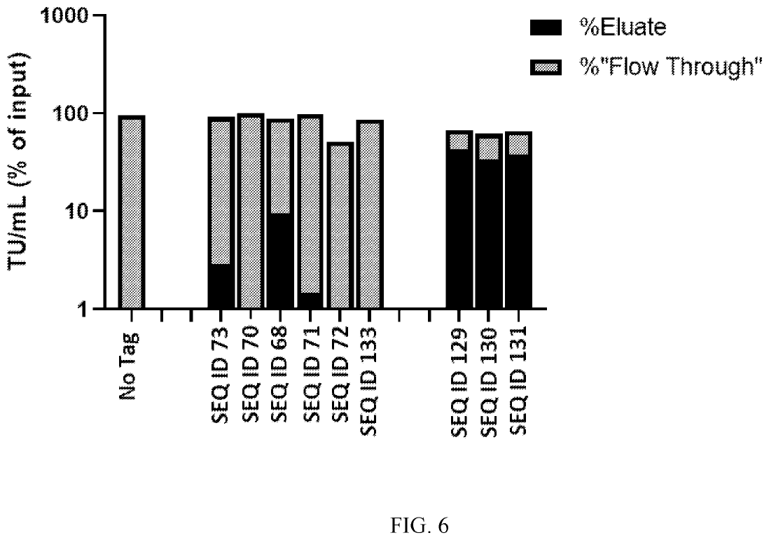

As illustrated in FIG. 6, viral recovery was more efficient using magnetic beads as compared to column purification, with all constructs tested returning a sum of eluate and flow through equal to or close to the input level. Three of six Strep Tag II:VSV-G constructs resulted in functional virus detected in the eluate, and all three Strep Tag II:targeting moiety constructs resulted in functional virus detected in the eluate. Further, the Strep Tag II:targeting moiety constructs facilitated about a 40% recovery of total input using the magnetic beads.

The data from the present example illustrates the proof of concept that functional virus may be recovered using magnetic beads. The present example also illustrates that use of magnetic beads may help reduce product loss as compared to column purification.

### Example 6: Binding Characteristics of Strep Tag II Constructs

The binding characteristics of select Strep Tag II: glycoprotein and Strep Tag II: targeting moiety constructs were assessed. In brief, concentrated lentiviral suspensions for each condition starting at 200 ng of p24 per mL were diluted in a 5-fold dilution series in TSSM buffer (Tris, NaCl, sucrose, mannitol pH 7.3). Diluted vectors were analyzed in a 96-Channel Ultra High Throughput Octet System loaded with Streptavidin (SAX) biosensors per manufacturer's recommended protocols. TSSM buffer was utilized for baseline and sample loading, while PBS with 1 mM biotin was utilized for dissociation steps.

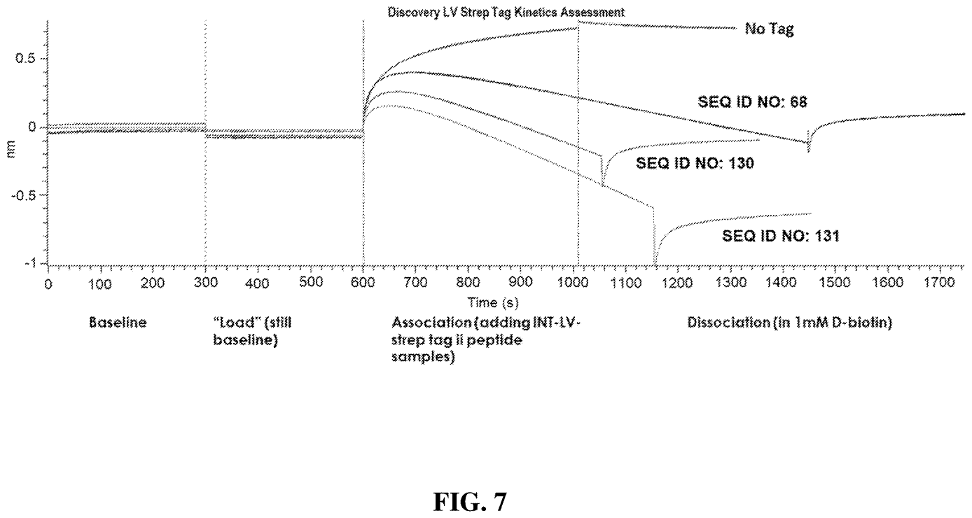

As shown in FIG. 7 and Table 8 below, the control viral construct with no Strep Tag II affinity tag showed non-specific binding to the streptavidin coated label free sensograms which was not altered by the addition of 1 mM biotin. The glycoprotein-tagged and targeting moiety tagged constructs showed binding to the streptavidin coated label free sensograms, and a corresponding sharp dissociation in response to addition of 1 mM biotin, indicating that the viral particles harboring the Strep Tag II polypeptide were binding to the streptavidin coated label free sensors. Further, for each construct the association step showed slow on-rate binding events, indicating that the Strep Tag II labeled viral particles move slowly to bind the streptavidin coated label free sensograms. The slow on-rate kinetics could be due to Strep Tag II polypeptide density on the viral surface, the size of the lentiviral particles which would naturally diffuse slower than smaller antibodies or peptides, or a combination of these and other factors.

TABLE 8

| | | Dissociation | | |
|---|---|---|---|---|
| Sample | Response | Rate (kd, 1/s) | Chi squared | R squared |
| No Tag Control | 0.697 | 4.49E−03 | 0.00157 | 0.994 |
| SEQ ID NO: 68 | −0.132 | 1.47E−02 | 0.562 | 0.786 |
| SEQ ID NO: 130 | −0.589 | 1.00E−02 | 1.733 | 0.745 |
| SEQ ID NO: 131 | −0.22 | 1.68E−02 | 1.578 | 0.654 |

The data from the present example indicates that the label free biosensor system may be used to further characterize and optimize the binding properties of Strep Tag II labeled viral particles. The data also provide further evidence that Strep Tag II labeled viral particles are able to bind to streptavidin coated particles, providing support that viral particle may be purified through affinity binding polypeptide labeling.

### Example 7: Construction of Ebola Glycoprotein:Affinity Binding Polypeptide Constructs

Pseudotyped lentiviral constructs harboring an affinity binding polypeptide in the viral glycoprotein were designed. An Ebola glycoprotein was modified to contain a Strep Tag II polypeptide at various locations at or near the N-terminus of the glycoprotein (SEQ ID NO: 26, SEQ ID NO: 27, SEQ ID NO: 28, SEQ ID NO: 137, SEQ ID NO: 147, SEQ ID NO: 148, SEQ ID NO: 151, and SEQ ID NO: 156). The constructs were designed to utilize the full length Ebola glycoprotein (SEQ ID NO: 26 and 147) to utilize an Ebola glycoprotein construct with the MLD removed (SEQ ID NO: 27 and 148), or to utilize an Ebola glycoprotein construct wherein the MLD is replaced by a targeting moiety as provided for herein (SEQ ID NO: 28, 137, 151, and 156). The Ebola constructs provided are then purified and verified in a similar manner as detailed in Examples 3-6 above.

### Example 8: Construction of Additional Ebola Glycoprotein:Affinity Binding Polypeptide Constructs

Additional pseudotyped lentiviral constructs harboring an affinity binding polypeptide in the Ebola virus glycoprotein were designed. The Ebola glycoprotein was modified to contain a Strep Tag II polypeptide within the glycoprotein, specifically by replacing all or part of the glycan cap, the mucin like domain, or both the glycan cap and mucin like domain as provided for herein. These constructs include, but are not limited to, SEQ ID NO: 135, SEQ ID NO: 136, SEQ ID NO: 149, SEQ ID NO: 150, SEQ ID NO: 152, SEQ ID NO: 153, SEQ ID NO: 154, and SEQ ID NO: 155. The Ebola glycoprotein constructs were further modified to optionally contain a targeting moiety as provided for herein. These constructs include, but are not limited to, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 188, SEQ ID NO: 189, SEQ ID NO: 190, and SEQ ID NO: 191. These constructs were rationally designed to determine whether incorporation of the affinity binding polypeptide in place of all or part of the glycan cap, the mucin like domain, or both the glycan cap and mucin like domain as provided for herein provides a viable affinity tagged construct that will not have a significant deleterious effect on the glycoprotein function. The constructs were also rationally designed to compare the inclusion of a single affinity binding polypeptide (SEQ ID NO: 135, 144, 145, 149, 153, 163, and 164) vs multiple affinity binding polypeptides (SEQ ID NO: 136, 138, 139, 140, 141, 142, 143, 150, 152, 154, 155, 157, 158, 159, 160, 161, 162, 188, 189, 190, or 191), to compare the absence (SEQ ID NO: 135, 136, 149, 150, 152, 153, 154, and 155) or presence (SEQ ID NO: 138, 139, 140, 141, 142, 143, 144, 145, 157, 158, 159, 160, 161, 162, 163, 164,188, 189, 190, and 191) of a targeting moiety in place of the MLD as provided for herein, and to compare the positional orientation of the affinity binding peptide with respect to an incorporated targeting moiety (SEQ ID NO: 142, 143, 144, 145, 161, 162, 163, and 164 with the affinity binding polypeptide(s) upstream of the targeting moiety; SEQ ID NO: 138, 139, 140, 141, 157, 158, 159, and 160 with the affinity binding polypeptide(s) downstream of the targeting moiety; or SEQ ID NO: 188, 189, 190, and 191 with the affinity binding polypeptides both upstream and downstream of the targeting moiety). The Ebola constructs provided are then purified and verified in a similar manner as detailed in Examples 3-6 above.

What is claimed is:

1. A pseudotyped viral-like particle or viral vector comprising:
an envelope comprising a recombinant virus glycoprotein, a targeting moiety for binding to a target cell, and at least a first affinity binding polypeptide, wherein the first affinity binding polypeptide is fused to the glycoprotein, the targeting moiety, or any combination thereof; and
a nucleic acid molecule encoding a heterologous molecule of interest.

2. The pseudotyped viral-like particle or viral vector of claim 1, wherein the virus glycoprotein is derived from a virus from the group consisting of Human Immunodeficiency Virus (HIV), Simian Immunodeficiency Virus (SIV), Ebola virus (EbV), Nipah Virus (NiV), Measles virus (MeV), Vesicular stomatitis virus (VSV), Spring viremia of carp virus (SVCV) or a combination thereof.

3. The pseudotyped viral-like particle or viral vector of claim 1, wherein the virus glycoprotein is HIV glycoprotein gp120, SIV glycoprotein gp120, EbV glycoprotein, NiV-G, NiV-F, MeV-H, MeV-F, VSV-G, SVCV-G, any variant thereof, or any combination thereof.

4. The pseudotyped viral-like particle or viral vector of claim 1, wherein the viral-like particle is a retroviral-like particle or retroviral vector.

5. The pseudotyped viral-like particle or viral vector of claim 4, wherein the retroviral-like particle is a lentivirus based viral particle or viral vector.

6. The pseudotyped viral-like particle or viral vector of claim 1, wherein the at least a first affinity binding polypeptide is an affinity tag selected from the group consisting of a polyhistidine tag, a polyarginine tag, a FLAG tag, a streptavidin tag, a calmodulin binding peptide, or a variant or combination thereof.

7. The pseudotyped viral-like particle or viral vector of claim 6, wherein the streptavidin tag is selected from the group consisting of a streptavidin-binding peptide, a streptavidin binding tag, a strep-tag II, a twin-strep tag, a tag of Formula I, a tag of Formula II, or a variant or combination thereof.

8. The pseudotyped viral-like particle or viral vector of claim 1, wherein the at least a first affinity binding polypeptide is a strep-tag II polypeptide sequence.

9. The pseudotyped viral-like particle or viral vector of claim 1, wherein the at least a first affinity binding polypeptide is fused to the glycoprotein.

10. The pseudotyped viral-like particle or viral vector of claim 9, wherein the at least a first affinity binding polypeptide is located at the N-terminus, at the C-terminus, or within the glycoprotein.

11. The pseudotyped viral-like particle or viral vector of claim 9, wherein the pseudotyped viral-like particle or viral vector comprises at least a second affinity binding polypeptide.

12. The pseudotyped viral-like particle or viral vector of claim 11, wherein the at least a second affinity binding polypeptide is located at the N-terminus, at the C-terminus, or within the glycoprotein.

13. The pseudotyped viral-like particle or viral vector of claim 11, wherein the pseudotyped viral-like particle or viral vector comprises at least a third, at least a fourth, or at least a fifth affinity binding polypeptide.

14. The pseudotyped viral-like particle or viral vector of claim 13, wherein each of the at least a third, the at least a fourth, or the at least a fifth affinity binding polypeptide is located, independently, at the N-terminus, at the C-terminus, or within the glycoprotein.

15. The pseudotyped viral-like particle or viral vector of claim 9, wherein the glycoprotein is Ebola glycoprotein.

16. The pseudotyped viral-like particle or viral vector of claim 15, wherein the at least a first affinity binding polypeptide is fused to the N-terminus of the Ebola glycoprotein.

17. The pseudotyped viral-like particle or viral vector of claim 15, wherein the at least a first affinity binding polypeptide replaces or is within the MLD domain of the Ebola glycoprotein.

18. The pseudotyped viral-like particle or viral vector of claim 15, wherein the Ebola glycoprotein fused to the first affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 26, SEQ ID NO: 27, SEQ ID NO: 28, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 188, SEQ ID NO: 189, SEQ ID NO: 190, or SEQ ID NO: 191.

19. The pseudotyped viral-like particle or viral vector of claim 9, wherein the glycoprotein is VSV-G glycoprotein.

20. The pseudotyped viral-like particle or viral vector of claim 19, wherein the VSV-G glycoprotein comprises an amino acid sequence having at least 85%, 86%, 87%, 88%, 89%, 90%, 91%, 92%, 93%, 94%, 95%, 96%, 97%, 98%, or 99% identity to SEQ ID NO: 37 and comprises a mutation at position 182 as compared to SEQ ID NO: 37.

21. The pseudotyped viral-like particle or viral vector of claim 19, wherein the VSV-G glycoprotein comprises an amino acid sequence of SEQ ID NO: 39, SEQ ID NO: 40, SEQ ID NO: 41, SEQ ID NO: 64, or SEQ ID NO: 65.

22. The pseudotyped viral-like particle or viral vector of claim 19, wherein the VSV-G glycoprotein fused to the first affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 68, SEQ ID NO: 69, SEQ ID NO: 70, SEQ ID NO: 71, SEQ ID NO: 72, SEQ ID NO: 73, SEQ ID NO: 132, SEQ ID NO: 133, SEQ ID NO: 167, or SEQ ID NO: 168.

23. The pseudotyped viral-like particle or viral vector of claim 9, wherein the glycoprotein is SVCV-G glycoprotein.

24. The pseudotyped viral-like particle or viral vector of claim 23, wherein the SVCV-G glycoprotein fused to the first affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 81 or SEQ ID NO: 172.

25. The pseudotyped viral-like particle or viral vector of any one of claims 1-24, wherein the engineered envelope comprises a second viral glycoprotein.

26. The pseudotyped viral-like particle or viral vector of any one of claims 1-25, wherein the targeting moiety is an scFv.

27. The pseudotyped viral-like particle or viral vector of any one of claims 1-26, wherein the targeting moiety binds to CD7, CD8, cKit (CD 117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsgl); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-la); Carbonic anhydrase IX (CA1X); Carcinoembryonic antigen (CEA); CCCTC-Binding Factor (Zinc Finger Protein)-Like (BORIS or Brother of the Regulator of Imprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECL1); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLV1-Tax; Human papilloma virus E6 (HPV E6); Human papilloma virus E7 (HPV E7); Human Telomerase reverse transcriptase (hTERT); IgE; IL13Ra2; IL1 IRa; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-I (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-I or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAPI; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member 1A (XAGE1).

28. The pseudotyped viral-like particle or viral vector of claim 27, wherein the targeting moiety binds to CD7.

29. The pseudotyped viral-like particle or viral vector of claim 28, wherein the targeting moiety comprises an amino acid sequence having at least 85% identity to SEQ ID NO: 101 or SEQ ID NO: 102.

30. The pseudotyped viral-like particle or viral vector of claim 27, wherein the targeting moiety binds to CD8.

31. The pseudotyped viral-like particle or viral vector of claim 190, wherein the targeting moiety comprises an amino acid sequence having at least 85% identity to SEQ ID NO: 119 or SEQ ID NO: 120.

32. The pseudotyped viral-like particle or viral vector of claim 1, wherein the at least a first affinity binding polypeptide is fused to the targeting moiety.

33. The pseudotyped viral-like particle or viral vector of claim 32, wherein the targeting moiety is an scFv, an antigen binding domain, a VHH, a DARPin, an adnectin, an affibody, an affilin, an affimer, an affitin, an alphabody, an anticalin, an aptamer, an armadillo repeat protein-based scaffold, an atrimer, an avimer, a fynomer, a knottin, a kunitz domain peptide, a monobody, a nanofitin, or any combination thereof.

34. The pseudotyped viral-like particle or viral vector of claim 32, wherein the targeting moiety is an scFv.

35. The pseudotyped viral-like particle or viral vector of claim 32, wherein the targeting moiety binds to CD7, CD8, cKit (CD 117), CD4, CD3, CD5, CD6, CD2, TCR alpha, TCR beta, TCR gamma, TCR delta, CD10, CD34, CD110, CD33, CD14, CD68, CCR7, CD62L, CD25, CCR2, CCR3, CCR4, CCR5, CCR6, CCR7, or CXCR3, A glycosylated CD43 epitope expressed on acute leukemia or lymphoma but not on hematopoietic progenitors; A glycosylated CD43 epitope expressed on non-hematopoietic cancers; A kinase anchor protein 4 (AKAP-4); Adrenoceptor beta 3 (ADRB3); AFP; Anaplastic lymphoma kinase (ALK); Androgen receptor; Angiopoietin-binding cell surface receptor 2 (Tie 2); Auto antibody to desmoglein 1 (Dsgl); Auto antibody to desmoglein 3 (Dsg3); B7H3 (CD276); Biotin; Bone marrow stromal cell antigen 2 (BST2); BST1/CD157; Cancer/testis antigen 1 (NY-ESO-1); Cancer/testis antigen 2 (LAGE-la); Carbonic anhydrase IX (CA1X); Carcinoembryonic antigen (CEA); CCCTC-Binding Factor (Zinc Finger Protein)-Like (BORIS or Brother of the Regulator of Imprinted Sites); CCR4; CD5; CD19; CD20; CD22; CD24; CD30; CD32 (FCGR2A); CD33; CD34; CD38; CD44v6; CD72; CD79a; CD79b; CD97; CD99; CD123; CD171; CD179a; CD179b-IGLll; CD200R; CD276/B7H3; CD300 molecule-like family member f (CD300LF); CDH1-CD324; CDH6; CDH17; CDH19; Chromosome X open reading frame 61 (CXORF61); Claudin 6 (CLDN6); Claudinl8.2 (CLD18A2 or CLDN18A.2); CMV pp65; C-MYC epitope Tag; Cripto; CS1 (also referred to as CD2 subset 1 or CRACC or SLAMF7 or CD319 or 19A24); CSF2RA (GM-CSFR-alpha); C-type lectin domain family 12 member A (CLEC12A); C-type lectin-like molecule-1 (CLL-1 or CLECL1); Cyclin B1; Cytochrome P450 IB 1 (CYP1B 1); DLL3; EBV-EBNA3c; EGF-bke module-containing mucin-like hormone receptor-like 2 (EMR2); Elongation factor 2 mutated (ELF2M); Ephrin B2; Ephrin type-A receptor 2 (EphA2); Epidermal growth factor receptor (EGFR); Epidermal growth factor receptor variant III (EGFRviii); Epithelial cell adhesion molecule (EPCAM); ERG; ETS translocation-variant gene 6 located on chromosome 12p (ETV6-AML); Fc fragment of IgA receptor (FCAR or CD89); Fc receptor-like 5 (FCRL5); Fibroblast activation protein alpha (FAP); FITC; Fms Like Tyrosine Kinase 3 (FLT3); Folate receptor alpha (FRa or FR1); Folate receptor beta (FRb); Follicle stimulating hormone receptor (FSHR); Fos-related antigen 1; Fucosyl-GMl; G protein coupled receptor class C group 5 member D (GPRC5D); G protein-coupled receptor 20 (GPR20); GAD; Ganglioside G2 (GD2); Ganglioside GD3 (aNeu5Ac(2-8)aNeu5Ac(2-3)bDGalp(1-4)bDGlcp(1-1)Cer); Ganglioside GM3 (aNeu5Ac(2-3)bDClalp(1-4)bDGlcp(1-1)Cer); GD3; GFRalpha4; Glycoprotein 100 (gplOO); Glypican-3 (GPC3); Gonadotropin Hormone receptor (CGHR or GR); GpA33; GpNMB; GPRC5D; Guanylyl cyclase C (GCC); Heat shock protein 70-2 mutated (mut hsp70-2); Hepatitis A virus cellular receptor 1 (HAVCR1); Hexasaccharide portion of globoH glycoceramide (GloboH); High molecular weight-melanoma associated antigen (HMWMAA); HIV1 envelope glycoprotein; HLA; HLA-DOA; HLA-A; HLA-A2; HLA-B; HLA-C; HLA-DM; HLA-DOB; HLA-DP; HLA-DQ; HLA-DR; HLA-G; HTLV1-Tax; Human papilloma virus E6 (HPV E6); Human papilloma virus E7 (HPV E7); Human Telomerase reverse transcriptase (hTERT); IgE; IL13Ra2; IL1 1Ra; Immunoglobulin lambda-like polypeptide 1 (IGLL1); Influenza A hemagglutinin (HA); Insulin-like growth factor 1 receptor (IGF-I receptor); Interleukin 11 receptor alpha (IL-llRa); Interleukin-13 receptor subunit alpha-2 (IL-13Ra2 or CD213A2); Intestinal carboxyl esterase; KIT (CD117); KSHV K8.1; KSHV-gH; LAMP1; Legumain; Leukocyte immunoglobulin-like receptor subfamily A member 2 (LILRA2); Leukocyte-associated immunoglobulin-like receptor 1 (LAIR1); Leutenizing hormone receptor (LHR); Lewis(Y) antigen; Lews Ag; Livl; Locus K 9 (LY6K); Low conductance chloride channel; Lymphocyte antigen 6 complex; Lymphocyte antigen 75 (LY75); Lymphocyte-specific protein tyrosine kinase (LCK); Mammary gland differentiation antigen (NY-BR-1); Melanoma antigen recognized by T cells 1 (MelanA or MARTI); Melanoma-associated antigen 1 (MAGE-A1); Melanoma cancer testis antigen-I (MAD-CT-1); Melanoma cancer testis antigen-2 (MAD-CT-2); Melanoma inhibitor of apoptosis (ML-IAP); Mesothelin; MPL; Mucin 1 cell surface associated (MUC1); N-Acetyl glucosaminyl-transferase V (NA17); Nectin-4; Neural cell adhesion molecule (NCAM); NKG2D; NYBR1; O-acetyl-GD2 ganglioside (OAcGD2); Olfactory receptor 51E2 (OR51E2); Oncogene fusion protein consisting of breakpoint cluster region (BCR) and Abelson murine leukemia viral oncogene homolog 1 (Abl) (bcr-abl); P53 mutant; Paired box protein Pax-3 (PAX3); Paired box protein Pax-5 (PAX5); Pannexin 3 (PANX3); PDL1; P-glycoprotein; Placenta-specific 1 (PLAC1); Platelet-derived growth factor receptor beta (PDGFR-beta); Polysialic acid; Proacrosin binding protein sp32 (OY-TES1); Prostase; Prostate carcinoma tumor antigen-1 (PCT A-1 or Galectin 8); Prostate stem cell antigen (PSCA); Prostate-specific membrane antigen (PSMA); Prostatic acid phosphatase (PAP); Prostein; Protease Serine 21 (Testisin or PRSS21); Proteasome (Prosome Macropain) Subunit Beta Type 9 (LMP2); PTK7; Ras G12V; Ras Homolog Family Member C (RhoC); Rat sarcoma (Ras) mutant; Receptor for Advanced Gly cation Endproducts (RAGE-1); Receptor tyrosine kinase-like orphan receptor 1 (ROR1); Receptor tyrosine-protein kinase ERBB2 or Her-22/neu; Renal ubiquitous 1 (RU1); Renal ubiquitous 2 (RU2); Sarcoma translocation breakpoints; Serine 2 (TMPRSS2) ETS fusion gene; Sialyl Lewis adhesion molecule (sLe); SLAMF4; SLAMF6; Slea (CA19.9 or Sialyl Lewis Antigen); Sperm protein 17 (SPA17); Squamous Cell Carcinoma Antigen Recognized By T Cells 3 (SART3); Stage-specific embryonic antigen-4 (SSEA-4); STEAPI; Survivin; Synovial sarcoma X breakpoint 2 (SSX2); TCR Gamma Alternate Reading Frame Protein (TARP); TCR-beta1 chain; TCR-beta2 chain; TCR-delta chain; TCR-gamma chain; TCRgamma-delta; Telomerase; TGFbetaR2; The antigen recognized by TNT antibody; Thyroid stimulating hormone receptor (TSHR); Timl-/HVCR1; Tissue Factor 1 (TF1); Tn ag; Tn antigen ((Tn Ag) or (GalNAca-Ser/Thr)); TNF receptor family member B cell maturation (BCMA); Transglutaminase 5 (TGS5); Transmembrane protease; TROP2; Tumor endothelial marker 1 (TEM1/CD248); Tumor endothelial marker 7-related (TEM7R); Tumor protein p53 (p53); Tumor-associated glycoprotein 72 (TAG72); Tyrosinase; Tyrosinase-related protein 2 (TRP-2); Uroplakin 2 (UPK2); Vascular endothelial growth factor receptor 2 (VEGFR2); V-myc avian myelocytomatosis viral oncogene neuroblastoma derived homolog (MYCN); Wilms tumor protein (WT1); or X Antigen Family Member 1A (XAGE1).

36. The pseudotyped viral-like particle or viral vector of claim 35, wherein the targeting moiety binds to CD7.

37. The pseudotyped viral-like particle or viral vector of claim 36, wherein the targeting moiety comprises an amino acid sequence having at least 90%, at least 95%, at least 98%, or 100% identity to SEQ ID NO: 101 or SEQ ID NO: 102.

38. The pseudotyped viral-like particle or viral vector of claim 35, wherein the targeting moiety binds to CD8.

39. The pseudotyped viral-like particle or viral vector of claim 38, wherein the targeting moiety comprises an amino acid sequence having at least 90%, at least 95%, at least 98%, or 100% identity to SEQ ID NO: 119 or SEQ ID NO: 120.

40. The pseudotyped viral-like particle or viral vector of any one of claims 32-39, wherein the at least a first affinity binding polypeptide is located at the N-terminus, at the C-terminus, or within the targeting moiety.

41. The pseudotyped viral-like particle or viral vector of claim 40, wherein the targeting moiety fused to the first affinity binding polypeptide comprises an amino acid sequence having at least 85%, 86%, 87%, 88%, 89%, 90%, 91%, 92%, 93%, 94%, 95%, 96%, 97%, 98%, 99%, or 100% identity to SEQ ID NO: 121, SEQ ID NO: 126, SEQ ID NO: 129, SEQ ID NO: 130, SEQ ID NO: 131, SEQ ID NO: 174, SEQ ID NO: 175, SEQ ID NO: 176, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, or SEQ ID NO: 187.

42. The pseudotyped viral-like particle or viral vector of any one of claims 32-41, wherein the pseudotyped viral-like particle or viral vector further comprises at least a second affinity binding polypeptide.

43. The pseudotyped viral-like particle or viral vector of any one of claims 32-42, wherein the virus glycoprotein comprises an amino acid sequence as set forth in SEQ ID NO: 25, SEQ ID NO: 29, SEQ ID NO: 30, SEQ ID NO: 33, SEQ ID NO: 34, SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 66, SEQ ID NO: 67, SEQ ID NO: 74; SEQ ID NO: 77, SEQ ID NO: 79, or SEQ ID NO: 80.

44. A pseudotyped viral-like particle or viral vector comprising:
an engineered envelope comprising a recombinant virus glycoprotein, an engineered targeting moiety for binding to a target cell, and at least a first affinity binding polypeptide, wherein the at least a first affinity binding polypeptide is fused to the glycoprotein, and the glycoprotein comprises an amino acid sequence as set forth in SEQ ID NO: 25, SEQ ID NO: 29, SEQ ID NO: 30, SEQ ID NO: 33, SEQ ID NO: 34, SEQ ID NO: 36, SEQ ID NO: 37, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 58, SEQ ID NO: 59, SEQ ID NO: 61, SEQ ID NO: 62, SEQ ID NO: 64, SEQ ID NO: 65, SEQ ID NO: 66, SEQ ID NO: 67, SEQ ID NO: 74; SEQ ID NO: 77, SEQ ID NO: 79, or SEQ ID NO: 80; and
a nucleic acid encoding a heterologous molecule of interest.

45. A pseudotyped viral-like particle or viral vector comprising:
an engineered envelope comprising a recombinant virus glycoprotein, an engineered targeting moiety for binding to a target cell, and at least a first affinity binding polypeptide, wherein the at least a first affinity binding polypeptide is fused to the glycoprotein, and the at least a first affinity binding polypeptide fused to the glycoprotein comprises an amino acid sequence as set forth in SEQ ID NO: 26, SEQ ID NO: 27, SEQ ID NO: 28, SEQ ID NO: 31, SEQ ID NO: 32, SEQ ID NO: 35; SEQ ID NO: 68, SEQ ID NO: 69, SEQ ID NO: 70, SEQ ID NO: 71, SEQ ID NO: 72, SEQ ID NO: 73, SEQ ID NO: 75, SEQ ID NO: 76, SEQ ID NO: 78, SEQ ID NO: 81, SEQ ID NO: 132, SEQ ID NO: 133, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 166, SEQ ID NO: 167, SEQ ID NO: 168, SEQ ID NO: 170, SEQ ID NO: 172, SEQ ID NO: 188, SEQ ID NO: 189, SEQ ID NO: 190, or SEQ ID NO: 191; and
a nucleic acid encoding a heterologous molecule of interest.

46. A pseudotyped viral-like particle or viral vector comprising:
an engineered envelope comprising a recombinant virus glycoprotein, an engineered targeting moiety for binding to a target cell, and at least a first affinity binding polypeptide, wherein the targeting moiety is an scFv comprising an amino acid sequence of SEQ ID NO: 101, SEQ ID NO: 102, SEQ ID NO: 119 or SEQ ID NO: 120, and the at least a first affinity binding polypeptide is fused to the targeting moiety; and
a nucleic acid encoding a heterologous molecule of interest.

47. A pseudotyped viral-like particle or viral vector comprising:
an engineered envelope comprising a recombinant virus glycoprotein, an engineered targeting moiety for binding to a target cell, and at least a first affinity binding polypeptide, wherein the targeting moiety is an scFv, and wherein the at least a first affinity binding polypeptide is fused to the targeting moiety and comprises an amino acid sequence of SEQ ID NO: 121, SEQ ID NO: 126, SEQ ID NO: 129, SEQ ID NO: 130, SEQ ID NO: 131, SEQ ID NO: 174, SEQ ID NO: 175, SEQ ID NO: 176, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, or SEQ ID NO: 187; and
a nucleic acid encoding a heterologous molecule of interest.

48. A pseudotyped viral-like particle or viral vector comprising:
an engineered envelope comprising a recombinant virus glycoprotein, an engineered targeting moiety for binding to a target cell, and at least two affinity binding polypeptides,
wherein the recombinant virus glycoprotein is fused to a first affinity binding polypeptide and comprises an amino acid sequence of SEQ ID NO: 26, SEQ ID NO: 27, SEQ ID NO: 28, SEQ ID NO: 31, SEQ ID NO: 32, SEQ ID NO: 35; SEQ ID NO: 68, SEQ ID NO: 69, SEQ ID NO: 70, SEQ ID NO: 71, SEQ ID NO: 72, SEQ ID NO: 73, SEQ ID NO: 75, SEQ ID NO: 76, SEQ ID NO: 78, SEQ ID NO: 81, SEQ ID NO: 132, SEQ ID NO: 133, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 166, SEQ ID NO: 167, SEQ ID NO: 168, SEQ ID NO: 170, SEQ ID NO: 172, SEQ ID NO: 188, SEQ ID NO: 189, SEQ ID NO: 190, or SEQ ID NO: 191,
wherein the targeting moiety is an scFv,
wherein the targeting moiety is fused to a second affinity binding polypeptide and comprises an amino acid sequence of SEQ ID NO: 121, SEQ ID NO: 126, SEQ ID NO: 129, SEQ ID NO: 130, SEQ ID NO: 131, SEQ ID NO: 174, SEQ ID NO: 175, SEQ ID NO: 176, SEQ ID NO: 178, SEQ ID NO: 179, SEQ ID NO: 180, SEQ ID NO: 181, SEQ ID NO: 182, SEQ ID NO: 183, SEQ ID NO: 184, SEQ ID NO: 185, SEQ ID NO: 186, or SEQ ID NO: 187; and
a nucleic acid encoding a heterologous molecule of interest.

49. The pseudotyped viral-like particle or viral vector of any one of claims 1-48, wherein the heterologous molecule of interest is a chimeric antigen receptor (“CAR”).

50. A method of purifying a pseudotyped viral-like particle or viral vector, the method comprising transfecting or transducing a packaging cell line with a nucleic acid molecule encoding for a recombinant virus glycoprotein, an engineered targeting moiety for binding to a target cell, and at least a first affinity binding polypeptide, wherein the first affinity binding polypeptide is fused to the glycoprotein, the targeting moiety, or any combination thereof; culturing the packaging cell line under conditions sufficient to produce the pseudotyped viral-like particle or viral vector; isolating the pseudotyped viral-like particle or viral vector; and purifying the pseudotyped viral-like particle or viral vector via use of the first affinity binding polypeptide included in the glycoprotein, the targeting moiety, or both.

51. The method of any one of claim 50, wherein the pseudotyped viral-like particle or viral vector comprises the pseudotyped viral-like particle or viral vector of any one of claims 1-49.


SEQUENCE LISTING (199 sequences)

SEQ ID NO: 1 - protein, 8 aa, synthetic construct
WSHPQFEK

SEQ ID NO: 2 - protein, 5 aa, synthetic construct
GGGGS

SEQ ID NO: 3 - protein, 15 aa, synthetic construct
GGGGSGGGGSGGGGS

SEQ ID NO: 4 - protein, 5 aa, synthetic construct
  REPEAT 1..5: note = Sequence may be present n times, wherein n is 1, 2, 3, or 4
GGGGS

SEQ ID NO: 5 - protein, 8 aa, synthetic construct
GGGGGGGG

SEQ ID NO: 6 - protein, 6 aa, synthetic construct
GGGGGG

SEQ ID NO: 7 - protein, 15 aa, synthetic construct
EAAAKEAAAKEAAAK

SEQ ID NO: 8 - protein, 4 aa, synthetic construct
  REPEAT 1..4: note = Sequence may be present n times, wherein n is 1, 2, or 3
EAAK

SEQ ID NO: 9 - protein, 46 aa, synthetic construct
AEAAAKEAAAKEAAAKEAAAKALEAEAAAKEAAAKEAAAKEAAAKA

SEQ ID NO: 10 - protein, 12 aa, synthetic construct
AEAAAKEAAAKA

SEQ ID NO: 11 - protein, 5 aa, synthetic construct
PAPAP

SEQ ID NO: 12 - protein, 4 aa, synthetic construct
  REPEAT 1..2: note = The sequence AP may be present n times, wherein n is an integer from 5 to 17, for a minimum sequence length of 10 and a maximum sequence length of 34
APAP

SEQ ID NO: 13 - protein, 17 aa, synthetic construct
VSQTSKLTRAETVFPDV

SEQ ID NO: 14 - protein, 6 aa, synthetic construct
PLGLWA

SEQ ID NO: 15 - protein, 6 aa, synthetic construct
RVLAEA

SEQ ID NO: 16 - protein, 10 aa, synthetic construct
EDVVCCSMSY

SEQ ID NO: 17 - protein, 8 aa, synthetic construct
GGIEGRGS

SEQ ID NO: 18 - protein, 10 aa, synthetic construct
TRHRQPRGWE

SEQ ID NO: 19 - protein, 10 aa, synthetic construct
AGNRVRRSVG

SEQ ID NO: 20 - protein, 9 aa, synthetic construct
RRRRRRRRR

SEQ ID NO: 21 - protein, 4 aa, synthetic construct
GFLG

SEQ ID NO: 22 - protein, 4 aa, synthetic construct
GGGS

SEQ ID NO: 23 - protein, 4 aa, synthetic construct
GGSA

SEQ ID NO: 24 - protein, 28 aa, synthetic construct
WSHPQFEKGGGSGGGSGGSAWSHPQFEK

SEQ ID NO: 25 - protein, 676 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 26 - protein, 687 aa, synthetic construct
MGVTGILQLPRDRFWSHPQFEKGGSKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 27 - protein, 511 aa, synthetic construct
MGVTGILQLPRDRFWSHPQFEKGGSKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 28 - protein, 796 aa, synthetic construct
MGVTGILQLPRDRFWSHPQFEKGGSKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESAGGGGSGGGGSGGGGSASQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRTGGGGGSGGGGSGGGGSSANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 29 - protein, 602 aa, synthetic construct
MPTESKKVRFENTASDKGKNPSKVIKSYYGTMDIKKINEGLLDSKILSAFNTVIALLGSIVIIVMNIMIIQNYTRSTDNQAMIKDALQSIQQQIKGLADKIGTEIGPKVSLIDTSSTITIPANIGLLGSKISQSTASINENVNEKCKFTLPPLKIHECNISCPNPLPFREYKPQTEGVSNLVGLPNNICLQKTSNQILKPKLISYTLPVVGQSGTCITDPLLAMDEGYFAYSHLEKIGSCSRGVSKQRIIGVGEVLDRGDEVPSLFMTNVWTPSNPNTVYHCSAVYNNEFYYVLCAVSVVGDPILNSTYWSGSLMMTRLAVKPKNNGESYNQHQFALRNIEKGKYDKVMPYGPSGIKQGDTLYFPAVGFLVRTEFTYNDSNCPIAECQYSKPENCRLSMGIRPNSHYILRSGLLKYNLSDEENSKIVFIEISDQRLSIGSPSKIYDSLGQPVFYQASFSWDTMIKFGDVQTVNPLVVNWRDNTVISRPGQSQCPRFNKCPEVCWEGVYNDAFLIDRINWISAGVFLDSNQTAENPVFTVFKDNEVLYRAQLASEDTNAQKTITNCFLLKNKIWCISLVEIYDTGDNVIRPKLFAVKIPEQCT

SEQ ID NO: 30 - protein, 569 aa, synthetic construct
MKKINEGLLDSKILSAFNTVIALLGSIVIIVMNIMIIQNYTRSTDNQAMIKDALQSIQQQIKGLADKIGTEIGPKVSLIDTSSTITIPANIGLLGSKISQSTASINENVNEKCKFTLPPLKIHECNISCPNPLPFREYKPQTEGVSNLVGLPNNICLQKTSNQILKPKLISYTLPVVGQSGTCITDPLLAMDEGYFAYSHLEKIGSCSRGVSKQRIIGVGEVLDRGDEVPSLFMTNVWTPSNPNTVYHCSAVYNNEFYYVLCAVSVVGDPILNSTYWSGSLMMTRLAVKPKNNGESYNQHQFALRNIEKGKYDKVMPYGPSGIKQGDTLYFPAVGFLVRTEFTYNDSNCPIAECQYSKPENCRLSMGIRPNSHYILRSGLLKYNLSDEENSKIVFIEISDQRLSIGSPSKIYDSLGQPVFYQASFSWDTMIKFGDVQTVNPLVVNWRDNTVISRPGQSQCPRFNKCPEVCWEGVYNDAFLIDRINWISAGVFLDSNQTAENPVFTVFKDNEVLYRAQLASEDTNAQKTITNCFLLKNKIWCISLVEIYDTGDNVIRPKLFAVKIPEQCT

SEQ ID NO: 31 - protein, 580 aa, synthetic construct
MKKINEGLLDSKILSAFNTVIALLGSIVIIVMNIMIIQNYTRSTDNQAMIKDALQSIQQQIKGLADKIGTEIGPKVSLIDTSSTITIPANIGLLGSKISQSTASINENVNEKCKFTLPPLKIHECNISCPNPLPFREYKPQTEGVSNLVGLPNNICLQKTSNQILKPKLISYTLPVVGQSGTCITDPLLAMDEGYFAYSHLEKIGSCSRGVSKQRIIGVGEVLDRGDEVPSLFMTNVWTPSNPNTVYHCSAVYNNEFYYVLCAVSVVGDPILNSTYWSGSLMMTRLAVKPKNNGESYNQHQFALRNIEKGKYDKVMPYGPSGIKQGDTLYFPAVGFLVRTEFTYNDSNCPIAECQYSKPENCRLSMGIRPNSHYILRSGLLKYNLSDEENSKIVFIEISDQRLSIGSPSKIYDSLGQPVFYQASFSWDTMIKFGDVQTVNPLVVNWRDNTVISRPGQSQCPRFNKCPVVCAEGVYNDAFLIDRINWISAGVFLDSNATAANPVFTVFKDNEVLYRAQLASEDTNAQKTITNCFLLKNKIWCISLVEIYDTGDNVIRPKLFAVKIPEQCTGGSWSHPQFEK

SEQ ID NO: 32 - protein, 851 aa, synthetic construct
MKKINEGLLDSKILSAFNTVIALLGSIVIIVMNIMIIQNYTRSTDNQAMIKDALQSIQQQIKGLADKIGTEIGPKVSLIDTSSTITIPANIGLLGSKISQSTASINENVNEKCKFTLPPLKIHECNISCPNPLPFREYKPQTEGVSNLVGLPNNICLQKTSNQILKPKLISYTLPVVGQSGTCITDPLLAMDEGYFAYSHLEKIGSCSRGVSKQRIIGVGEVLDRGDEVPSLFMTNVWTPSNPNTVYHCSAVYNNEFYYVLCAVSVVGDPILNSTYWSGSLMMTRLAVKPKNNGESYNQHQFALRNIEKGKYDKVMPYGPSGIKQGDTLYFPAVGFLVRTEFTYNDSNCPIAECQYSKPENCRLSMGIRPNSHYILRSGLLKYNLSDEENSKIVFIEISDQRLSIGSPSKIYDSLGQPVFYQASFSWDTMIKFGDVQTVNPLVVNWRDNTVISRPGQSQCPRFNKCPVVCAEGVYNDAFLIDRINWISAGVFLDSNATAANPVFTVFKDNEVLYRAQLASEDTNAQKTITNCFLLKNKIWCISLVEIYDTGDNVIRPKLFAVKIPEQCTDIGGGGSGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGSWSHPQFEK

SEQ ID NO: 33 - protein, 546 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 34 - protein, 524 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 35 - protein, 535 aa, synthetic construct
MVVILDKRCYCNLLILILMISECSVGWSHPQFEKGGSILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 36 - protein, 511 aa, synthetic construct
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 37 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 38 - protein, 16 aa, synthetic construct
MKCLLYLAFLFIGVNC

SEQ ID NO: 39 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLASMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 40 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLDSMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 41 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 42 - protein, 495 aa, synthetic construct
KFTIVFPANQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 43 - protein, 495 aa, synthetic construct
KFTIVFPHNAKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 44 - protein, 495 aa, synthetic construct
KFTIVFPHNRKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 45 - protein, 495 aa, synthetic construct
KFTIVFPHNKKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 46 - protein, 517 aa, synthetic construct
MLSYLIFALVVSPILGKIEIVFPQHTTGDWKRVPHEYNYCPTSADKNSHGTQTGIPVELTMPKGLTTHQVDGFMCHSALWMTTCDFRWYGPKYITHSIHNEEPTDYQCLEAIKAYKDGVSFNPGFPPQSCGYGTVTDAEAHIVTVTPHSVKVDEYTGEWIDPHFIGGRCKGQICETVHNSTKWFTSSDGESVCSQLFTLVGGTFFSDSEEITSMGLPETGIRSNYFPYVSTEGICKMPFCRKPGYKLKNDLWFQITDPDLDKTVRDLPHIKDCDLSSSIVTPGEHATDISLISDVERILDYALCQNTWSKIEAGEPITPVDLSYLGPKNPGAGPVFTIINGSLHYFMSKYLRVELESPVIPRMEGKVAGTRIVRQLWDQWFPFGEVEIGPNGVLKTKQGYKFPLHIIGTGEVDNDIKMERIVKHWEHPHIEAAQTFLKKDDTEEVLYYGDTGVSKNPVELVEGWFSGWRSSIMGVLAVIIGFVILIFLIRLIGVLSSLFRQKRRPIYKSDVEMAHFR

SEQ ID NO: 47 - protein, 501 aa, synthetic construct
KIEIVFPQHTTGDWKRVPHEYNYCPTSADKNSHGTQTGIPVELTMPKGLTTHQVDGFMCHSALWMTTCDFRWYGPKYITHSIHNEEPTDYQCLEAIKAYKDGVSFNPGFPPQSCGYGTVTDAEAHIVTVTPHSVKVDEYTGEWIDPHFIGGRCKGQICETVHNSTKWFTSSDGESVCSQLFTLVGGTFFSDSEEITSMGLPETGIRSNYFPYVSTEGICKMPFCRKPGYKLKNDLWFQITDPDLDKTVRDLPHIKDCDLSSSIVTPGEHATDISLISDVERILDYALCQNTWSKIEAGEPITPVDLSYLGPKNPGAGPVFTIINGSLHYFMSKYLRVELESPVIPRMEGKVAGTRIVRQLWDQWFPFGEVEIGPNGVLKTKQGYKFPLHIIGTGEVDNDIKMERIVKHWEHPHIEAAQTFLKKDDTEEVLYYGDTGVSKNPVELVEGWFSGWRSSIMGVLAVIIGFVILIFLIRLIGVLSSLFRQKRRPIYKSDVEMAHFR

SEQ ID NO: 48 - protein, 16 aa, synthetic construct
MLSYLIFALVVSPILG

SEQ ID NO: 49 - protein, 512 aa, synthetic construct
MLRLFLFCFLALGAHSKFTIVFPHHQKGNWKNVPSTYHYCPSSSDQNWHNDLTGVSLHVKIPKSHKAIQADGWMCHAAKWVTTCDFRWYGPKYITHSIHSMSPTLEQCKTSIEQTKQGVWINPGFPPQSCGYATVTDAEVVVVQATPHHVLVDEYTGEWIDSQLVGGKCSKEVCQTVHNSTVWHADYKITGLCESNLASVDITFFSEDGQKTSLGKPNTGFRSNHFAYESGEKACRMQYCTQWGIRLPSGVWFELVDKDLFQAAKLPECPRGSSISAPSQTSVDVSLIQDVERILDYSLCQETWSKIRAKLPVSPVDLSYLAPKNPGSGPAFTIINGTLKYFETRYIRVDISNPIIPHMVGTMSGTTTERELWNDWYPYEDVEIGPNGVLKTPTGFKFPLYMIGHGMLDSDLHKSSQAQVFEHPHAKDAASQLPDDETLFFGDTGLSKNPVELVEGWFSSWKSTLASFFLIIGLGVALIFIIRIIVAIRYKYKGRKTQKIYNDVEMSRLGNK

SEQ ID NO: 50 - protein, 496 aa, synthetic construct
KFTIVFPHHQKGNWKNVPSTYHYCPSSSDQNWHNDLTGVSLHVKIPKSHKAIQADGWMCHAAKWVTTCDFRWYGPKYITHSIHSMSPTLEQCKTSIEQTKQGVWINPGFPPQSCGYATVTDAEVVVVQATPHHVLVDEYTGEWIDSQLVGGKCSKEVCQTVHNSTVWHADYKITGLCESNLASVDITFFSEDGQKTSLGKPNTGFRSNHFAYESGEKACRMQYCTQWGIRLPSGVWFELVDKDLFQAAKLPECPRGSSISAPSQTSVDVSLIQDVERILDYSLCQETWSKIRAKLPVSPVDLSYLAPKNPGSGPAFTIINGTLKYFETRYIRVDISNPIIPHMVGTMSGTTTERELWNDWYPYEDVEIGPNGVLKTPTGFKFPLYMIGHGMLDSDLHKSSQAQVFEHPHAKDAASQLPDDETLFFGDTGLSKNPVELVEGWFSSWKSTLASFFLIIGLGVALIFIIRIIVAIRYKYKGRKTQKIYNDVEMSRLGNK

SEQ ID NO: 51 - protein, 16 aa, synthetic construct
MLRLFLFCFLALGAHS

SEQ ID NO: 52 - protein, 523 aa, synthetic construct
MKMKMVIAGLILCIGILPAIGKITISFPQSLKGDWRPVPKGYNYCPTSADKNLHGDLIDIGLRLRAPKSFKGISADGWMCHAARWITTCDFRWYGPKYITHSIHSFRPSNDQCKEAIRLTNEGNWINPGFPPQSCGYASVTDSESVVVTVTKHQVLVDEYSGSWIDSQFPGGSCTSPICDTVHNSTLWHADHTLDSICDQEFVAMDAVLFTESGKFEEFGKPNSGIRSNYFPYESLKDVCQMDFCKRKGFKLPSGVWFEIEDAEKSHKAQVELKIKRCPHGAVISAPNQNAADINLIMDVERILDYSLCQATWSKIQNKEALTPIDISYLGPKNPGPGPAFTIINGTLHYFNTRYIRVDIAGPVTKEITGFVSGTSTSRVLWDQWFPYGENSIGPNGLLKTASGYKYPLFMVGTGVLDADIHKLGEATVIEHPHAKEAQKVVDDSEVIFFGDTGVSKNPVEVVEGWFSGWRSSLMSIFGIILLIVCLVLIVRILIALKYCCVRHKKRTIYKEDLEMGRIPRRA

SEQ ID NO: 53 - protein, 502 aa, synthetic construct
KITISFPQSLKGDWRPVPKGYNYCPTSADKNLHGDLIDIGLRLRAPKSFKGISADGWMCHAARWITTCDFRWYGPKYITHSIHSFRPSNDQCKEAIRLTNEGNWINPGFPPQSCGYASVTDSESVVVTVTKHQVLVDEYSGSWIDSQFPGGSCTSPICDTVHNSTLWHADHTLDSICDQEFVAMDAVLFTESGKFEEFGKPNSGIRSNYFPYESLKDVCQMDFCKRKGFKLPSGVWFEIEDAEKSHKAQVELKIKRCPHGAVISAPNQNAADINLIMDVERILDYSLCQATWSKIQNKEALTPIDISYLGPKNPGPGPAFTIINGTLHYFNTRYIRVDIAGPVTKEITGFVSGTSTSRVLWDQWFPYGENSIGPNGLLKTASGYKYPLFMVGTGVLDADIHKLGEATVIEHPHAKEAQKVVDDSEVIFFGDTGVSKNPVEVVEGWFSGWRSSLMSIFGIILLIVCLVLIVRILIALKYCCVRHKKRTIYKEDLEMGRIPRRA

SEQ ID NO: 54 - protein, 21 aa, synthetic construct
MKMKMVIAGLILCIGILPAIG

SEQ ID NO: 55 - protein, 511 aa, synthetic construct
MTPAFILCMLLAGSSWAKFTIVFPQSQKGDWKDVPPNYRYCPSSADQNWHGDLLGVNIRAKMPKVHKAIKADGWMCHAAKWVTTCDYRWYGPQYITHSIHSFIPTKAQCEESIKQTKEGVWINPGFPPKNCGYASVSDAESIIVQATAHSVMIDEYSGDWLDSQFPTGRCTGSTCETIHNSTLWYADYQVTGLCDSALVSTEVTFYSEDGLMTSIGRQNTGYRSNYFPYEKGAAACRMKYCTHEGIRLPSGVWFEMVDKELLESVQMPECPAGLTISAPTQTSVDVSLILDVERMLDYSLCQETWSKVHSGLPISPVDLGYIAPKNPGAGPAFTIVNGTLKYFDTRYLRIDIEGPVLKKMTGKVSGTPTKRELWTEWFPYDDVEIGPNGVLKTPEGYKFPLYMIGHGLLDSDLQKTSQAEVFHHPQIAEAVQKLPDDETLFFGDTGISKNPVEVIEGWFSNWRSSVMAIVFAILLLVITVLMVRLCVAFRHFCCQKRHKIYNDLEMNQLRR

SEQ ID NO: 56 - protein, 494 aa, synthetic construct
KFTIVFPQSQKGDWKDVPPNYRYCPSSADQNWHGDLLGVNIRAKMPKVHKAIKADGWMCHAAKWVTTCDYRWYGPQYITHSIHSFIPTKAQCEESIKQTKEGVWINPGFPPKNCGYASVSDAESIIVQATAHSVMIDEYSGDWLDSQFPTGRCTGSTCETIHNSTLWYADYQVTGLCDSALVSTEVTFYSEDGLMTSIGRQNTGYRSNYFPYEKGAAACRMKYCTHEGIRLPSGVWFEMVDKELLESVQMPECPAGLTISAPTQTSVDVSLILDVERMLDYSLCQETWSKVHSGLPISPVDLGYIAPKNPGAGPAFTIVNGTLKYFDTRYLRIDIEGPVLKKMTGKVSGTPTKRELWTEWFPYDDVEIGPNGVLKTPEGYKFPLYMIGHGLLDSDLQKTSQAEVFHHPQIAEAVQKLPDDETLFFGDTGISKNPVEVIEGWFSNWRSSVMAIVFAILLLVITVLMVRLCVAFRHFCCQKRHKIYNDLEMNQLRR

SEQ ID NO: 57 - protein, 17 aa, synthetic construct
MTPAFILCMLLAGSSWA

SEQ ID NO: 58 - protein, 512 aa, synthetic construct
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 59 - protein, 495 aa, synthetic construct
KFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 60 - protein, 17 aa, synthetic construct
MNFLLLTFIVLPLCSHA

SEQ ID NO: 61 - protein, 513 aa, synthetic construct
MLVLYLLLSLLALGAQCKFTIVFPHNQKGNWKNVPANYQYCPSSSDLNWHNGLIGTSLQVKMPKSHKAIQADGWMCHAAKWVTTCDFRWYGPKYVTHSIKSMIPTVDQCKESIAQTKQGTWLNPGFPPQSCGYASVTDAEAVIVKATPHQVLVDEYTGEWVDSQFPTGKCNKDICPTVHNSTTWHSDYKVTGLCDANLISMDITFFSEDGKLTSLGKEGTGFRSNYFAYENGDKACRMQYCKHWGVRLPSGVWFEMADKDIYNDAKFPDCPEGSSIAAPSQTSVDVSLIQDVERILDYSLCQETWSKIRAHLPISPVDLSYLSPKNPGTGPAFTIINGTLKYFETRYIRVDIAGPIIPQMRGVISGTTTERELWTDWYPYEDVEIGPNGVLKTATGYKFPLYMIGHGMLDSDLHISSKAQVFEHPHIQDAASQLPDDETLFFGDTGLSKNPIELVEGWFSGWKSTIASFFFIIGLVIGLYLVLRIGIALCIKCRVQEKRPKIYTDVEMNRLDR

SEQ ID NO: 62 - protein, 496 aa, synthetic construct
KFTIVFPHNQKGNWKNVPANYQYCPSSSDLNWHNGLIGTSLQVKMPKSHKAIQADGWMCHAAKWVTTCDFRWYGPKYVTHSIKSMIPTVDQCKESIAQTKQGTWLNPGFPPQSCGYASVTDAEAVIVKATPHQVLVDEYTGEWVDSQFPTGKCNKDICPTVHNSTTWHSDYKVTGLCDANLISMDITFFSEDGKLTSLGKEGTGFRSNYFAYENGDKACRMQYCKHWGVRLPSGVWFEMADKDIYNDAKFPDCPEGSSIAAPSQTSVDVSLIQDVERILDYSLCQETWSKIRAHLPISPVDLSYLSPKNPGTGPAFTIINGTLKYFETRYIRVDIAGPIIPQMRGVISGTTTERELWTDWYPYEDVEIGPNGVLKTATGYKFPLYMIGHGMLDSDLHISSKAQVFEHPHIQDAASQLPDDETLFFGDTGLSKNPIELVEGWFSGWKSTIASFFFIIGLVIGLYLVLRIGIALCIKCRVQEKRPKIYTDVEMNRLDR

SEQ ID NO: 63 - protein, 17 aa, synthetic construct
MLVLYLLLSLLALGAQC

SEQ ID NO: 64 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLDSMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 65 - protein, 495 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 66 - protein, 511 aa, synthetic construct
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLDSMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 67 - protein, 511 aa, synthetic construct
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 68 - protein, 522 aa, synthetic construct
MKCLLYLAFLFIGVNCWSHPQFEKGGSKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 69 - protein, 506 aa, synthetic construct
WSHPQFEKGGSKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 70 - protein, 503 aa, synthetic construct
WSHPQFEKKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 71 - protein, 503 aa, synthetic construct
KWSHPQFEKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 72 - protein, 503 aa, synthetic construct
KFTIVFPWSHPQFEKHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 73 - protein, 503 aa, synthetic construct
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKWSHPQFEKGLCDSNLESMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 74 - protein, 601 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGEDCHAPTYLPAEVDGDVKLSSNLVILPGQDLQYVLATYDTSAVEHAVVYYVYSPSRLSSYFYPFRLPIKGVPIELQVECFTWDQKLWCRHFCVLADSESGGHITHSGMVGMGVSCTVTREDGTNRRGG

SEQ ID NO: 75 - protein, 614 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGEDCHAPTYLPAEVDGDVKLSSNLVILPGQDLQYVLATYDTSAVEHAVVYYVYSPSRLSSYFYPFRLPIKGVPIELQVECFTWDQKLWCRHFCVLADSESGGHITHSGMVGMGVSCTVTREDGTNRRGGDIGGSWSHPQFEK

SEQ ID NO: 76 - protein, 883 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPALFNVPIKEAGEDCHAPTYLPAEVDGDVKLSSNLVILPGQDLQYVLATYDTSAVEHAVVYYVYSPSRLSSYFYPFRLPIKGVPIELQVECFTWDQKLWCRHFCVLADSESGGHITHSGMVGMGVSCTVTREDGTNRRGGDIGGGGSGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGSWSHPQFEK

SEQ ID NO: 77 - protein, 520 aa, synthetic construct
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYLIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPILLERLDVGTNLGNAIAKLEDAKELLESSDQILRSMKGLSSTCIVYILIAVCLGGLIGIPALICCCRGR

SEQ ID NO: 78 - protein, 531 aa, synthetic construct
MGLKVNVSAIFMAVLLTLQTPTGWSHPQFEKGGSQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGR

SEQ ID NO: 79 - protein, 509 aa, synthetic construct
MSIISYIAFLLLIDSNLGIPIFVPSGRNISWQPVIQPFDYQCPIHGNLPNTMGLSATKLTIKSPSVFSTDKVSGWICHAAEWKTTCDYRWYGPQYITHSIHPISPTIDECRRIIQRIASGTDEDLGFPPQSCGWASVTTVSNTNYRVVPHSVHLEPYGGHWIDHEFNGGECREKVCEMKGNHSIWITEETVQHECAKHIEEVEGIMYGNVPRGDVMYANNFIIDRHHRVYRFGGSCQMKFCNKDGIKFARGDWVEKTAGTLTTIHDNVPKCVDGTLVSGHRPGLDLIDTVFNLENVVEYTLCEGTKRKINKQEKLTSVDLSYLAPRIGGFGSVFRVRNGTLERGSTTYIRIEVEGPIVDSLNGTDPRTNASRVFWDDWELDGNIYQGFNGVYKGKDGKIHIPLNMIESGIIDDELQHAFQADIIPHPHYDDDEIREDDIFFDNTGENGNPVDAVVEWVSGWGTSLKFFGMTLVALILIFLLIRCCVACTYLMKRSKRPATESHEMRSLV

SEQ ID NO: 80 - protein, 491 aa, synthetic construct
IPIFVPSGRNISWQPVIQPFDYQCPIHGNLPNTMGLSATKLTIKSPSVFSTDKVSGWICHAAEWKTTCDYRWYGPQYITHSIHPISPTIDECRRIIQRIASGTDEDLGFPPQSCGWASVTTVSNTNYRVVPHSVHLEPYGGHWIDHEFNGGECREKVCEMKGNHSIWITEETVQHECAKHIEEVEGIMYGNVPRGDVMYANNFIIDRHHRVYRFGGSCQMKFCNKDGIKFARGDWVEKTAGTLTTIHDNVPKCVDGTLVSGHRPGLDLIDTVFNLENVVEYTLCEGTKRKINKQEKLTSVDLSYLAPRIGGFGSVFRVRNGTLERGSTTYIRIEVEGPIVDSLNGTDPRTNASRVFWDDWELDGNIYQGFNGVYKGKDGKIHIPLNMIESGIIDDELQHAFQADIIPHPHYDDDEIREDDIFFDNTGENGNPVDAVVEWVSGWGTSLKFFGMTLVALILIFLLIRCCVACTYLMKRSKRPATESHEMRSLV

SEQ ID NO: 81 - protein, 520 aa, synthetic construct
MSIISYIAFLLLIDSNLGWSHPQFEKGGSIPIFVPSGRNISWQPVIQPFDYQCPIHGNLPNTMGLSATKLTIKSPSVFSTDKVSGWICHAAEWKTTCDYRWYGPQYITHSIHPISPTIDECRRIIQRIASGTDEDLGFPPQSCGWASVTTVSNTNYRVVPHSVHLEPYGGHWIDHEFNGGECREKVCEMKGNHSIWITEETVQHECAKHIEEVEGIMYGNVPRGDVMYANNFIIDRHHRVYRFGGSCQMKFCNKDGIKFARGDWVEKTAGTLTTIHDNVPKCVDGTLVSGHRPGLDLIDTVFNLENVVEYTLCEGTKRKINKQEKLTSVDLSYLAPRIGGFGSVFRVRNGTLERGSTTYIRIEVEGPIVDSLNGTDPRTNASRVFWDDWELDGNIYQGFNGVYKGKDGKIHIPLNMIESGIIDDELQHAFQADIIPHPHYDDDEIREDDIFFDNTGENGNPVDAVVEWVSGWGTSLKFFGMTLVALILIFLLIRCCVACTYLMKRSKRPATESHEMRSLV

SEQ ID NO: 82 - protein, 240 aa, Homo sapiens
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 83 - protein, 330 aa, Homo sapiens [sequence omitted]

SEQ ID NO: 84 - protein, 325 aa, Homo sapiens
STKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 85 - protein, 326 aa, Homo sapiens
STKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 7 aa, synthetic construct
GYPFTSY

SEQ ID NO: 87 - protein, 6 aa, synthetic construct
DPNSGD

SEQ ID NO: 88 - protein, 12 aa, synthetic construct
SPYYSNDNSMDY

SEQ ID NO: 89 - protein, 11 aa, synthetic construct
RASQSIGTSIH

SEQ ID NO: 90 - protein, 7 aa, synthetic construct
YASESIS

SEQ ID NO: 91 - protein, 9 aa, synthetic construct
QQSNSWPTT

SEQ ID NO: 92 - protein, 5 aa, synthetic construct
SYWIH

SEQ ID NO: 93 - protein, 17 aa, synthetic construct
RIDPNSGDTKYNEKFKN

SEQ ID NO: 94 - protein, 8 aa, synthetic construct
GYPFTSYW

SEQ ID NO: 95 - protein, 8 aa, synthetic construct
IDPNSGDT

SEQ ID NO: 96 - protein, 14 aa, synthetic construct
ARSPYYSNDNSMDY

SEQ ID NO: 97 - protein, 6 aa, synthetic construct
QSIGTS

SEQ ID NO: 98 - protein, 121 aa, synthetic construct
QVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSS

SEQ ID NO: 99 - protein, 108 aa, synthetic construct
DILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKR

SEQ ID NO: 100 - protein, 20 aa, synthetic construct
GGGGSGGGGSGGGGSGGGGS

SEQ ID NO: 101 - protein, 249 aa, synthetic construct
DILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSS

SEQ ID NO: 102 - protein, 249 aa, synthetic construct
QVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKR

SEQ ID NO: 103 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGCYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 104 - protein, 210 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLYK

SEQ ID NO: 105 - protein, 7 aa, synthetic construct
RYTFTDY

SEQ ID NO: 106 - protein, 6 aa, synthetic construct
YPYNGG

SEQ ID NO: 107 - protein, 12 aa, synthetic construct
DHRYNEGVSFDY

SEQ ID NO: 108 - protein, 15 aa, synthetic construct
RASESVDGFGNSFMN

SEQ ID NO: 109 - protein, 7 aa, synthetic construct
LASNLES

SEQ ID NO: 110 - protein, 9 aa, synthetic construct
QQNNEDPYT

SEQ ID NO: 111 - protein, 5 aa, synthetic construct
DYNLH

SEQ ID NO: 112 - protein, 17 aa, synthetic construct
FIYPYNGGTGYNQKFKN

SEQ ID NO: 113 - protein, 8 aa, synthetic construct
RYTFTDYN

SEQ ID NO: 114 - protein, 8 aa, synthetic construct
IYPYNGGT

SEQ ID NO: 115 - protein, 14 aa, synthetic construct
ARDHRYNEGVSFDY

SEQ ID NO: 116 - protein, 10 aa, synthetic construct
ESVDGFGNSF

SEQ ID NO: 117 - protein, 121 aa, synthetic construct
EVQLQQSGPELVKPGASVKISCKASRYTFTDYNLHWVKLSHEKSLEWIGFIYPYNGGTGYNQKFKNKAKLTVDYSSSTAYMELRSLTSVDAAVYYCARDHRYNEGVSFDYWGQGTTLTVSS

SEQ ID NO: 118 - protein, 112 aa, synthetic construct
NIVLTQSPASLAVSLGQRATISCRASESVDGFGNSFMNWYQQKPGQSPKLLIYLASNLESGVPARFSGSGSRTDFTLTIDPVEADDAATYYCQQNNEDPYTFGGGTKLEIKR

SEQ ID NO: 119 - protein, 253 aa, synthetic construct
NIVLTQSPASLAVSLGQRATISCRASESVDGFGNSFMNWYQQKPGQSPKLLIYLASNLESGVPARFSGSGSRTDFTLTIDPVEADDAATYYCQQNNEDPYTFGGGTKLEIKRGGGGSGGGGSGGGGSGGGGSEVQLQQSGPELVKPGASVKISCKASRYTFTDYNLHWVKLSHEKSLEWIGFIYPYNGGTGYNQKFKNKAKLTVDYSSSTAYMELRSLTSVDAAVYYCARDHRYNEGVSFDYWGQGTTLTVSS

SEQ ID NO: 120 - protein, 253 aa, synthetic construct
EVQLQQSGPELVKPGASVKISCKASRYTFTDYNLHWVKLSHEKSLEWIGFIYPYNGGTGYNQKFKNKAKLTVDYSSSTAYMELRSLTSVDAAVYYCARDHRYNEGVSFDYWGQGTTLTVSSGGGGSGGGGSGGGGSGGGGSNIVLTQSPASLAVSLGQRATISCRASESVDGFGNSFMNWYQQKPGQSPKLLIYLASNLESGVPARFSGSGSRTDFTLTIDPVEADDAATYYCQQNNEDPYTFGGGTKLEIKR

SEQ ID NO: 121 - protein, 620 aa, synthetic construct
METDTLLLWVLLLWVPGSTGDWSHPQFEKGGSSAQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRASGGGGSGGGGSGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 122 - protein, 5 aa, synthetic construct
  REPEAT 1..5: note = Sequence may be present n times, wherein n is 1, 2, 3, 4, 5, 6, 7, 8, 9, or 10
GGGGA

SEQ ID NO: 123 - protein, 12 aa, synthetic construct
GSAGSAAGSGEF

SEQ ID NO: 124 - protein, 18 aa, synthetic construct
KESGSVSSEQLAQFRSLD

SEQ ID NO: 125 - protein, 14 aa, synthetic construct
EGKSSGSGSESKST

SEQ ID NO: 126 - protein, 359 aa, synthetic construct
MALPVTALLLPLALLLHAARPWSHPQFEKGGSGSDIQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRVNGGGGSGGGGSGGGGSGGGGSAVHTRGLDFACDIYIWAPLAGTCGVLLLSLVITLYCNHRNRRRVNRVRQGYS

SEQ ID NO: 127 - protein, 7 aa, synthetic construct
ENLYFQG

SEQ ID NO: 128 - protein, 7 aa, synthetic construct
ENLYFQS

SEQ ID NO: 129 - protein, 627 aa, synthetic construct
METDTLLLWVLLLWVPGSTGDWSHPQFEKGGGGSGGGGSSAQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRASGGGGSGGGGSGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 130 - protein, 617 aa, synthetic construct
METDTLLLWVLLLWVPGSTGDSAQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSWSHPQFEKGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRASGGGGSGGGGSGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 131 - protein, 617 aa, synthetic construct
METDTLLLWVLLLWVPGSTGDSAQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRASGGGGSGGGGSWSHPQFEKGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 132 - protein, 525 aa, synthetic construct
MKCLLYLAFLFIGVNCKFSGGWSHPQFEKGGSTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 133 - protein, 525 aa, synthetic construct
MKCLLYLAFLFIGVNCKFTIVFPSGGWSHPQFEKGGSHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 134 - protein, 4 aa, synthetic construct
AAAA

SEQ ID NO: 135 - protein, 508 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESWSHPQFEKANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 136 - protein, 534 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESWSHPQFEKKESGSVSSEQLAQFRSLDWSHPQFEKANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 137 - protein, 788 aa, synthetic construct
MGVTGILQLPRDRFWSHPQFEKGGSKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 138 - protein, 819 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESAGGGGSGGGGSGGGGSASQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRTGGGGGSGGGGSGGGGSWSHPQFEKKESGSVSSEQLAQFRSLDWSHPQFEKSANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 139 - protein, 811 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSWSHPQFEKKESGSVSSEQLAQFRSLDWSHPQFEKNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 140 - protein, 793 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESAGGGGSGGGGSGGGGSASQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRTGGGGGSGGGGSGGGGSWSHPQFEKSANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 141 - protein, 785 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSWSHPQFEKNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 142 - protein, 819 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESAWSHPQFEKKESGSVSSEQLAQFRSLDWSHPQFEKGGGGSGGGGSGGGGSASQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRTGGGGGSGGGGSGGGGSSANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 143 - protein, 811 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEWSHPQFEKKESGSVSSEQLAQFRSLDWSHPQFEKGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 144 - protein, 793 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESAWSHPQFEKGGGGSGGGGSGGGGSASQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRTGGGGGSGGGGSGGGGSSANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 145 - protein, 785 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEWSHPQFEKGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 146 - protein, 14 aa, synthetic construct
MGVTGILQLPRDRF

SEQ ID NO: 147 - protein, 673 aa, synthetic construct
WSHPQFEKGGSKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 148 - protein, 497 aa, synthetic construct
WSHPQFEKGGSKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 149 - protein, 494 aa, synthetic construct
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESWSHPQFEKANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 150 - protein, 520 aa, synthetic construct
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESWSHPQFEKKESGSVSSEQLAQFRSLDWSHPQFEKANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 151 - protein, 782 aa, synthetic construct
WSHPQFEKGGSKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESAGGGGSGGGGSGGGGSASQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRTGGGGGSGGGGSGGGGSSANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 152 - protein, 506 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEWSHPQFEKNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 153 - protein, 492 aa, synthetic construct
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEWSHPQFEKNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 154 - protein, 532 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEWSHPQFEKKESGSVSSEQLAQFRSLDWSHPQFEKNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 155 - protein, 518 aa, synthetic construct
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEWSHPQFEKKESGSVSSEQLAQFRSLDWSHPQFEKNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 156 - protein, 774 aa, synthetic construct
WSHPQFEKGGSKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 157 - protein, 805 aa, synthetic construct
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESAGGGGSGGGGSGGGGSASQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRTGGGGGSGGGGSGGGGSWSHPQFEKKESGSVSSEQLAQFRSLDWSHPQFEKSANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 158 - protein, 797 aa, synthetic construct
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSWSHPQFEKKESGSVSSEQLAQFRSLDWSHPQFEKNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 159 - protein, 779 aa, synthetic construct
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESAGGGGSGGGGSGGGGSASQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRTGGGGGSGGGGSGGGGSWSHPQFEKSANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 160 - protein, 771 aa, synthetic construct
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSWSHPQFEKNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 161 - protein, 805 aa, synthetic construct
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESAWSHPQFEKKESGSVSSEQLAQFRSLDWSHPQFEKGGGGSGGGGSGGGGSASQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRTGGGGGSGGGGSGGGGSSANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 162 - protein, 797 aa, synthetic construct
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEWSHPQFEKKESGSVSSEQLAQFRSLDWSHPQFEKGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 163 - protein, 779 aa, synthetic construct
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESAWSHPQFEKGGGGSGGGGSGGGGSASQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRTGGGGGSGGGGSGGGGSSANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 164 - protein, 771 aa, synthetic construct
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEWSHPQFEKGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 165 - protein, 26 aa, synthetic construct
MVVILDKRCYCNLLILILMISECSVG

SEQ ID NO: 166 - protein, 509 aa, synthetic construct
WSHPQFEKGGSILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 167 - protein, 509 aa, synthetic construct
KFSGGWSHPQFEKGGSTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 168 - protein, 509 aa, synthetic construct
KFTIVFPSGGWSHPQFEKGGSHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLESMDITFFSEDGELSSLGKEGTGFRSNYFAYENGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTAERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 169 - protein, 23 aa, synthetic construct
MGLKVNVSAIFMAVLLTLQTPTG

SEQ ID NO: 170 - protein, 508 aa, synthetic construct
WSHPQFEKGGSQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGR

SEQ ID NO: 171 - protein, 18 aa, synthetic construct
MSIISYIAFLLLIDSNLG

SEQ ID NO: 172 - protein, 502 aa, synthetic construct
WSHPQFEKGGSIPIFVPSGRNISWQPVIQPFDYQCPIHGNLPNTMGLSATKLTIKSPSVFSTDKVSGWICHAAEWKTTCDYRWYGPQYITHSIHPISPTIDECRRIIQRIASGTDEDLGFPPQSCGWASVTTVSNTNYRVVPHSVHLEPYGGHWIDHEFNGGECREKVCEMKGNHSIWITEETVQHECAKHIEEVEGIMYGNVPRGDVMYANNFIIDRHHRVYRFGGSCQMKFCNKDGIKFARGDWVEKTAGTLTTIHDNVPKCVDGTLVSGHRPGLDLIDTVFNLENVVEYTLCEGTKRKINKQEKLTSVDLSYLAPRIGGFGSVFRVRNGTLERGSTTYIRIEVEGPIVDSLNGTDPRTNASRVFWDDWELDGNIYQGFNGVYKGKDGKIHIPLNMIESGIIDDELQHAFQADIIPHPHYDDDEIREDDIFFDNTGENGNPVDAVVEWVSGWGTSLKFFGMTLVALILIFLLIRCCVACTYLMKRSKRPATESHEMRSLV

SEQ ID NO: 173 - protein, 21 aa, synthetic construct
METDTLLLWVLLLWVPGSTGD

SEQ ID NO: 174 - protein, 599 aa, synthetic construct
WSHPQFEKGGSSAQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRASGGGGSGGGGSGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 175 - protein, 616 aa, synthetic construct
METDTLLLWVLLLWVPGSTGDWSHPQFEKGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 176 - protein, 595 aa, synthetic construct
WSHPQFEKGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 177 - protein, 21 aa, synthetic construct
MALPVTALLLPLALLLHAARP

SEQ ID NO: 178 - protein, 338 aa, synthetic construct
WSHPQFEKGGSGSDIQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRVNGGGGSGGGGSGGGGSGGGGSAVHTRGLDFACDIYIWAPLAGTCGVLLLSLVITLYCNHRNRRRVNRVRQGYS

SEQ ID NO: 179 - protein, 606 aa, synthetic construct
WSHPQFEKGGGGSGGGGSSAQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRASGGGGSGGGGSGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 180 - protein, 623 aa, synthetic construct
METDTLLLWVLLLWVPGSTGDWSHPQFEKGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 181 - protein, 602 aa, synthetic construct
WSHPQFEKGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 182 - protein, 596 aa, synthetic construct
SAQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSWSHPQFEKGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRASGGGGSGGGGSGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 183 - protein, 613 aa, synthetic construct
METDTLLLWVLLLWVPGSTGDQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSWSHPQFEKGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 184 - protein, 592 aa, synthetic construct
QVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSWSHPQFEKGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 185 - protein, 596 aa, synthetic construct
SAQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRASGGGGSGGGGSWSHPQFEKGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 186 - protein, 613 aa, synthetic construct
METDTLLLWVLLLWVPGSTGDQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSWSHPQFEKGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 187 - protein, 592 aa, synthetic construct
QVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSWSHPQFEKGGGGSEPKSCDKTHTCPPCPAPEAAGGPSVFLFPPKPKDTLMASRTPEVTCVVVDVSHEDPEVKFNWYVDGVEVHNAKTKPREEQYASTYRVVSVLTVLAQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGKKIEVMYPPPYLDNEKSNGTIIHVKGKHLCPSPLFPGPSKPFWVLVVVGGVLACYSLLVTVAFIIFWVRSKRSRLLHSDYMNRVRQGYS

SEQ ID NO: 188 - protein, 801 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESAWSHPQFEKGGGGSGGGGSGGGGSASQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRTGGGGGSGGGGSGGGGSWSHPQFEKSANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 189 - protein, 787 aa, synthetic construct
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEESAWSHPQFEKGGGGSGGGGSGGGGSASQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRTGGGGGSGGGGSGGGGSWSHPQFEKSANTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 190 - protein, 793 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEWSHPQFEKGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSWSHPQFEKNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 191 - protein, 779 aa, synthetic construct
KRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEEWSHPQFEKGGGGSGGGGSGGGGSQVQLQQPGAELVKPGASVKLSCKASGYPFTSYWIHWVKQRPGRGLEWLGRIDPNSGDTKYNEKFKNKATLTVDKSSTTAYMQLSSLTSEDSAVYYCARSPYYSNDNSMDYWGQGTSVTVSSGGGGSGGGGSGGGGSGGGGSDILLTQSPAILSVSPGERVSFSCRASQSIGTSIHWYQQRTNDSPRLLIKYASESISGIPSRFSGSGSGTDFTLSINSVESEDIADYYCQQSNSWPTTFGGGTKLEIKRGGGGSGGGGSGGGGSWSHPQFEKNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 192 - protein, 122 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 193 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 194 - protein, 244 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 195 - protein, 244 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 196 - protein, 110 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTS

SEQ ID NO: 197 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 198 - protein, 248 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGGSGGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 199 - protein, 248 aa, synthetic construct
SEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTS